(19) United States
(12) Patent Application Publication
(10) Pub. No.: US 2024/0208986 A1
(43) Pub. Date: Jun. 27, 2024

(54) PHENYL AZEPINES AS RIPK1 INHIBITORS AND METHODS OF USE THEREOF

(71) Applicant: Merck Sharp & Dohme LLC, Rahway, NJ (US)

(72) Inventors: Joanna L. Chen, Braintree, MA (US); Erin F. DiMauro, Cambridge, MA (US); Kevin D. Dykstra, West Milford, NJ (US); Xavier Fradera, Boston, MA (US); Peter H. Fuller, Ashland, MA (US); Charles A. Lesburg, Waban, MA (US); Min Lu, Brookline, MA (US); Joey L. Methot, Westwood, MA (US); Matthew J. Mitcheltree, Jamaica Plain, MA (US); Jing Su, Scotch Plains, NJ (US)

(73) Assignee: Merck Sharp & Dohme LLC, Rahway, NJ (US)

(21) Appl. No.: 18/556,226

(22) PCT Filed: Apr. 21, 2022
(86) PCT No.: PCT/US2022/025665

§ 371 Date: Oct. 19, 2023

Related U.S. Application Data
(60) Provisional application No. 63/180,221, filed on Apr. 27, 2021.

(51) Int. Cl. C07D 491/08; A61K 31/553; C07D 487/08; C07D 519/00

(52) U.S. Cl. CPC C07D 491/08; A61K 31/553; C07D 487/08; C07D 519/00

(57) ABSTRACT

Described herein are compounds of Formula I: (I), or a pharmaceutically acceptable salt thereof. The compounds of Formula I act as RIPK1 inhibitors and can be useful in preventing, treating or acting as a remedial agent for RIPK1-related diseases.

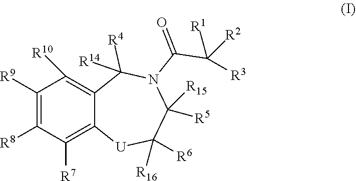

### FIELD OF THE INVENTION

The present invention is directed to RIPK1 inhibitors. Specifically, the RIPK1 inhibitors described herein can be useful in preventing, treating or acting as a remedial agent for RIPK1-related diseases.

### BACKGROUND OF THE INVENTION

Receptor-interacting protein-1 kinase (RIPK1) belongs to the family serine/threonine protein kinase involved in innate immune signaling. RIPK1 has emerged as a promising therapeutic target for the treatment of a wide range of human neurodegenerative, autoimmune, and inflammatory diseases. This is supported by extensive studies which have demonstrated that RIPK1 is a key mediator of apoptotic and necrotic cell death as well as inflammatory pathways.

For example, RIPK1 inhibition has been found to be useful as a treatment of acute kidney injury (AKI), a destructive clinical condition induced by multiple insults including ischemic reperfusion, nephrotoxic drugs and sepsis. It has been found that RIPK1-mediated necroptosis plays an important role in AKI and a RIPK1 inhibitor may serve as a promising clinical candidate for AKI treatment. Wang J N, Liu M M, Wang F, Wei B, Yang Q, Cai Y T, Chen X, Liu X Q, Jiang L, Li C, Hu X W, Yu J T, Ma T T, Jin J, Wu Y G, Li J, Meng X M, RIPK1 Inhibitor Cpd-71 Attenuates Renal Dysfunction in Cisplatin-Treated Mice via Attenuating Necroptosis, Inflammation and Oxidative Stress. Clin Sci (Lond). 2019 Jul. 25; 133(14):1609-1627.

Additionally, human genetic evidence has linked the dysregulation of RIPK1 to the pathogenesis of amyotrophic lateral sclerosis (ALS), Alzheimer's disease and multiple sclerosis as well as other inflammatory and neurodegenerative diseases. Alexei Degterev, Dimitry Ofengeim, and Junying Yuan, Targeting RIPK1 for the treatment of human diseases, PNAS, May 14, 2019, 116 (20), 9714-9722; Ito Y, Ofengeim D, Najafov A, Das S, Saberi S, Li Y, et al., RIPK1 mediates axonal degeneration by promoting inflammation and necroptosis in ALS, Science, 2016, 353:603-8; Caccamo A, Branca C, Piras I S, Ferreira E, Huentelman M J, Liang W S, et al., Necroptosis activation in Alzheimer's disease, Nat Neurosci, 2017, 20:1236-46; Ofengeim D, Ito Y, Najafov A, Zhang Y, Shan B, DeWitt J P, et al., Activation of necroptosis in multiple sclerosis, Cell Rep., 2015, 10:1836-49.

It also has been demonstrated that necroptosis is a delayed component of ischemic neuronal injury, thus RIPK1 inhibition may also play a promising role as a treatment for stroke. Degterev A, et al., Chemical inhibitor of nonapoptotic cell death with therapeutic potential for ischemic brain injury, Nat Chem Biol 2005, 1(2): 112-119.

Therefore, there is a need for inhibitors of RIPK1 that offer high selectivity which can penetrate the blood-brain barrier, thus offering the possibility to target neuroinflammation and cell death which drive various neurologic conditions including Alzheimer's disease, ALS, and multiple sclerosis as well as acute neurological diseases such as stroke and traumatic brain injuries.

### SUMMARY OF THE INVENTION

Described herein are compounds of Formula I:

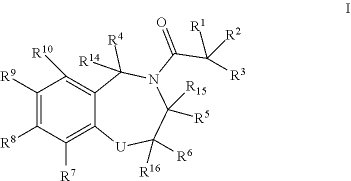

and pharmaceutically acceptable salts thereof, wherein U, R¹, R², R³, R⁴, R⁵, R⁶, R⁷, R⁸, R⁹, R¹⁰, R¹⁴, R¹⁵ and R¹⁶ are described below.

The compounds described herein are RIPK1 inhibitors, which can be useful in the prevention, treatment or amelioration of neurodegenerative, autoimmune, inflammatory diseases and other RIPK1-related diseases.

Also described herein are methods of treating neurodegenerative, autoimmune, and inflammatory diseases comprising administering to a patient in need thereof a compound described herein, or a pharmaceutically acceptable salt thereof.

Also described herein are uses of a compound described herein, or a pharmaceutically acceptable salt thereof, to treat neurodegenerative, autoimmune, and inflammatory diseases in a patient in need thereof.

Also described herein are pharmaceutical compositions comprising a compound described herein, or a pharmaceutically acceptable salt thereof and a pharmaceutically acceptable carrier.

Also described herein are pharmaceutical compositions comprising a compound described herein and a pharmaceutically acceptable carrier.

Also described herein are methods of treating neurodegenerative, autoimmune, and inflammatory diseases comprising administering to a patient in need thereof a compound described herein, or a pharmaceutically acceptable salt thereof, and at least one additional therapeutic agent.

Also described herein are uses of a compound described herein, or a pharmaceutically acceptable salt thereof, in combination with at least one additional agent, to treat neurodegenerative, autoimmune, and inflammatory diseases in a patient in need thereof.

Also described herein are pharmaceutical compositions comprising a compound described herein, or a pharmaceutically acceptable salt thereof, at least one additional therapeutic agent and a pharmaceutically acceptable carrier.

Also described herein are pharmaceutical compositions comprising a compound described herein, at least one additional therapeutic agent and a pharmaceutically acceptable carrier.

### BRIEF DESCRIPTION OF THE DRAWINGS

FIG. 1 shows the absolute stereochemistry of 1-((2S,5S)-2,3-dihydro-2,5-methanobenzo[f][1,4]oxazepin-4(5H)-yl)-3,3-difluoro-2,2-dimethylpropan-1-one (1-2), confirmed by crystallography.

### DETAILED DESCRIPTION OF THE INVENTION

Described herein are compounds of Formula I:

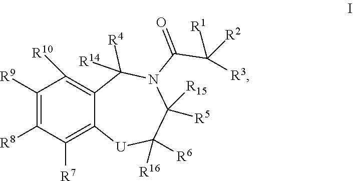

or a pharmaceutically acceptable salt thereof, wherein:
U is O, S, NR¹¹ or CR¹²R¹³.
R¹ is hydrogen, OH, C₁-C₆alkylOH, CN, C₁-C₆alkylCN, C₁-C₆alkyl, C₃-C₆cycloalkyl, haloC₁-C₆alkyl, halogen, NH₂, N(C₁-C₆alkyl)₂, NH(C₁-C₆alkyl) or alkoxy, or wherein R¹ is taken with R² and forms an oxo group, or wherein when R¹ is taken with R² or R² and R³ and forms a C₃-C₁₀cycloalkyl, aryl, heteroaryl or heterocycloalkyl, wherein the C₃-C₁₀cycloalkyl, aryl, heteroaryl or heterocycloalkyl is unsubstituted or substituted with one to four substituents selected from the group consisting of halogen, C₁-C₆alkyl, haloC₁-C₆alkyl, alkoxy, CN, COOC₁-C₆alkyl, COaryl, aryl, and heteroaryl, wherein the heteroaryl is unsubstituted or substituted with one to four substituents selected from the group consisting of halogen, CN, C₁-C₆alkyl, alkoxy, aryl, C₃-C₆cycloalkyl and haloC₁-C₆alkyl;
R² is hydrogen, OH, C₁-C₆alkylOH, CN, C₁-C₆alkylCN, C₁-C₆alkyl, C₃-C₆cycloalkyl, haloC₁-C₆alkyl, halogen, NH₂, N(C₁-C₆alkyl)₂, NH(C₁-C₆alkyl) or alkoxy, or wherein R² is taken with R¹ and forms an oxo group, or wherein when R² is taken with R¹ or R¹ and R³ and forms a C₃-C₁₀cycloalkyl, aryl, heteroaryl or heterocycloalkyl, wherein the C₃-C₁₀cycloalkyl, aryl, heteroaryl or heterocycloalkyl is unsubstituted or substituted with one to four substituents selected from the group consisting of halogen, C₁-C₆alkyl, haloC₁-C₆alkyl, alkoxy, CN, COOC₁-C₆alkyl, COaryl, aryl, and heteroaryl, wherein the heteroaryl is unsubstituted or substituted with one to four substituents selected from the group consisting of halogen, CN, C₁-C₆alkyl, alkoxy, aryl, C₃-C₆cycloalkyl and haloC₁-C₆alkyl;
R³ is OH, C₁-C₆alkylOH, CN, C₁-C₆alkylCN, C₁-C₆alkyl, C₃-C₆cycloalkyl, haloC₁-C₆alkyl, halogen, C₁-C₆alkylOhaloC₁-C₆alkyl, alkoxy, NHC₁-C₆alkylaryl, CONHC₁-C₆alkylaryl, aryl, or C₁-C₆alkylaryl, wherein the NHC₁-C₆alkylaryl, CONHC₁-C₆alkylaryl, aryl, or C₁-C₆alkylaryl is unsubstituted or substituted with one to four substituents selected from the group consisting of halogen, C₁-C₆alkyl, alkoxy and haloC₁-C₆alkyl, or wherein when R³ is taken with R² or R¹ and R², forms a C₃-C₁₀cycloalkyl, aryl, heteroaryl or heterocycloalkyl, wherein the C₃-C₁₀cycloalkyl, aryl, heteroaryl or heterocycloalkyl is unsubstituted or substituted with one to four substituents selected from the group consisting of halogen, C₁-C₆alkyl, haloC₁-C₆alkyl, alkoxy, CN, COOC₁-C₆alkyl, COaryl, aryl, and heteroaryl, wherein the heteroaryl is unsubstituted or substituted with one to four substituents selected from the group consisting of halogen, CN, C₁-C₆alkyl, alkoxy, aryl, C₃-C₆cycloalkyl and haloC₁-C₆alkyl;
R⁴ is hydrogen, C₁-C₆alkyl, or wherein when taken with R⁵ or R⁶ form a —CH₂— or —CH₂CH₂— bridge;
R⁵ is hydrogen, C₁-C₆alkyl, or wherein when taken with R⁴ form a —CH₂— or —CH₂CH₂— bridge;
R⁶ is hydrogen, C₁-C₆alkyl, or wherein when taken with R⁴ form a —CH₂— or —CH₂CH₂— bridge;
R⁷ is hydrogen, OH, C₁-C₆alkylOH, CN, C₁-C₆alkylCN, C₁-C₆alkyl, C₁-C₆alkylaryl, C₂-C₆alkynyl, haloC₁-C₆alkyl, halogen, or alkoxy, wherein the C₂-C₆alkynyl is unsubstituted or substituted with one to three substituents selected from the group consisting of OH, C₁-C₆alkylOH, CN, C₁-C₆alkylCN, NH₂, NHC₁-C₆alkyl, N(C₁-C₆alkyl)₂, haloC₁-C₆alkyl, halogen, alkoxy, haloalkoxy, aryl, C₃-C₁₀cycloalkyl, heteroaryl and heterocycloalkyl, wherein the aryl, C₃-C₁₀cycloalkyl, heteroaryl and heterocycloalkyl are unsubstituted or substituted with one to three substituents selected from the group consisting of OH, C₁-C₆alkylOH, CN, C₁-C₆alkylCN, C₁-C₆alkyl, NH₂, NHC₁-C₆alkyl, N(C₁-C₆alkyl)₂, haloC₁-C₆alkyl, halogen, alkoxy, haloalkoxy;
R⁸ is hydrogen, OH, C₁-C₆alkylOH, CN, C₁-C₆alkylCN, C₁-C₆alkyl, C₁-C₆alkylaryl, C₂-C₆alkynyl, haloC₁-C₆alkyl, halogen, or alkoxy;
R⁹ is hydrogen, OH, C₁-C₆alkylOH, CN, C₁-C₆alkylCN, C₁-C₆alkyl, C₁-C₆alkylaryl, C₂-C₆alkynyl, haloC₁-C₆alkyl, halogen, or alkoxy;
R¹⁰ is hydrogen, OH, C₁-C₆alkylOH, CN, C₁-C₆alkylCN, C₁-C₆alkyl, C₁-C₆alkylaryl, C₂-C₆alkynyl, haloC₁-C₆alkyl, halogen, or alkoxy;
R¹¹ is hydrogen, C₁-C₆alkyl, or C₃-C₆cycloalkyl;
R¹² is hydrogen, OH, C₁-C₆alkylOH, CN, C₁-C₆alkylCN, C₁-C₆alkyl, haloC₁-C₆alkyl, halogen, or alkoxy;
R¹³ is hydrogen, OH, C₁-C₆alkylOH, CN, C₁-C₆alkylCN, C₁-C₆alkyl, haloC₁-C₆alkyl, halogen, or alkoxy;
R¹⁴ is hydrogen, OH, C₁-C₆alkylOH, CN, C₁-C₆alkylCN, C₁-C₆alkyl, haloC₁-C₆alkyl, halogen, or alkoxy;
R¹⁵ is hydrogen, OH, C₁-C₆alkylOH, CN, C₁-C₆alkylCN, C₁-C₆alkyl, haloC₁-C₆alkyl, halogen, or alkoxy; and
R¹⁶ is hydrogen, OH, C₁-C₆alkylOH, CN, C₁-C₆alkylCN, C₁-C₆alkyl, haloC₁-C₆alkyl, halogen, or alkoxy.

In regard to the compounds described herein, U is O, S, NR¹¹ or CR¹²R¹³. In certain embodiments, U is O. In other embodiments, U is S. In still other embodiments, U is NR¹¹. In still other embodiments, U is CR¹²R¹³.

When U is NR¹¹, R¹¹ is hydrogen, C₁-C₆alkyl, or C₃-C₆cycloalkyl. In certain embodiments, R¹¹ is hydrogen. In other embodiments R¹¹ is C₁-C₆alkyl. Suitable alkyls include, but are not limited to, methyl, ethyl, n-propyl, isopropyl, n-butyl, isobutyl, sec-butyl, tert-butyl, n-pentyl, isopentyl, neopentyl, tert-pentyl, 1-methylbutyl, 2-methylbutyl, 1,2-dimethylpropyl, 1-ethylpropyl, n-hexyl, isohexyl, 1-methylpentyl, 2-methylpentyl, 3-methylpentyl, 1,1-dimethylbutyl, 1,2-dimethylbutyl, 2,2-dimethylbutyl, 1-ethylbutyl, 1,1,2-trimethylpropyl, 1,2,2-trimethylpropyl, 1-ethyl-2-methylpropyl and 1-ethyl-1-methylpropyl. In certain embodiments, R¹¹ is methyl or ethyl. In other embodiments, R¹¹ is C₃-C₆cycloalkyl. Suitable cycloalkyls include, but are not limited to, cyclopropyl, cyclobutyl, cyclopentyl and cyclohexyl.

When U is CR¹²R¹³, R¹² is hydrogen, OH, C₁-C₆alkylOH, CN, C₁-C₆alkylCN, C₁-C₆alkyl, haloC₁-C₆alkyl, halogen, or alkoxy. In certain embodiments, R¹² is hydrogen. In certain embodiments, R¹² is OH. In certain embodiments, R¹² is C₁-C₆alkylOH. Suitable alcohols include, but are not limited to, methanol, ethanol, propanol and butanol.

In certain embodiments, R¹² is CN. In certain embodiments, R¹² is C₁-C₆alkylCN. In certain embodiments, R¹² is

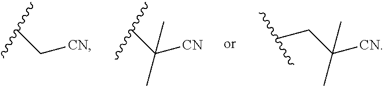

In certain embodiments, R¹² is C₁-C₆alkyl. Suitable alkyls include, but are not limited to, methyl, ethyl, n-propyl, isopropyl, n-butyl, isobutyl, sec-butyl, tert-butyl, n-pentyl, isopentyl, neopentyl, tert-pentyl, 1-methylbutyl, 2-methylbutyl, 1,2-dimethylpropyl, 1-ethylpropyl, n-hexyl, isohexyl, 1-methylpentyl, 2-methylpentyl, 3-methylpentyl, 1,1-dimethylbutyl, 1,2-dimethylbutyl, 2,2-dimethylbutyl, 1-ethylbutyl, 1,1,2-trimethylpropyl, 1,2,2-trimethylpropyl, 1-ethyl-2-methylpropyl and 1-ethyl-1-methylpropyl. In certain embodiments, R¹² is methyl or ethyl.

In certain embodiments, R¹² is haloC₁-C₆alkyl. Suitable examples of haloalkyls include, but are not limited to, fluoromethyl, difluoromethyl, trifluoromethyl, 2-fluoroethyl, 1,2-difluoroethyl and 2,2-difluoroethyl. In certain embodiments, R¹² is difluoromethyl. In certain embodiments, R¹² is trifluoromethyl. In certain embodiments, R¹² is difluoromethyl or trifluoromethyl. In certain embodiments, R¹² is halogen. Suitable halogens include, but are not limited to, fluorine, chlorine, bromine or iodine. In certain embodiments, R¹² is fluorine or chlorine. In certain embodiments, R¹² is alkoxy. Suitable alkoxys include, but are not limited to, methoxy, ethoxy, n-propoxy, isopropoxy and n-butoxy. In certain embodiments, R¹² is methoxy.

When U is CR¹²R¹³, R¹³ is hydrogen, OH, C₁-C₆alkylOH, CN, C₁-C₆alkylCN, C₁-C₆alkyl, haloC₁-C₆alkyl, halogen, or alkoxy. In certain embodiments, R¹³ is hydrogen. In certain embodiments, R¹³ is OH. In certain embodiments, R¹³ is C₁-C₆alkylOH. Suitable alcohols include, but are not limited to, methanol, ethanol, propanol and butanol.

In certain embodiments, R¹³ is CN. In certain embodiments, R¹³ is C₁-C₆alkylCN. In certain embodiments, R¹³ is

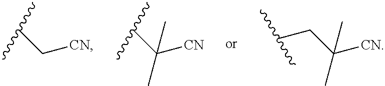

In certain embodiments, R¹³ is C₁-C₆alkyl. Suitable alkyls include, but are not limited to, methyl, ethyl, n-propyl, isopropyl, n-butyl, isobutyl, sec-butyl, tert-butyl, n-pentyl, isopentyl, neopentyl, tert-pentyl, 1-methylbutyl, 2-methylbutyl, 1,2-dimethylpropyl, 1-ethylpropyl, n-hexyl, isohexyl, 1-methylpentyl, 2-methylpentyl, 3-methylpentyl, 1,1-dimethylbutyl, 1,2-dimethylbutyl, 2,2-dimethylbutyl, 1-ethylbutyl, 1,1,2-trimethylpropyl, 1,2,2-trimethylpropyl, 1-ethyl-2-methylpropyl and 1-ethyl-1-methylpropyl. In certain embodiments, R¹³ is methyl or ethyl.

In certain embodiments, R¹³ is haloC₁-C₆alkyl. Suitable examples of haloalkyls include, but are not limited to, fluoromethyl, difluoromethyl, trifluoromethyl, 2-fluoroethyl, 1,2-difluoroethyl and 2,2-difluoroethyl. In certain embodiments, R¹³ is difluoromethyl. In certain embodiments, R¹³ is trifluoromethyl. In certain embodiments, R¹³ is difluoromethyl or trifluoromethyl. In certain embodiments, R¹³ is halogen. Suitable halogens include, but are not limited to, fluorine, chlorine, bromine or iodine. In certain embodiments, R¹³ is fluorine or chlorine. In certain embodiments, R¹³ is alkoxy. Suitable alkoxys include, but are not limited to, methoxy, ethoxy, n-propoxy, isopropoxy and n-butoxy. In certain embodiments, R¹³ is methoxy.

In regard to the compounds described R¹ is hydrogen, OH, C₁-C₆alkylOH, CN, C₁-C₆alkylCN, C₁-C₆alkyl, C₃-C₆cycloalkyl, haloC₁-C₆alkyl, halogen, NH₂, N(C₁-C₆alkyl)₂, NH(C₁-C₆alkyl) or alkoxy, or wherein R¹ is taken with R² and forms an oxo group, or wherein when R¹ is taken with R² or R² and R³ and forms a C₃-C₁₀cycloalkyl, aryl, heteroaryl or heterocycloalkyl, wherein the C₃-C₁₀cycloalkyl, aryl, heteroaryl or heterocycloalkyl is unsubstituted or substituted with one to four substituents selected from the group consisting of halogen, C₁-C₆alkyl, haloC₁-C₆alkyl, alkoxy, CN, COOC₁-C₆alkyl, COaryl, aryl, and heteroaryl, wherein the heteroaryl is unsubstituted or substituted with one to four substituents selected from the group consisting of halogen, CN, C₁-C₆alkyl, alkoxy, aryl, C₃-C₆cycloalkyl and haloC₁-C₆alkyl.

In certain embodiments, R¹ is hydrogen. In certain embodiments, R¹ is OH. In certain embodiments, R¹ is C₁-C₆alkylOH. Suitable alcohols include, but are not limited to, methanol, ethanol, propanol and butanol.

In certain embodiments, R¹ is CN. In certain embodiments, R¹ is C₁-C₆alkylCN. In certain embodiments, R¹ is

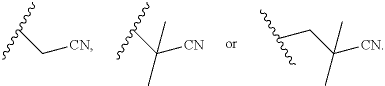

In other embodiments, R¹ is

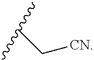 [SMILES: *CC#N]

In certain embodiments, R¹ is C₁-C₆alkyl. Suitable alkyls include, but are not limited to, methyl, ethyl, n-propyl, isopropyl, n-butyl, isobutyl, sec-butyl, tert-butyl, n-pentyl, isopentyl, neopentyl, tert-pentyl, 1-methylbutyl, 2-methylbutyl, 1,2-dimethylpropyl, 1-ethylpropyl, n-hexyl, isohexyl, 1-methylpentyl, 2-methylpentyl, 3-methylpentyl, 1,1-dimethylbutyl, 1,2-dimethylbutyl, 2,2-dimethylbutyl, 1-ethylbutyl, 1,1,2-trimethylpropyl, 1,2,2-trimethylpropyl, 1-ethyl-2-methylpropyl and 1-ethyl-1-methylpropyl. In particular embodiments. R¹ is methyl, ethyl, or t-butyl. In certain embodiments, R¹ is C₃-C₆cycloalkyl. Suitable cycloalkyls include, but are not limited to, cyclopropyl, cyclobutyl, cyclopentyl and cyclohexyl. In certain embodiments, R¹ is cyclopropyl. In certain embodiments, R¹ is cyclobutyl.

In certain embodiments, R¹ is haloC₁-C₆alkyl. Suitable examples of haloalkyls include, but are not limited to, fluoromethyl, difluoromethyl, trifluoromethyl, 2-fluoroethyl, 1,2-difluoroethyl and 2,2-difluoroethyl. In certain embodiments, R¹ is fluoromethyl. In certain embodiments, R¹ is difluoromethyl. In certain embodiments, R¹ is trifluoromethyl. In certain embodiments, R¹ is difluoromethyl, fluoromethyl, difluoroethyl or trifluoromethyl. In certain embodiments, R¹ is halogen. Suitable halogens include, but are not limited to, fluorine, chlorine, bromine or iodine. In certain embodiments, R¹ is fluorine or chlorine. In other embodiments, R¹ is fluorine.

In certain embodiments. R¹ is NH₂. In certain embodiments, R¹ is N(C₁-C₆alkyl)₂. In certain embodiments, R¹ is N(CH₃)₂. In certain embodiments, R¹ is NH(C₁-C₆alkyl). In certain embodiments, R¹ is alkoxy. Suitable alkoxys include, but are not limited to, methoxy, ethoxy, n-propoxy, isopropoxy and n-butoxy. In certain embodiments, R¹ is methoxy.

In certain embodiments, R¹ is hydrogen, cyclopropyl, t-butyl, methyl, difluoromethyl, fluorine, difluoroethyl, trifluoromethyl, ethyl, N(CH₃)₂, CN or CH₂CN.

In certain embodiments, R¹ is taken with R² and forms an oxo group.

In certain embodiments, R¹ is taken with R² or R² and R³ and forms a C₃-C₁₀cycloalkyl, aryl, heteroaryl or heterocycloalkyl, wherein the C₃-C₁₀cycloalkyl, aryl, heteroaryl or heterocycloalkyl is unsubstituted or substituted with one to four substituents selected from the group consisting of halogen, C₁-C₆alkyl, haloC₁-C₆alkyl, alkoxy, CN, COOC₁-C₆alkyl, aryl, COaryl, aryl and heteroaryl, wherein the heteroaryl is unsubstituted or substituted with one to four substituents selected from the group consisting of halogen, CN, C₁-C₆alkyl, alkoxy, aryl, C₃-C₆cycloalkyl and haloC₁-C₆alkyl.

In certain embodiments. R¹ is taken with R² and forms a C₃-C₁₀cycloalkyl, aryl, heteroaryl or heterocycloalkyl, wherein the C₃-C₁₀cycloalkyl, aryl, heteroaryl or heterocycloalkyl is unsubstituted or substituted with one to four substituents selected from the group consisting of halogen, C₁-C₆alkyl, haloC₁-C₆alkyl, alkoxy, CN, COOC₁-C₆alkyl, aryl, COaryl, aryl and heteroaryl, wherein the heteroaryl is unsubstituted or substituted with one to four substituents selected from the group consisting of halogen, CN, C₁-C₆alkyl, alkoxy, aryl, C₃-C₆cycloalkyl and haloC₁-C₆alkyl.

In certain embodiments, R¹ is taken with R² and R³ and forms a C₃-C₁₀cycloalkyl or heterocycloalkyl, wherein the C₃-C₁₀cycloalkyl or heterocycloalkyl is unsubstituted or substituted with one to four substituents selected from the group consisting of halogen, C₁-C₆alkyl, haloC₁-C₆alkyl, alkoxy, CN, COOC₁-C₆alkyl, aryl, COaryl, aryl and heteroaryl, wherein the heteroaryl is unsubstituted or substituted with one to four substituents selected from the group consisting of halogen, CN, C₁-C₆alkyl, alkoxy, aryl, C₃-C₆cycloalkyl and haloC₁-C₆alkyl.

In certain embodiments, R¹ is taken with R² or R² and R³ and forms a C₃-C₁₀cycloalkyl, wherein the C₃-C₁₀cycloalkyl is unsubstituted or substituted with one to four substituents selected from the group consisting of halogen, C₁-C₆alkyl, haloC₁-C₆alkyl, alkoxy, CN, COOC₁-C₆alkyl, aryl, COaryl, aryl and heteroaryl, wherein the heteroaryl is unsubstituted or substituted with one to four substituents selected from the group consisting of halogen, CN, C₁-C₆alkyl, alkoxy, aryl, C₃-C₆cycloalkyl and haloC₁-C₆alkyl.

In certain embodiments, the C₃-C₁₀cycloalkyl is

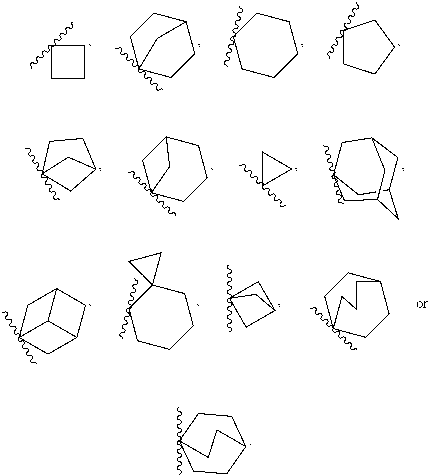

In certain embodiments, the C₃-C₁₀cycloalkyl is

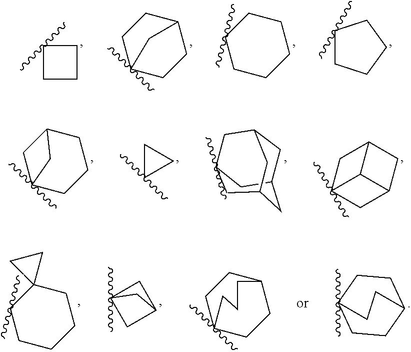

In certain embodiments, R¹ is taken with R² or R³ and forms a C₃-C₁₀cycloalkyl, wherein the C₃-C₁₀cycloalkyl is

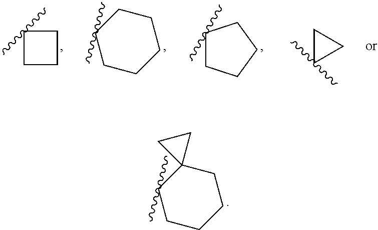

In certain embodiments, R¹ is taken with R² and R³ and forms a C₃-C₁₀cycloalkyl, wherein the C₃-C₁₀cycloalkyl is

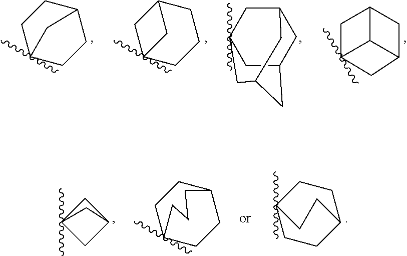

In certain embodiments, R¹ is taken with R² and R³ and forms a C₃-C₁₀cycloalkyl, wherein the C₃-C₁₀cycloalkyl is

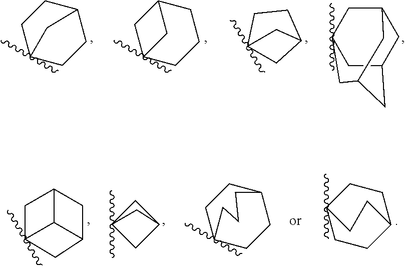

In certain embodiments, the C₃-C₁₀cycloalkyl is unsubstituted. In certain embodiments, the C₃-C₁₀cycloalkyl is substituted with one substituent. In certain embodiments, the C₃-C₁₀cycloalkyl is substituted with two substituents. In certain embodiments, the C₃-C₁₀cycloalkyl is substituted with three substituents. In certain embodiments, the C₃-C₁₀cycloalkyl is substituted with four substituents.

In certain embodiments, the C₃-C₁₀cycloalkyl is substituted with CN. In certain embodiments, the C₃-C₁₀cycloalkyl is substituted with C₁-C₆alkyl. Suitable alkyls include, but are not limited to, methyl, ethyl, n-propyl, isopropyl, n-butyl, isobutyl, sec-butyl, tert-butyl, n-pentyl, isopentyl, neopentyl, tert-pentyl, 1-methylbutyl, 2-methylbutyl, 1,2-dimethylpropyl, 1-ethylpropyl, n-hexyl, isohexyl, 1-methylpentyl, 2-methylpentyl, 3-methylpentyl, 1,1-dimethylbutyl, 1,2-dimethylbutyl, 2,2-dimethylbutyl, 1-ethylbutyl, 1,1,2-trimethylpropyl, 1,2,2-trimethylpropyl, 1-ethyl-2-methylpropyl and 1-ethyl-1-methylpropyl. In certain embodiments, the C₃-C₁₀cycloalkyl is substituted with methyl.

In certain embodiments, the C₃-C₁₀cycloalkyl is substituted with haloC₁-C₆alkyl. Suitable examples of haloalkyls include, but are not limited to, fluoromethyl, difluoromethyl, trifluoromethyl, 2-fluoroethyl, 1,2-difluoroethyl and 2,2-difluoroethyl. In certain embodiments, the C₃-C₁₀cycloalkyl is substituted with difluoromethyl and trifluoromethyl.

In certain embodiments, the C₃-C₁₀cycloalkyl is substituted with halogen. Suitable halogens include, but are not limited to, fluorine, chlorine, bromine or iodine. In certain embodiments, the C₃-C₁₀cycloalkyl is substituted with fluorine or chlorine.

In certain embodiments, the C₃-C₁₀cycloalkyl is substituted with alkoxy. In certain embodiments, the C₃-C₁₀cycloalkyl is substituted with methoxy.

In certain embodiments, the C₃-C₁₀cycloalkyl is substituted with COOC₁-C₆alkyl. In certain embodiments, the C₃-C₁₀cycloalkyl is substituted with

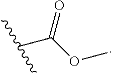 [SMILES: *C(=O)OC]

In certain embodiments, the C₃-C₁₀cycloalkyl is substituted with COaryl.

In certain embodiments, the C₃-C₁₀cycloalkyl is substituted with aryl. In certain embodiments, the C₃-C₁₀cycloalkyl is substituted with phenyl.

In certain embodiments, the C₃-C₁₀cycloalkyl is substituted with

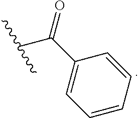 [SMILES: *C(=O)c1ccccc1]

In certain embodiments, the C₃-C₁₀cycloalkyl is substituted with heteroaryl, wherein the heteroaryl is unsubstituted or substituted. Suitable heteroaryls include, but are not limited to, pyridyl (pyridinyl), oxazolyl, imidazolyl, triazolyl, furyl, triazinyl, thienyl, pyrimidyl, pyridazinyl, indolizinyl, cinnolinyl, phthalazinyl, quinazolinyl, naphthyridinyl, quinoxalinyl, purinyl, benzimidazolyl, quinolyl, and isoquinolyl. In certain embodiments, the C₃-C₁₀cycloalkyl is substituted with heteroaryl, wherein the heteroaryl is pyrimidyl, [1,3]thiazolo[5,4-d]pyrimidyl, triazolopiperidinyl.

In certain embodiments, the heteroaryl is substituted with one substituent. In certain embodiments, the heteroaryl is substituted with two substituents. In certain embodiments, the heteroaryl is substituted with three substituents. In certain embodiments, the heteroaryl is substituted with four substituents.

In certain embodiments, the heteroaryl is substituted with halogen. Suitable halogens include, but are not limited to, fluorine, chlorine, bromine or iodine. In certain embodiments, the heteroaryl is substituted with CN. In certain embodiments, the heteroaryl is substituted with C₁-C₆alkyl. Suitable alkyls include, but are not limited to, methyl, ethyl, n-propyl, isopropyl, n-butyl, isobutyl, sec-butyl, tert-butyl, n-pentyl, isopentyl, neopentyl, tert-pentyl, 1-methylbutyl, 2-methylbutyl, 1,2-dimethylpropyl, 1-ethylpropyl, n-hexyl, isohexyl, 1-methylpentyl, 2-methylpentyl, 3-methylpentyl, 1,1-dimethylbutyl, 1,2-dimethylbutyl, 2,2-dimethylbutyl, 1-ethylbutyl, 1,1,2-trimethylpropyl, 1,2,2-trimethylpropyl, 1-ethyl-2-methylpropyl and 1-ethyl-1-methylpropyl. In certain embodiments, the heteroaryl is substituted with alkoxy. Suitable alkoxys include, but are not limited to, methoxy, ethoxy, n-propoxy, isopropoxy and n-butoxy. In certain embodiments, the heteroaryl is substituted with aryl. Suitable aryls include, but are not limited to, phenyl and naphthyl. In certain embodiments, the heteroaryl is substituted with C₃-C₆cycloalkyl. Suitable examples of cycloalkyls include, but are not limited to, cyclopropyl, cyclobutyl, cyclopentyl and cyclohexyl. In certain embodiments, the heteroaryl is substituted with haloC₁-C₆alkyl. Suitable examples of haloalkyls include, but are not limited to, fluoromethyl, difluoromethyl, trifluoromethyl, 2-fluoroethyl, 1,2-difluoroethyl and 2,2-difluoroethyl.

In certain embodiments, R¹ is taken with R² or R² and R³, and forms a C₃-C₁₀cycloalkyl, the C₃-C₁₀cycloalkyl is

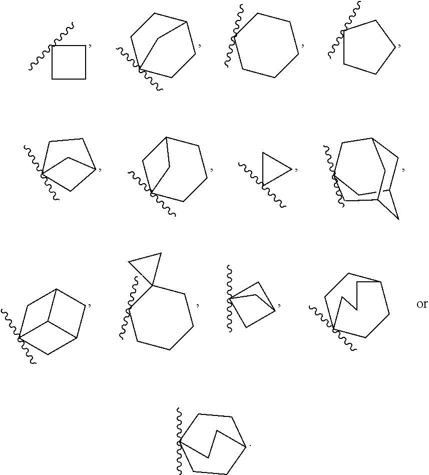

and wherein the C₃-C₁₀cycloalkyl is unsubstituted or substituted with one to four substituents selected from the group consisting of halogen, C₁-C₆alkyl, haloC₁-C₆alkyl, alkoxy, CN, COOC₁-C₆alkyl, aryl and heteroaryl, wherein the heteroaryl is unsubstituted or substituted with CN.

In certain embodiments, R¹ is taken with R² or R² and R³, and forms a C₃-C₁₀cycloalkyl, the C₃-C₁₀cycloalkyl is

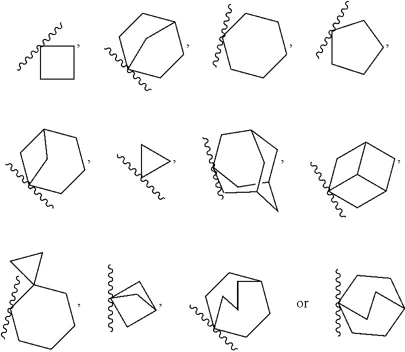

and wherein the C₃-C₁₀cycloalkyl is unsubstituted or substituted with one to four substituents selected from the group consisting of halogen, C₁-C₆alkyl, haloC₁-C₆alkyl, alkoxy, CN, COOC₁-C₆alkyl, aryl and heteroaryl, wherein the heteroaryl is unsubstituted or substituted with CN.

In certain embodiments, R¹ is taken with R² and forms an aryl, wherein the aryl is unsubstituted or substituted with one to four substituents selected from the group consisting of halogen, C₁-C₆alkyl, haloC₁-C₆alkyl, alkoxy, CN, COOC₁-C₆alkyl, COaryl, aryl and heteroaryl, wherein the heteroaryl is unsubstituted or substituted with one to four substituents selected from the group consisting of halogen, CN, C₁-C₆alkyl, alkoxy, aryl, C₃-C₆cycloalkyl and haloC₁-C₆alkyl.

In certain embodiments, the aryl is

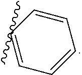

In certain embodiments, the aryl is unsubstituted. In certain embodiments, the aryl is substituted with one substituent. In certain embodiments, the aryl is substituted with two substituents. In certain embodiments, the aryl is substituted with three substituents. In certain embodiments, the aryl is substituted with four substituents.

In certain embodiments, the aryl is substituted with CN. In certain embodiments, the aryl is substituted with C₁-C₆alkyl. Suitable alkyls include, but are not limited to, methyl, ethyl, n-propyl, isopropyl, n-butyl, isobutyl, sec-butyl, tert-butyl, n-pentyl, isopentyl, neopentyl, tert-pentyl, 1-methylbutyl, 2-methylbutyl, 1,2-dimethylpropyl, 1-ethylpropyl, n-hexyl, isohexyl, 1-methylpentyl, 2-methylpentyl, 3-methylpentyl, 1,1-dimethylbutyl, 1,2-dimethylbutyl, 2,2-dimethylbutyl, 1-ethylbutyl, 1,1,2-trimethylpropyl, 1,2,2-trimethylpropyl, 1-ethyl-2-methylpropyl and 1-ethyl-1-methylpropyl. In certain embodiments, the aryl is substituted with haloC₁-C₆alkyl. Suitable examples of haloalkyls include, but are not limited to, fluoromethyl, difluoromethyl, trifluoromethyl, 2-fluoroethyl, 1,2-difluoroethyl and 2,2-difluoroethyl. In certain embodiments, the aryl is substituted with halogen. Suitable halogens include, but are not limited to, fluorine, chlorine, bromine or iodine.

In certain embodiments, the aryl is substituted with COOC₁-C₆alkyl. In certain embodiments, the aryl is substituted with

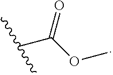 [SMILES: *C(=O)OC]

In certain embodiments, the aryl is substituted with COaryl. In certain embodiments, the aryl is substituted with

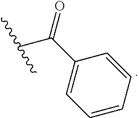 [SMILES: *C(=O)c1ccccc1]

In certain embodiments, the aryl is substituted with aryl. In certain embodiments, the aryl is substituted with phenyl. In certain embodiments, the aryl is substituted with alkoxy. In certain embodiments, the aryl is substituted with methoxy.

In certain embodiments, the aryl is substituted with heteroaryl, wherein the heteroaryl is unsubstituted or substituted. Suitable heteroaryls include, but are not limited to, pyridyl (pyridinyl), oxazolyl, imidazolyl, triazolyl, furyl, triazinyl, thienyl, pyrimidyl, pyridazinyl, indolizinyl, cinnolinyl, phthalazinyl, quinazolinyl, naphthyridinyl, quinoxalinyl, purinyl, benzimidazolyl, quinolyl, and isoquinolyl.

In certain embodiments, the heteroaryl is substituted with one substituent. In certain embodiments, the heteroaryl is substituted with two substituents. In certain embodiments, the heteroaryl is substituted with three substituents. In certain embodiments, the heteroaryl is substituted with four substituents.

In certain embodiments, the heteroaryl is substituted with halogen. Suitable halogens include, but are not limited to, fluorine, chlorine, bromine or iodine. In certain embodiments, the heteroaryl is substituted with CN. In certain embodiments, the heteroaryl is substituted with C₁-C₆alkyl. Suitable alkyls include, but are not limited to, methyl, ethyl, n-propyl, isopropyl, n-butyl, isobutyl, sec-butyl, tert-butyl, n-pentyl, isopentyl, neopentyl, tert-pentyl, 1-methylbutyl, 2-methylbutyl, 1,2-dimethylpropyl, 1-ethylpropyl, n-hexyl, isohexyl, 1-methylpentyl, 2-methylpentyl, 3-methylpentyl, 1,1-dimethylbutyl, 1,2-dimethylbutyl, 2,2-dimethylbutyl, 1-ethylbutyl, 1,1,2-trimethylpropyl, 1,2,2-trimethylpropyl, 1-ethyl-2-methylpropyl and 1-ethyl-1-methylpropyl. In certain embodiments, the heteroaryl is substituted with alkoxy. Suitable alkoxys include, but are not limited to, methoxy, ethoxy, n-propoxy, isopropoxy and n-butoxy. In certain embodiments, the heteroaryl is substituted with aryl. Suitable aryls include, but are not limited to, phenyl and naphthyl. In certain embodiments, the heteroaryl is substituted with C₃-C₆cycloalkyl. Suitable examples of cycloalkyls include, but are not limited to, cyclopropyl, cyclobutyl, cyclopentyl and cyclohexyl. In certain embodiments, the heteroaryl is substituted with haloC₁-C₆alkyl. Suitable examples of haloalkyls include, but are not limited to, fluoromethyl, difluoromethyl, trifluoromethyl, 2-fluoroethyl, 1,2-difluoroethyl and 2,2-difluoroethyl.

In certain embodiments, R¹ is taken with R² and forms a heteroaryl, wherein the heteroaryl is unsubstituted or substituted with one to four substituents selected from the group consisting of halogen, C₁-C₆alkyl, haloC₁-C₆alkyl, alkoxy, CN, COOC₁-C₆alkyl, COaryl, aryl and heteroaryl, wherein the heteroaryl is unsubstituted or substituted with one to four substituents selected from the group consisting of halogen, CN, C₁-C₆alkyl, alkoxy, aryl, C₃-C₆cycloalkyl and haloC₁-C₆alkyl.

In certain embodiments, the heteroaryl is pyridyl (pyridinyl), oxazolyl, imidazolyl, triazolyl, furyl, triazinyl, thienyl, pyrimidyl, pyridazinyl, indolizinyl, cinnolinyl, phthalazinyl, quinazolinyl, naphthyridinyl, quinoxalinyl, purinyl, benzimidazolyl, quinolyl, 2-oxabicyclo[2.1.1.]hexanyl, oxolanyl or isoquinolyl. In certain embodiments, the heteroaryl is piperidinyl, 2-oxabicyclo[2.1.1.]hexanyl or oxolanyl.

In certain embodiments, the heteroaryl is unsubstituted. In certain embodiments, the heteroaryl is substituted with one substituent. In certain embodiments, the heteroaryl is substituted with two substituents. In certain embodiments, the heteroaryl is substituted with three substituents. In certain embodiments, the heteroaryl is substituted with four substituents.

In certain embodiments, the heteroaryl is substituted with CN. In certain embodiments, the heteroaryl is substituted with C₁-C₆alkyl. Suitable alkyls include, but are not limited to, methyl, ethyl, n-propyl, isopropyl, n-butyl, isobutyl, sec-butyl, tert-butyl, n-pentyl, isopentyl, neopentyl, tert-pentyl, 1-methylbutyl, 2-methylbutyl, 1,2-dimethylpropyl, 1-ethylpropyl, n-hexyl, isohexyl, 1-methylpentyl, 2-methylpentyl, 3-methylpentyl, 1,1-dimethylbutyl, 1,2-dimethylbutyl, 2,2-dimethylbutyl, 1-ethylbutyl, 1,1,2-trimethylpropyl, 1,2,2-trimethylpropyl, 1-ethyl-2-methylpropyl and 1-ethyl-1-methylpropyl. In certain embodiments, the heteroaryl is substituted with haloC₁-C₆alkyl. Suitable examples of haloalkyls include, but are not limited to, fluoromethyl, difluoromethyl, trifluoromethyl, 2-fluoroethyl, 1,2-difluoroethyl and 2,2-difluoroethyl. In certain embodiments, the heteroaryl is substituted with halogen. Suitable halogens include, but are not limited to, fluorine, chlorine, bromine or iodine.

In certain embodiments, the heteroaryl is substituted with COOC₁-C₆alkyl. In certain embodiments, the heteroaryl is substituted with

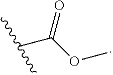 [SMILES: *C(=O)OC]

In certain embodiments, the heteroaryl is substituted with COaryl. In certain embodiments, the heteroaryl is substituted with

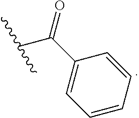 [SMILES: *C(=O)c1ccccc1]

In certain embodiments, the heteroaryl is substituted with aryl. In certain embodiments, the heteroaryl is substituted with phenyl. In certain embodiments, the heteroaryl is substituted with alkoxy. In certain embodiments, the heteroaryl is substituted with methoxy.

In certain embodiments, the heteroaryl is substituted with heteroaryl, wherein the heteroaryl is unsubstituted or substituted. Suitable heteroaryls include, but are not limited to, pyridyl (pyridinyl), oxazolyl, imidazolyl, triazolyl, furyl, triazinyl, thienyl, pyrimidyl, pyridazinyl, indolizinyl, cinnolinyl, phthalazinyl, quinazolinyl, naphthyridinyl, quinoxalinyl, purinyl, benzimidazolyl, quinolyl, and isoquinolyl. In certain embodiments, the heteroaryl is pyrimidinyl, [1,3]thiazolo[5,4-d]pyrimidyl or triazolopiperidinyl.

In certain embodiments, the heteroaryl is substituted with one substituent. In certain embodiments, the heteroaryl is substituted with two substituents. In certain embodiments, the heteroaryl is substituted with three substituents. In certain embodiments, the heteroaryl is substituted with four substituents.

In certain embodiments, the heteroaryl is substituted with halogen. Suitable halogens include, but are not limited to, fluorine, chlorine, bromine or iodine. In certain embodiments, the heteroaryl is substituted with CN. In certain embodiments, the heteroaryl is substituted with C₁-C₆alkyl. Suitable alkyls include, but are not limited to, methyl, ethyl, n-propyl, isopropyl, n-butyl, isobutyl, sec-butyl, tert-butyl, n-pentyl, isopentyl, neopentyl, tert-pentyl, 1-methylbutyl, 2-methylbutyl, 1,2-dimethylpropyl, 1-ethylpropyl, n-hexyl, isohexyl, 1-methylpentyl, 2-methylpentyl, 3-methylpentyl, 1,1-dimethylbutyl, 1,2-dimethylbutyl, 2,2-dimethylbutyl, 1-ethylbutyl, 1,1,2-trimethylpropyl, 1,2,2-trimethylpropyl, 1-ethyl-2-methylpropyl and 1-ethyl-1-methylpropyl. In certain embodiments, the heteroaryl is substituted with alkoxy. Suitable alkoxys include, but are not limited to, methoxy, ethoxy, n-propoxy, isopropoxy and n-butoxy. In certain embodiments, the heteroaryl is substituted with aryl. Suitable aryls include, but are not limited to, phenyl and naphthyl. In certain embodiments, the heteroaryl is substituted with C₃-C₆cycloalkyl. Suitable examples of cycloalkyls include, but are not limited to, cyclopropyl, cyclobutyl, cyclopentyl and cyclohexyl. In certain embodiments, the heteroaryl is substituted with haloC₁-C₆alkyl. Suitable examples of haloalkyls include, but are not limited to, fluoromethyl, difluoromethyl, trifluoromethyl, 2-fluoroethyl, 1,2-difluoroethyl and 2,2-difluoroethyl.

In certain embodiments, R¹ is taken with R² or R² and R³ and forms a heterocycloalkyl, wherein the heterocycloalkyl is unsubstituted or substituted with one to four substituents selected from the group consisting of halogen, C₁-C₆alkyl, haloC₁-C₆alkyl, alkoxy, CN, COOC₁-C₆alkyl, COaryl, aryl and heteroaryl, wherein the heteroaryl is unsubstituted or substituted with one to four substituents selected from the group consisting of halogen, CN, C₁-C₆alkyl, alkoxy, aryl, C₃-C₆cycloalkyl and haloC₁-C₆alkyl.

In certain embodiments, R¹ is taken with R² and forms a heterocycloalkyl, wherein the heterocycloalkyl is unsubstituted or substituted with one to four substituents selected from the group consisting of halogen, C₁-C₆alkyl, haloC₁-C₆alkyl, alkoxy, CN, COOC₁-C₆alkyl, COaryl, aryl and heteroaryl, wherein the heteroaryl is unsubstituted or substituted with one to four substituents selected from the group consisting of halogen, CN, C₁-C₆alkyl, alkoxy, aryl, C₃-C₆cycloalkyl and haloC₁-C₆alkyl.

In certain embodiments, R¹ is taken with R² and R³ and forms a heterocycloalkyl, wherein the heterocycloalkyl is unsubstituted or substituted with one to four substituents selected from the group consisting of halogen, C₁-C₆alkyl, haloC₁-C₆alkyl, alkoxy, CN, COOC₁-C₆alkyl, COaryl, aryl and heteroaryl, wherein the heteroaryl is unsubstituted or substituted with one to four substituents selected from the group consisting of halogen, CN, C₁-C₆alkyl, alkoxy, aryl, C₃-C₆cycloalkyl and haloC₁-C₆alkyl.

In certain embodiments, the heterocycloalkyl is

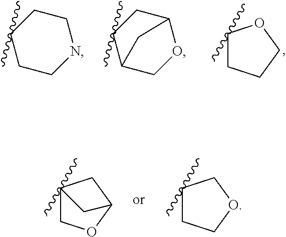

In certain embodiments, the heterocycloalkyl is

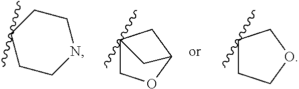

In certain embodiments, the heterocycloalkyl is unsubstituted. In certain embodiments, the heterocycloalkyl is substituted with one substituent. In certain embodiments, the heterocycloalkyl is substituted with two substituents. In certain embodiments, the heterocycloalkyl is substituted with three substituents. In certain embodiments, the heterocycloalkyl is substituted with four substituents.

In certain embodiments, the heterocycloalkyl is substituted with CN. In certain embodiments, the heterocycloalkyl is substituted with C₁-C₆alkyl. Suitable alkyls include, but are not limited to, methyl, ethyl, n-propyl, isopropyl, n-butyl, isobutyl, sec-butyl, tert-butyl, n-pentyl, isopentyl, neopentyl, tert-pentyl, 1-methylbutyl, 2-methylbutyl, 1,2-dimethylpropyl, 1-ethylpropyl, n-hexyl, isohexyl, 1-methylpentyl, 2-methylpentyl, 3-methylpentyl, 1,1-dimethylbutyl, 1,2-dimethylbutyl, 2,2-dimethylbutyl, 1-ethylbutyl, 1,1,2-trimethylpropyl, 1,2,2-trimethylpropyl, 1-ethyl-2-methylpropyl and 1-ethyl-1-methylpropyl. In certain embodiments, the heterocycloalkyl is substituted with haloC₁-C₆alkyl. Suitable examples of haloalkyls include, but are not limited to, fluoromethyl, difluoromethyl, trifluoromethyl, 2-fluoroethyl, 1,2-difluoroethyl and 2,2-difluoroethyl. In certain embodiments, the heterocycloalkyl is substituted with halogen. Suitable halogens include, but are not limited to, fluorine, chlorine, bromine or iodine.

In certain embodiments, the heterocycloalkyl is substituted with COOC₁-C₆alkyl. In certain embodiments, the heterocycloalkyl is substituted with

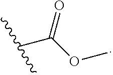 [SMILES: *C(=O)OC]

In certain embodiments, the heterocycloalkyl is substituted with COaryl. In certain embodiments, the heterocycloalkyl is substituted with

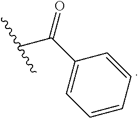 [SMILES: *C(=O)c1ccccc1]

In certain embodiments, the heterocycloalkyl is substituted with aryl. In certain embodiments, the heterocycloalkyl is substituted with phenyl. In certain embodiments, the heterocycloalkyl is substituted with alkoxy. In certain embodiments, the heterocycloalkyl is substituted with methoxy.

In certain embodiments, the heterocycloalkyl is substituted with heteroaryl, wherein the heteroaryl is unsubstituted or substituted. Suitable heteroaryls include, but are not limited to, pyridyl (pyridinyl), oxazolyl, imidazolyl, triazolyl, furyl, triazinyl, thienyl, pyrimidyl, pyridazinyl, indolizinyl, cinnolinyl, phthalazinyl, quinazolinyl, naphthyridinyl, quinoxalinyl, purinyl, benzimidazolyl, quinolyl, and isoquinolyl. In certain embodiments, the heterocycloalkyl is pyrimidinyl, [1,3]thiazolo[5,4-d]pyrimidyl or triazolopiperidinyl.

In certain embodiments, the heterocycloalkyl is substituted with one substituent. In certain embodiments, the heterocycloalkyl is substituted with two substituents. In certain embodiments, the heterocycloalkyl is substituted with three substituents. In certain embodiments, the heterocycloalkyl is substituted with four substituents.

In certain embodiments, the heteroaryl is substituted with halogen. Suitable halogens include, but are not limited to, fluorine, chlorine, bromine or iodine. In certain embodiments, the heteroaryl is substituted with fluorine and chlorine. In certain embodiments, the heteroaryl is substituted with CN. In certain embodiments, the heteroaryl is substituted with C₁-C₆alkyl. Suitable alkyls include, but are not limited to, methyl, ethyl, n-propyl, isopropyl, n-butyl, isobutyl, sec-butyl, tert-butyl, n-pentyl, isopentyl, neopentyl, tert-pentyl, 1-methylbutyl, 2-methylbutyl, 1,2-dimethylpropyl, 1-ethylpropyl, n-hexyl, isohexyl, 1-methylpentyl, 2-methylpentyl, 3-methylpentyl, 1,1-dimethylbutyl, 1,2-dimethylbutyl, 2,2-dimethylbutyl, 1-ethylbutyl, 1,1,2-trimethylpropyl, 1,2,2-trimethylpropyl, 1-ethyl-2-methylpropyl and 1-ethyl-1-methylpropyl. In certain embodiments, the heteroaryl is substituted with alkoxy. Suitable alkoxys include, but are not limited to, methoxy, ethoxy, n-propoxy, isopropoxy and n-butoxy. In certain embodiments, the heteroaryl is substituted with aryl. Suitable aryls include, but are not limited to, phenyl and naphthyl. In certain embodiments, the heteroaryl is substituted with C₃-C₆cycloalkyl. Suitable examples of cycloalkyls include, but are not limited to, cyclopropyl, cyclobutyl, cyclopentyl and cyclohexyl. In certain embodiments, the heteroaryl is substituted with haloC₁-C₆alkyl. Suitable examples of haloalkyls include, but are not limited to, fluoromethyl, difluoromethyl, trifluoromethyl, 2-fluoroethyl, 1,2-difluoroethyl and 2,2-difluoroethyl.

In certain embodiments, when R¹ is taken with R² or R² and R³ and forms a heterocycloalkyl, the heterocycloalkyl is

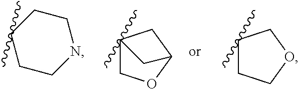

wherein the heterocycloalkyl is unsubstituted or substituted with one to four substituents selected from the group consisting of halogen, C₁-C₆alkyl, haloC₁-C₆alkyl, alkoxy, CN, COOC₁-C₆alkyl, COaryl, aryl and heteroaryl, wherein the heteroaryl is unsubstituted or substituted with one to four substituents selected from the group consisting of halogen, CN, C₁-C₆alkyl and haloC₁-C₆alkyl.

In certain embodiments, wherein when the heterocycloalkyl is

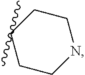

and wherein the heterocycloalkyl is substituted with heteroaryl, the heteroaryl is

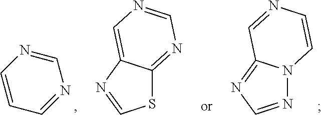

wherein the heteroaryl is unsubstituted or substituted with CN.

With regard to the compounds described herein, R² is hydrogen, OH, C₁-C₆alkylOH, CN, C₁-C₆alkylCN, C₁-C₆alkyl, C₃-C₆cycloalkyl, haloC₁-C₆alkyl, halogen, NH₂, N(C₁-C₆alkyl)₂, NH(C₁-C₆alkyl) or alkoxy, or wherein when R² is taken with R¹ and forms an oxo group, or wherein when R² is taken with R¹ or R¹ and R³, forms a C₃-C₁₀cycloalkyl, aryl, heteroaryl or heterocycloalkyl, wherein the C₃-C₁₀cycloalkyl, aryl, heteroaryl or heterocycloalkyl is unsubstituted or substituted with one to four substituents selected from the group consisting of halogen, C₁-C₆alkyl, haloC₁-C₆alkyl, alkoxy, CN, COOC₁-C₆alkyl, aryl, COaryl and heteroaryl, wherein the heteroaryl is unsubstituted or substituted with one to four substituents selected from the group consisting of halogen, CN, C₁-C₆alkyl, alkoxy, aryl, C₃-C₆cycloalkyl and haloC₁-C₆alkyl.

In certain embodiments, R² is hydrogen. In certain embodiments, R² is OH. In certain embodiments, R² is C₁-C₆alkylOH. Suitable alcohols include, but are not limited to, methanol, ethanol, propanol and butanol.

In certain embodiments, R² is CN. In certain embodiments, R² is C₁-C₆alkylCN. In certain embodiments, R² is

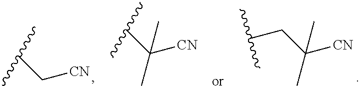

In certain embodiments, R² is

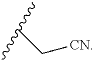 [SMILES: *CC#N]

In certain embodiments, R² is C₁-C₆alkyl. Suitable alkyls include, but are not limited to, methyl, ethyl, n-propyl, isopropyl, n-butyl, isobutyl, sec-butyl, tert-butyl, n-pentyl, isopentyl, neopentyl, tert-pentyl, 1-methylbutyl, 2-methylbutyl, 1,2-dimethylpropyl, 1-ethylpropyl, n-hexyl, isohexyl, 1-methylpentyl, 2-methylpentyl, 3-methylpentyl, 1,1-dimethylbutyl, 1,2-dimethylbutyl, 2,2-dimethylbutyl, 1-ethylbutyl, 1,1,2-trimethylpropyl, 1,2,2-trimethylpropyl, 1-ethyl-2-methylpropyl and 1-ethyl-1-methylpropyl. In certain embodiments, R² is methyl, ethyl or t-butyl.

In certain embodiments, R² is C₃-C₆cycloalkyl. Suitable cycloalkyls include, but are not limited to, cyclopropyl, cyclobutyl, cyclopentyl and cyclohexyl. In certain embodiments, R² is cyclopropyl.

In certain embodiments, R² is haloC₁-C₆alkyl. Suitable examples of haloalkyls include, but are not limited to, fluoromethyl, difluoromethyl, trifluoromethyl, 2-fluoroethyl, 1,2-difluoroethyl and 2,2-difluoroethyl. In certain embodiments, R² is difluoromethyl. In certain embodiments, R² is trifluoromethyl. In certain embodiments, R² is difluoromethyl or trifluoromethyl. In certain embodiments, R² is fluoromethyl, difluoromethyl, difluoroethyl or trifluoromethyl. In certain embodiments, R² is halogen. Suitable halogens include, but are not limited to, fluorine, chlorine, bromine or iodine. In certain embodiments, R² is fluorine or chlorine. In certain embodiments, R² is fluorine.

In certain embodiments. R² is NH₂. In certain embodiments, R² is N(C₁-C₆alkyl)₂. In certain embodiments, R² is N(CH₃)₂. In certain embodiments, R² is NH(C₁-C₆alkyl). In certain embodiments, R² is alkoxy. Suitable alkoxys include, but are not limited to, methoxy, ethoxy, n-propoxy, isopropoxy and n-butoxy. In certain embodiments, R² is methoxy.

In certain embodiments, R² is methyl, ethyl, t-butyl, fluoromethyl, difluoromethyl, fluorine, difluoroethyl, trifluoromethyl, N(CH₃)₂, CN or CH₂CN.

In certain embodiments, R² is taken with R¹ and forms an oxo group.

In certain embodiments, R² is taken with R¹ or R¹ and R³ and forms a C₃-C₁₀cycloalkyl, aryl, heteroaryl or heterocycloalkyl, wherein the C₃-C₁₀cycloalkyl, aryl, heteroaryl or heterocycloalkyl is unsubstituted or substituted with one to four substituents selected from the group consisting of halogen, C₁-C₆alkyl, haloC₁-C₆alkyl, alkoxy, CN, COOC₁-C₆alkyl, COaryl, aryl and heteroaryl, wherein the heteroaryl is unsubstituted or substituted with one to four substituents selected from the group consisting of halogen, CN, C₁-C₆alkyl, alkoxy, aryl, C₃-C₆cycloalkyl and haloC₁-C₆alkyl.

In certain embodiments, R² is taken with R¹ and forms a C₃-C₁₀cycloalkyl, aryl, heteroaryl or heterocycloalkyl, wherein the C₃-C₁₀cycloalkyl, aryl, heteroaryl or heterocycloalkyl is unsubstituted or substituted with one to four substituents selected from the group consisting of halogen, C₁-C₆alkyl, haloC₁-C₆alkyl, alkoxy, CN, COOC₁-C₆alkyl, aryl, COaryl, aryl and heteroaryl, wherein the heteroaryl is unsubstituted or substituted with one to four substituents selected from the group consisting of halogen, CN, C₁-C₆alkyl, alkoxy, aryl, C₃-C₆cycloalkyl and haloC₁-C₆alkyl.

In certain embodiments, R² is taken with R¹ and R³ and forms a C₃-C₁₀cycloalkyl or heterocycloalkyl, wherein the C₃-C₁₀cycloalkyl or heterocycloalkyl is unsubstituted or substituted with one to four substituents selected from the group consisting of halogen, C₁-C₆alkyl, haloC₁-C₆alkyl, alkoxy, CN, COOC₁-C₆alkyl, COaryl, aryl and heteroaryl, wherein the heteroaryl is unsubstituted or substituted with one to four substituents selected from the group consisting of halogen, CN, C₁-C₆alkyl, alkoxy, aryl, C₃-C₆cycloalkyl and haloC₁-C₆alkyl.

In certain embodiments, R² is taken with R¹ or R¹ and R³ and forms a C₃-C₁₀cycloalkyl, wherein the C₃-C₁₀cycloalkyl is unsubstituted or substituted with one to four substituents selected from the group consisting of halogen, C₁-C₆alkyl, haloC₁-C₆alkyl, alkoxy, CN, COOC₁-C₆alkyl, COaryl, aryl and heteroaryl, wherein the heteroaryl is unsubstituted or substituted with one to four substituents selected from the group consisting of halogen, CN, C₁-C₆alkyl, alkoxy, aryl, C₃-C₆cycloalkyl and haloC₁-C₆alkyl.

In certain embodiments, R² is taken with R¹ and R³ and forms a C₃-C₁₀cycloalkyl, wherein the C₃-C₁₀cycloalkyl is

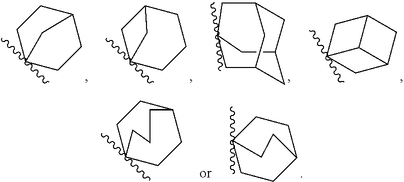

In certain embodiments, R² is taken with R¹ or R³ and forms a C₃-C₁₀cycloalkyl, wherein the C₃-C₁₀cycloalkyl is

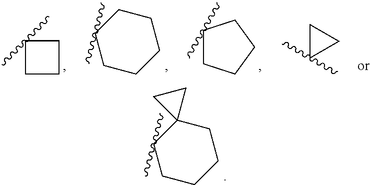

In certain embodiments, the C₃-C₁₀cycloalkyl is

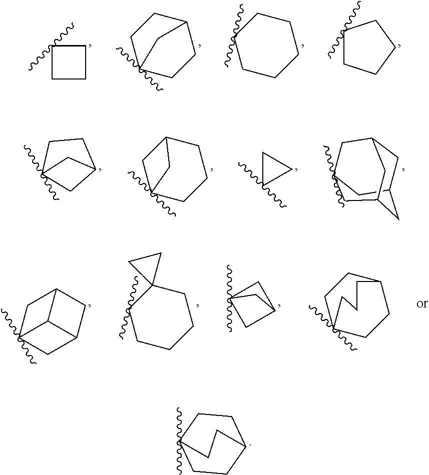

In certain embodiments, the C₃-C₁₀cycloalkyl is

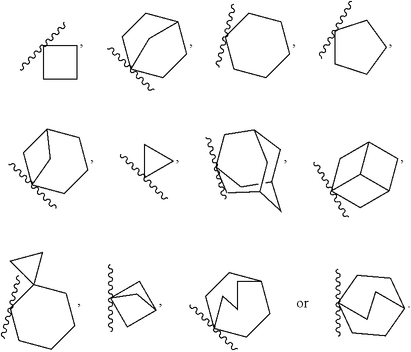

In certain embodiments, the C₃-C₁₀cycloalkyl is unsubstituted. In certain embodiments, the C₃-C₁₀cycloalkyl is substituted with one substituent. In certain embodiments, the C₃-C₁₀cycloalkyl is substituted with two substituents. In certain embodiments, the C₃-C₁₀cycloalkyl is substituted with three substituents. In certain embodiments, the C₃-C₁₀cycloalkyl is substituted with four substituents.

In certain embodiments, the C₃-C₁₀cycloalkyl is substituted with CN. In certain embodiments, the C₃-C₁₀cycloalkyl is substituted with C₁-C₆alkyl. Suitable alkyls include, but are not limited to, methyl, ethyl, n-propyl, isopropyl, n-butyl, isobutyl, sec-butyl, tert-butyl, n-pentyl, isopentyl, neopentyl, tert-pentyl, 1-methylbutyl, 2-methylbutyl, 1,2-dimethylpropyl, 1-ethylpropyl, n-hexyl, isohexyl, 1-methylpentyl, 2-methylpentyl, 3-methylpentyl, 1,1-dimethylbutyl, 1,2-dimethylbutyl, 2,2-dimethylbutyl, 1-ethylbutyl, 1,1,2-trimethylpropyl, 1,2,2-trimethylpropyl, 1-ethyl-2-methylpropyl and 1-ethyl-1-methylpropyl. In certain embodiments, the C₃-C₁₀cycloalkyl is substituted with methyl, ethyl or t-butyl.

In certain embodiments, the C₃-C₁₀cycloalkyl is substituted with haloC₁-C₆alkyl. Suitable examples of haloalkyls include, but are not limited to, fluoromethyl, difluoromethyl, trifluoromethyl, 2-fluoroethyl, 1,2-difluoroethyl and 2,2-difluoroethyl. In certain embodiments, the C₃-C₁₀cycloalkyl is substituted with fluoromethyl, difluoromethyl, difluoroethyl and trifluoromethyl.

In certain embodiments, the C₃-C₁₀cycloalkyl is substituted with halogen. Suitable halogens include, but are not limited to, fluorine, chlorine, bromine or iodine. In certain embodiments, the C₃-C₁₀cycloalkyl is substituted with fluorine and chlorine. In certain embodiments, the C₃-C₁₀cycloalkyl is substituted with fluorine.

In certain embodiments, the C₃-C₁₀cycloalkyl is substituted with COOC₁-C₆alkyl. In certain embodiments, the C₃-C₁₀cycloalkyl is substituted with

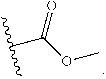 [SMILES: *C(=O)OC]

In certain embodiments, the C₃-C₁₀cycloalkyl is substituted with alkoxy. In certain embodiments, the C₃-C₁₀cycloalkyl is substituted with methoxy. In certain embodiments, the C₃-C₁₀cycloalkyl is substituted with COaryl. In certain embodiments, the C₃-C₁₀cycloalkyl is substituted with

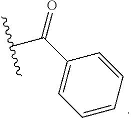 [SMILES: *C(=O)c1ccccc1]

In certain embodiments, the C₃-C₁₀cycloalkyl is substituted with aryl. In certain embodiments, the C₃-C₁₀cycloalkyl is substituted with phenyl.

In certain embodiments, the C₃-C₁₀cycloalkyl is substituted with heteroaryl, wherein the heteroaryl is unsubstituted or substituted. Suitable heteroaryls include, but are not limited to, pyridyl (pyridinyl), oxazolyl, imidazolyl, triazolyl, furyl, triazinyl, thienyl, pyrimidyl, pyridazinyl, indolizinyl, cinnolinyl, phthalazinyl, quinazolinyl, naphthyridinyl, quinoxalinyl, purinyl, benzimidazolyl, quinolyl, and isoquinolyl. In certain embodiments, the C₃-C₁₀cycloalkyl is substituted with pyrimidyl, [1,3]thiazolo[5,4-d]pyrimidyl or triazolopiperidinyl.

In certain embodiments, the heteroaryl is substituted with one substituent. In certain embodiments, the heteroaryl is substituted with two substituents. In certain embodiments, the heteroaryl is substituted with three substituents. In certain embodiments, the heteroaryl is substituted with four substituents.

In certain embodiments, the heteroaryl is substituted with halogen. Suitable halogens include, but are not limited to, fluorine, chlorine, bromine or iodine. In certain embodiments, the heteroaryl is substituted with CN. In certain embodiments, the heteroaryl is substituted with C₁-C₆alkyl. Suitable alkyls include, but are not limited to, methyl, ethyl, n-propyl, isopropyl, n-butyl, isobutyl, sec-butyl, tert-butyl, n-pentyl, isopentyl, neopentyl, tert-pentyl, 1-methylbutyl, 2-methylbutyl, 1,2-dimethylpropyl, 1-ethylpropyl, n-hexyl, isohexyl, 1-methylpentyl, 2-methylpentyl, 3-methylpentyl, 1,1-dimethylbutyl, 1,2-dimethylbutyl, 2,2-dimethylbutyl, 1-ethylbutyl, 1,1,2-trimethylpropyl, 1,2,2-trimethylpropyl, 1-ethyl-2-methylpropyl and 1-ethyl-1-methylpropyl. In certain embodiments, the heteroaryl is substituted with alkoxy. Suitable alkoxys include, but are not limited to, methoxy, ethoxy, n-propoxy, isopropoxy and n-butoxy. In certain embodiments, the heteroaryl is substituted with aryl. Suitable aryls include, but are not limited to, phenyl and naphthyl. In certain embodiments, the heteroaryl is substituted with C₃-C₆cycloalkyl. Suitable examples of cycloalkyls include, but are not limited to, cyclopropyl, cyclobutyl, cyclopentyl and cyclohexyl. In certain embodiments, the heteroaryl is substituted with haloC₁-C₆alkyl. Suitable examples of haloalkyls include, but are not limited to, fluoromethyl, difluoromethyl, trifluoromethyl, 2-fluoroethyl, 1,2-difluoroethyl and 2,2-difluoroethyl.

In certain embodiments, when R² is taken with R¹ or R¹ and R³, the C₃-C₁₀cycloalkyl is

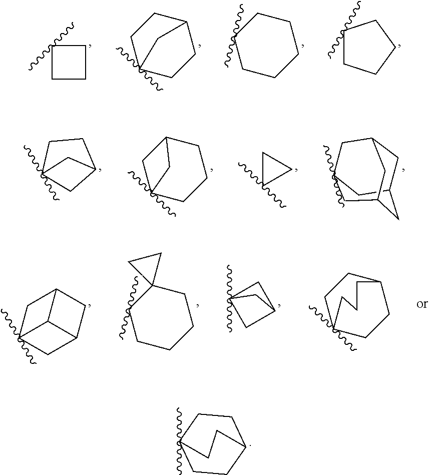

wherein the C₃-C₁₀cycloalkyl is unsubstituted or substituted with one to four substituents selected from the group consisting of halogen, C₁-C₆alkyl, haloC₁-C₆alkyl, alkoxy, CN, COOC₁-C₆alkyl.

In certain embodiments, when R² is taken with R¹ or R¹ and R³, the C₃-C₁₀cycloalkyl is

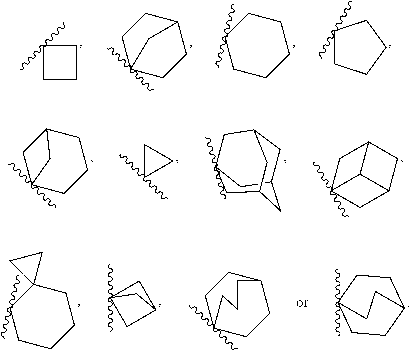

wherein the C₃-C₁₀cycloalkyl is unsubstituted or substituted with one to four substituents selected from the group consisting of halogen, C₁-C₆alkyl, haloC₁-C₆alkyl, alkoxy, CN, COOC₁-C₆alkyl.

In certain embodiments, R² is taken with R¹ and forms an aryl, wherein the aryl is unsubstituted or substituted with one to four substituents selected from the group consisting of halogen, C₁-C₆alkyl, haloC₁-C₆alkyl, alkoxy, CN, COOC₁-C₆alkyl, COaryl, aryl and heteroaryl, wherein the heteroaryl is unsubstituted or substituted with one to four substituents selected from the group consisting of halogen, CN, C₁-C₆alkyl, alkoxy, aryl, C₃-C₆cycloalkyl and haloC₁-C₆alkyl.

In certain embodiments, the aryl is

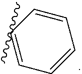

In certain embodiments, the aryl is unsubstituted. In certain embodiments, the aryl is substituted with one substituent. In certain embodiments, the aryl is substituted with two substituents. In certain embodiments, the aryl is substituted with three substituents. In certain embodiments, the aryl is substituted with four substituents.

In certain embodiments, the aryl is substituted with CN. In certain embodiments, the aryl is substituted with C₁-C₆alkyl. Suitable alkyls include, but are not limited to, methyl, ethyl, n-propyl, isopropyl, n-butyl, isobutyl, sec-butyl, tert-butyl, n-pentyl, isopentyl, neopentyl, tert-pentyl, 1-methylbutyl, 2-methylbutyl, 1,2-dimethylpropyl, 1-ethylpropyl, n-hexyl, isohexyl, 1-methylpentyl, 2-methylpentyl, 3-methylpentyl, 1,1-dimethylbutyl, 1,2-dimethylbutyl, 2,2-dimethylbutyl, 1-ethylbutyl, 1,1,2-trimethylpropyl, 1,2,2-trimethylpropyl, 1-ethyl-2-methylpropyl and 1-ethyl-1-methylpropyl. In certain embodiments, the aryl is substituted with CN. In certain embodiments, the aryl is substituted methyl, ethyl or t-butyl.

In certain embodiments, the aryl is substituted with haloC₁-C₆alkyl. Suitable examples of haloalkyls include, but are not limited to, fluoromethyl, difluoromethyl, trifluoromethyl, 2-fluoroethyl, 1,2-difluoroethyl and 2,2-difluoroethyl. In certain embodiments, the aryl is substituted with fluoromethyl, difluoromethyl, difluoroethyl or trifluoromethyl. In certain embodiments, the aryl is substituted with halogen. Suitable halogens include, but are not limited to, fluorine, chlorine, bromine or iodine. In certain embodiments, the aryl is substituted with fluorine.

In certain embodiments, the aryl is substituted with COOC₁-C₆alkyl. In certain embodiments, the aryl is substituted with

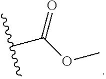 [SMILES: *C(=O)OC]

In certain embodiments, the aryl is substituted with alkoxy. In certain embodiments, the aryl is substituted with methoxy. In certain embodiments, the aryl is substituted with aryl. In certain embodiments, the aryl is substituted with phenyl. In certain embodiments, the aryl is substituted with COaryl. In certain embodiments, the aryl is substituted with

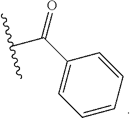 [SMILES: *C(=O)c1ccccc1]

In certain embodiments, the aryl is substituted with heteroaryl, wherein the heteroaryl is unsubstituted or substituted. Suitable heteroaryls include, but are not limited to, pyridyl (pyridinyl), oxazolyl, imidazolyl, triazolyl, furyl, triazinyl, thienyl, pyrimidyl, pyridazinyl, indolizinyl, cinnolinyl, phthalazinyl, quinazolinyl, naphthyridinyl, quinoxalinyl, purinyl, benzimidazolyl, quinolyl, and isoquinolyl. In certain embodiments, the aryl is substituted with pyrimidyl, [1,3]thiazolo[5,4-d]pyrimidyl or triazolopiperidinyl.

In certain embodiments, the heteroaryl is substituted with one substituent. In certain embodiments, the heteroaryl is substituted with two substituents. In certain embodiments, the heteroaryl is substituted with three substituents. In certain embodiments, the heteroaryl is substituted with four substituents.

In certain embodiments, the heteroaryl is substituted with halogen. Suitable halogens include, but are not limited to, fluorine, chlorine, bromine or iodine. In certain embodiments, the heteroaryl is substituted with CN. In certain embodiments, the heteroaryl is substituted with C₁-C₆alkyl. Suitable alkyls include, but are not limited to, methyl, ethyl, n-propyl, isopropyl, n-butyl, isobutyl, sec-butyl, tert-butyl, n-pentyl, isopentyl, neopentyl, tert-pentyl, 1-methylbutyl, 2-methylbutyl, 1,2-dimethylpropyl, 1-ethylpropyl, n-hexyl, isohexyl, 1-methylpentyl, 2-methylpentyl, 3-methylpentyl, 1,1-dimethylbutyl, 1,2-dimethylbutyl, 2,2-dimethylbutyl, 1-ethylbutyl, 1,1,2-trimethylpropyl, 1,2,2-trimethylpropyl, 1-ethyl-2-methylpropyl and 1-ethyl-1-methylpropyl. In certain embodiments, the heteroaryl is substituted with alkoxy. Suitable alkoxys include, but are not limited to, methoxy, ethoxy, n-propoxy, isopropoxy and n-butoxy. In certain embodiments, the heteroaryl is substituted with aryl. Suitable aryls include, but are not limited to, phenyl and naphthyl. In certain embodiments, the heteroaryl is substituted with C₃-C₆cycloalkyl. Suitable examples of cycloalkyls include, but are not limited to, cyclopropyl, cyclobutyl, cyclopentyl and cyclohexyl. In certain embodiments, the heteroaryl is substituted with haloC₁-C₆alkyl. Suitable examples of haloalkyls include, but are not limited to, fluoromethyl, difluoromethyl, trifluoromethyl, 2-fluoroethyl, 1,2-difluoroethyl and 2,2-difluoroethyl.

In certain embodiments, R² is taken with R¹ and forms a heteroaryl, wherein the heteroaryl is unsubstituted or substituted with one to four substituents selected from the group consisting of halogen, C₁-C₆alkyl, haloC₁-C₆alkyl, alkoxy, CN, COOC₁-C₆alkyl, COaryl, aryl and heteroaryl, wherein the heteroaryl is unsubstituted or substituted with one to four substituents selected from the group consisting of halogen, CN, C₁-C₆alkyl, alkoxy, aryl, C₃-C₆cycloalkyl and haloC₁-C₆alkyl.

In certain embodiments, the heteroaryl is pyridyl (pyridinyl), oxazolyl, imidazolyl, triazolyl, furyl, triavinyl, thienyl, pyrimidyl, pyridavinyl, indolizinyl, cinnolinyl, phthalazinyl, quinazolinyl, naphthyridinyl, quinoxalinyl, purinyl, benzimidazolyl, quinolyl or isoquinolyl.

In certain embodiments, the heteroaryl is unsubstituted. In certain embodiments, the heteroaryl is substituted with one substituent. In certain embodiments, the heteroaryl is substituted with two substituents. In certain embodiments, the heteroaryl is substituted with three substituents. In certain embodiments, the heteroaryl is substituted with four substituents.

In certain embodiments, the heteroaryl is substituted with CN. In certain embodiments, the heteroaryl is substituted with C₁-C₆alkyl. Suitable alkyls include, but are not limited to, methyl, ethyl, n-propyl, isopropyl, n-butyl, isobutyl, sec-butyl, tert-butyl, n-pentyl, isopentyl, neopentyl, tert-pentyl, 1-methylbutyl, 2-methylbutyl, 1,2-dimethylpropyl, 1-ethylpropyl, n-hexyl, isohexyl, 1-methylpentyl, 2-methylpentyl, 3-methylpentyl, 1,1-dimethylbutyl, 1,2-dimethylbutyl, 2,2-dimethylbutyl, 1-ethylbutyl, 1,1,2-trimethylpropyl, 1,2,2-trimethylpropyl, 1-ethyl-2-methylpropyl and 1-ethyl-1-methylpropyl. In certain embodiments, the heteroaryl is substituted with methyl, ethyl or t-butyl.

In certain embodiments, the heteroaryl is substituted with haloC₁-C₆alkyl. Suitable examples of haloalkyls include, but are not limited to, fluoromethyl, difluoromethyl, trifluoromethyl, 2-fluoroethyl, 1,2-difluoroethyl and 2,2-difluoroethyl. In certain embodiments, the heteroaryl is substituted with halogen. In certain embodiments, the heteroaryl is substituted with fluoromethyl, difluoromethyl, difluoroethyl or trifluoromethyl. Suitable halogens include, but are not limited to, fluorine, chlorine, bromine or iodine. In certain embodiments, the heteroaryl is substituted with fluorine.

In certain embodiments, the heteroaryl is substituted with COOC₁-C₆alkyl. In certain embodiments, the heteroaryl is substituted with

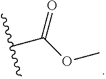 [SMILES: *C(=O)OC]

In certain embodiments, the heteroaryl is substituted with COaryl. In certain embodiments, the heteroaryl is substituted with

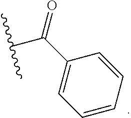 [SMILES: *C(=O)c1ccccc1]

In certain embodiments, the heteroaryl is substituted with aryl. In certain embodiments, the heteroaryl is substituted with phenyl. In certain embodiments, the heteroaryl is substituted with alkoxy. In certain embodiments, the heteroaryl is substituted with methoxy.

In certain embodiments, the heteroaryl is substituted with heteroaryl, wherein the heteroaryl is unsubstituted or substituted. Suitable heteroaryls include, but are not limited to, pyridyl (pyridinyl), oxazolyl, imidazolyl, triazolyl, furyl, triazinyl, thienyl, pyrimidyl, pyridazinyl, indolizinyl, cinnolinyl, phthalazinyl, quinazolinyl, naphthyridinyl, quinoxalinyl, purinyl, benzimidazolyl, quinolyl, and isoquinolyl. In certain embodiments, the heteroaryl is substituted with pyrimidyl, [1,3]thiazolo[5,4-d]pyrimidyl or triazolopiperidinyl.

In certain embodiments, the heteroaryl is substituted with one substituent. In certain embodiments, the heteroaryl is substituted with two substituents. In certain embodiments, the heteroaryl is substituted with three substituents. In certain embodiments, the heteroaryl is substituted with four substituents.

In certain embodiments, the heteroaryl is substituted with halogen. Suitable halogens include, but are not limited to, fluorine, chlorine, bromine or iodine. In certain embodiments, the heteroaryl is substituted with CN. In certain embodiments, the heteroaryl is substituted with C₁-C₆alkyl. Suitable alkyls include, but are not limited to, methyl, ethyl, n-propyl, isopropyl, n-butyl, isobutyl, sec-butyl, tert-butyl, n-pentyl, isopentyl, neopentyl, tert-pentyl, 1-methylbutyl, 2-methylbutyl, 1,2-dimethylpropyl, 1-ethylpropyl, n-hexyl, isohexyl, 1-methylpentyl, 2-methylpentyl, 3-methylpentyl, 1,1-dimethylbutyl, 1,2-dimethylbutyl, 2,2-dimethylbutyl, 1-ethylbutyl, 1,1,2-trimethylpropyl, 1,2,2-trimethylpropyl, 1-ethyl-2-methylpropyl and 1-ethyl-1-methylpropyl. In certain embodiments, the heteroaryl is substituted with alkoxy. Suitable alkoxys include, but are not limited to, methoxy, ethoxy, n-propoxy, isopropoxy and n-butoxy. In certain embodiments, the heteroaryl is substituted with aryl. Suitable aryls include, but are not limited to, phenyl and naphthyl. In certain embodiments, the heteroaryl is substituted with C₃-C₆cycloalkyl. Suitable examples of cycloalkyls include, but are not limited to, cyclopropyl, cyclobutyl, cyclopentyl and cyclohexyl. In certain embodiments, the heteroaryl is substituted with haloC₁-C₆alkyl. Suitable examples of haloalkyls include, but are not limited to, fluoromethyl, difluoromethyl, trifluoromethyl, 2-fluoroethyl, 1,2-difluoroethyl and 2,2-difluoroethyl.

In certain embodiments. R² is taken with R¹ or R¹ and R³ and forms a heterocycloalkyl, wherein the heterocycloalkyl is unsubstituted or substituted with one to four substituents selected from the group consisting of halogen, C₁-C₆alkyl, haloC₁-C₆alkyl, alkoxy, CN, COOC₁-C₆alkyl, COaryl, aryl and heteroaryl, wherein the heteroaryl is unsubstituted or substituted with one to four substituents selected from the group consisting of halogen, CN, C₁-C₆alkyl, alkoxy, aryl, C₃-C₆cycloalkyl and haloC₁-C₆alkyl.

In certain embodiments, R² is taken with R¹ and forms a heterocycloalkyl, wherein the heterocycloalkyl is unsubstituted or substituted with one to four substituents selected from the group consisting of halogen, C₁-C₆alkyl, haloC₁-C₆alkyl, alkoxy, CN, COOC₁-C₆alkyl, COaryl, aryl and heteroaryl, wherein the heteroaryl is unsubstituted or substituted with one to four substituents selected from the group consisting of halogen, CN, C₁-C₆alkyl, alkoxy, aryl, C₃-C₆cycloalkyl and haloC₁-C₆alkyl.

In certain embodiments, R² is taken with R¹ and R³ and forms a heterocycloalkyl, wherein the heterocycloalkyl is unsubstituted or substituted with one to four substituents selected from the group consisting of halogen, C₁-C₆alkyl, haloC₁-C₆alkyl, alkoxy, CN, COOC₁-C₆alkyl, COaryl, aryl and heteroaryl, wherein the heteroaryl is unsubstituted or substituted with one to four substituents selected from the group consisting of halogen, CN, C₁-C₆alkyl, alkoxy, aryl, C₃-C₆cycloalkyl and haloC₁-C₆alkyl.

In certain embodiments, the heterocycloalkyl is

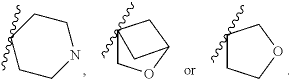

In certain embodiments, the heterocycloalkyl is unsubstituted. In certain embodiments, the heterocycloalkyl is substituted with one substituent. In certain embodiments, the heterocycloalkyl is substituted with two substituents. In certain embodiments, the heterocycloalkyl is substituted with three substituents. In certain embodiments, the heterocycloalkyl is substituted with four substituents.

In certain embodiments, the heterocycloalkyl is substituted with CN. In certain embodiments, the heterocycloalkyl is substituted with C₁-C₆alkyl. Suitable alkyls include, but are not limited to, methyl, ethyl, n-propyl, isopropyl, n-butyl, isobutyl, sec-butyl, tert-butyl, n-pentyl, isopentyl, neopentyl, tert-pentyl, 1-methylbutyl, 2-methylbutyl, 1,2-dimethylpropyl, 1-ethylpropyl, n-hexyl, isohexyl, 1-methylpentyl, 2-methylpentyl, 3-methylpentyl, 1,1-dimethylbutyl, 1,2-dimethylbutyl, 2,2-dimethylbutyl, 1-ethylbutyl, 1,1,2-trimethylpropyl, 1,2,2-trimethylpropyl, 1-ethyl-2-methylpropyl and 1-ethyl-1-methylpropyl. In certain embodiments, the heterocycloalkyl is substituted with methyl, ethyl or t-butyl.

In certain embodiments, the heterocycloalkyl is substituted with haloC₁-C₆alkyl. Suitable examples of haloalkyls include, but are not limited to, fluoromethyl, difluoromethyl, trifluoromethyl, 2-fluoroethyl, 1,2-difluoroethyl and 2,2-difluoroethyl. In certain embodiments, the heterocycloalkyl is substituted with fluoromethyl, difluoromethyl, trifluoromethyl or difluoroethyl. In certain embodiments, the heterocycloalkyl is substituted with halogen. Suitable halogens include, but are not limited to, fluorine, chlorine, bromine or iodine. In certain embodiments, the heterocycloalkyl is substituted with fluorine.

In certain embodiments, the heterocycloalkyl is substituted with COOC₁-C₆alkyl. In certain embodiments, the heterocycloalkyl is substituted with

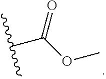 [SMILES: *C(=O)OC]

In certain embodiments, the heterocycloalkyl is substituted with alkoxy. In certain embodiments, the heterocycloalkyl is substituted with methoxy. In certain embodiments, the heterocycloalkyl is substituted with aryl. In certain embodiments, the heterocycloalkyl is substituted with phenyl. In certain embodiments, the heterocycloalkyl is substituted with COaryl. In certain embodiments, the heterocycloalkyl is substituted with

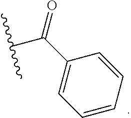 [SMILES: *C(=O)c1ccccc1]

In certain embodiments, the heterocycloalkyl is substituted with heteroaryl, wherein the heteroaryl is unsubstituted or substituted. Suitable heteroaryls include, but are not limited to, pyridyl (pyridinyl), oxazolyl, imidazolyl, triazolyl, furyl, triazinyl, thienyl, pyrimidyl, pyridazinyl, indolizinyl, cinnolinyl, phthalazinyl, quinazolinyl, naphthyridinyl, quinoxalinyl, purinyl, benzimidazolyl, quinolyl, and isoquinolyl. In certain embodiments, the heteroaryl is substituted with pyrimidyl, [1,3]thiazolo[5,4-d]pyrimidyl or triazolopiperidinyl.

In certain embodiments, the heteroaryl is substituted with one substituent. In certain embodiments, the heteroaryl is substituted with two substituents. In certain embodiments, the heteroaryl is substituted with three substituents. In certain embodiments, the heteroaryl is substituted with four substituents.

In certain embodiments, the heteroaryl is substituted with halogen. Suitable halogens include, but are not limited to, fluorine, chlorine, bromine or iodine. In certain embodiments, the heteroaryl is substituted with CN. In certain embodiments, the heteroaryl is substituted with C₁-C₆alkyl. Suitable alkyls include, but are not limited to, methyl, ethyl, n-propyl, isopropyl, n-butyl, isobutyl, sec-butyl, tert-butyl, n-pentyl, isopentyl, neopentyl, tert-pentyl, 1-methylbutyl, 2-methylbutyl, 1,2-dimethylpropyl, 1-ethylpropyl, n-hexyl, isohexyl, 1-methylpentyl, 2-methylpentyl, 3-methylpentyl, 1,1-dimethylbutyl, 1,2-dimethylbutyl, 2,2-dimethylbutyl, 1-ethylbutyl, 1,1,2-trimethylpropyl, 1,2,2-trimethylpropyl, 1-ethyl-2-methylpropyl and 1-ethyl-1-methylpropyl. In certain embodiments, the heteroaryl is substituted with alkoxy. Suitable alkoxys include, but are not limited to, methoxy, ethoxy, n-propoxy, isopropoxy and n-butoxy. In certain embodiments, the heteroaryl is substituted with aryl. Suitable aryls include, but are not limited to, phenyl and naphthyl. In certain embodiments, the heteroaryl is substituted with C₃-C₆cycloalkyl. Suitable examples of cycloalkyls include, but are not limited to, cyclopropyl, cyclobutyl, cyclopentyl and cyclohexyl. In certain embodiments, the heteroaryl is substituted with haloC₁-C₆alkyl. Suitable examples of haloalkyls include, but are not limited to, fluoromethyl, difluoromethyl, trifluoromethyl, 2-fluoroethyl, 1,2-difluoroethyl and 2,2-difluoroethyl.

In certain embodiments, when R² is taken with R¹ or R¹ and R³ and forms a heterocycloalkyl, the heterocycloalkyl is

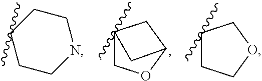

wherein the heterocycloalkyl is unsubstituted or substituted with one to four substituents selected from the group consisting of halogen, C₁-C₆alkyl, haloC₁-C₆alkyl, alkoxy, CN, COOC₁-C₆alkyl, aryl and heteroaryl, wherein the heteroaryl is unsubstituted or substituted with one to four substituents selected from the group consisting of halogen, CN, C₁-C₆alkyl and haloC₁-C₆alkyl.

In certain embodiments, wherein when the heterocycloalkyl is

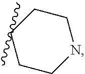

and wherein the heterocycloalkyl is substituted with heteroaryl, the heteroaryl is

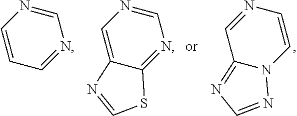

wherein the heteroaryl is unsubstituted or substituted with CN.

With regard to the compounds described herein, R³ is OH, C₁-C₆alkylOH, CN, C₁-C₆alkylCN, C₁-C₆alkyl, C₃-C₆cycloalkyl, haloC₁-C₆alkyl, halogen, C₁-C₆alkylOhaloC₁-C₆alkyl, alkoxy, CONHC₁-C₆alkylaryl, NHC₁-C₆alkylaryl, aryl, or C₁-C₆alkylaryl, wherein the NHC₁-C₆alkylaryl, aryl, CONHC₁-C₆alkylaryl, aryl, or C₁-C₆alkylaryl is unsubstituted or substituted with one to four substituents selected from the group consisting of halogen, C₁-C₆alkyl, alkoxy and haloC₁-C₆alkyl, or wherein when R³ is taken with R² or R¹ and R², forms a C₃-C₁₀cycloalkyl, aryl, heteroaryl or heterocycloalkyl, wherein the C₃-C₁₀cycloalkyl, aryl, heteroaryl or heterocycloalkyl is unsubstituted or substituted with one to four substituents selected from the group consisting of halogen, C₁-C₆alkyl, haloC₁-C₆alkyl, alkoxy, CN, COOC₁-C₆alkyl, COaryl and heteroaryl, wherein the heteroaryl is unsubstituted or substituted with one to four substituents selected from the group consisting of halogen, CN, C₁-C₆alkyl, alkoxy, aryl, C₃-C₆cycloalkyl and haloC₁-C₆alkyl.

In certain embodiments, R³ is OH. In certain embodiments, R³ is C₁-C₆alkylOH. Suitable alcohols include, but are not limited to, methanol, ethanol, propanol and butanol.

In certain embodiments, R³ is CN. In certain embodiments, R³ is C₁-C₆alkylCN. In certain embodiments, R³ is

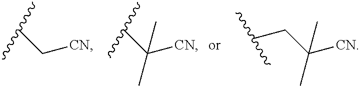

In certain embodiments, R³ is

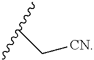 [SMILES: *CC#N]

In certain embodiments, R³ is C₁-C₆alkyl. Suitable alkyls include, but are not limited to, methyl, ethyl, n-propyl, isopropyl, n-butyl, isobutyl, sec-butyl, tert-butyl, n-pentyl, isopentyl, neopentyl, tert-pentyl, 1-methylbutyl, 2-methylbutyl, 1,2-dimethylpropyl, 1-ethylpropyl, n-hexyl, isohexyl, 1-methylpentyl, 2-methylpentyl, 3-methylpentyl, 1,1-dimethylbutyl, 1,2-dimethylbutyl, 2,2-dimethylbutyl, 1-ethylbutyl, 1,1,2-trimethylpropyl, 1,2,2-trimethylpropyl, 1-ethyl-2-methylpropyl and 1-ethyl-1-methylpropyl. In certain embodiments, R³ is methyl, ethyl and t-butyl. In certain embodiments, R³ is C₃-C₆cycloalkyl. Suitable cycloalkyls include, but are not limited to, cyclopropyl, cyclobutyl, cyclopentyl and cyclohexyl. In certain embodiments, R³ is cyclopropyl.

In certain embodiments, R³ is haloC₁-C₆alkyl. Suitable examples of haloalkyls include, but are not limited to, fluoromethyl, difluoromethyl, trifluoromethyl, 2-fluoroethyl, 1,2-difluoroethyl and 2,2-difluoroethyl. In certain embodiments, R³ is difluoromethyl. In certain embodiments, R³ is trifluoromethyl. In certain embodiments, R³ is difluoromethyl or trifluoromethyl. In certain embodiments, R³ is fluoromethyl, difluoromethyl, difluoroethyl or trifluoromethyl. In certain embodiments, R³ is halogen. Suitable halogens include, but are not limited to, fluorine, chlorine, bromine or iodine. In certain embodiments, R³ is fluorine or chlorine. In certain embodiments, R³ is fluorine.

In certain embodiments, R³ is C₁-C₆alkylOhaloC₁-C₆alkyl. In certain embodiments, R³ is CH₂OCF₃. In certain embodiments, R³ is alkoxy. Suitable alkoxy's include, but are not limited to, methoxy, ethoxy, n-propoxy, isopropoxy and n-butoxy. In certain embodiments, R³ is methoxy. In certain embodiments, R³ is CONHC₁-C₆alkylaryl. In certain embodiments, R³ is CONHCH₂phenyl. In certain embodiments, R³ is NHC₁-C₆alkylaryl. In certain embodiments, R³ is NHCH₂phenyl. In certain embodiments, R³ is aryl. Suitable aryls include, but are not limited to, phenyl and naphthyl. In certain embodiments, R³ is C₁-C₆alkylaryl. In certain embodiments, R³ is CH₂phenyl.

In certain embodiments, the NHC₁-C₆alkylaryl, CONHC₁-C₆alkylaryl, aryl, or C₁-C₆alkylaryl is unsubstituted or substituted with one to four substituents selected from the group consisting of halogen, C₁-C₆alkyl, alkoxy and haloC₁-C₆alkyl. In certain embodiments, the NHC₁-C₆alkylaryl, CONHC₁-C₆alkylaryl, aryl, or C₁-C₆alkylaryl is substituted with one substituent selected from the group consisting of halogen, C₁-C₆alkyl, alkoxy and haloC₁-C₆alkyl. In certain embodiments, the NHC₁-C₆alkylaryl, CONHC₁-C₆alkylaryl, aryl, or C₁-C₆alkylaryl is two substituents selected from the group consisting of halogen, C₁-C₆alkyl, alkoxy and haloC₁-C₆alkyl. In certain embodiments, the NHC₁-C₆alkylaryl, CONHC₁-C₆alkylaryl, aryl, or C₁-C₆alkylaryl is substituted with three substituents selected from the group consisting of halogen, C₁-C₆alkyl, alkoxy and haloC₁-C₆alkyl. In certain embodiments, the NHC₁-C₆alkylaryl, CONHC₁-C₆alkylaryl, aryl, or C₁-C₆alkylaryl is substituted with four substituents selected from the group consisting of halogen, C₁-C₆alkyl, alkoxy and haloC₁-C₆alkyl.

In certain embodiments, the NHC₁-C₆alkylaryl, CONHC₁-C₆alkylaryl, aryl, or C₁-C₆alkylaryl is substituted with halogen. Suitable halogens include, but are not limited to, fluorine, chlorine, bromine or iodine. In certain embodiments, the NHC₁-C₆alkylaryl, CONHC₁-C₆alkylaryl, aryl, or C₁-C₆alkylaryl is substituted with chlorine. In certain embodiments, the NHC₁-C₆alkylaryl, CONHC₁-C₆alkylaryl, aryl, or C₁-C₆alkylaryl is substituted with C₁-C₆alkyl. Suitable alkyls include, but are not limited to, methyl, ethyl, n-propyl, isopropyl, n-butyl, isobutyl, sec-butyl, tert-butyl, n-pentyl, isopentyl, neopentyl, tert-pentyl, 1-methylbutyl, 2-methylbutyl, 1,2-dimethylpropyl, 1-ethylpropyl, n-hexyl, isohexyl, 1-methylpentyl, 2-methylpentyl, 3-methylpentyl, 1,1-dimethylbutyl, 1,2-dimethylbutyl, 2,2-dimethylbutyl, 1-ethylbutyl, 1,1,2-trimethylpropyl, 1,2,2-trimethylpropyl, 1-ethyl-2-methylpropyl and 1-ethyl-1-methylpropyl. In certain embodiments, the NHC₁-C₆alkylaryl, CONHC₁-C₆alkylaryl, aryl, or C₁-C₆alkylaryl is substituted with alkoxy. In certain embodiments, the NHC₁-C₆alkylaryl, CONHC₁-C₆alkylaryl, aryl, or C₁-C₆alkylaryl is substituted with methoxy. In certain embodiments, the NHC₁-C₆alkylaryl, CONHC₁-C₆alkylaryl, aryl, or C₁-C₆alkylaryl is substituted with haloC₁-C₆alkyl.

In certain embodiments, R³ is methyl, ethyl, t-butyl, fluoromethyl, difluoromethyl, fluorine, difluoroethyl, trifluoromethyl, CH₂OCH₃, or cyclopropyl.

In certain embodiments, R³ is taken with R² or R² and R¹ and forms a C₃-C₁₀cycloalkyl, aryl, heteroaryl or heterocycloalkyl, wherein the C₃-C₁₀cycloalkyl, aryl, heteroaryl or heterocycloalkyl is unsubstituted or substituted with one to four substituents selected from the group consisting of halogen, C₁-C₆alkyl, haloC₁-C₆alkyl, alkoxy, CN, COOC₁-C₆alkyl, COaryl, aryl and heteroaryl, wherein the heteroaryl is unsubstituted or substituted with one to four substituents selected from the group consisting of halogen, CN, C₁-C₆alkyl, alkoxy, aryl, C₃-C₆cycloalkyl and haloC₁-C₆alkyl.

In certain embodiments, R³ is taken with R² and forms a C₃-C₁₀cycloalkyl or heterocycloalkyl, wherein the C₃-C₁₀cycloalkyl or heterocycloalkyl is unsubstituted or substituted with one to four substituents selected from the group consisting of halogen, C₁-C₆alkyl, haloC₁-C₆alkyl, alkoxy, CN, COOC₁-C₆alkyl, COaryl, aryl and heteroaryl, wherein the heteroaryl is unsubstituted or substituted with one to four substituents selected from the group consisting of halogen, CN, C₁-C₆alkyl, alkoxy, aryl, C₃-C₆cycloalkyl and haloC₁-C₆alkyl.

In certain embodiments, R³ is taken with R² and R¹ and forms a C₃-C₁₀cycloalkyl or heterocycloalkyl, wherein the C₃-C₁₀cycloalkyl or heterocycloalkyl is unsubstituted or substituted with one to four substituents selected from the group consisting of halogen, C₁-C₆alkyl, haloC₁-C₆alkyl, alkoxy, CN, COOC₁-C₆alkyl, COaryl, aryl and heteroaryl, wherein the heteroaryl is unsubstituted or substituted with one to four substituents selected from the group consisting of halogen, CN, C₁-C₆alkyl, alkoxy, aryl, C₃-C₆cycloalkyl and haloC₁-C₆alkyl.

In certain embodiments, R³ is taken with R² or R² and R¹ and forms a C₃-C₁₀cycloalkyl, wherein the C₃-C₁₀cycloalkyl is unsubstituted or substituted with one to four substituents selected from the group consisting of halogen, C₁-C₆alkyl, haloC₁-C₆alkyl, alkoxy, CN, COOC₁-C₆alkyl, COaryl, aryl and heteroaryl, wherein the heteroaryl is unsubstituted or substituted with one to four substituents selected from the group consisting of halogen, CN, C₁-C₆alkyl, alkoxy, aryl, C₃-C₆cycloalkyl and haloC₁-C₆alkyl.

In certain embodiments, R³ is taken with R² and R¹ and forms a C₃-C₁₀cycloalkyl, wherein the C₃-C₁₀cycloalkyl is

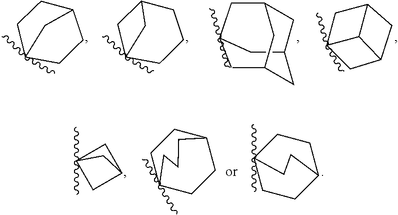

In certain embodiments, R³ is taken with R² or R¹ and forms a C₃-C₁₀cycloalkyl, wherein the C₃-C₁₀cycloalkyl is

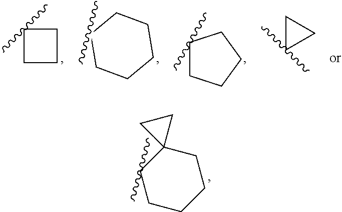

In certain embodiments, the C₃-C₁₀cycloalkyl is

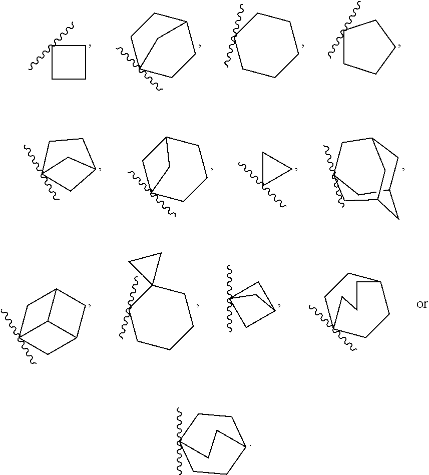

In certain embodiments, the C₃-C₁₀cycloalkyl is unsubstituted. In certain embodiments, the C₃-C₁₀cycloalkyl is substituted with one substituent. In certain embodiments, the C₃-C₁₀cycloalkyl is substituted with two substituents. In certain embodiments, the C₃-C₁₀cycloalkyl is substituted with three substituents. In certain embodiments, the C₃-C₁₀cycloalkyl is substituted with four substituents.

In certain embodiments, the C₃-C₁₀cycloalkyl is substituted with CN. In certain embodiments, the C₃-C₁₀cycloalkyl is substituted with C₁-C₆alkyl. Suitable alkyls include, but are not limited to, methyl, ethyl, n-propyl, isopropyl, n-butyl, isobutyl, sec-butyl, tert-butyl, n-pentyl, isopentyl, neopentyl, tert-pentyl, 1-methylbutyl, 2-methylbutyl, 1,2-dimethylpropyl, 1-ethylpropyl, n-hexyl, isohexyl, 1-methylpentyl, 2-methylpentyl, 3-methylpentyl, 1,1-dimethylbutyl, 1,2-dimethylbutyl, 2,2-dimethylbutyl, 1-ethylbutyl, 1,1,2-trimethylpropyl, 1,2,2-trimethylpropyl, 1-ethyl-2-methylpropyl and 1-ethyl-1-methylpropyl. In certain embodiments, the C₃-C₁₀cycloalkyl is substituted with methyl, ethyl or t-butyl.

In certain embodiments, the C₃-C₁₀cycloalkyl is substituted with haloC₁-C₆alkyl. Suitable examples of haloalkyls include, but are not limited to, fluoromethyl, difluoromethyl, trifluoromethyl, 2-fluoroethyl, 1,2-difluoroethyl and 2,2-difluoroethyl. In certain embodiments, the C₃-C₁₀cycloalkyl is substituted with fluoromethyl, difluoromethyl, trifluoromethyl, difluoroethyl. In certain embodiments, the C₃-C₁₀cycloalkyl is substituted with halogen. Suitable halogens include, but are not limited to, fluorine, chlorine, bromine or iodine. In certain embodiments, the C₃-C₁₀cycloalkyl is substituted with fluorine and chlorine.

In certain embodiments, the C₃-C₁₀cycloalkyl is substituted with COOC₁-C₆alkyl. In certain embodiments, the C₃-C₁₀cycloalkyl is substituted with

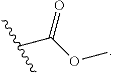 [SMILES: *C(=O)OC]

In certain embodiments, the C₃-C₁₀cycloalkyl is substituted with alkoxy. In certain embodiments, the C₃-C₁₀cycloalkyl is substituted with methoxy. In certain embodiments, the C₃-C₁₀cycloalkyl is substituted with COaryl. In certain embodiments, the C₃-C₁₀cycloalkyl is substituted with

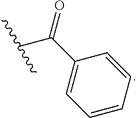 [SMILES: *C(=O)c1ccccc1]

In certain embodiments, the C₃-C₁₀cycloalkyl is substituted with aryl. In certain embodiments, the C₃-C₁₀cycloalkyl is substituted with phenyl.

In certain embodiments, the C₃-C₁₀cycloalkyl is substituted with heteroaryl, wherein the heteroaryl is unsubstituted or substituted. Suitable heteroaryls include, but are not limited to, pyridyl (pyridinyl), oxazolyl, imidazolyl, triazolyl, furyl, triazinyl, thienyl, pyrimidyl, pyridazinyl, indolizinyl, cinnolinyl, phthalazinyl, quinazolinyl, naphthyridinyl, quinoxalinyl, purinyl, benzimidazolyl, quinolyl, and isoquinolyl. In certain embodiments, the heteroaryl is pyrimidinyl, [1,3]thiazolo[5,4-d]pyrimidyl or triazolopiperidinyl.

In certain embodiments, the heteroaryl is substituted with one substituent. In certain embodiments, the heteroaryl is substituted with two substituents. In certain embodiments, the heteroaryl is substituted with three substituents. In certain embodiments, the heteroaryl is substituted with four substituents.

In certain embodiments, the heteroaryl is substituted with halogen. Suitable halogens include, but are not limited to, fluorine, chlorine, bromine or iodine. In certain embodiments, the heteroaryl is substituted with chlorine or fluorine. In certain embodiments, the heteroaryl is substituted with CN. In certain embodiments, the heteroaryl is substituted with C₁-C₆alkyl. Suitable alkyls include, but are not limited to, methyl, ethyl, n-propyl, isopropyl, n-butyl, isobutyl, sec-butyl, tert-butyl, n-pentyl, isopentyl, neopentyl, tert-pentyl, 1-methylbutyl, 2-methylbutyl, 1,2-dimethylpropyl, 1-ethylpropyl, n-hexyl, isohexyl, 1-methylpentyl, 2-methylpentyl, 3-methylpentyl, 1,1-dimethylbutyl, 1,2-dimethylbutyl, 2,2-dimethylbutyl, 1-ethylbutyl, 1,1,2-trimethylpropyl, 1,2,2-trimethylpropyl, 1-ethyl-2-methylpropyl and 1-ethyl-1-methylpropyl. In certain embodiments, the heteroaryl is substituted with methyl.

In certain embodiments, the heteroaryl is substituted with alkoxy. Suitable alkoxys include, but are not limited to, methoxy, ethoxy, n-propoxy, isopropoxy and n-butoxy. In certain embodiments, the heteroaryl is substituted with methoxy. In certain embodiments, the heteroaryl is substituted with aryl. Suitable aryls include, but are not limited to, phenyl and naphthyl. In certain embodiments, the heteroaryl is substituted with C₃-C₆cycloalkyl. Suitable examples of cycloalkyls include, but are not limited to, cyclopropyl, cyclobutyl, cyclopentyl and cyclohexyl. In certain embodiments, the heteroaryl is substituted with haloC₁-C₆alkyl. Suitable examples of haloalkyls include, but are not limited to, fluoromethyl, difluoromethyl, trifluoromethyl, 2-fluoroethyl, 1,2-difluoroethyl and 2,2-difluoroethyl. In certain embodiments, the heteroaryl is substituted with difluoromethyl and trifluoromethyl.

In certain embodiments, R³ is taken with R¹ or R¹ and R² and forms a C₃-C₁₀cycloalkyl, the C₃-C₁₀cycloalkyl is

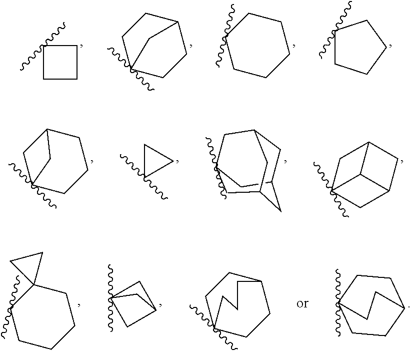

wherein the C₃-C₁₀cycloalkyl is unsubstituted or substituted with one to four substituents selected from the group consisting of halogen, C₁-C₆alkyl, haloC₁-C₆alkyl, alkoxy, CN and COOC₁-C₆alkyl.

In certain embodiments, R³ is taken with R² and forms an aryl, wherein the aryl is unsubstituted or substituted with one to four substituents selected from the group consisting of halogen, C₁-C₆alkyl, haloC₁-C₆alkyl, alkoxy, CN, COOC₁-C₆alkyl, COaryl, aryl and heteroaryl, wherein the heteroaryl is unsubstituted or substituted with one to four substituents selected from the group consisting of halogen, CN, C₁-C₆alkyl, alkoxy, aryl, C₃-C₆cycloalkyl and haloC₁-C₆alkyl.

In certain embodiments, the aryl is

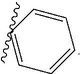

In certain embodiments, the aryl is unsubstituted. In certain embodiments, the aryl is substituted with one substituent. In certain embodiments, the aryl is substituted with two substituents. In certain embodiments, the aryl is substituted with three substituents. In certain embodiments, the aryl is substituted with four substituents.

In certain embodiments, the aryl is substituted with CN. In certain embodiments, the aryl is substituted with C₁-C₆alkyl. Suitable alkyls include, but are not limited to, methyl, ethyl, n-propyl, isopropyl, n-butyl, isobutyl, sec-butyl, tert-butyl, n-pentyl, isopentyl, neopentyl, tert-pentyl, 1-methylbutyl, 2-methylbutyl, 1,2-dimethylpropyl, 1-ethylpropyl, n-hexyl, isohexyl, 1-methylpentyl, 2-methylpentyl, 3-methylpentyl, 1,1-dimethylbutyl, 1,2-dimethylbutyl, 2,2-dimethylbutyl, 1-ethylbutyl, 1,1,2-trimethylpropyl, 1,2,2-trimethylpropyl, 1-ethyl-2-methylpropyl and 1-ethyl-1-methylpropyl. In certain embodiments, the aryl is substituted with methyl, ethyl or t-butyl.

In certain embodiments, the aryl is substituted with haloC₁-C₆alkyl. Suitable examples of haloalkyls include, but are not limited to, fluoromethyl, difluoromethyl, trifluoromethyl, 2-fluoroethyl, 1,2-difluoroethyl and 2,2-difluoroethyl. In certain embodiments, the aryl is substituted with fluoromethyl, difluoromethyl, difluoroethyl and trifluoromethyl. In certain embodiments, the aryl is substituted with halogen. Suitable halogens include, but are not limited to, fluorine, chlorine, bromine or iodine. In certain embodiments, the aryl is substituted with chlorine or fluorine.

In certain embodiments, the aryl is substituted with COOC₁-C₆alkyl. In certain embodiments, the aryl is substituted with

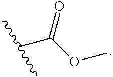 [SMILES: *C(=O)OC]

In certain embodiments, the aryl is substituted with alkoxy. In certain embodiments, the aryl is substituted with methoxy. In certain embodiments, the aryl is substituted with aryl. In certain embodiments, the aryl is substituted with phenyl. In certain embodiments, the aryl is substituted with COaryl. In certain embodiments, the aryl is substituted with

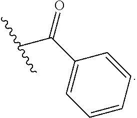 [SMILES: *C(=O)c1ccccc1]

In certain embodiments, the aryl is substituted with heteroaryl, wherein the heteroaryl is unsubstituted or substituted. Suitable heteroaryls include, but are not limited to, pyridyl (pyridinyl), oxazolyl, imidazolyl, triazolyl, furyl, triazinyl, thienyl, pyrimidyl, pyridazinyl, indolizinyl, cinnolinyl, phthalazinyl, quinazolinyl, naphthyridinyl, quinoxalinyl, purinyl, benzimidazolyl, quinolyl, and isoquinolyl. In certain embodiments, the aryl is substituted with pyrimidinyl, [1,3]thiazolo[5,4-d]pyrimidyl, or triazolopiperidinyl

In certain embodiments, the heteroaryl is substituted with one substituent. In certain embodiments, the heteroaryl is substituted with two substituents. In certain embodiments, the heteroaryl is substituted with three substituents. In certain embodiments, the heteroaryl is substituted with four substituents.

In certain embodiments, the heteroaryl is substituted with halogen. Suitable halogens include, but are not limited to, fluorine, chlorine, bromine or iodine. In certain embodiments, the heteroaryl is substituted with CN. In certain embodiments, the heteroaryl is substituted with C₁-C₆alkyl. Suitable alkyls include, but are not limited to, methyl, ethyl, n-propyl, isopropyl, n-butyl, isobutyl, sec-butyl, tert-butyl, n-pentyl, isopentyl, neopentyl, tert-pentyl, 1-methylbutyl, 2-methylbutyl, 1,2-dimethylpropyl, 1-ethylpropyl, n-hexyl, isohexyl, 1-methylpentyl, 2-methylpentyl, 3-methylpentyl, 1,1-dimethylbutyl, 1,2-dimethylbutyl, 2,2-dimethylbutyl, 1-ethylbutyl, 1,1,2-trimethylpropyl, 1,2,2-trimethylpropyl, 1-ethyl-2-methylpropyl and 1-ethyl-1-methylpropyl. In certain embodiments, the heteroaryl is substituted with alkoxy. Suitable alkoxys include, but are not limited to, methoxy, ethoxy, n-propoxy, isopropoxy and n-butoxy. In certain embodiments, the heteroaryl is substituted with aryl. Suitable aryls include, but are not limited to, phenyl and naphthyl. In certain embodiments, the heteroaryl is substituted with C₃-C₆cycloalkyl. Suitable examples of cycloalkyls include, but are not limited to, cyclopropyl, cyclobutyl, cyclopentyl and cyclohexyl. In certain embodiments, the heteroaryl is substituted with haloC₁-C₆alkyl. Suitable examples of haloalkyls include, but are not limited to, fluoromethyl, difluoromethyl, trifluoromethyl, 2-fluoroethyl, 1,2-difluoroethyl and 2,2-difluoroethyl.

In certain embodiments, R³ is taken with R² and forms a heteroaryl, wherein the heteroaryl is unsubstituted or substituted with one to four substituents selected from the group consisting of halogen, C₁-C₆alkyl, haloC₁-C₆alkyl, alkoxy, CN, COOC₁-C₆alkyl, COaryl, aryl and heteroaryl, wherein the heteroaryl is unsubstituted or substituted with one to four substituents selected from the group consisting of halogen, CN, C₁-C₆alkyl, alkoxy, aryl, C₃-C₆cycloalkyl and haloC₁-C₆alkyl.

In certain embodiments, the heteroaryl is pyridyl (pyridinyl), oxazolyl, imidazolyl, triazolyl, furyl, triazinyl, thienyl, pyrimidyl, pyridazinyl, indolizinyl, cinnolinyl, phthalazinyl, quinazolinyl, naphthyridinyl, quinoxalinyl, purinyl, benzimidazolyl, quinolyl or isoquinolyl. In certain embodiments, the heteroaryl is piperidinyl, 2-oxabicyclo[2.1.1.]hexanyl or oxolanyl.

In certain embodiments, the heteroaryl is unsubstituted. In certain embodiments, the heteroaryl is substituted with one substituent. In certain embodiments, the heteroaryl is substituted with two substituents. In certain embodiments, the heteroaryl is substituted with three substituents. In certain embodiments, the heteroaryl is substituted with four substituents.

In certain embodiments, the heteroaryl is substituted with CN. In certain embodiments, the heteroaryl is substituted with C₁-C₆alkyl. Suitable alkyls include, but are not limited to, methyl, ethyl, n-propyl, isopropyl, n-butyl, isobutyl, sec-butyl, tert-butyl, n-pentyl, isopentyl, neopentyl, tert-pentyl, 1-methylbutyl, 2-methylbutyl, 1,2-dimethylpropyl, 1-ethylpropyl, n-hexyl, isohexyl, 1-methylpentyl, 2-methylpentyl, 3-methylpentyl, 1,1-dimethylbutyl, 1,2-dimethylbutyl, 2,2-dimethylbutyl, 1-ethylbutyl, 1,1,2-trimethylpropyl, 1,2,2-trimethylpropyl, 1-ethyl-2-methylpropyl and 1-ethyl-1-methylpropyl. In certain embodiments, the heteroaryl is substituted with methyl. In certain embodiments, the heteroaryl is substituted with haloC₁-C₆alkyl. Suitable examples of haloalkyls include, but are not limited to, fluoromethyl, difluoromethyl, trifluoromethyl, 2-fluoroethyl, 1,2-difluoroethyl and 2,2-difluoroethyl. In certain embodiments, the heteroaryl is substituted with difluoromethyl or trifluoromethyl. In certain embodiments, the heteroaryl is substituted with halogen. Suitable halogens include, but are not limited to, fluorine, chlorine, bromine or iodine. In certain embodiments, the heteroaryl is substituted with fluorine or chlorine.

In certain embodiments, the heteroaryl is substituted with COOC₁-C₆alkyl. In certain embodiments, the heteroaryl is substituted with

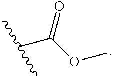 [SMILES: *C(=O)OC]

In certain embodiments, the heteroaryl is substituted with alkoxy. In certain embodiments, the heteroaryl is substituted with methoxy. In certain embodiments, the heteroaryl is substituted with aryl. In certain embodiments, the heteroaryl is substituted with phenyl. In certain embodiments, the heteroaryl is substituted with COaryl. In certain embodiments, the heteroaryl is substituted with

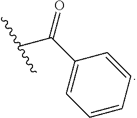 [SMILES: *C(=O)c1ccccc1]

In certain embodiments, the heteroaryl is substituted with heteroaryl, wherein the heteroaryl is unsubstituted or substituted. Suitable heteroaryls include, but are not limited to, pyridyl (pyridinyl), oxazolyl, imidazolyl, triazolyl, furyl, triazinyl, thienyl, pyrimidyl, pyridazinyl, indolizinyl, cinnolinyl, phthalazinyl, quinazolinyl, naphthyridinyl, quinoxalinyl, purinyl, benzimidazolyl, quinolyl, and isoquinolyl. In certain embodiments, the heteroaryl is substituted with pyrimidinyl, [1,3]thiazolo[5,4-d]pyrimidyl or triazolopiperidinyl.

In certain embodiments, the heteroaryl is substituted with one substituent. In certain embodiments, the heteroaryl is substituted with two substituents. In certain embodiments, the heteroaryl is substituted with three substituents. In certain embodiments, the heteroaryl is substituted with four substituents.

In certain embodiments, the heteroaryl is substituted with halogen. Suitable halogens include, but are not limited to, fluorine, chlorine, bromine or iodine. In certain embodiments, the heteroaryl is substituted with CN. In certain embodiments, the heteroaryl is substituted with C₁-C₆alkyl. Suitable alkyls include, but are not limited to, methyl, ethyl, n-propyl, isopropyl, n-butyl, isobutyl, sec-butyl, tert-butyl, n-pentyl, isopentyl, neopentyl, tert-pentyl, 1-methylbutyl, 2-methylbutyl, 1,2-dimethylpropyl, 1-ethylpropyl, n-hexyl, isohexyl, 1-methylpentyl, 2-methylpentyl, 3-methylpentyl, 1,1-dimethylbutyl, 1,2-dimethylbutyl, 2,2-dimethylbutyl, 1-ethylbutyl, 1,1,2-trimethylpropyl, 1,2,2-trimethylpropyl, 1-ethyl-2-methylpropyl and 1-ethyl-1-methylpropyl. In certain embodiments, the heteroaryl is substituted with alkoxy. Suitable alkoxys include, but are not limited to, methoxy, ethoxy, n-propoxy, isopropoxy and n-butoxy. In certain embodiments, the heteroaryl is substituted with aryl. Suitable aryls include, but are not limited to, phenyl and naphthyl. In certain embodiments, the heteroaryl is substituted with C₃-C₆cycloalkyl. Suitable examples of cycloalkyls include, but are not limited to, cyclopropyl, cyclobutyl, cyclopentyl and cyclohexyl. In certain embodiments, the heteroaryl is substituted with haloC₁-C₆alkyl. Suitable examples of haloalkyls include, but are not limited to, fluoromethyl, difluoromethyl, trifluoromethyl, 2-fluoroethyl, 1,2-difluoroethyl and 2,2-difluoroethyl.

In certain embodiments, R³ is taken with R² or R² and R¹ and forms a heterocycloalkyl, wherein the heterocycloalkyl is unsubstituted or substituted with one to four substituents selected from the group consisting of halogen, C₁-C₆alkyl, haloC₁-C₆alkyl, alkoxy, CN, COOC₁-C₆alkyl, COaryl, aryl and heteroaryl, wherein the heteroaryl is unsubstituted or substituted with one to four substituents selected from the group consisting of halogen, CN, C₁-C₆alkyl, alkoxy, aryl, C₃-C₆cycloalkyl and haloC₁-C₆alkyl.

In certain embodiments, R³ is taken with R² and forms a heterocycloalkyl, wherein the heterocycloalkyl is unsubstituted or substituted with one to four substituents selected from the group consisting of halogen, C₁-C₆alkyl, haloC₁-C₆alkyl, alkoxy, CN, COOC₁-C₆alkyl, COaryl, aryl and heteroaryl, wherein the heteroaryl is unsubstituted or substituted with one to four substituents selected from the group consisting of halogen, CN, C₁-C₆alkyl, alkoxy, aryl, C₃-C₆cycloalkyl and haloC₁-C₆alkyl.

In certain embodiments, R³ is taken with R² and R¹ and forms a heterocycloalkyl, wherein the heterocycloalkyl is unsubstituted or substituted with one to four substituents selected from the group consisting of halogen, C₁-C₆alkyl, haloC₁-C₆alkyl, alkoxy, CN, COOC₁-C₆alkyl, COaryl, aryl and heteroaryl, wherein the heteroaryl is unsubstituted or substituted with one to four substituents selected from the group consisting of halogen, CN, C₁-C₆alkyl, alkoxy, aryl, C₃-C₆cycloalkyl and haloC₁-C₆alkyl.

In certain embodiments, the heterocycloalkyl is

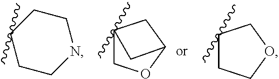

In certain embodiments, the heterocycloalkyl is unsubstituted. In certain embodiments, the heterocycloalkyl is substituted with one substituent. In certain embodiments, the heterocycloalkyl is substituted with two substituents. In certain embodiments, the heterocycloalkyl is substituted with three substituents. In certain embodiments, the heterocycloalkyl is substituted with four substituents.

In certain embodiments, the heterocycloalkyl is substituted with CN. In certain embodiments, the heterocycloalkyl is substituted with C₁-C₆alkyl. Suitable alkyls include, but are not limited to, methyl, ethyl, n-propyl, isopropyl, n-butyl, isobutyl, sec-butyl, tert-butyl, n-pentyl, isopentyl, neopentyl, tert-pentyl, 1-methylbutyl, 2-methylbutyl, 1,2-dimethylpropyl, 1-ethylpropyl, n-hexyl, isohexyl, 1-methylpentyl, 2-methylpentyl, 3-methylpentyl, 1,1-dimethylbutyl, 1,2-dimethylbutyl, 2,2-dimethylbutyl, 1-ethylbutyl, 1,1,2-trimethylpropyl, 1,2,2-trimethylpropyl, 1-ethyl-2-methylpropyl and 1-ethyl-1-methylpropyl. In certain embodiments, the heterocycloalkyl is substituted with methyl, ethyl or t-butyl.

In certain embodiments, the heterocycloalkyl is substituted with haloC₁-C₆alkyl. Suitable examples of haloalkyls include, but are not limited to, fluoromethyl, difluoromethyl, trifluoromethyl, 2-fluoroethyl, 1,2-difluoroethyl and 2,2-difluoroethyl. In certain embodiments, the heterocycloalkyl is substituted with difluoromethyl or trifluoromethyl. In certain embodiments, the heterocycloalkyl is substituted with halogen. Suitable halogens include, but are not limited to, fluorine, chlorine, bromine or iodine. In certain embodiments, the heterocycloalkyl is substituted with fluorine or chlorine.

In certain embodiments, the heterocycloalkyl is substituted with COOC₁-C₆alkyl. In certain embodiments, the heterocycloalkyl is substituted with

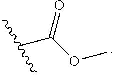 [SMILES: *C(=O)OC]

In certain embodiments, the heterocycloalkyl is substituted with alkoxy. In certain embodiments, the heterocycloalkyl is substituted with methoxy. In certain embodiments, the heterocycloalkyl is substituted with aryl. In certain embodiments, the heterocycloalkyl is substituted with phenyl. In certain embodiments, the heterocycloalkyl is substituted with COaryl. In certain embodiments, the heterocycloalkyl is substituted with

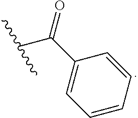 [SMILES: *C(=O)c1ccccc1]

In certain embodiments, the heterocycloalkyl is substituted with heteroaryl, wherein the heteroaryl is unsubstituted or substituted. Suitable heteroaryls include, but are not limited to, pyridyl (pyridinyl), oxazolyl, imidazolyl, triazolyl, furyl, triazinyl, thienyl, pyrimidyl, pyridazinyl, indolizinyl, cinnolinyl, phthalazinyl, quinazolinyl, naphthyridinyl, quinoxalinyl, purinyl, benzimidazolyl, quinolyl, and isoquinolyl. In certain embodiments, the heterocycloalkyl is substituted with pyrimidinyl, [1,3]thiazolo[5,4-d]pyrimidyl or triazolopiperidinyl.

In certain embodiments, the heteroaryl is substituted with one substituent. In certain embodiments, the heteroaryl is substituted with two substituents. In certain embodiments, the heteroaryl is substituted with three substituents. In certain embodiments, the heteroaryl is substituted with four substituents.

In certain embodiments, the heteroaryl is substituted with halogen. Suitable halogens include, but are not limited to, fluorine, chlorine, bromine or iodine. In certain embodiments, the heteroaryl is substituted with CN. In certain embodiments, the heteroaryl is substituted with C₁-C₆alkyl. Suitable alkyls include, but are not limited to, methyl, ethyl, n-propyl, isopropyl, n-butyl, isobutyl, sec-butyl, tert-butyl, n-pentyl, isopentyl, neopentyl, tert-pentyl, 1-methylbutyl, 2-methylbutyl, 1,2-dimethylpropyl, 1-ethylpropyl, n-hexyl, isohexyl, 1-methylpentyl, 2-methylpentyl, 3-methylpentyl, 1,1-dimethylbutyl, 1,2-dimethylbutyl, 2,2-dimethylbutyl, 1-ethylbutyl, 1,1,2-trimethylpropyl, 1,2,2-trimethylpropyl, 1-ethyl-2-methylpropyl and 1-ethyl-1-methylpropyl. In certain embodiments, the heteroaryl is substituted with alkoxy. Suitable alkoxys include, but are not limited to, methoxy, ethoxy, n-propoxy, isopropoxy and n-butoxy. In certain embodiments, the heteroaryl is substituted with aryl. Suitable aryls include, but are not limited to, phenyl and naphthyl. In certain embodiments, the heteroaryl is substituted with C₃-C₆cycloalkyl. Suitable examples of cycloalkyls include, but are not limited to, cyclopropyl, cyclobutyl, cyclopentyl and cyclohexyl. In certain embodiments, the heteroaryl is substituted with haloC₁-C₆alkyl. Suitable examples of haloalkyls include, but are not limited to, fluoromethyl, difluoromethyl, trifluoromethyl, 2-fluoroethyl, 1,2-difluoroethyl and 2,2-difluoroethyl.

In certain embodiments, R³ is taken with R² or R² and R¹ and forms a heterocycloalkyl, the heterocycloalkyl is

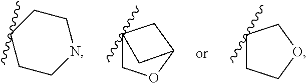

wherein the heterocycloalkyl is unsubstituted or substituted with one to four substituents selected from the group consisting of halogen, C₁-C₆alkyl, haloC₁-C₆alkyl, alkoxy, CN, COOC₁-C₆alkyl, and heteroaryl, wherein the heteroaryl is unsubstituted or substituted with one to four substituents selected from the group consisting of halogen, CN, C₁-C₆alkyl and haloC₁-C₆alkyl.

In certain embodiments, wherein when the heterocycloalkyl is

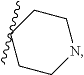

and wherein the heterocycloalkyl is substituted with heteroaryl, the heteroaryl is

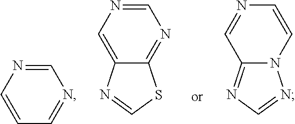

wherein the heteroaryl is unsubstituted or substituted with CN.

In certain embodiments, R¹ and R² are independently selected from the group consisting of methyl, difluoromethyl, fluorine, difluoroethyl, trifluoromethyl, ethyl, N(CH₃)₂, CN and CH₂CN, and R³ is independently selected from the group consisting of methyl, difluoromethyl, fluorine, difluoroethyl, trifluoromethyl and ethyl.

In certain embodiments, R² is taken with R³ or R¹ and R³ and forms a C₃-C₁₀cycloalkyl or heterocycloalkyl, wherein the C₃-C₁₀cycloalkyl or heterocycloalkyl is unsubstituted or substituted with one to four substituents selected from the group consisting of halogen, C₁-C₆alkyl, haloC₁-C₆alkyl, alkoxy, CN, COOC₁-C₆alkyl, and heteroaryl, wherein the heteroaryl is unsubstituted or substituted with one to four substituents selected from the group consisting of halogen, CN, C₁-C₆alkyl and haloC₁-C₆alkyl.

In certain embodiments, R³ is taken with R² or R¹ and R² and forms a C₃-C₁₀cycloalkyl or heterocycloalkyl, wherein the C₃-C₁₀cycloalkyl or heterocycloalkyl is unsubstituted or substituted with one to four substituents selected from the group consisting of halogen, C₁-C₆alkyl, haloC₁-C₆alkyl, alkoxy, CN, COOC₁-C₆alkyl, and heteroaryl, wherein the heteroaryl is unsubstituted or substituted with one to four substituents selected from the group consisting of halogen, CN, C₁-C₆alkyl and haloC₁-C₆alkyl.

In certain embodiments, R³ is methyl, difluoromethyl, CH₂OCF₃, cyclobutyl, difluoroethyl, ethyl, cyclopropyl, CN, CH₂CN, CH₂phenyl, CONHCH₂phenyl, wherein the CH₂phenyl or CONHCH₂phenyl is substituted with one substituent selected from the group consisting of chlorine and methoxy.

In regard to the compounds described herein, R⁴ is hydrogen, C₁-C₆alkyl, or wherein when taken with R⁵ or R⁶ forms a —CH₂— or —CH₂CH₂— bridge. In certain embodiments, R⁴ is hydrogen. In certain embodiments, R⁴ is C₁-C₆alkyl. Suitable alkyls include, but are not limited to, methyl, ethyl, n-propyl, isopropyl, n-butyl, isobutyl, sec-butyl, tert-butyl, n-pentyl, isopentyl, neopentyl, tert-pentyl, 1-methylbutyl, 2-methylbutyl, 1,2-dimethylpropyl, 1-ethylpropyl, n-hexyl, isohexyl, 1-methylpentyl, 2-methylpentyl, 3-methylpentyl, 1,1-dimethylbutyl, 1,2-dimethylbutyl, 2,2-dimethylbutyl, 1-ethylbutyl, 1,1,2-trimethylpropyl, 1,2,2-trimethylpropyl, 1-ethyl-2-methylpropyl and 1-ethyl-1-methylpropyl. In certain embodiments, R⁴ is methyl.

In certain embodiments, R⁴ is taken with R⁵ or R⁶ and forms a —CH₂— or —CH₂CH₂— bridge. In certain embodiments, R⁴ is taken with R⁵ and forms a —CH₂— bridge. In certain embodiments, R⁴ is taken with R⁵ and forms a —CH₂CH₂— bridge. In certain embodiments, R⁴ is taken with R⁶ and forms a —CH₂— bridge. In certain embodiments, R⁴ is taken with R⁶ and forms a —CH₂CH₂— bridge. In certain embodiments, R⁴ is taken with R⁵ to form a CH₂CH₂ bridge, or R⁴ is taken with R⁶ to form a CH₂ bridge.

In regard to the compounds described herein, R⁵ is hydrogen, C₁-C₆alkyl, or wherein when taken with R⁴ form a —CH₂— or —CH₂CH₂— bridge. In regard to the compounds described herein, R⁵ is hydrogen, C₁-C₆alkyl, or wherein when taken with R⁴ form a —CH₂CH₂— bridge. In certain embodiments, R⁵ is hydrogen. In certain embodiments, R⁵ is C₁-C₆alkyl. Suitable alkyls include, but are not limited to, methyl, ethyl, n-propyl, isopropyl, n-butyl, isobutyl, sec-butyl, tert-butyl, n-pentyl, isopentyl, neopentyl, tert-pentyl, 1-methylbutyl, 2-methylbutyl, 1,2-dimethylpropyl, 1-ethylpropyl, n-hexyl, isohexyl, 1-methylpentyl, 2-methylpentyl, 3-methylpentyl, 1,1-dimethylbutyl, 1,2-dimethylbutyl, 2,2-dimethylbutyl, 1-ethylbutyl, 1,1,2-trimethylpropyl, 1,2,2-trimethylpropyl, 1-ethyl-2-methylpropyl and 1-ethyl-1-methylpropyl. In certain embodiments, R⁵ is methyl. In certain embodiments, R⁵ is taken with R⁴ and forms a —CH₂— bridge. In certain embodiments, R⁵ is taken with R⁴ and forms a —CH₂CH₂— bridge.

In regard to the compounds described herein, R⁶ is hydrogen, C₁-C₆alkyl, or wherein when taken with R⁴ form a —CH₂— or —CH₂CH₂— bridge. In regard to the compounds described herein, R⁶ is hydrogen, C₁-C₆alkyl, or wherein when taken with R⁴ form a —CH₂— bridge. In certain embodiments, R⁶ is hydrogen. In certain embodiments, R⁶ is C₁-C₆alkyl. Suitable alkyls include, but are not limited to, methyl, ethyl, n-propyl, isopropyl, n-butyl, isobutyl, sec-butyl, tert-butyl, n-pentyl, isopentyl, neopentyl, tert-pentyl, 1-methylbutyl, 2-methylbutyl, 1,2-dimethylpropyl, 1-ethylpropyl, n-hexyl, isohexyl, 1-methylpentyl, 2-methylpentyl, 3-methylpentyl, 1,1-dimethylbutyl, 1,2-dimethylbutyl, 2,2-dimethylbutyl, 1-ethylbutyl, 1,1,2-trimethylpropyl, 1,2,2-trimethylpropyl, 1-ethyl-2-methylpropyl and 1-ethyl-1-methylpropyl. In certain embodiments, R⁶ is methyl. In certain embodiments, R⁶ is taken with R⁴ and forms a —CH₂— bridge. In certain embodiments, R⁶ is taken with R⁴ and forms a —CH₂CH₂— bridge.

Such embodiments, are shown below:

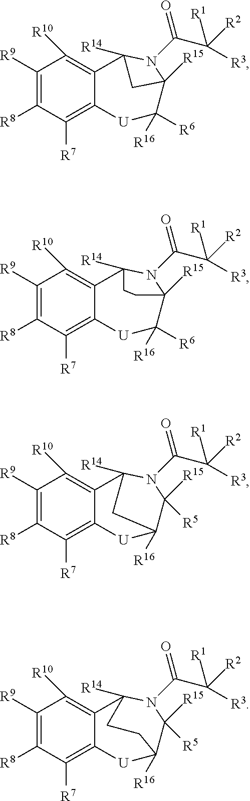

In certain embodiments, when U is O or CR¹²R¹³, R⁴ forms a —CH₂— or —CH₂CH₂— bridge with R⁵ or R⁶.

In regard to the compounds described herein, R⁷ is hydrogen, OH, C₁-C₆alkylOH, CN, C₁-C₆alkylCN, C₁-C₆alkyl, C₁-C₆alkylaryl, C₂-C₆alkynyl, haloC₁-C₆alkyl, halogen, or alkoxy, wherein the C₂-C₆alkynyl is unsubstituted or substituted with one to three substituents selected from the group consisting of OH, C₁-C₆alkylOH, CN, C₁-C₆alkylCN, NH₂, NHC₁-C₆alkyl, N(C₁-C₆alkyl)₂, haloC₁-C₆alkyl, halogen, alkoxy, haloalkoxy, aryl, C₃-C₁₀cycloalkyl, heteroaryl and heterocycloalkyl, wherein the aryl, C₃-C₁₀cycloalkyl, heteroaryl and heterocycloalkyl are unsubstituted or substituted with one to three substituents selected from the group consisting of OH, C₁-C₆alkylOH, CN, C₁-C₆alkylCN, C₁-C₆alkyl, NH₂, NHC₁-C₆alkyl, N(C₁-C₆alkyl)₂, haloC₁-C₆alkyl, halogen, alkoxy, haloalkoxy.

In certain embodiments, R⁷ is hydrogen. In certain embodiments, R⁷ is OH. In certain embodiments, R⁷ is C₁-C₆alkylOH. Suitable alcohols include, but are not limited to, methanol, ethanol, propanol and butanol.

In certain embodiments, R⁷ is CN. In certain embodiments, R⁷ is C₁-C₆alkylCN. In certain embodiments, R⁷ is

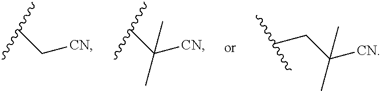

In certain embodiments, R⁷ is C₁-C₆alkyl. Suitable alkyls include, but are not limited to, methyl, ethyl, n-propyl, isopropyl, n-butyl, isobutyl, sec-butyl, tert-butyl, n-pentyl, isopentyl, neopentyl, tert-pentyl, 1-methylbutyl, 2-methylbutyl, 1,2-dimethylpropyl, 1-ethylpropyl, n-hexyl, isohexyl, 1-methylpentyl, 2-methylpentyl, 3-methylpentyl, 1,1-dimethylbutyl, 1,2-dimethylbutyl, 2,2-dimethylbutyl, 1-ethylbutyl, 1,1,2-trimethylpropyl, 1,2,2-trimethylpropyl, 1-ethyl-2-methylpropyl and 1-ethyl-1-methylpropyl. In certain embodiments, R⁷ is methyl or ethyl. In certain embodiments, R⁷ is C₁-C₆alkylaryl. In certain embodiments, R⁷ is

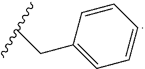 [SMILES: *Cc1ccccc1]

In certain embodiments, R⁷ is C₂-C₆alkynyl. Suitable alkynyls include, but are not limited to, ethynyl, propynyl, butynyl, and hexynyl. In certain embodiments, the C₂-C₆alkynyl is unsubstituted. In other embodiments, the C₂-C₆alkynyl is substituted.

In certain embodiments, the C₂-C₆alkynyl is substituted with one to three substituents selected from the group consisting of OH, C₁-C₆alkylOH, CN, C₁-C₆alkylCN, NH₂, NHC₁-C₆alkyl, N(C₁-C₆alkyl)₂, haloC₁-C₆alkyl, halogen, alkoxy, haloalkoxy, aryl, C₃-C₁₀cycloalkyl, heteroaryl and heterocycloalkyl, wherein the aryl, C₃-C₁₀cycloalkyl, heteroaryl and heterocycloalkyl are unsubstituted or substituted with one to three substituents selected from the group consisting of OH, C₁-C₆alkylOH, CN, C₁-C₆alkylCN, C₁-C₆alkyl, NH₂, NHC₁-C₆alkyl, N(C₁-C₆alkyl)₂, haloC₁-C₆alkyl, halogen, alkoxy, haloalkoxy.

In certain embodiments, the C₂-C₆alkynyl is substituted with one substituent selected from the group consisting of OH, C₁-C₆alkylOH, CN, C₁-C₆alkylCN, NH₂, NHC₁-C₆alkyl, N(C₁-C₆alkyl)₂, haloC₁-C₆alkyl, halogen, alkoxy, haloalkoxy, aryl, C₃-C₁₀cycloalkyl, heteroaryl and heterocycloalkyl, wherein the aryl, C₃-C₁₀cycloalkyl, heteroaryl and heterocycloalkyl are unsubstituted or substituted with one to three substituents selected from the group consisting of OH, C₁-C₆alkylOH, CN, C₁-C₆alkylCN, C₁-C₆alkyl, NH₂, NHC₁-C₆alkyl, N(C₁-C₆alkyl)₂, haloC₁-C₆alkyl, halogen, alkoxy, haloalkoxy.

In certain embodiments, the C₂-C₆alkynyl is substituted with two substituents selected from the group consisting of OH, C₁-C₆alkylOH, CN, C₁-C₆alkylCN, NH₂, NHC₁-C₆alkyl, N(C₁-C₆alkyl)₂, haloC₁-C₆alkyl, halogen, alkoxy, haloalkoxy, aryl, C₃-C₁₀cycloalkyl, heteroaryl and heterocycloalkyl, wherein the aryl, C₃-C₁₀cycloalkyl, heteroaryl and heterocycloalkyl are unsubstituted or substituted with one to three substituents selected from the group consisting of OH, C₁-C₆alkylOH, CN, C₁-C₆alkylCN, C₁-C₆alkyl, NH₂, NHC₁-C₆alkyl, N(C₁-C₆alkyl)₂, haloC₁-C₆alkyl, halogen, alkoxy, haloalkoxy.

In certain embodiments, the C₂-C₆alkynyl is substituted with three substituents selected from the group consisting of OH, C₁-C₆alkylOH, CN, C₁-C₆alkylCN, NH₂, NHC₁-C₆alkyl, N(C₁-C₆alkyl)₂, haloC₁-C₆alkyl, halogen, alkoxy, haloalkoxy, aryl, C₃-C₁₀cycloalkyl, heteroaryl and heterocycloalkyl, wherein the aryl, C₃-C₁₀cycloalkyl, heteroaryl and heterocycloalkyl are unsubstituted or substituted with one to three substituents selected from the group consisting of OH, C₁-C₆alkylOH, CN, C₁-C₆alkylCN, C₁-C₆alkyl, NH₂, NHC₁-C₆alkyl, N(C₁-C₆alkyl)₂, haloC₁-C₆alkyl, halogen, alkoxy, haloalkoxy.

In certain embodiments, the C₂-C₆alkynyl is substituted with OH. In certain embodiments, the C₂-C₆alkynyl is substituted with C₁-C₆alkylOH. Suitable alcohols include, but are not limited to, methanol, ethanol, propanol and butanol. In certain embodiments, the C₂-C₆alkynyl is substituted with CN. In certain embodiments, the C₁-C₆alkynyl is substituted with C₁-C₆alkylCN. In certain embodiments, C₂-C₆alkynyl is substituted with

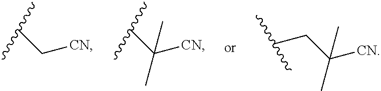

In certain embodiments, the C₂-C₆alkynyl is substituted with NH₂. In certain embodiments, the C₂-C₆alkynyl is substituted with NHC₁-C₆alkyl. In certain embodiments, the C₂-C₆alkynyl is substituted with N(C₁-C₆alkyl)₂. Suitable examples include but are not limited to N(CH₃)₂ and N(CH₂CH₃)₂.

In certain embodiments, the C₂-C₆alkynyl is substituted with haloC₁-C₆alkyl. Suitable examples of haloalkyls include, but are not limited to, fluoromethyl, difluoromethyl, trifluoromethyl, 2-fluoroethyl, 1,2-difluoroethyl and 2,2-difluoroethyl. In certain embodiments, the C₂-C₆alkynyl is substituted with halogen. Suitable halogens include, but are not limited to, fluorine, chlorine, bromine or iodine. In certain embodiments, the C₂-C₆alkynyl is substituted with alkoxy. In certain embodiments, the C₂-C₆alkynyl is substituted with haloalkoxy. Suitable alkoxys include, but are not limited to, methoxy, ethoxy, n-propoxy, isopropoxy and n-butoxy.

In certain embodiments, the C₂-C₆alkynyl is substituted with aryl. Suitable alkoxys include, but are not limited to, phenyl and naphthyl. In certain embodiments, the C₂-C₆alkynyl is substituted with C₃-C₁₀cycloalkyl. Suitable examples of cycloalkyls include, but are not limited to, cyclopropyl, cyclobutyl, cyclopentyl and cyclohexyl. In certain embodiments, the C₂-C₆alkynyl is substituted with heteroaryl. Suitable heteroaryls include, but are not limited to, pyridyl (pyridinyl), oxazolyl, imidazolyl, triazolyl, furyl, triazinyl, thienyl, pyrimidyl, pyridazinyl, indolizinyl, cinnolinyl, phthalazinyl, quinazolinyl, naphthyridinyl, quinoxalinyl, purinyl, benzimidazolyl, quinolyl, and isoquinolyl. In certain embodiments, the C₂-C₆alkynyl is substituted with heterocycloalkyl. Suitable examples of heterocycloalkyls include, but are not limited to, piperidyl, oxetanyl, pyrrolyl, piperazinyl, morpholinyl, thiomorpholinyl, thiazolidinyl, 1,4-dioxanyl, tetrahydrofuranyl, tetrahydrothiophenyl, beta lactam, gamma lactam, delta lactam, beta lactone, gamma lactone, delta lactone, and pyrrolidinone, and oxides thereof.

In certain embodiments, when R⁷ is alkynyl and the alkynyl is substituted with aryl, C₃-C₁₀cycloalkyl, heteroaryl and heterocycloalkyl, the aryl, C₃-C₁₀cycloalkyl, heteroaryl and heterocycloalkyl are unsubstituted or substituted with one to three substituents selected from the group consisting of OH, C₁-C₆alkylOH, CN, C₁-C₆alkylCN, C₁-C₆alkyl, NH₂, NHC₁-C₆alkyl, N(C₁-C₆alkyl)₂, haloC₁-C₆alkyl, halogen, alkoxy, haloalkoxy. In certain embodiments, the aryl, C₃-C₁₀cycloalkyl, heteroaryl and heterocycloalkyl are unsubstituted. In certain embodiments, the aryl, C₃-C₁₀cycloalkyl, heteroaryl and heterocycloalkyl are substituted with one substituent selected from the group consisting of OH, C₁-C₆alkylOH, CN, C₁-C₆alkylCN, C₁-C₆alkyl, NH₂, NHC₁-C₆alkyl, N(C₁-C₆alkyl)₂, haloC₁-C₆alkyl, halogen, alkoxy, haloalkoxy. In certain embodiments, the aryl, C₃-C₁₀cycloalkyl, heteroaryl and heterocycloalkyl are substituted with two substituents selected from the group consisting of OH, C₁-C₆alkylOH, CN, C₁-C₆alkylCN, C₁-C₆alkyl, NH₂, NHC₁-C₆alkyl, N(C₁-C₆alkyl)₂, haloC₁-C₆alkyl, halogen, alkoxy, haloalkoxy. In certain embodiments, the aryl, C₃-C₁₀cycloalkyl, heteroaryl and heterocycloalkyl are substituted with three substituents selected from the group consisting of OH, C₁-C₆alkylOH, CN, C₁-C₆alkylCN, C₁-C₆alkyl, NH₂, NHC₁-C₆alkyl, N(C₁-C₆alkyl)₂, haloC₁-C₆alkyl, halogen, alkoxy, haloalkoxy. In certain embodiments, the aryl, C₃-C₁₀cycloalkyl, heteroaryl and heterocycloalkyl are unsubstituted or substituted with one to three substituents selected from the group consisting of OH, fluorine, trifluoromethyl, methyl, CN, NH₂, N(CH₃)₂, methoxy and trifluoromethoxy.

In certain embodiments, R⁷ is

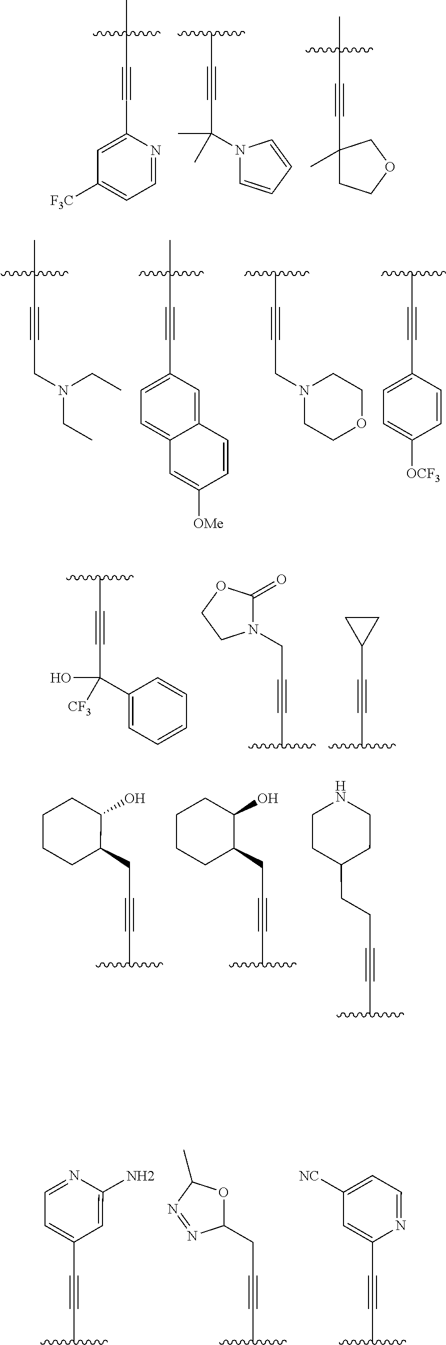

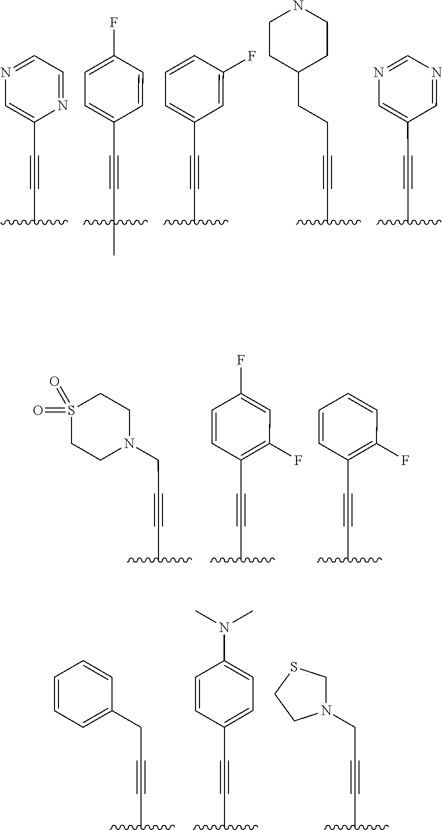

In certain embodiments, R⁷ is haloC₁-C₆alkyl. Suitable examples of haloalkyls include, but are not limited to, fluoromethyl, difluoromethyl, trifluoromethyl, 2-fluoroethyl, 1,2-difluoroethyl and 2,2-difluoroethyl. In certain embodiments, R⁷ is difluoromethyl. In certain embodiments, R⁷ is trifluoromethyl. In certain embodiments, R⁷ is difluoromethyl or trifluoromethyl. In certain embodiments, R⁷ is halogen. Suitable halogens include, but are not limited to, fluorine, chlorine, bromine or iodine. In certain embodiments, R⁷ is fluorine or chlorine. In certain embodiments, R⁷ is alkoxy. Suitable alkoxys include, but are not limited to, methoxy, ethoxy, n-propoxy, isopropoxy and n-butoxy. In certain embodiments, R⁷ is methoxy.

In certain embodiments, R⁷ is hydrogen, chlorine, CN, methyl or fluorine.

In regard to the compounds described herein, R⁸ is hydrogen, OH, C₁-C₆alkylOH, CN, C₁-C₆alkylCN, C₁-C₆alkyl, C₁-C₆alkylaryl, C₂-C₆alkynyl, haloC₁-C₆alkyl, halogen, or alkoxy. In certain embodiments, R⁸ is hydrogen. In certain embodiments, R⁸ is OH. In certain embodiments, R⁸ is C₁-C₆alkylOH. Suitable alcohols include, but are not limited to, methanol, ethanol, propanol and butanol.

In certain embodiments, R⁸ is CN. In certain embodiments, R⁸ is C₁-C₆alkylCN. In certain embodiments, R⁸ is

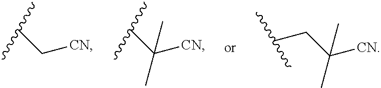

In certain embodiments, R⁸ is C₁-C₆alkyl. Suitable alkyls include, but are not limited to, methyl, ethyl, n-propyl, isopropyl, n-butyl, isobutyl, sec-butyl, tert-butyl, n-pentyl, isopentyl, neopentyl, tert-pentyl, 1-methylbutyl, 2-methylbutyl, 1,2-dimethylpropyl, 1-ethylpropyl, n-hexyl, isohexyl, 1-methylpentyl, 2-methylpentyl, 3-methylpentyl, 1,1-dimethylbutyl, 1,2-dimethylbutyl, 2,2-dimethylbutyl, 1-ethylbutyl, 1,1,2-trimethylpropyl, 1,2,2-trimethylpropyl, 1-ethyl-2-methylpropyl and 1-ethyl-1-methylpropyl. In certain embodiments, R⁸ is methyl or ethyl. In certain embodiments, R⁸ is methyl. In certain embodiments, R⁸ is C₁-C₆alkylaryl. In certain embodiments, R⁸ is

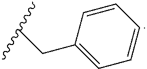 [SMILES: *Cc1ccccc1]

In certain embodiments, R⁸ is C₂-C₆alkynyl. Suitable alkynyls include, but are not limited to, ethynyl, propynyl, butynyl, and hexynyl.

In certain embodiments, R⁸ is haloC₁-C₆alkyl. Suitable examples of haloalkyls include, but are not limited to, fluoromethyl, difluoromethyl, trifluoromethyl, 2-fluoroethyl, 1,2-difluoroethyl and 2,2-difluoroethyl. In certain embodiments, R⁸ is difluoromethyl. In certain embodiments, R⁸ is trifluoromethyl. In certain embodiments, R⁸ is difluoromethyl or trifluoromethyl. In certain embodiments, R⁸ is halogen. Suitable halogens include, but are not limited to, fluorine, chlorine, bromine or iodine. In certain embodiments, R⁸ is fluorine or chlorine. In certain embodiments, R⁸ is alkoxy. Suitable alkoxys include, but are not limited to, methoxy, ethoxy, n-propoxy, isopropoxy and n-butoxy. In certain embodiments, R⁸ is methoxy.

In certain embodiments, R⁸ is hydrogen or chlorine.

In certain embodiments, R⁸ is hydrogen, CN or chlorine.

In regard to the compounds described herein, R⁹ is hydrogen, OH, C₁-C₆alkylOH, CN, C₁-C₆alkylCN, C₁-C₆alkyl, C₁-C₆alkylaryl, C₂-C₆alkynyl, haloC₁-C₆alkyl, halogen, or alkoxy. In certain embodiments, R⁹ is hydrogen. In certain embodiments, R⁹ is OH. In certain embodiments, R⁹ is C₁-C₆alkylOH. Suitable alcohols include, but are not limited to, methanol, ethanol, propanol and butanol.

In certain embodiments, R⁹ is CN. In certain embodiments, R⁹ is C₁-C₆alkylCN. In certain embodiments, R⁹ is

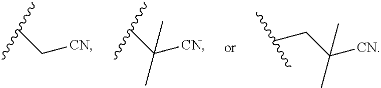

In certain embodiments, R⁹ is C₁-C₆alkyl. Suitable alkyls include, but are not limited to, methyl, ethyl, n-propyl, isopropyl, n-butyl, isobutyl, sec-butyl, tert-butyl, n-pentyl, isopentyl, neopentyl, tert-pentyl, 1-methylbutyl, 2-methylbutyl, 1,2-dimethylpropyl, 1-ethylpropyl, n-hexyl, isohexyl, 1-methylpentyl, 2-methylpentyl, 3-methylpentyl, 1,1-dimethylbutyl, 1,2-dimethylbutyl, 2,2-dimethylbutyl, 1-ethylbutyl, 1,1,2-trimethylpropyl, 1,2,2-trimethylpropyl, 1-ethyl-2-methylpropyl and 1-ethyl-1-methylpropyl. In certain embodiments, R⁹ is methyl or ethyl. In certain embodiments, R⁹ is methyl. In certain embodiments, R⁹ is C₁-C₆alkylaryl. In certain embodiments, R⁹ is

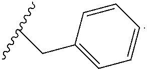 [SMILES: *Cc1ccccc1]

In certain embodiments, R⁹ is C₂-C₆alkynyl. Suitable alkynyls include, but are not limited to, ethynyl, propynyl, butynyl, and hexynyl.

In certain embodiments, R⁹ is haloC₁-C₆alkyl. Suitable examples of haloalkyls include, but are not limited to, fluoromethyl, difluoromethyl, trifluoromethyl, 2-fluoroethyl, 1,2-difluoroethyl and 2,2-difluoroethyl. In certain embodiments, R⁹ is difluoromethyl. In certain embodiments, R⁹ is trifluoromethyl. In certain embodiments, R⁹ is difluoromethyl or trifluoromethyl. In certain embodiments, R⁹ is halogen. Suitable halogens include, but are not limited to, fluorine, chlorine, bromine or iodine. In certain embodiments, R⁹ is fluorine or chlorine. In certain embodiments, R⁹ is alkoxy. Suitable alkoxys include, but are not limited to, methoxy, ethoxy, n-propoxy, isopropoxy and n-butoxy. In certain embodiments, R⁹ is methoxy.

In certain embodiments, R⁹ is hydrogen chlorine, CN, methyl or fluorine.

In regard to the compounds described herein, R¹⁰ is hydrogen, OH, C₁-C₆alkylOH, CN, C₁-C₆alkylCN, C₁-C₆alkyl, C₁-C₆alkylaryl, C₂-C₆alkynyl, haloC₁-C₆alkyl, halogen, or alkoxy. In certain embodiments, R¹⁰ is hydrogen. In certain embodiments, R¹⁰ is OH. In certain embodiments, R¹⁰ is C₁-C₆alkylOH. Suitable alcohols include, but are not limited to, methanol, ethanol, propanol and butanol.

In certain embodiments. R¹⁰ is CN. In certain embodiments. R¹⁰ is C₁-C₆alkylCN. In certain embodiments, R¹⁰ is

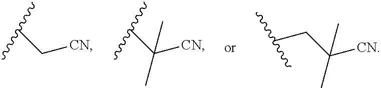

In certain embodiments, R¹⁰ is C₁-C₆alkyl. Suitable alkyls include, but are not limited to, methyl, ethyl, n-propyl, isopropyl, n-butyl, isobutyl, sec-butyl, tert-butyl, n-pentyl, isopentyl, neopentyl, tert-pentyl, 1-methylbutyl, 2-methylbutyl, 1,2-dimethylpropyl, 1-ethylpropyl, n-hexyl, isohexyl, 1-methylpentyl, 2-methylpentyl, 3-methylpentyl, 1,1-dimethylbutyl, 1,2-dimethylbutyl, 2,2-dimethylbutyl, 1-ethylbutyl, 1,1,2-trimethylpropyl, 1,2,2-trimethylpropyl, 1-ethyl-2-methylpropyl and 1-ethyl-1-methylpropyl. In certain embodiments, R¹⁰ is methyl or ethyl. In certain embodiments, R¹⁰ is C₁-C₆alkylaryl. In certain embodiments, R¹⁰ is

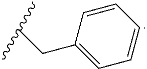 [SMILES: *Cc1ccccc1]

In certain embodiments, R¹⁰ is C₂-C₆alkynyl. Suitable alkynyls include, but are not limited to, ethynyl, propynyl, butynyl, and hexynyl.

In certain embodiments, R¹⁰ is haloC₁-C₆alkyl. Suitable examples of haloalkyls include, but are not limited to, fluoromethyl, difluoromethyl, trifluoromethyl, 2-fluoroethyl, 1,2-difluoroethyl and 2,2-difluoroethyl. In certain embodiments, R¹⁰ is difluoromethyl. In certain embodiments, R¹⁰ is trifluoromethyl. In certain embodiments, R¹⁰ is difluoromethyl or trifluoromethyl. In certain embodiments, R¹⁰ is halogen. Suitable halogens include, but are not limited to, fluorine, chlorine, bromine or iodine. In certain embodiments, R¹⁰ is fluorine or chlorine. In certain embodiments, R¹⁰ is alkoxy. Suitable alkoxys include, but are not limited to, methoxy, ethoxy, n-propoxy, isopropoxy and n-butoxy. In certain embodiments, R¹⁰ is methoxy.

In regard to the compounds described herein, R 14 is hydrogen, OH, C₁-C₆alkylOH, CN, C₁-C₆alkylCN, C₁-C₆alkyl, haloC₁-C₆alkyl, halogen, or alkoxy. In certain embodiments, R¹⁴ is hydrogen. In certain embodiments, R¹⁴ is OH. In certain embodiments, R¹⁴ is C₁-C₆alkylOH. Suitable alcohols include, but are not limited to, methanol, ethanol, propanol and butanol.

In certain embodiments, R¹⁴ is CN. In certain embodiments, R¹⁴ is C₁-C₆alkylCN. In certain embodiments, R¹⁴ is

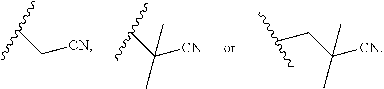

In certain embodiments, R¹⁴ is C₁-C₆alkyl. Suitable alkyls include, but are not limited to, methyl, ethyl, n-propyl, isopropyl, n-butyl, isobutyl, sec-butyl, tert-butyl, n-pentyl, isopentyl, neopentyl, tert-pentyl, 1-methylbutyl, 2-methylbutyl, 1,2-dimethylpropyl, 1-ethylpropyl, n-hexyl, isohexyl, 1-methylpentyl, 2-methylpentyl, 3-methylpentyl, 1,1-dimethylbutyl, 1,2-dimethylbutyl, 2,2-dimethylbutyl, 1-ethylbutyl, 1,1,2-trimethylpropyl, 1,2,2-trimethylpropyl, 1-ethyl-2-methylpropyl and 1-ethyl-1-methylpropyl. In certain embodiments, R¹⁴ is methyl or ethyl.

In certain embodiments, R¹⁴ is haloC₁-C₆alkyl. Suitable examples of haloalkyls include, but are not limited to, fluoromethyl, difluoromethyl, trifluoromethyl, 2-fluoroethyl, 1,2-difluoroethyl and 2,2-difluoroethyl. In certain embodiments, R¹⁴ is difluoromethyl. In certain embodiments, R¹⁴ is trifluoromethyl. In certain embodiments, R¹⁴ is difluoromethyl or trifluoromethyl. In certain embodiments, R¹⁴ is halogen. Suitable halogens include, but are not limited to, fluorine, chlorine, bromine or iodine. In certain embodiments, R¹⁴ is fluorine or chlorine. In certain embodiments, R¹⁴ is alkoxy. Suitable alkoxys include, but are not limited to, methoxy, ethoxy, n-propoxy, isopropoxy and n-butoxy. In certain embodiments, R¹⁴ is methoxy.

In regard to the compounds described herein, R¹⁵ is hydrogen, OH, C₁-C₆alkylOH, CN, C₁-C₆alkylCN, C₁-C₆alkyl, haloC₁-C₆alkyl, halogen, or alkoxy. In certain embodiments, R¹⁵ is hydrogen. In certain embodiments, R¹⁵ is OH. In certain embodiments, R¹⁵ is C₁-C₆alkylOH. Suitable alcohols include, but are not limited to, methanol, ethanol, propanol and butanol.

In certain embodiments, R¹⁵ is CN. In certain embodiments, R¹⁵ is C₁-C₆alkylCN. In certain embodiments, R¹⁵ is

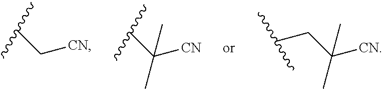

In certain embodiments, R¹⁵ is C₁-C₆alkyl. Suitable alkyls include, but are not limited to, methyl, ethyl, n-propyl, isopropyl, n-butyl, isobutyl, sec-butyl, tert-butyl, n-pentyl, isopentyl, neopentyl, tert-pentyl, 1-methylbutyl, 2-methylbutyl, 1,2-dimethylpropyl, 1-ethylpropyl, n-hexyl, isohexyl, 1-methylpentyl, 2-methylpentyl, 3-methylpentyl, 1,1-dimethylbutyl, 1,2-dimethylbutyl, 2,2-dimethylbutyl, 1-ethylbutyl, 1,1,2-trimethylpropyl, 1,2,2-trimethylpropyl, 1-ethyl-2-methylpropyl and 1-ethyl-1-methylpropyl. In certain embodiments, R¹⁵ is methyl or ethyl. In certain embodiments, R¹⁵ is methyl.

In certain embodiments, R¹⁵ is haloC₁-C₆alkyl. Suitable examples of haloalkyls include, but are not limited to, fluoromethyl, difluoromethyl, trifluoromethyl, 2-fluoroethyl, 1,2-difluoroethyl and 2,2-difluoroethyl. In certain embodiments, R¹⁵ is difluoromethyl. In certain embodiments, R¹⁵ is trifluoromethyl. In certain embodiments, R¹⁵ is difluoromethyl or trifluoromethyl. In certain embodiments, R¹⁵ is halogen. Suitable halogens include, but are not limited to, fluorine, chlorine, bromine or iodine. In certain embodiments, R¹⁵ is fluorine or chlorine. In certain embodiments, R¹⁵ is alkoxy. Suitable alkoxys include, but are not limited to, methoxy, ethoxy, n-propoxy, isopropoxy and n-butoxy. In certain embodiments, R¹⁵ is methoxy.

In regard to the compounds described herein, R¹⁶ is hydrogen, OH, C₁-C₆alkylOH, CN, C₁-C₆alkylCN, C₁-C₆alkyl, haloC₁-C₆alkyl, halogen, or alkoxy. In certain embodiments, R¹⁶ is hydrogen. In certain embodiments, R¹⁶ is OH. In certain embodiments, R¹⁶ is C₁-C₆alkylOH. Suitable alcohols include, but are not limited to, methanol, ethanol, propanol and butanol.

In certain embodiments. R¹⁶ is CN. In certain embodiments. R¹⁶ is C₁-C₆alkylCN. In certain embodiments, R¹⁶ is

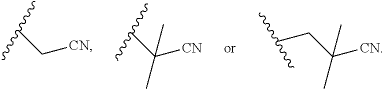

In certain embodiments, R¹⁶ is C₁-C₆alkyl. Suitable alkyls include, but are not limited to, methyl, ethyl, n-propyl, isopropyl, n-butyl, isobutyl, sec-butyl, tert-butyl, n-pentyl, isopentyl, neopentyl, tert-pentyl, 1-methylbutyl, 2-methylbutyl, 1,2-dimethylpropyl, 1-ethylpropyl, n-hexyl, isohexyl, 1-methylpentyl, 2-methylpentyl, 3-methylpentyl, 1,1-dimethylbutyl, 1,2-dimethylbutyl, 2,2-dimethylbutyl, 1-ethylbutyl, 1,1,2-trimethylpropyl, 1,2,2-trimethylpropyl, 1-ethyl-2-methylpropyl and 1-ethyl-1-methylpropyl. In certain embodiments, R¹⁶ is methyl or ethyl.

In certain embodiments, R¹⁶ is haloC₁-C₆alkyl. Suitable examples of haloalkyls include, but are not limited to, fluoromethyl, difluoromethyl, trifluoromethyl, 2-fluoroethyl, 1,2-difluoroethyl and 2,2-difluoroethyl. In certain embodiments, R¹⁶ is difluoromethyl. In certain embodiments, R¹⁶ is trifluoromethyl. In certain embodiments, R¹⁶ is difluoromethyl or trifluoromethyl. In certain embodiments, R¹⁶ is halogen. Suitable halogens include, but are not limited to, fluorine, chlorine, bromine or iodine. In certain embodiments, R¹⁶ is fluorine or chlorine. In certain embodiments, R¹⁶ is alkoxy. Suitable alkoxys include, but are not limited to, methoxy, ethoxy, n-propoxy, isopropoxy and n-butoxy. In certain embodiments, R¹⁶ is methoxy.

Also described herein are compounds of Formula II:

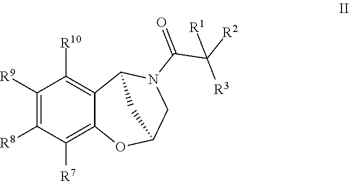

or a pharmaceutically acceptable salt thereof, wherein:
R¹ is hydrogen, OH, C₁-C₆alkylOH, CN, C₁-C₆alkylCN, C₁-C₆alkyl, C₃-C₆cycloalkyl, haloC₁-C₆alkyl, halogen, NH₂, N(C₁-C₆alkyl)₂, NH(C₁-C₆alkyl) or alkoxy, or wherein when R¹ is taken with R² or R² and R³, forms a C₃-C₁₀cycloalkyl, aryl, heteroaryl or heterocycloalkyl, wherein the C₃-C₁₀cycloalkyl, aryl, heteroaryl or heterocycloalkyl is unsubstituted or substituted with one to four substituents selected from the group consisting of halogen, C₁-C₆alkyl, haloC₁-C₆alkyl, alkoxy, CN, COOC₁-C₆alkyl, COaryl, aryl and heteroaryl, wherein the heteroaryl is unsubstituted or substituted with one to four substituents selected from the group consisting of halogen, CN, C₁-C₆alkyl, alkoxy, aryl, C₃-C₆cycloalkyl and haloC₁-C₆alkyl;
R² is hydrogen, OH, C₁-C₆alkylOH, CN, C₁-C₆alkylCN, C₁-C₆alkyl, C₃-C₆cycloalkyl, haloC₁-C₆alkyl, halogen, NH₂, N(C₁-C₆alkyl)₂, NH(C₁-C₆alkyl) or alkoxy, or wherein when R² is taken with R¹ or R¹ and R³, forms a C₃-C₁₀cycloalkyl, aryl, heteroaryl or heterocycloalkyl, wherein the C₃-C₁₀cycloalkyl, aryl, heteroaryl or heterocycloalkyl is unsubstituted or substituted with one to four substituents selected from the group consisting of halogen, C₁-C₆alkyl, haloC₁-C₆alkyl, alkoxy, CN, COOC₁-C₆alkyl, COaryl, aryl and heteroaryl, wherein the heteroaryl is unsubstituted or substituted with one to four substituents selected from the group consisting of halogen, CN, C₁-C₆alkyl, alkoxy, aryl, C₃-C₆cycloalkyl and haloC₁-C₆alkyl;
R³ is OH, C₁-C₆alkylOH, CN, C₁-C₆alkylCN, C₁-C₆alkyl, C₃-C₆cycloalkyl, haloC₁-C₆alkyl, halogen, C₁-C₆alkylOhaloC₁-C₆alkyl, alkoxy, CONHC₁-C₆alkylaryl, aryl, or C₁-C₆alkylaryl, wherein the CONHC₁-C₆alkylaryl, aryl, or C₁-C₆alkylaryl is unsubstituted or substituted with one to four substituents selected from the group consisting of halogen, C₁-C₆alkyl and haloC₁-C₆alkyl, or wherein when R³ is taken with R² or R¹ and R², forms a C₃-C₁₀cycloalkyl, aryl, heteroaryl or heterocycloalkyl, wherein the C₃-C₁₀cycloalkyl, aryl, heteroaryl or heterocycloalkyl is unsubstituted or substituted with one to four substituents selected from the group consisting of halogen, C₁-C₆alkyl, haloC₁-C₆alkyl, alkoxy, CN, COOC₁-C₆alkyl, COaryl and heteroaryl, wherein the heteroaryl is unsubstituted or substituted with one to four substituents selected from the group consisting of halogen, CN, C₁-C₆alkyl, alkoxy, aryl, C₃-C₆cycloalkyl and haloC₁-C₆alkyl;
R⁷ is hydrogen, OH, C₁-C₆alkylOH, CN, C₁-C₆alkylCN, C₁-C₆alkyl, C₁-C₆alkylaryl, C₁-C₆alkylaryl, C₂-C₆alkynyl, haloC₁-C₆alkyl, halogen, or alkoxy, wherein the C₂-C₆alkynyl is unsubstituted or substituted with one to three substituents selected from the group consisting of OH, C₁-C₆alkylOH, CN, C₁-C₆alkylCN, NH₂, NHC₁-C₆alkyl, N(C₁-C₆alkyl)₂, haloC₁-C₆alkyl, halogen, alkoxy, haloalkoxy, aryl, C₃-C₁₀cycloalkyl, heteroaryl and heterocycloalkyl, wherein the aryl, C₃-C₁₀cycloalkyl, heteroaryl and heterocycloalkyl are unsubstituted or substituted with one to three substituents selected from the group consisting of OH, C₁-C₆alkylOH, CN, C₁-C₆alkylCN, C₁-C₆alkyl, NH₂, NHC₁-C₆alkyl, N(C₁-C₆alkyl)₂, haloC₁-C₆alkyl, halogen, alkoxy, haloalkoxy;
R⁸ is hydrogen, OH, C₁-C₆alkylOH, CN, C₁-C₆alkylCN, C₁-C₆alkyl, C₁-C₆alkylaryl, alkynyl, alkynylaryl, haloC₁-C₆alkyl, halogen, or alkoxy;
R⁹ is hydrogen, OH, C₁-C₆alkylOH, CN, C₁-C₆alkylCN, C₁-C₆alkyl, C₁-C₆alkylaryl, alkynyl, alkynylaryl, haloC₁-C₆alkyl, halogen, or alkoxy;
R¹⁰ is hydrogen, OH, C₁-C₆alkylOH, CN, C₁-C₆alkylCN, C₁-C₆alkyl, C₁-C₆alkylaryl, alkynyl, alkynylaryl, haloC₁-C₆alkyl, halogen, or alkoxy.

Also described herein are compounds of Formula III:

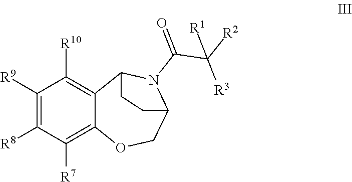

or a pharmaceutically acceptable salt thereof, wherein:
R¹ is hydrogen, OH, C₁-C₆alkylOH, CN, C₁-C₆alkylCN, C₁-C₆alkyl, C₃-C₆cycloalkyl, haloC₁-C₆alkyl, halogen, NH₂, N(C₁-C₆alkyl)₂, NH(C₁-C₆alkyl) or alkoxy, or wherein when R¹ is taken with R² or R² and R³, forms a C₃-C₁₀cycloalkyl, aryl, heteroaryl or heterocycloalkyl, wherein the C₃-C₁₀cycloalkyl, aryl, heteroaryl or heterocycloalkyl is unsubstituted or substituted with one to four substituents selected from the group consisting of halogen, C₁-C₆alkyl, haloC₁-C₆alkyl, alkoxy, CN, COOC₁-C₆alkyl, COaryl, aryl and heteroaryl, wherein the heteroaryl is unsubstituted or substituted with one to four substituents selected from the group consisting of halogen, CN, C₁-C₆alkyl, alkoxy, aryl, C₃-C₆cycloalkyl and haloC₁-C₆alkyl;
R² is hydrogen, OH, C₁-C₆alkylOH, CN, C₁-C₆alkylCN, C₁-C₆alkyl, C₃-C₆cycloalkyl, haloC₁-C₆alkyl, halogen, NH₂, N(C₁-C₆alkyl)₂, NH(C₁-C₆alkyl) or alkoxy, or wherein when R² is taken with R¹ or R¹ and R³, forms a C₃-C₁₀cycloalkyl, aryl, heteroaryl or heterocycloalkyl, wherein the C₃-C₁₀cycloalkyl, aryl, heteroaryl or heterocycloalkyl is unsubstituted or substituted with one to four substituents selected from the group consisting of halogen, C₁-C₆alkyl, haloC₁-C₆alkyl, alkoxy, CN, COOC₁-C₆alkyl, COaryl, aryl and heteroaryl, wherein the heteroaryl is unsubstituted or substituted with one to four substituents selected from the group consisting of halogen, CN, C₁-C₆alkyl, alkoxy, aryl, C₃-C₆cycloalkyl and haloC₁-C₆alkyl;
R³ is OH, C₁-C₆alkylOH, CN, C₁-C₆alkylCN, C₁-C₆alkyl, C₃-C₆cycloalkyl, haloC₁-C₆alkyl, halogen, C₁-C₆alkylOhaloC₁-C₆alkyl, alkoxy, CONHC₁-C₆alkylaryl, aryl, or C₁-C₆alkylaryl, wherein the CONHC₁-C₆alkylaryl, aryl, or C₁-C₆alkylaryl is unsubstituted or substituted with one to four substituents selected from the group consisting of halogen, C₁-C₆alkyl and haloC₁-C₆alkyl, or wherein when R³ is taken with R² or R¹ and R², forms a C₃-C₁₀cycloalkyl, aryl, heteroaryl or heterocycloalkyl, wherein the C₃-C₁₀cycloalkyl, aryl, heteroaryl or heterocycloalkyl is unsubstituted or substituted with one to four substituents selected from the group consisting of halogen, C₁-C₆alkyl, haloC₁-C₆alkyl, alkoxy, CN, COOC₁-C₆alkyl, COaryl and heteroaryl, wherein the heteroaryl is unsubstituted or substituted with one to four substituents selected from the group consisting of halogen, CN, C₁-C₆alkyl, alkoxy, aryl, C₃-C₆cycloalkyl and haloC₁-C₆alkyl;
R⁷ is hydrogen, OH, C₁-C₆alkylOH, CN, C₁-C₆alkylCN, C₁-C₆alkyl, C₁-C₆alkylaryl, C₁-C₆alkylaryl, C₂-C₆alkynyl, haloC₁-C₆alkyl, halogen, or alkoxy, wherein the C₂-C₆alkynyl is unsubstituted or substituted with one to three substituents selected from the group consisting of OH, C₁-C₆alkylOH, CN, C₁-C₆alkylCN, NH₂, NHC₁-C₆alkyl, N(C₁-C₆alkyl)₂, haloC₁-C₆alkyl, halogen, alkoxy, haloalkoxy, aryl, C₃-C₁₀cycloalkyl, heteroaryl and heterocycloalkyl, wherein the aryl, C₃-C₁₀cycloalkyl, heteroaryl and heterocycloalkyl are unsubstituted or substituted with one to three substituents selected from the group consisting of OH, C₁-C₆alkylOH, CN, C₁-C₆alkylCN, C₁-C₆alkyl, NH₂, NHC₁-C₆alkyl, N(C₁-C₆alkyl)₂, haloC₁-C₆alkyl, halogen, alkoxy, haloalkoxy;
R⁸ is hydrogen, OH, C₁-C₆alkylOH, CN, C₁-C₆alkylCN, C₁-C₆alkyl, C₁-C₆alkylaryl, alkynyl, alkynylaryl, haloC₁-C₆alkyl, halogen, or alkoxy;
R⁹ is hydrogen, OH, C₁-C₆alkylOH, CN, C₁-C₆alkylCN, C₁-C₆alkyl, C₁-C₆alkylaryl, alkynyl, alkynylaryl, haloC₁-C₆alkyl, halogen, or alkoxy;
R¹⁰ is hydrogen, OH, C₁-C₆alkylOH, CN, C₁-C₆alkylCN, C₁-C₆alkyl, C₁-C₆alkylaryl, alkynyl, alkynylaryl, haloC₁-C₆alkyl, halogen, or alkoxy.

Also, described herein are the following compounds:

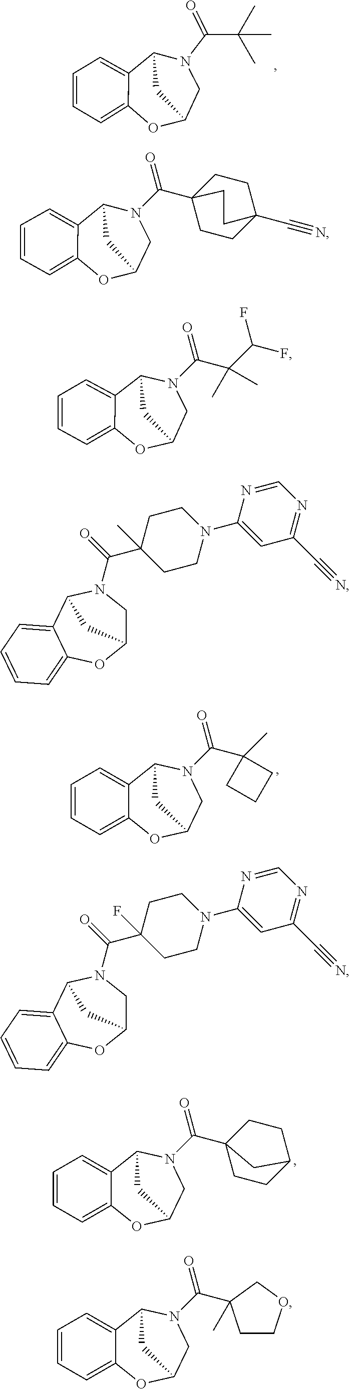

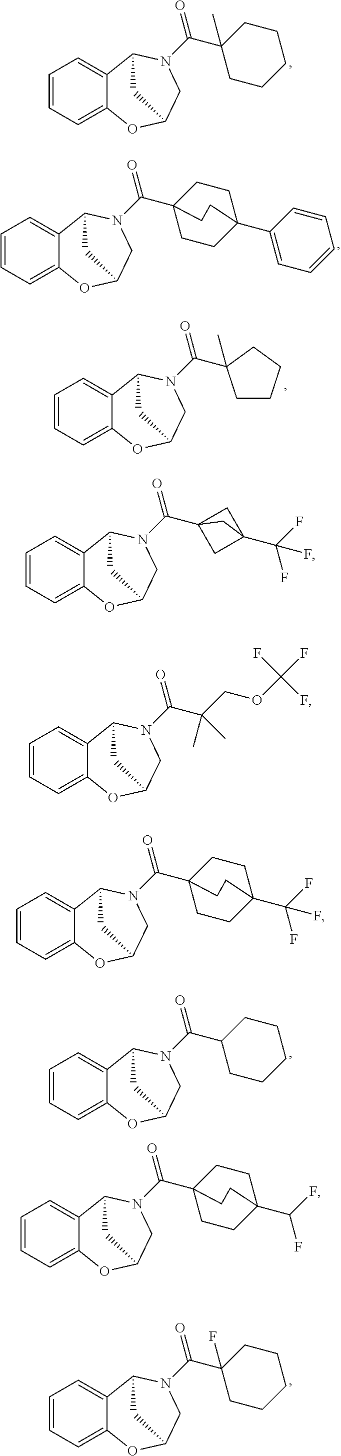

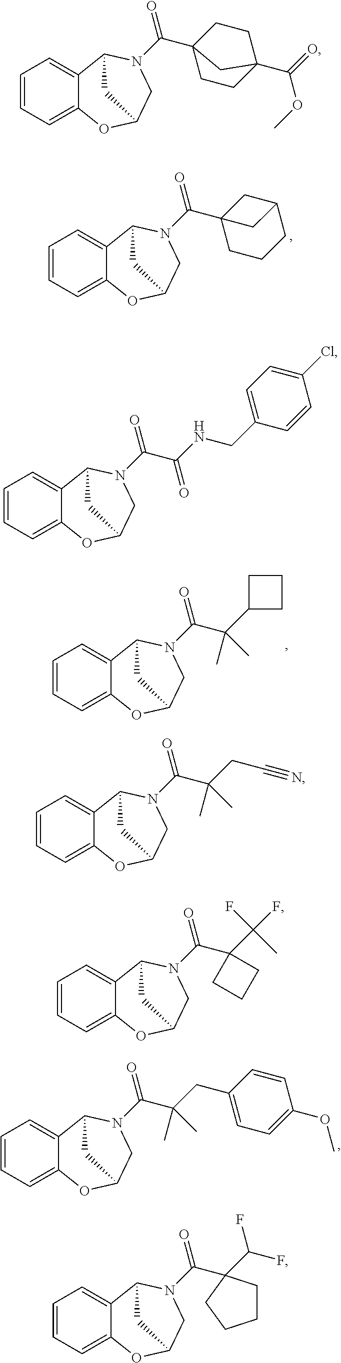

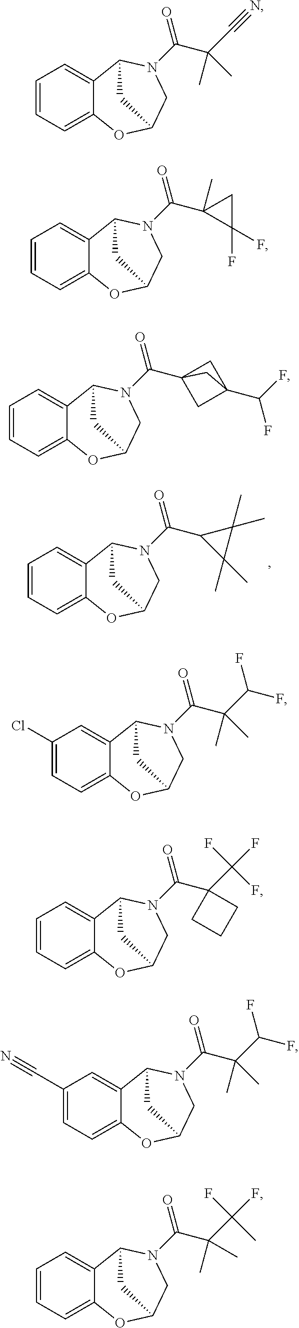

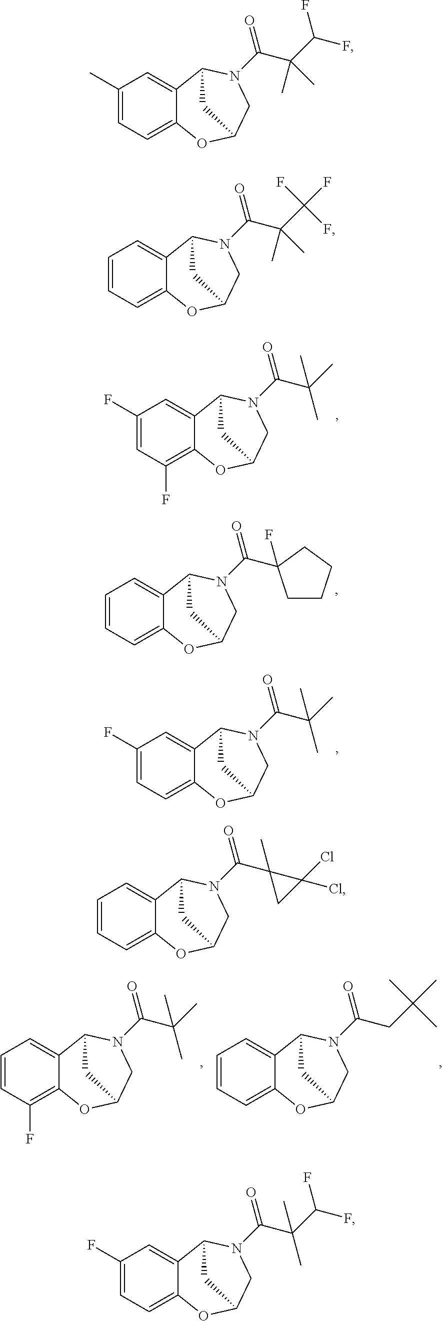

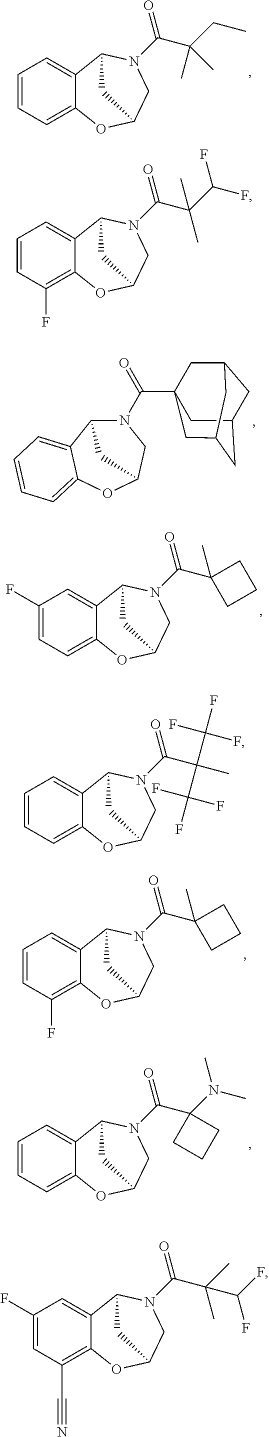

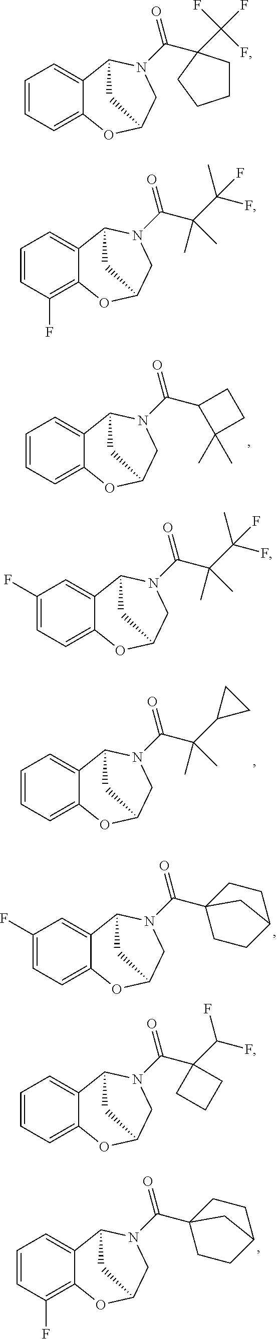

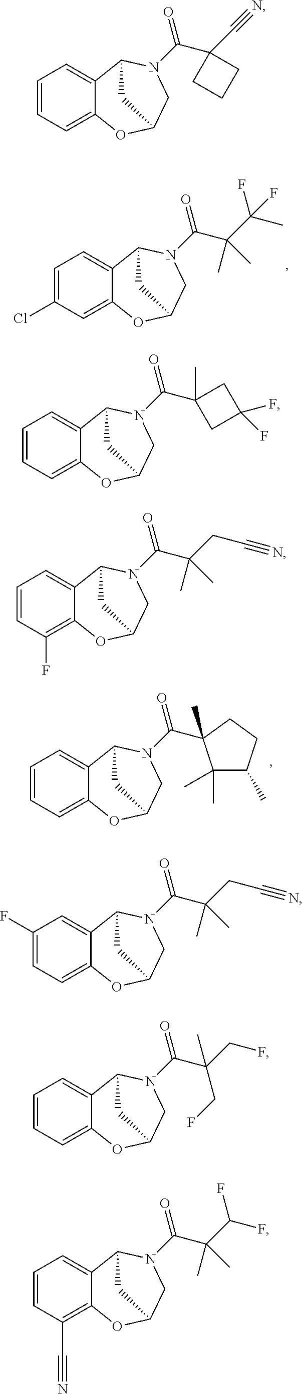

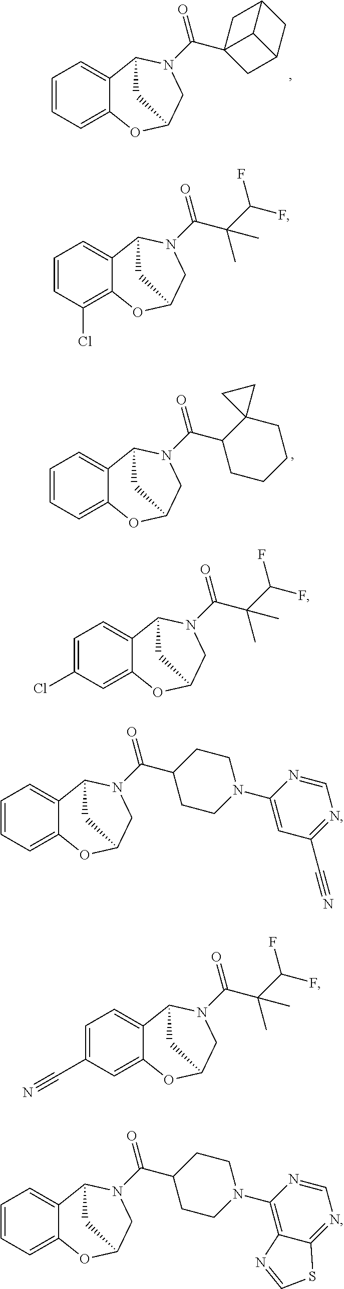

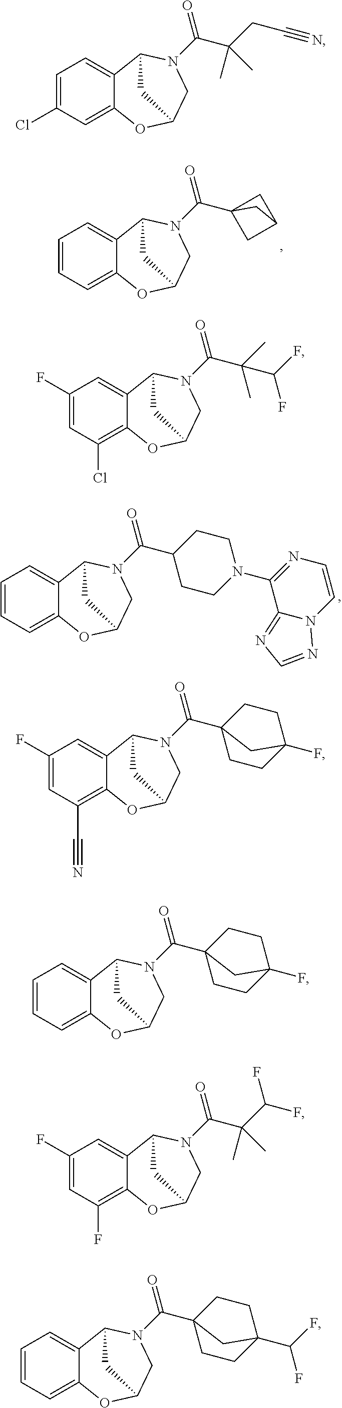

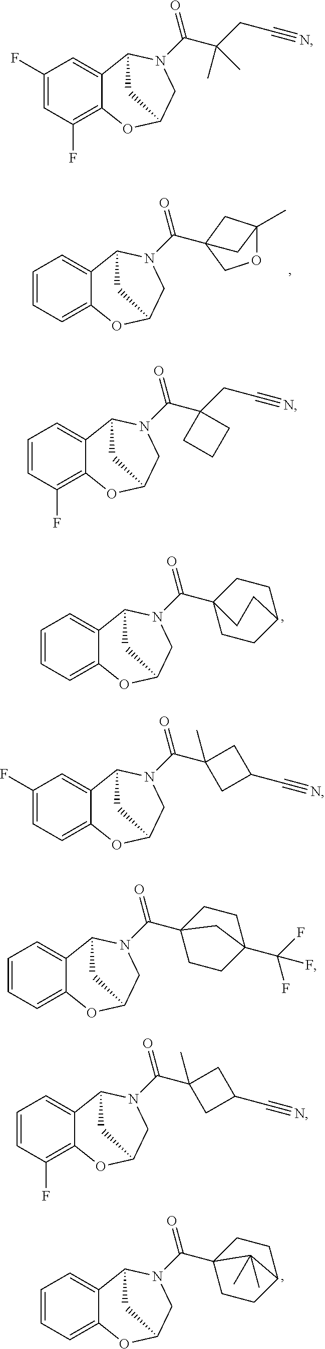

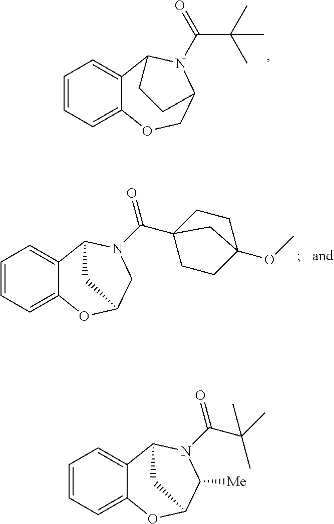 [SMILES: CC(C)(C)C(=O)N1C2CCC1c1ccccc1OC2 ; COC12CCC(C(=O)N3C[C@@H]4C[C@H]3c3ccccc3O4)(CC1)C2 ; C[C@@H]1[C@@H]2C[C@@H](c3ccccc3O2)N1C(=O)C(C)(C)C]

or a pharmaceutically acceptable salt thereof.

### Definitions

“Alkoxy” means an alkyl-O— group in which the alkyl group encompasses straight alkyl having a carbon number of 1 to 10 and branched alkyl having a carbon number of 3 to 10. Non-limiting examples of suitable alkoxy groups include methoxy, ethoxy, n-propoxy, isopropoxy and n-butoxy. The bond to the parent moiety is through the ether oxygen.

The term “halogen” includes fluorine, chlorine, bromine or iodine.

The term “C₁-C₆alkyl” encompasses straight alkyl having a carbon number of 1 to 6 and branched alkyl having a carbon number of 3 to 6. Specific examples thereof include methyl, ethyl, n-propyl, isopropyl, n-butyl, isobutyl, sec-butyl, tert-butyl, n-pentyl, isopentyl, neopentyl, tert-pentyl, 1-methylbutyl, 2-methylbutyl, 1,2-dimethylpropyl, 1-ethylpropyl, n-hexyl, isohexyl, 1-methylpentyl, 2-methylpentyl, 3-methylpentyl, 1,1-dimethylbutyl, 1,2-dimethylbutyl, 2,2-dimethylbutyl, 1-ethylbutyl, 1,1,2-trimethylpropyl, 1,2,2-trimethylpropyl, 1-ethyl-2-methylpropyl, 1-ethyl-1-methylpropyl, and the like.

The term “C₃-C₆cycloalkyl” encompasses bridged, saturated or unsaturated cycloalkyl groups having 3 to 6 carbons. Examples of cycloalkyl include cyclopropyl, cyclobutyl, cyclopentyl and cyclohexyl.

The term “C₃-C₁₀cycloalkyl” encompasses bridged, saturated or unsaturated cycloalkyl groups having 3 to 10 carbons. “Cycloalkyl” also includes non-aromatic rings as well as monocyclic, non-aromatic rings fused to a saturated cycloalkyl group. Examples of cycloalkyl include cyclopropyl, cyclobutyl, cyclopentyl, cyclohexyl, cycloheptyl, tetrahydronaphthyl, decahydronaphthyl, indanyl and the like. Examples described by structure include,

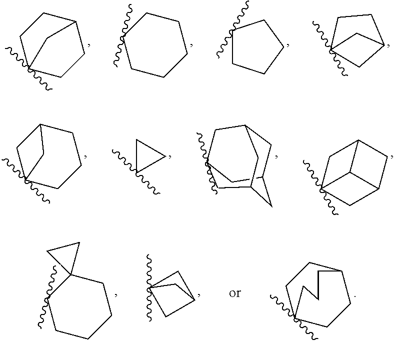

The term “heteroaryl” means a monocyclic or multicyclic, including bicyclic, aromatic cycloheteroalkyl that contains at least one ring heteroatom selected from O, S and N. Examples of heteroaryl groups include pyridyl (pyridinyl), oxazolyl, azabenzothiazole, benzothiazole, imidazolyl, triazolyl, furyl, triavinyl, thienyl, pyrimidyl, pyridazinyl, indolizinyl, cinnolinyl, phthalazinyl, quinazolinyl, naphthyridinyl, quinoxalinyl, purinyl, benzimidazolyl, quinolyl, isoquinolyl, and the like.

The term “cycloheteroalkyl” means mono- or bicyclic or bridged partially unsaturated and saturated rings containing at least one heteroatom selected from N, S and O, each of said rings having from 3 to 10 atoms in which the point of attachment may be carbon or nitrogen. Examples include azetidine, tetrahydropyranyl, tetrahydrofuranyl, pyrrolidinyl, piperidinyl, piperazinyl, dioxanyl, imidazolidinyl, 2,3-dihydrofuro(2,3-b)pyridyl, benzoxazinyl, benzoxazolinyl, 2-H-phthalazinyl, isoindolinyl, benzoxazepinyl, 5,6-dihydroimidazo[2,1-b]thiazolyl, tetrahydroquinolinyl, morpholinyl, tetrahydroisoquinolinyl, dihydroindolyl, and the like. The term also includes partially unsaturated monocyclic rings that are not aromatic, such as 2- or 4-pyridones attached through the nitrogen or n-substituted-(1H, 3H)-pyrimidine-2,4-diones (N-substituted uracils). The term also includes bridged rings such as 5-azabicyclo[2.2.1]heptyl, 2,5-diazabicyclo[2.2.1]heptyl, 2-azabicyclo[2.2.1]heptyl, 7-azabicyclo[2.2.1]heptyl, 2,5-diazabicyclo[2.2.2]octyl, 2-azabicyclo[2.2.2]octyl, and 3-azabicyclo[3.2.2]nonyl, and azabicyclo[2.2.1]heptanyl. Examples described by structure include,

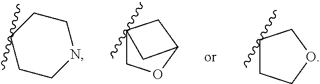

The term “pharmaceutically acceptable salt” refers to salts prepared from pharmaceutically acceptable non-toxic bases or acids including inorganic or organic bases and inorganic or organic acids. Salts of basic compounds encompassed within the term “pharmaceutically acceptable salt” refer to non-toxic salts of the compounds of this invention which are generally prepared by reacting the free base with a suitable organic or inorganic acid. Representative salts of basic compounds of the present invention include, but are not limited to, the following: acetate, benzenesulfonate, benzoate, bicarbonate, bisulfate, bitartrate, borate, bromide, camsylate, carbonate, chloride, clavulanate, citrate, dihydrochloride, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isothionate, lactate, lactobionate, laurate, malate, maleate, mandelate, mesylate, methylbromide, methylnitrate, methylsulfate, mucate, napsylate, nitrate, n-methylglucamine ammonium salt, oleate, oxalate, pamoate (embonate), palmitate, pantothenate, phosphate/diphosphate, polygalacturonate, salicylate, stearate, sulfate, subacetate, succinate, tannate, tartrate, teoclate, tosylate, triethiodide and valerate. Furthermore, where the compounds of the invention carry an acidic moiety, suitable pharmaceutically acceptable salts thereof include, but are not limited to, salts derived from inorganic bases including aluminum, ammonium, calcium, copper, ferric, ferrous, lithium, magnesium, manganic, mangamous, potassium, sodium, zinc, and the like. Particularly preferred are the ammonium, calcium, magnesium, potassium, and sodium salts. Salts derived from pharmaceutically acceptable organic non-toxic bases include salts of primary, secondary, and tertiary amines, cyclic amines, and basic ion-exchange resins, such as arginine, betaine, caffeine, choline, N,N-dibenzylethylenediamine, diethylamine, 2-diethylaminoethanol, 2-dimethylaminoethanol, ethanolamine, ethylenediamine, n-ethylmorpholine, n-ethylpiperidinyl, glucamine, glucosamine, histidine, hydrabamine, isopropylamine, lysine, methylglucamine, morpholine, piperazine, piperidinyl, polyamine resins, procaine, purines, theobromine, triethylamine, trimethylamine, tripropylamine, tromethamine, and the like.

The term “patient” refers to a mammalian patient, preferably a human patient, receiving or about to receive medical treatment.

The compounds of the present invention may contain one or more asymmetric centers and can thus occur as racemates, racemic mixtures, single enantiomers, diastereomeric mixtures, and individual diastereomers. The present invention is meant to comprehend all such isomeric forms of these compounds.

Some of the compounds described herein contain olefinic double bonds, and unless specified otherwise, are meant to include both E and Z geometric isomers.

Some of the compounds described herein contain substituted cycloalkanes having cis- and trans-isomers, and unless specified otherwise, are meant to include both cis- and trans-geometric isomers.

The independent syntheses of these diastereomers or their chromatographic separations may be achieved as known in the art by appropriate modification of the methodology disclosed herein. Their absolute stereochemistry may be determined by the X-ray crystallography of crystalline products or crystalline intermediates which are derivatized, if necessary, with a reagent containing an asymmetric center of known absolute configuration. If desired, racemic mixtures of the compounds may be separated so that the individual enantiomers are isolated. The separation can be carried out by methods well known in the art, such as the coupling of a racemic mixture of compounds to an enantiomerically pure compound to form a diastereomeric mixture, followed by separation of the individual diastereomers by standard methods, such as fractional crystallization or chromatography. The coupling reaction is often the formation of salts using an enantiomerically pure acid or base. The diastereomeric derivatives may then be converted to the pure enantiomers by cleavage of the added chiral residue. The racemic mixture of the compounds can also be separated directly by chromatographic methods utilizing chiral stationary phases, which methods are well known in the art.

Alternatively, any enantiomer of a compound may be obtained by stereoselective synthesis using optically pure starting materials or reagents of known configuration by methods well known in the art.

It will be understood that the present invention is meant to include the pharmaceutically acceptable salts, and also salts that are not pharmaceutically acceptable, of the compounds described herein, when they are used as precursors to the free compounds or their pharmaceutically acceptable salts or in other synthetic manipulations.

Solvates, and in particular, the hydrates of the compounds of the structural formulas described herein are included in the present invention as well.

Some of the compounds described herein may exist as tautomers, which have different points of attachment of hydrogen accompanied by one or more double bond shifts. For example, a ketone and its enol form are keto-enol tautomers. The individual tautomers as well as mixtures thereof are encompassed with compounds of the present invention.

In the compounds described herein, the atoms may exhibit their natural isotopic abundances, or one or more of the atoms may be artificially enriched in a particular isotope having the same atomic number, but an atomic mass or mass number different from the atomic mass or mass number predominantly found in nature. The present invention is meant to include all suitable isotopic variations of the compounds of the formulas described herein. For example, different isotopic forms of hydrogen (H) include protium (¹H) and deuterium (²H). Protium is the predominant hydrogen isotope found in nature. Enriching for deuterium may afford certain therapeutic advantages, such as increasing in vivo half-life or reducing dosage requirements, or may provide a compound useful as a standard for characterization of biological samples. A ³H, ¹¹C, ¹⁸F labeled compound may be used for PET or SPECT or other imaging studies. Isotopically-enriched compounds can be prepared without undue experimentation by conventional techniques well known to those skilled in the art or by processes analogous to those described in the Schemes and Examples herein using appropriate isotopically-enriched reagents or Intermediates.

It should be noted that chemically unstable compounds are excluded from the embodiments contained herein.

### Methods of Treatment

The compounds described herein may be particularly useful for the prevention, treatment or amelioration of RIPK1-mediated diseases or disorders. Such RIPK1-mediated diseases or disorders are likely to be regulated at least in part by programmed necrosis, apoptosis or the production of inflammatory cytokines, particularly inflammatory bowel disease (including Crohn's disease and ulcerative colitis), psoriasis, retinal detachment, retinal degeneration, retinitis pigmentosa, macular degeneration, age-related macular degeneration, pancreatitis, atopic dermatitis, arthritis (including rheumatoid arthritis, spondyloarthritis, gout, juvenile idiopathic arthritis (systemic onset juvenile idiopathic arthritis (SoJIA)), psoriatic arthritis), lupus, systemic lupus erythematosus (SLE), Sjogren's syndrome, systemic scleroderma, anti-phospholipid syndrome (APS), vasculitis, osteoarthritis, liver damage/diseases (non-alcoholic steatohepatitis (NASH), alcoholic steatohepatitis (ASH), autoimmune hepatitis, autoimmune hepatobiliary diseases, primary sclerosing cholangitis (PSC), acetaminophen toxicity, hepatotoxicity), autoimmune hepatitis, non-alcoholic fatty liver disease (NAFL D), kidney damage/injury (nephritis, renal transplant, surgery, administration of nephrotoxic drugs e.g. cisplatin, acute kidney injury (AKI)), Celiac disease, autoimmune idiopathic thrombocytopenia purpura (autoimmune ITP), transplant rejection (rejection of transplant organs, tissues and cells), ischemia reperfusion injury of solid organs, sepsis, systemic inflammatory response syndrome (SIRS), cerebrovascular accident (CV A, stroke), myocardial infarction (MI), atherosclerosis, Huntington's disease, Alzheimer's disease, Parkinson's disease, amyotrophic lateral sclerosis (ALS), progressive supranuclear palsy (PSP), neonatal brain injury, neonatal hypoxic brain injury, ischemic brain injury, traumatic brain injury allergic diseases (including asthma and atopic dermatitis), peripheral nerve injury, burns, multiple sclerosis, type I diabetes, type II diabetes, obesity, Wegener's granulomatosis, pulmonary sarcoidosis, Behcet's disease, interleukin-I converting enzyme (ICE, also known as caspase-1) associated fever syndrome, chronic obstructive pulmonary disease (COPD), cigarette smoke-induced damage, cystic fibrosis, tumor necrosis factor receptor-associated periodic syndrome (TRAPS), a neoplastic tumor, peridontitis, NEMO-mutations (mutations of NF-kappa-B essential modulator gene (also known as IKK gamma or IKKG)), particularly, NEMO-deficiency syndrome, HOIL-1 deficiency (also known as RBCK1) heme-oxidized IRP 2 ubiquitin ligase-1 deficiency), linear ubiquitin chain assembly complex (LUBAC) deficiency syndrome, hematological and solid organ malignancies, bacterial infections and viral infections (such as influenza, Staphylococcus, and Mycobacterium (tuberculosis)), and Lysosomal storage diseases (particularly, Gaucher disease, and including GM2 gangliosidosis, alpha-mannosidosis, aspartylglucosaminuria, cholesteryl ester storage disease, chronic hexosaminidase A deficiency, cystinosis, Danon disease, Fabry disease, Farber disease, fucosidosis, galactosialidosis, GM1 gangliosidosis, mucolipidosis, infantile free sialic acid storage disease, juvenile hexosaminidase A deficiency, Krabbe disease, lysosomal acid lipase deficiency, metachromatic leukodystrophy, mucopolysaccharidoses disorders, multiple sulfatase deficiency, Niemann-Pick disease, neuronal ceroid lipofuscinoses, Pompe disease, pycnodysostosis, Sandhoff disease, Schindler disease, sialic acid storage disease, Tay-Sachs, and Wolman disease), Stevens-Johnson syndrome, toxic epidermal necrolysis, glaucoma, spinal cord injury, fibrosis, complement-mediated cytotoxicity, pancreatic ductal adenocarcinoma, hepatocellular carcinoma, mesothelioma, melanoma, metastasis, breast cancer, non-small cell lung carcinoma (NSCLC), radiation induced necrosis, ischemic kidney damage, ophthalmologic ischemia, intracerebral hemorrhage, subarachnoid hemorrhage, acute liver failure and radiation protection/mitigation, auditory disorders such as noise-induced hearing loss and drugs associated with ototoxicity such as cisplatin, or for the treatment of cells ex vivo to preserve vitality and function.

The compounds of the Formulae described herein, or a pharmaceutically acceptable salt thereof, may be particularly useful for the treatment of the following RIPK1-mediated diseases or disorders: inflammatory bowel disease (including Crohn's disease and ulcerative colitis), psoriasis, retinal detachment, retinal degeneration, retinitis pigmentosa, macular degeneration, age-related macular degeneration, pancreatitis, atopic dermatitis, arthritis (including rheumatoid arthritis, spondyloarthritis, gout, systemic onset juvenile idiopathic arthritis (SoJIA), psoriatic arthritis), lupus, systemic lupus erythematosus (SLE), Sjogren's syndrome, systemic scleroderma, anti-phospholipid syndrome (APS), vasculitis, osteoarthritis, liver damage/diseases, autoimmune hepatitis, autoimmune hepatobiliary diseases, primary sclerosing cholangitis (PSC), acetaminophen toxicity, hepatotoxicity), non-alcoholic steatohepatitis (NASH), alcoholic steatohepatitis (ASH), autoimmune hepatitis, non-alcoholic fatty liver disease (NAFLD), kidney damage/injury (nephritis, renal transplant, surgery, administration of nephrotoxic drugs e.g. cisplatin, acute kidney injury (AKI)), Celiac disease, autoimmune idiopathic thrombocytopenic purpura (autoimmune ITP), transplant rejection (rejection of transplant organs, tissues and cells), ischemia reperfusion injury of solid organs, sepsis, systemic inflammatory response syndrome (SIRS), cerebrovascular accident (CVA, stroke), myocardial infarction (MI), atherosclerosis, Huntington's disease, Alzheimer's disease, Parkinson's disease, amyotrophic lateral sclerosis (ALS), progressive supranuclear palsy (PSP), neonatal brain injury, neonatal hypoxic brain injury, traumatic brain injury, allergic diseases (including asthma and atopic dermatitis), peripheral nerve injury, burns, multiple sclerosis, type I diabetes, type II diabetes, obesity, Wegener's granulomatosis, pulmonary sarcoidosis, Behcet's disease, interleukin-I converting enzyme (ICE, also known as caspase-1) associated fever syndrome, chronic obstructive pulmonary disease (COPD), cigarette smoke-induced damage, cystic fibrosis, tumor necrosis factor receptor-associated periodic syndrome (TRAPS), a neoplastic tumor, melanoma, metastasis, breast cancer, non-small cell lung carcinoma (NSCLC), radiation induced necrosis, ischemic kidney damage, ophthalmologic ischemia, intracerebral hemorrhage, subarachnoid hemorrhage, peridontitis, NEMO-mutations (mutations of NF-kappa-B essential modulator gene (also known as IKK gamma or IKKG)), particularly, NEMO-deficiency syndrome, HOIL-1 deficiency ((also known as RBCK1) heme-oxidized IRP 2 ubiquitin ligase-1 deficiency), linear ubiquitin chain assembly complex (LUBAC) deficiency syndrome, hematological and solid organ malignancies, bacterial infections and viral infections (such as influenza, Staphylococcus, and Mycobacterium (tuberculosis)), and Lysosomal storage diseases (particularly, Gaucher disease, and including GM2 gangliosidosis, alpha-mannosidosis, aspartylglucosaminuria, cholesteryl ester storage disease, chronic hexosaminidase A deficiency, cystinosis, Danon disease, Fabry disease, Farber disease, fucosidosis, galactosialidosis, GM1 gangliosidosis, mucolipidosis, infantile free sialic acid storage disease, juvenile hexosaminidase A deficiency, Krabbe disease, lysosomal acid lipase deficiency, metachromatic leukodystrophy, mucopolysaccharidoses disorders, multiple sulfatase deficiency, Niemann-Pick disease, neuronal ceroid lipofuscinoses, Pompe disease, pycnodysostosis, Sandhoff disease, Schindler disease, sialic acid storage disease, Tay-Sachs, and Wolman disease), spinal cord injury, Stevens-Johnson syndrome, fibrosis, complement-mediated cytotoxicity, toxic epidermal necrolysis, and/or for the treatment of cells ex vivo to preserve vitality and function.

The compounds of the Formulae described herein, or a pharmaceutically acceptable salt thereof, may be useful for the treatment of glaucoma.

The compounds of the Formulae described herein, or a pharmaceutically acceptable salt thereof, may be particularly useful for treatment of pancreatic ductal adenocarcinoma, hepatocellular carcinoma, mesothelioma, or melanoma.

The compounds of the Formulae described herein, or a pharmaceutically acceptable salt thereof, may be particularly useful for the treatment of the following RIPK1-mediated disease or disorder: rheumatoid arthritis, inflammatory bowel disease (including Crohn's disease and ulcerative colitis), and psoriasis.

The treatment of the above-noted diseases/disorders may concern, more specifically, the amelioration of organ injury or damage sustained as a result of the noted diseases/disorders. For example, the compounds of this invention may be particularly useful for amelioration of brain tissue injury or damage following ischemic brain injury or traumatic brain injury, or for amelioration of heart tissue injury or damage following myocardial infarction, or for amelioration of brain tissue injury or damage associated with Huntington's disease, Alzheimer's disease or Parkinson's disease, or for amelioration of liver tissue injury or damage associated with non-alcoholic steatohepatitis, alcoholic steatohepatitis, autoimmune hepatitis autoimmune hepatobiliary diseases, or primary sclerosing cholangitis, or overdose of acetaminophen.

The compounds of this invention may be particularly useful for the amelioration of organ injury or damage sustained as a result of radiation therapy, or amelioration of spinal tissue injury or damage following spinal cord injury or amelioration of liver tissue injury or damage associated acute liver failure. The compounds of this invention may be particularly useful for amelioration of auditory disorders, such as noise-induced hearing loss or auditory disorders following the administration of ototoxic drugs or substances e.g. cisplatin.

The compounds of this invention may be particularly useful for amelioration of solid organ tissue (particularly kidney, liver, and heart and/or lung) injury or damage following transplant or the administration of nephrotoxic drugs or substances e.g. cisplatin. It will be understood that amelioration of such tissue damage may be achieved where possible, by pre-treatment with a compound of the Formulae described herein, or a pharmaceutically acceptable salt thereof; for example, by pre-treatment of a patient prior to administration of cisplatin or pre-treatment of an organ or the organ recipient prior to transplant surgery. Amelioration of such tissue damage may be achieved by treatment with a compound of the Formulae described herein, or a pharmaceutically acceptable salt thereof, during transplant surgery.

Amelioration of such tissue damage may also be achieved by short-term treatment of a patient with a compound of the Formulae described herein, or a pharmaceutically acceptable salt thereof, after transplant surgery.

In one embodiment, the compounds of the Formulae described herein, or a pharmaceutically acceptable salt thereof, may be useful for the treatment of retinal detachment, macular degeneration, and retinitis pigmentosa.

In another embodiment, the compounds of the Formulae described herein, or a pharmaceutically acceptable salt thereof, may be useful for the treatment of multiple sclerosis.

In one embodiment, the compounds of the Formulae described herein, or a pharmaceutically acceptable salt thereof, may be useful for the treatment of traumatic brain injury.

In another embodiment, the compounds of the Formulae described herein, or a pharmaceutically acceptable salt thereof, may be useful for the treatment of Huntington's Disease or Niemann-Pick disease.

In another embodiment, the compounds of the Formulae described herein, or a pharmaceutically acceptable salt thereof, may be useful for the treatment of amyotrophic lateral sclerosis (ALS), progressive supranuclear palsy (PSP), and Alzheimer's disease.

In another embodiment, the compounds of the Formulae described herein, or a pharmaceutically acceptable salt thereof, may be useful for the treatment of age-related macular degeneration.

The treatment of retinal detachment, macular degeneration, retinitis pigmentosa, multiple sclerosis, traumatic brain injury, Huntington's Disease, Alzheimer's Disease, amyotrophic lateral sclerosis, and Niemann-Pick disease may concern, more specifically, the amelioration of organ injury or damage sustained as a result of these diseases/disorders. For example, the compounds described herein may be particularly useful for amelioration of brain tissue injury or damage following traumatic brain injury, or for amelioration of brain tissue injury or damage associated of Huntington's Disease, Alzheimer's Disease, amyotrophic lateral sclerosis, and Niemann-Pick disease.

In another embodiment, the compounds of the Formulae described herein, or a pharmaceutically acceptable salt thereof, may be useful for the treatment of retinal detachment, macular degeneration, and retinitis pigmentosa, and the amelioration of brain tissue injury or damage as a result of multiple sclerosis, traumatic brain injury, Huntington's Disease, Alzheimer's Disease, amyotrophic lateral sclerosis, and Niemann-Pick disease.

In another embodiment, the compounds of the Formulae described herein, or a pharmaceutically acceptable salt thereof, may be useful for the treatment of Crohn's disease, ulcerative colitis, psoriasis, rheumatoid arthritis, spondyloarthritis, systemic onset juvenile idiopathic arthritis (SoJIA), and osteoarthritis.

In yet another embodiment, the compounds of the Formulae described herein, or a pharmaceutically acceptable salt thereof, may be useful for the treatment of psoriasis, rheumatoid arthritis, and ulcerative and colitis.

In another embodiment, the compounds of the Formulae described herein, or a pharmaceutically acceptable salt thereof, may be useful for the treatment of lupus, inflammatory bowel disease (IBD), Crohn's disease, and ulcerative colitis.

In another embodiment, the compounds of the Formulae described herein, or a pharmaceutically acceptable salt thereof, may be useful for the treatment of cerebrovascular accident (CVA, stroke), Huntington's disease, Alzheimer's disease, amyotrophic lateral sclerosis (ALS), traumatic brain injury, multiple sclerosis, Gaucher disease, Niemann-Pick disease, and spinal cord injury.

In another embodiment, the compounds of the Formulae described herein, or a pharmaceutically acceptable salt thereof, may be useful for the treatment of amyotrophic lateral sclerosis (ALS).

In another embodiment, the compounds of the Formulae described herein, or a pharmaceutically acceptable salt thereof, may be useful for the treatment of multiple sclerosis.

In another embodiment, the compounds of the Formulae described herein, or a pharmaceutically acceptable salt thereof, may be useful for the treatment of pancreatic ductal adenocarcinoma (PDAC), metastasis, melanoma, breast cancer, non-small cell lung carcinoma (NSCLC), and radiation induced necrosis.

In another embodiment, the compounds of the Formulae described herein, or a pharmaceutically acceptable salt thereof, may be useful for the treatment of pancreatic ductal adenocarcinoma (PDAC), metastasis, melanoma, breast cancer, and non-small cell lung carcinoma (NSCLC).

In another embodiment, the compounds of the Formulae described herein, or a pharmaceutically acceptable salt thereof, may be useful for the treatment of pancreatic ductal adenocarcinoma (PDAC).

In another embodiment, the compounds of the Formulae described herein, or a pharmaceutically acceptable salt thereof, may be useful for the treatment of intracerebral hemorrhage and subarachnoid hemorrhage.

In another embodiment, the compounds of the Formulae described herein, or a pharmaceutically acceptable salt thereof, may be useful for the treatment of type II diabetes and obesity.

In another embodiment, the compounds of the Formulae described herein, or a pharmaceutically acceptable salt thereof, may be useful for the treatment of atherosclerosis.

In another embodiment, the compounds of the Formulae described herein, or a pharmaceutically acceptable salt thereof, may be useful for the treatment of vasculitis.

In another embodiment, the compounds of the Formulae described herein, or a pharmaceutically acceptable salt thereof, may be useful for the treatment of dependent inflammation and cell death that occurs in inherited and sporadic diseases including Alzheimer's disease, amyotrophic lateral sclerosis, multiple sclerosis, Parkinson's disease, chronic traumatic encephalopathy, rheumatoid arthritis, ulcerative colitis, inflammatory bowel disease, psoriasis as well as acute tissue injury caused by stroke, traumatic brain injury, encephalitis.

In another embodiment, the compounds of the Formulae described herein, or pharmaceutically acceptable salt thereof, may be useful for the treatment of ischemic kidney damage, ophthalmologic ischemia, intracerebral hemorrhage, and subarachnoid hemorrhage.

In another embodiment, the compounds of the Formulae described herein, or pharmaceutically acceptable salt thereof, may be useful for the treatment of non-alcoholic steatohepatitis (NASH), alcoholic steatohepatitis (ASH), autoimmune hepatitis, and non-alcoholic fatty liver disease (NAFLD).

The compounds of the invention, particularly the compounds of the Formulae described herein, or a pharmaceutically acceptable salt thereof, may be particularly useful for the treatment of the RIPK1-mediated, cancer-related diseases or disorders. Gong et al., The role of necroptosis in cancer biology and therapy, Molecular Cancer (2019) 18:100. In one aspect the human has a solid tumor. In one aspect the tumor is selected from head and neck cancer, gastric cancer, melanoma, renal cell carcinoma (RCC), esophageal cancer, non-small cell lung carcinoma (NSCLC), prostate cancer, colorectal cancer, ovarian cancer, pancreatic cancer, and pancreatic ductal adenocarcinoma. In one aspect the human has one or more of the following: colorectal cancer (CRC), esophageal cancer, cervical, bladder, breast cancer, head and neck cancer, ovarian cancer, melanoma, renal cell carcinoma (RCC), EC squamous cell carcinoma, non-small cell lung carcinoma, mesothelioma, prostate cancer, and pancreatic ductal adenocarcinoma. In another aspect, the human has a liquid tumor such as diffuse large B cell lymphoma (DLBCL), multiple myeloma, chronic lymphoblastic leukemia (CLL), follicular lymphoma, acute myeloid leukemia and chronic myelogenous leukemia.

The present disclosure also relates to a method for treating or lessening the severity of a cancer selected from: brain (gliomas), glioblastomas, astrocytomas, Bannayan-Zonana syndrome, Cowden disease, Lhermitte-Duclos disease, breast cancer, triple negative breast cancer, inflammatory breast cancer, Wilm's tumor, Ewing's sarcoma, Rhabdomyosarcoma, ependymoma, medulloblastoma, colon cancer, head and neck cancer (including squamous cell carcinoma of head and neck), kidney cancer, lung cancer (including lung squamous cell carcinoma, lung adenocarcinoma, lung small cell carcinoma, and non-small cell lung carcinoma), liver cancer (including hepatocellular carcinoma), melanoma, ovarian cancer, pancreatic cancer (including squamous pancreatic cancer), prostate cancer, sarcoma, osteosarcoma, giant cell tumor of bone, thyroid cancer, lymphoblastic T-cell leukemia, chronic myelogenous leukemia, chronic lymphocytic leukemia, hairy-cell leukemia, acute lymphoblastic leukemia, acute myelogenous leukemia, chronic neutrophilic leukemia, acute lymphoblastic T-cell leukemia, plasmacytoma, immunoblastic large cell leukemia, mantle cell leukemia, multiple myeloma megakaryoblastic leukemia, multiple myeloma, acute megakaryocytic leukemia, promyelocytic leukemia, erythroleukemia, malignant lymphoma, Hodgkin's lymphoma, Non-Hodgkin's lymphoma, lymphoblastic T cell lymphoma, Burkitt's lymphoma, follicular lymphoma, neuroblastoma, bladder cancer, urothelial cancer, lung cancer, vulval cancer, cervical cancer, endometrial cancer, cancer of the uterus, renal cancer (including kidney clear cell cancer, kidney papillary cancer, renal cell carcinoma), mesothelioma, esophageal cancer, salivary gland cancer, hepatocellular cancer, gastric cancer, nasopharyngeal cancer, buccal cancer, cancer of the mouth, GIST (gastrointestinal stromal tumor) and testicular cancer.

Specific examples of clinical conditions based on hematologic tumors include leukemias such as chronic myelocytic leukemia, acute myelocytic leukemia, chronic lymphocytic leukemia and acute lymphocytic leukemia; plasma cell malignancies such as multiple myeloma, MGUS and Waldenstrom's macroglobulinemia; lymphomas such as non-Hodgkin's lymphoma, Hodgkin's lymphoma; and the like.

The cancer may be any cancer in which an abnormal number of blast cells or unwanted cell proliferation is present or that is diagnosed as a hematological cancer, including both lymphoid and myeloid malignancies. Myeloid malignancies include, but are not limited to, acute myeloid (or myelocytic or myelogenous or myeloblastic) leukemia (undifferentiated or differentiated), acute promyeloid (or promyelocytic or promyelogenous or promyeloblastic) leukemia, acute myelomonocytic (or myelomonoblastic) leukemia, acute monocytic (or monoblastic) leukemia, erythroleukemia and megakaryocytic (or megakaryoblastic) leukemia. These leukemias may be referred together as acute myeloid (or myelocytic or myelogenous) leukemia (AML). Myeloid malignancies also include myeloproliferative disorders (MPD) which include, but are not limited to, chronic myelogenous (or myeloid) leukemia (CML), chronic myelomonocytic leukemia (CMML), essential thrombocythemia (or thrombocytosis), and polycythemia vera (PCV). Myeloid malignancies also include myelodysplasia (or myelodysplastic syndrome or MDS), which may be referred to as refractory anemia (RA), refractory anemia with excess blasts (RAEB), and refractory anemia with excess blasts in transformation (RAEBT); as well as myelofibrosis (MFS) with or without agnogenic myeloid metaplasia.

Specific examples of clinical conditions based on hematologic tumors include leukemias such as chronic myelocytic leukemia, acute myelocytic leukemia, chronic lymphocytic leukemia and acute lymphocytic leukemia; plasma cell malignancies such as multiple myeloma. MGUS and Waldenstrom's macroglobulinemia; lymphomas such as non-Hodgkin's lymphoma. Hodgkin's lymphoma; and the like. Hematopoietic cancers also include lymphoid malignancies, which may affect the lymph nodes, spleens, bone marrow, peripheral blood, and/or extranodal sites. Lymphoid cancers include B-cell malignancies, which include, but are not limited to, B-cell non-Hodgkin's lymphomas (B-NHLs). B-NHLs may be indolent (or low-grade), intermediate grade (or aggressive) or high-grade (very aggressive). Indolent B cell lymphomas include follicular lymphoma (FL); small lymphocytic lymphoma (SLL); marginal zone lymphoma (MZL) including nodal MZL, extranodal MZL, splenic MZL and splenic MZL with villous lymphocytes; lymphoplasmacytic lymphoma (LPL); and mucosa-associated-lymphoid tissue (MALT or extranodal marginal zone) lymphoma. Intermediate-grade B-NHLs include mantle cell lymphoma (MCL) with or without leukemic involvement, diffuse large cell lymphoma (DLBCL), follicular large cell (or grade 3 or grade 3B) lymphoma, and primary mediastinal lymphoma (PML). High-grade B-NHLs include Burkitt's lymphoma (BL), Burkitt-like lymphoma, small non-cleaved cell lymphoma (SNCCL) and lymphoblastic lymphoma. Other B-NHLs include immunoblastic lymphoma (or immunocytoma), primary effusion lymphoma. HIV associated (or AIDS related) lymphomas, and post-transplant lymphoproliferative disorder (PTLD) or lymphoma, B-cell malignancies also include, but are not limited to, chronic lymphocytic leukemia (CLL), prolymphocytic leukemia (PLL). Waldenstrom's macroglobulinemia (WM), hairy cell leukemia (HCL), large granular lymphocyte (LGL) leukemia, acute lymphoid (or lymphocytic or lymphoblastic) leukemia, and Castleman's disease, NHL may also include T-cell non-Hodgkin's lymphomas (T-NHLs), which include, but are not limited to T-cell non-Hodgkin's lymphoma not otherwise specified (NOS), peripheral T-cell lymphoma (PTCL), anaplastic large cell lymphoma (ALCL), angioimmunoblastic lymphoid disorder (AILD), nasal natural killer (NK) cell/T-cell lymphoma, gamma/delta lymphoma, cutaneous T cell lymphoma, mycosis fungoides, and Sezary syndrome.

Hematopoietic cancers also include Hodgkin's lymphoma (or disease) including classical Hodgkin's lymphoma, nodular sclerosing Hodgkin's lymphoma, mixed cellularity Hodgkin's lymphoma, lymphocyte predominant (LP) Hodgkin's lymphoma, nodular LP Hodgkin's lymphoma, and lymphocyte depleted Hodgkin's lymphoma. Hematopoietic cancers also include plasma cell diseases or cancers such as multiple myeloma (MM) including smoldering MM, monoclonal gammopathy of undetermined (or unknown or unclear) significance (MGUS), plasmacytoma (bone, extramedullary), lymphoplasmacytic lymphoma (LPL), Waldenstrom's Macroglobulinemia, plasma cell leukemia, and primary amyloidosis (AL). Hematopoietic cancers may also include other cancers of additional hematopoietic cells, including polymorphonuclear leukocytes (or neutrophils), basophils, eosinophils, dendritic cells, platelets, erythrocytes and natural killer cells. Tissues which include hematopoietic cells referred herein to as “hematopoietic cell tissues” include bone marrow; peripheral blood; thymus; and peripheral lymphoid tissues, such as spleen, lymph nodes, lymphoid tissues associated with mucosa (such as the gut-associated lymphoid tissues), tonsils, Peyer's patches and appendix, and lymphoid tissues associated with other mucosa, for example, the bronchial linings.

### Pharmaceutical Compositions

Compounds described herein may be administered orally or parenterally. As formulated into a dosage form suitable for administration, the compounds described herein can be used as a pharmaceutical composition for the prevention, treatment, or remedy of the above diseases.

In clinical use of the compounds described herein, usually, the compound is formulated into various preparations together with pharmaceutically acceptable additives according to the dosage form and may then be administered. By “pharmaceutically acceptable” it is meant the additive, carrier, diluent or excipient must be compatible with the other ingredients of the formulation and not deleterious to the recipient thereof. As such, various additives ordinarily used in the field of pharmaceutical preparations are usable. Specific examples thereof include gelatin, lactose, sucrose, titanium oxide, starch, crystalline cellulose, hydroxypropyl methylcellulose, carboxymethylcellulose, corn starch, microcrystalline wax, white petrolatum, magnesium metasilicate aluminate, anhydrous calcium phosphate, citric acid, trisodium citrate, hydroxypropylcellulose, sorbitol, sorbitan fatty acid ester, polysorbate, sucrose fatty acid ester, polyoxyethylene, hardened castor oil, polyvinylpyrrolidone, magnesium stearate, light silicic acid anhydride, talc, vegetable oil, benzyl alcohol, gum arabic, propylene glycol, polyalkylene glycol, cyclodextrin, hydroxypropyl cyclodextrin, and the like.

Preparations to be formed with those additives include, for example, solid preparations such as tablets, capsules, granules, powders and suppositories; and liquid preparations such as syrups, elixirs and injections. These may be formulated according to conventional methods known in the field of pharmaceutical preparations. The liquid preparations may also be in such a form that may be dissolved or suspended in water or in any other suitable medium in their use. Especially for injections, if desired, the preparations may be dissolved or suspended in physiological saline or glucose liquid, and a buffer or a preservative may be optionally added thereto.

The pharmaceutical compositions may contain the compound of the invention in an amount of from 1 to 99.9% by weight, preferably from 1 to 60% by weight of the composition. The compositions may further contain any other therapeutically-effective compounds.

In case where the compounds of the invention are used for prevention or treatment for the above-mentioned diseases, the dose and the dosing frequency may be varied, depending on the sex, the age, the body weight and the disease condition of the patient and on the type and the range of the intended remedial effect. In general, when orally administered, the dose may be from 0.001 to 50 mg/kg of body weight/day, and it may be administered at a time or in several times. In specific embodiments, the dose is from about 0.01 to about 25 mg/kg/day, in particular embodiments, from about 0.05 to about 10 mg/kg/day. For oral administration, the compositions are preferably provided in the form of tablets or capsules containing from 0.01 mg to 1,000 mg. In specific embodiments, the dose is 0.01, 0.05, 0.1, 0.2, 0.5, 1.0, 2.5, 5, 10, 15, 20, 25, 30, 40, 50, 75, 100, 125, 150, 175, 200, 225, 250, 500, 750, 850 or 1,000 milligrams of a compound described herein. This dosage regimen may be adjusted to provide the optimal therapeutic response.

### Combination Therapy

The compounds of the present invention are further useful in methods for the prevention or treatment of the aforementioned diseases, disorders and conditions in combination with other therapeutic agents.

The compounds of the present invention may be used in combination with one or more other drugs in the treatment, prevention, suppression or amelioration of diseases or conditions for which compounds described herein or the other drugs may have utility, where the combination of the drugs together are safer or more effective than either drug alone. Such other drug(s) may be administered in an amount commonly used therefore, contemporaneously or sequentially with a compound described herein or a pharmaceutically acceptable salt thereof. When a compound described herein is used contemporaneously with one or more other drugs, the pharmaceutical composition may in specific embodiments contain such other drugs and the compound described herein or its pharmaceutically acceptable salt in unit dosage form. However, the combination therapy may also include therapies in which the compound described herein or its pharmaceutically acceptable salt and one or more other drugs are administered on different overlapping schedules. It is also contemplated that when used in combination with one or more other active ingredients, the compounds of the present invention and the other active ingredients may be used in lower doses than when each is used singly. Accordingly, the pharmaceutical compositions of the present invention include those that contain one or more other active ingredients, in addition to a compound described herein or a pharmaceutically acceptable salt thereof.

### Examples

### Abbreviations

The abbreviations used herein have the following tabulated meanings. Abbreviations not tabulated below have their meanings as commonly used unless specifically stated otherwise.

| | |
|---|---|
| ACN | acetonitrile |
| AcOH | acetic acid |
| BPin | 4,4,5,5-tetramethyl-1,3,2-dioxaborolan-2-yl |
| [BPin]₂ | 4,4,4′,4′,5,5,5′,5′-octamethyl-2,2′-bi(1,3,2- |
| | dioxaborolane) |
| Boc | tert-butoxycarbamate |
| Boc₂O | di-tert-butyl dicarbonate |
| DCM | dichloromethane |
| DEAD | diethyl azodicarboxylate |
| DIAD | diisopropyl azodicarboxylate |
| DIEA | diisopropylethylamine |
| DMA | dimethylacetamide |
| DMF | dimethylformamide |
| DMSO | dimethylsulfoxide |
| dtbpy | 4,4′-di-tert-butyl-2,2′-dipyridyl |
| EI | electron ionization |
| EtOAc | ethyl acetate |
| EtOH | ethanol |
| h | hour |
| H₂O | Water |
| HATU | (1-[bis(dimethylamino)methylene]-1H-1,2,3- |
| | triazolo[4,5-b]pyridinium 3-oxide |
| | hexafluorophosphate |
| HCI | Hydrochloric acid |
| ¹H NMR | proton nuclear magnetic resonance |
| HPLC | high performance liquid chromatography |
| i-Pr₂NEt | diisopropylethylamine |
| Ir[dF(CF₃)ppy]₂(dtbpy) | [4,4′-bis(1,1-dimethylethyl)-2,2′-bipyridine- |
| | N1,N1′]bis[3,5-difluoro-2-[5-(trifluoromethyl)- |
| | 2-pyridinyl-N]phenyl-C]Iridium(III) |
| K₂CO₃ | potassium carbonate |
| K₄Fe(CN)₆•3H₂O | Potassium ferrocyanide trihydrate |
| LC/MS | liquid chromatography coupled to mass |
| | spectrometer |
| MeCN | acetonitrile |
| MeOH | methanol |
| MHz | megahertz |
| min | minute(s) |
| mL | milliliter |
| MS | mass spectrum |
| Ms-Cl | Methanesulfonyl chloride (mesyl chloride) |
| MTBE | methyl tert-butyl ether |
| Na₂SO₄ | Sodium sulfate |
| NaOH | Sodium hydroxide |
| NaBH₄ | Sodium borohydride |
| NBS | N-bromosuccinamide |
| NCS | N-chlorosuccinamide |
| NH₄HCO₃ | Ammonium bicabonate |
| Ni(dtbpy)Cl₂ | [4,4′-Bis(1,1-dimethylethyl)-2,2′-bipyridine] |
| | nickel (II) dichloride |
| NiCl₂ | Nickel chloride |
| NMR | nuclear magnetic resonance |
| sat. | saturated |
| RED-Al | sodium bis(2-methoxyethoxy)aluminium hydride |
| RT | room temperature |
| Selectfluor | 1-chloromethyl-4-fluoro-1,4-diazoniabicyclo |
| | [2.2.2]octane bis(tetrafluoroborate) |
| TEA | Triethylamine |
| TFA | trifluoroacetic acid |
| THF | tetrahydrofuran |
| TLC | thin layer chromatography |
| TsCl | tosyl chloride |
| TsOH | toluenesulfonic acid |
| XPhos-Pd-G3 | (2-dicyclohexylphosphino-2′,4′,6′-triisopropyl- |
| | 1,1′-biphenyl)[2-(2′-amino-1,1′-biphenyl)] |
| | palladium(II) methanesulfonate |

### General Synthetic Schemes

One approach for the preparation of these inhibitors is generalized in General Synthetic Scheme I. A 2-(aminomethyl)chroman-4-one derivative I is converted to an acetamide, the ketone is reduced, and the resulting alcohol is subjected to acid and heat to provide cyclization products III. The acetamide was hydrolyzed and the racemic amine resolved into the undesired R,R enantiomer and the desired S,S enantiomer. The desired S,S enantiomer was converted into amide inhibitors with standard amide coupling conditions.

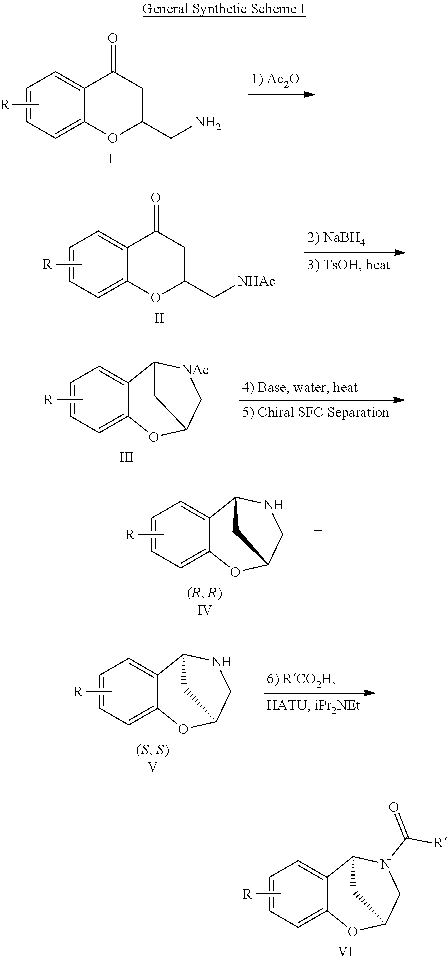

The bridged benzazepane amides could be functionalized by treatment with a halogenating reagent to install a fluorine atom, a chlorine atom or a bromine atom (General Synthetic Scheme II). Aryl chlorides and bromides were found to be potent inhibitors and were also good substrates to introduce greater diversity such as alkyl groups or nitriles. The core could be borylated with bis(pinacolato)diboron and an iridium catalyst, and the resulting boronic esters were handles to further functionalize.

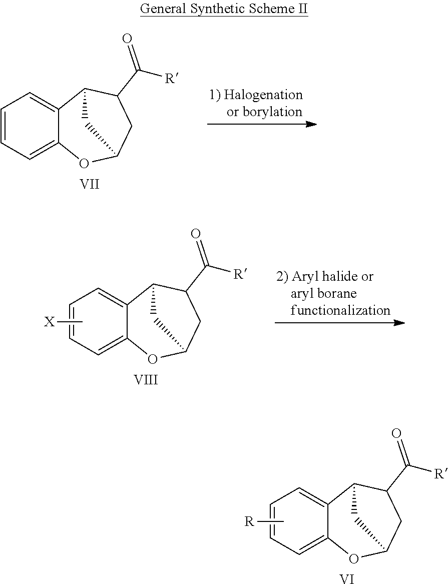

An alternative means to access the bridged benzazepane core is shown in General Scheme III. Here, a Grignard is formed and reacted with tert-butyl (R)-4-((tert-butyldimethylsilyl)oxy)-2-oxopyrrolidine-1-carboxylate, giving X after deprotection of the phenol. The ketone is reduced and the alcohol converted to a mesylate which undergoes cyclization with base to yield aryl pyrrolidines XI. The TBS ether is cleaved and the resulting hydroxy phenol XII undergoes Mitsunobu cyclization to provide the bridged benzazepine products VI. The intermediate hydroxy phenol XII could be optionally halogenated adjacent to the phenol, prior to the Mitsunobu reaction. When the core was halogenated, it could be a substrate for further functionalization to a nitrile, for example. Finally, the inhibitors were made by deprotection of the Boc protective group and amide bond formation.

An alternative entry into this scaffold is shown in General Scheme IV, were an Ir(I)-catalyzed photoredox arylation of a pyrrolidine carboxylic acid provides a more rapid entry to aryl pyrrolidines. This intermediate was subsequently converted to bridged benzazepane amide inhibitors via a sequence similar to that described in General Synthetic Scheme II.

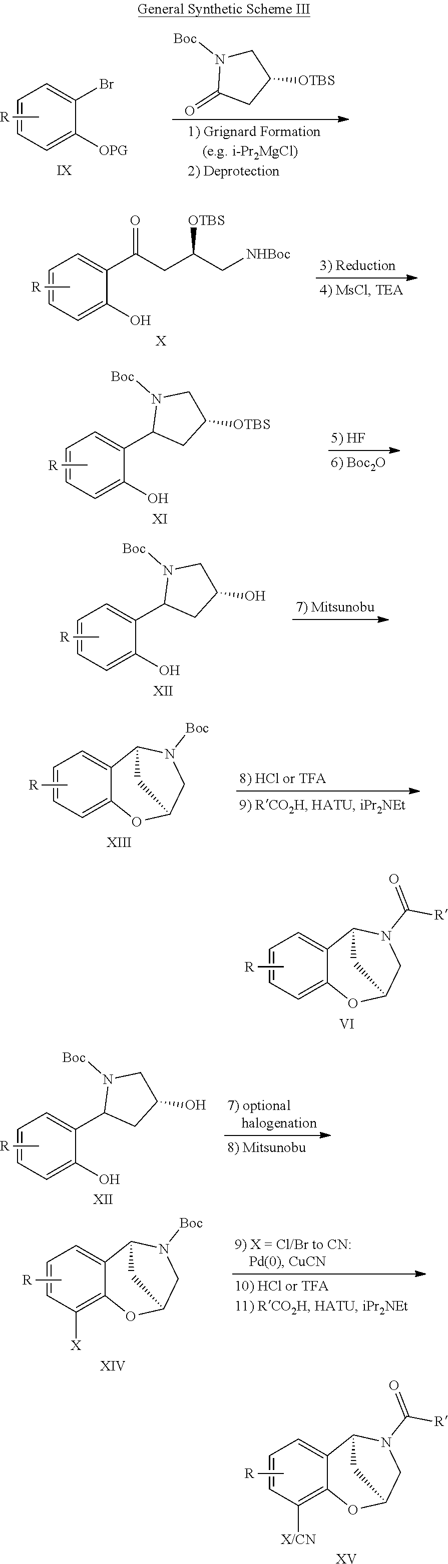

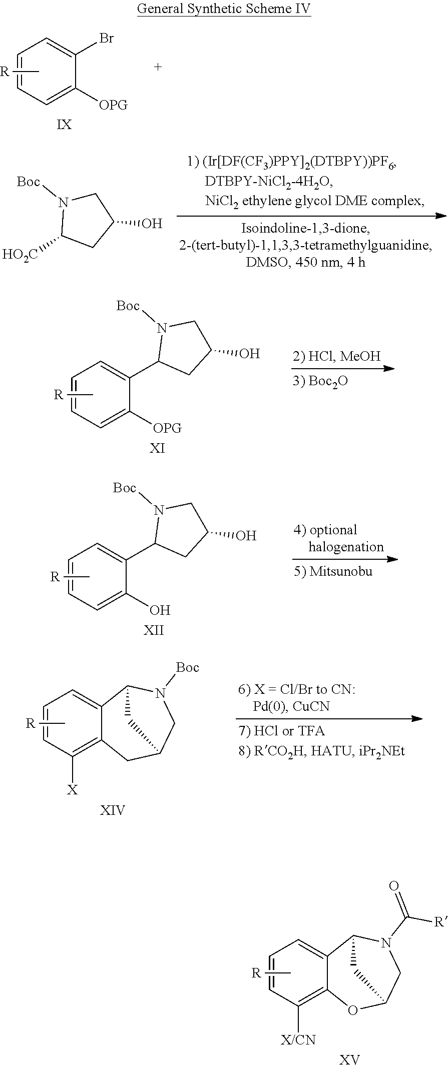

### Intermediates

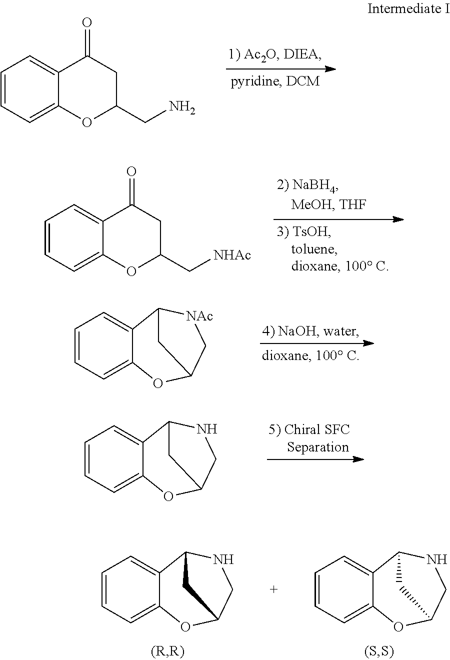

### Intermediate I. (2S,5S)-2,3,4,5-Tetrahydro-2,5-methanobenzo[f][1,4]oxazepine

Step 1. A stirred solution of 2-(aminomethyl)-2,3-dihydro-1-benzopyran-4-one (120 g, 678 mmol) and N,N-diisopropylethylamine (111 g, 860 mmol) in DCM (2 L) was treated at 0° C. with pyridine (14.5 g, 183 mmol) and acetic anhydride (80.9 g, 792 mmol). The resulting mixture was stirred for 2 h at room temperature, diluted with water (2 L) and extracted with DCM (2×2 L). The combined organic layers were washed with water (2×1 L), dried over anhydrous Na₂SO₄, filtered and concentrated. The crude product N-[(4-oxo-2,3-dihydro-1-benzopyran-2-yl)methyl]acetamide was used without further purification.

Step 2. A stirred solution of N-[(4-oxo-2,3-dihydro-1-benzopyran-2-yl)methyl]acetamide (126 g, 575 mmol) in THF (3 L) and MeOH (40 mL) was treated portion wise with NaBH₄ (26.1 g, 690 mmol). The mixture was stirred for 2 h, then quenched by the addition of MeOH (100 mL) and water (100 mL). The resulting mixture was stirred for 30 min, then treated with 3 L of 1 M citric acid dropwise over 30 min. The resulting mixture was stirred for additional 30 min and extracted with DCM (3×3 L). The combined organic layers were dried over anhydrous Na₂SO₄, filtered and concentrated to provide N-[(4-hydroxy-3,4-dihydro-2H-1-benzopyran-2-yl)methyl]acetamide which was used without further purification.

Step 3. A stirred solution of N-[(4-hydroxy-3,4-dihydro-2H-1-benzopyran-2-yl)methyl]acetamide (120 g, 542 mmol) in 1,4-dioxane (1 L) and toluene (1 L) was treated with TsOH (18.7 g, 108 mmol). The resulting mixture was stirred for overnight at 100° C. The residue was purified by dynamic axial compression (DAC) HPLC, eluting with 1:1 MeCN/H₂O (containing 0.1% NH₄HCO₃), to afford 1-(2,3-dihydro-2,5-methanobenzo[f][1,4]oxazepin-4(5H)-yl)ethan-1-one.

Step 4. A stirred solution of 1-(2,3-dihydro-2,5-methanobenzo[f][1,4]oxazepin-4(5H)-yl)ethan-1-one (81 g, 399 mmol) in 1,4-dioxane (1 L) and water (1 L) was treated with NaOH (159 g, 399 mol). The resulting mixture was stirred for 2 days at 100° C., and cooled to RT. The resulting mixture was extracted with DCM (3×2 L), washed with water (1 L), dried over anhydrous Na₂SO₄, filtered and concentrated to afford racemic 2,3,4,5-tetrahydro-2,5-methanobenzo[f][1,4]oxazepine.

Step 5. Racemic 2,3,4,5-tetrahydro-2,5-methanobenzo[f][1,4]oxazepine (55 g, 341 mmol) was then resolved by chiral SFC to obtain of (2R,5R)-2,3,4,5-tetrahydro-2,5-methanobenzo[f][1,4]oxazepine as the first eluting enantiomer, of (2S,5S)-2,3,4,5-tetrahydro-2,5-methanobenzo[f][1,4]oxazepine as the second eluting enantiomer.

### Analytical Data:

Enantiomer 1 (2R,5R)-2,3,4,5-tetrahydro-2,5-methanobenzo[f][1,4]oxazepine: Chiral SFC Ret Time 2.80 min [IG Column 100×4.6 mm, 3.0 μM, 10% MeOH with 20 mM NH₃]; ¹H-NMR (300 MHz, DMSO-d₆) δ 6.92-7.15 (m, 2H), 6.62-6.80 (m, 2H), 4.88 (t, J=3.6 Hz, 1H), 3.99 (d, J=4.4 Hz, 1H), 2.94-3.24 (m, 2H), 2.01 (d, J=11.7 Hz, 1H), 1.74-1.91 (m, 1H); MS (EI) calculated for C₁₀H₁₂NO [M+H]⁺, 162. found, 162.

Enantiomer 2 (2S,5S)-2,3,4,5-tetrahydro-2,5-methanobenzo[f][1,4]oxazepine: Chiral SFC Ret Time 3.57 min [IG Column 100×4.6 mm, 3.0 μM, 10% MeOH with 20 mM NH₃]; ¹H-NMR (300 MHz, DMSO-d₆) δ 6.92-7.14 (m, 2H), 6.59-6.80 (m, 2H), 4.88 (t, J=3.6 Hz, 1H), 3.99 (d, J=4.4 Hz, 1H), 2.95-3.24 (m, 2H), 2.00 (d, J=11.7 Hz, 1H), 1.74-1.90 (m, 1H); MS (EI) calculated for C₁₀H₁₂NO [M+H]⁺, 162. found, 162.

### Intermediate II. (2S,5S)-7,9-Difluoro-2,3,4,5-tetrahydro-2,5-methanobenzo[f][1,4]oxazepine

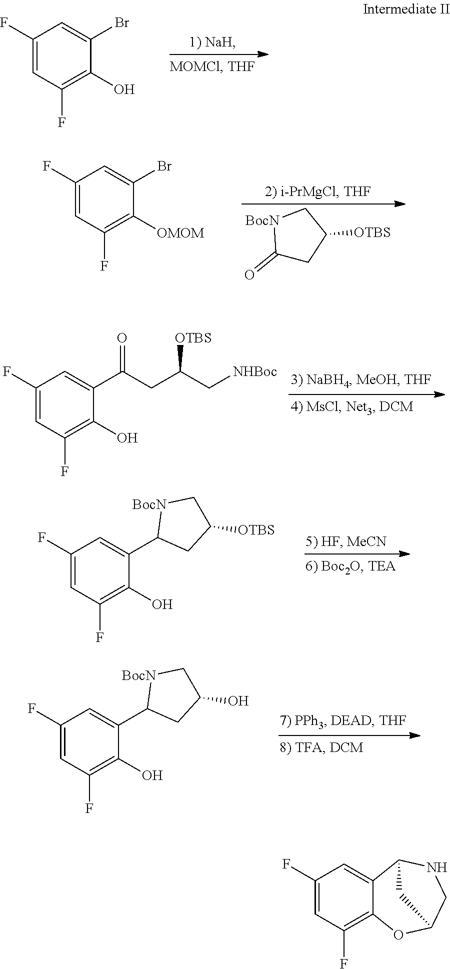

Step 1. A mixture containing 2-bromo-4,6-difluorophenol (4.80 g, 23.0 mmol) in THF (50 mL) was cooled to 0° C., treated with 60% NaH in mineral oil (1.10 g, 45.8 mmol) and stirred 10 min. Next, chloromethyl methyl ether (2.30 mL, 30.3 mmol) was added dropwise and the mixture stirred 1 hour. The cooling bath was removed and the mixture stirred for 18 hours. The mixture was diluted with DCM, washed with 1 N HCl, 1 N NaOH, dried over sodium sulfate and concentrated to afford 1-bromo-3,5-difluoro-2-(methoxymethoxy)benzene. ¹H NMR (600 MHz, DMSO-d₆) δ 7.75 (m, 1H), 7.47 (m, 1H), 5.12 (s, 2H), 3.54 (s, 3H).

Step 2. A mixture containing 1-bromo-3,5-difluoro-2-(methoxymethoxy)benzene (1.60 g, 6.32 mmol) in THF (10 mL) was cooled to 0° C. and treated dropwise with a 2 M solution of isopropylmagnesium chloride in THF (4.0 mL, 8.0 mmol). The mixture was stirred for 20 min at 0° C., and then a solution of tert-butyl (R)-4-((tert-butyldimethylsilyl)oxy)-2-oxopyrrolidine-1-carboxylate (1.00 g, 3.17 mmol) in THF (10 mL) was added, and the mixture stirred at RT for 18 h. The mixture was poured into DCM, washed with 1H HCl and 1 N NaOH, dried over Na₂SO₄, and concentrated. Chromatography on SiO₂ (0-30% MeOH/DCM, 80 g silica gel) gave tert-butyl (R)-(2-((tert-butyldimethylsilyl)oxy)-4-(3,5-difluoro-2-hydroxyphenyl)-4-oxobutyl)carbamate. ¹H NMR (500 MHz, DMSO-d₆) δ 11.60 (s, 1H), 7.65 (m, 1H), 7.53 (d, J=9.1 Hz, 1H), 6.90 (t, J=5.8 Hz, 1H), 4.26 (m, 1H), 3.49 (m, 1H), 3.20 (m, 1H), 3.04-3.16 (m, 1H), 2.99 (m, 1H), 1.38 (s, 9H), 0.75 (s, 9H), 0.04 (s, 3H), −0.12 (s, 3H); MS (EI) calculated for C₁₆H₂₆F₂NO₃Si [M-Boc+H]⁺, 346. found, 346.

Steps 3 and 4. A mixture containing tert-butyl (R)-(2-((tert-butyldimethylsilyl)oxy)-4-(5-fluoro-2-(methoxymethoxy)phenyl)-4-oxobutyl)carbamate (1.14 mg, 2.56 mmol) in THF (5 mL) and MeOH (0.5 mL) was treated with NaBH₄ (200 mg, 5.29 mmol) and stirred for 6 hours. The mixture was quenched with water, stirred another 1 h, extracted with DCM, and washed with water. The organic layer was dried over Na₂SO₄ and concentrated. The residue was then dissolved in DCM (5 mL), treated with TEA (1.40 mL, 10.0 mmol) and cooled to −78° C., followed by treatment with Ms-Cl (0.50 mL, 6.4 mmol) and removal of the cooling bath. The mixture was stirred for 3 h at RT, diluted with DCM, washed with 1 N HCl, 1 N NaOH, dried (Na₂SO₄) and concentrated. Chromatography on SiO₂ (0-80% EtOAc/DCM with 80 g silica gel) gave desired product tert-butyl (4R)-4-((tert-butyldimethylsilyl)oxy)-2-(3,5-difluoro-2-hydroxy phenyl)pyrrolidine-1-carboxy late as a 1:1 mixture of S,R and R,R diastereomers. MS (EI) calculated for C₂₁H₃₃F₂NO₄SiNa [M+Na]⁺, 452. found, 452.

Steps 5 and 6. A mixture containing tert-butyl (4R)-4-((tert-butyldimethylsilyl)oxy)-2-(3,5-difluoro-2-hydroxy phenyl)pyrrolidine-1-carboxy late (835 mg, 1.94 mmol) in MeCN (5 mL) was treated with 48% aqueous HF (0.20 mL, 5.57 mmol) and stirred overnight. Partial cleavage of the Boc protective group was observed. The mixture was concentrated to dryness, dissolved in DCM (5 mL), treated with triethylamine (0.81 mL, 5.8 mmol) and Boc₂O (424 mg, 1.94 mmol). The mixture was stirred overnight, diluted with DCM, and washed with 1 N HCl, dried over sodium sulfate and concentrated to provide tert-butyl (4R)-2-(3,5-difluoro-2-hydroxyphenyl)-4-hydroxypyrrolidine-1-carboxylate as a 1:1 mixture of diastereomers. MS (EI) calculated for C₁₅H₁₉F₂NO₄Na [M+Na]⁺, 338. found, 338.

Step 7. A mixture containing tert-butyl (4R)-2-(3,5-difluoro-2-hydroxyphenyl)-4-hydroxypyrrolidine-1-carboxylate (542 mg, 1.72 mmol) as a 1:1 mixture of diastereomers in THF (5 mL) was treated with triphenylphosphine (1.40 g, 5.34 mmol) and cooled to 0° C. Diethyl azodicarboxylate (0.80 mL, 5.1 mmol) was added dropwise to the mixture followed by stirring at 0° C. for 2 h, and for another 2 h at RT. The reaction mixture was concentrated and purified by chromatography on SiO₂ (gradient of 0-50% EtOAc/DCM with 24 g silica gel) to afford tert-butyl (2S,5S)-7,9-difluoro-2,3-dihydro-2,5-methanobenzo[f][1,4]oxazepine-4(5H)-carboxylate. ¹H NMR (600 MHz, CDCl3) δ 6.78 (m, 1H), 6.63 (m, 1H), 5.07 (m, 1H), 4.76 (m, 1H), 3.63-3.80 (m, 2H), 2.25 (m, 2H), 1.47, 1.44 (s, 9H); MS (EI) calculated for C₁₁H₁₀F₂NO₃ [M-tBu]⁺, 242. found, 242.

Step 8. To a solution of tert-butyl (2S,5S)-7,9-difluoro-2,3-dihydro-2,5-methanobenzo[f][1,4]oxazepine-4(5H)-carboxylate (200 mg, 0.673 mmol) in DCM (6727 μL) was added TFA (518 μL, 6.73 mmol). The reaction was aged at 23° C. overnight. The reaction was diluted with DCM and added water, quenched with 2M NaOH, and extracted with DCM. The combined organic layer was washed with brine, dried over Na₂SO₄, filtered and concentrated to dryness to afford (2S,5S)-7,9-difluoro-2,3,4,5-tetrahydro-2,5-methanobenzo[f][1,4]oxazepine.

### Intermediate III. 2,3,4,5-Tetrahydro-1H-1,4-methanobenzo[c]azepine

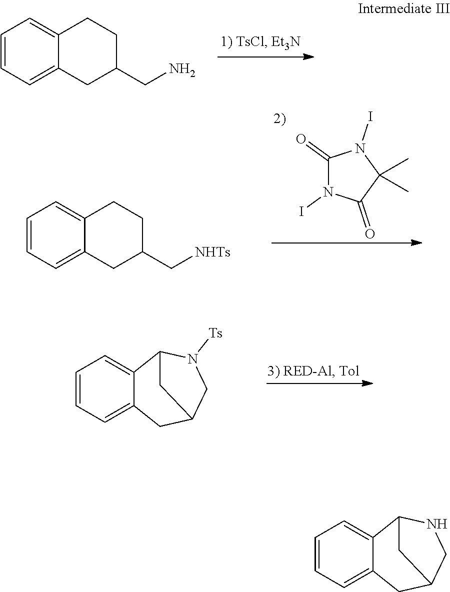

Step 1. A solution of (1,2,3,4-tetrahydronaphthalen-2-yl)methanamine (1 g, 6.2 mmol) in DCM (31 mL) was treated with Et₃N (2.16 mL, 15.5 mmol) then 4-methylbenzenesulfonyl chloride (1.42 g, 7.44 mmol) and allowed to stir at RT overnight. The reaction was quenched with a sat. NH₄Cl solution, then extracted 3 times with DCM. The combined organic extracts were washed with brine, dried over sodium sulfate, filtered, and concentrated. The residue was purified by chromatography on silica gel (40 g silica gel, 0-30% EtOAc/Hexanes) giving 4-methyl-N-((1,2,3,4-tetrahydronaphthalen-2-yl)methyl)benzenesulfonamide. MS (EI) calc'd for C₁₈H₂₂NO₂S [M+H]⁺, 316. found, 316.

Step 2. 4-Methyl-N-((1,2,3,4-tetrahydronaphthalen-2-yl)methyl)benzenesulfonamide (1.70 g, 5.38 mmol) and 1,3-diiodo-5,5-dimethylhydantoin (8.17 g, 21.5 mmol) were dissolved in DCE (108 ml) and stirred at RT for 5 min. The mixture was then heated to 80° C. and allowed to stir at that temperature overnight. The crude reaction was diluted with sat. sodium thiosulfate solution then extracted 3 times with EtOAc. The combined organic extracts were washed with brine, dried over sodium sulfate, filtered, and concentrated. The residue was purified by chromatography on silica gel (40 g silica gel, 0-30% EtOAc/Hexanes) giving 2-tosyl-2,3,4,5-tetrahydro-1H-1,4-methanobenzo[c]azepine. ¹H NMR (499 MHz, Chloroform-d) δ 7.50 (d, J=8.1 Hz, 2H), 7.16 (dd, J=12.8, 7.7 Hz, 4H), 7.09 (t, J=7.4 Hz, 1H), 6.98 (d, J=7.4 Hz, 1H), 4.82 (d, J=3.3 Hz, 1H), 3.52 (dd, J=8.6, 6.3 Hz, 1H), 3.12 (d, J=9.8 Hz, 1H), 3.08 (dd, J=17.3, 4.9 Hz, 1H), 2.74 (s, 1H), 2.69 (d, J=17.4 Hz, 1H), 2.37 (s, 3H), 1.77 (d, J=4.9 Hz, 2H). MS (EI) calc'd for C₁₈H₂₀NO₂S [M+H]⁺, 314. found, 314.

Step 3. 2-Tosyl-2,3,4,5-tetrahydro-1H-1,4-methanobenzo[c]azepine (0.922 g, 2.94 mmol) was dissolved in toluene (14.7 ml), then RED-Al in toluene (6.56 ml, 23.5 mmol) was added. The mixture was heated to 100° C. overnight, cooled to 0° C., diluted with Et₂O, and quenched with H₂O (6.8 mL), 1M NaOH (6.8 mL), then H₂O (20.4 mL). MgSO₄ (1.25 g) was added. The mixture was stirred vigorously for 1 h, then was filtered. The solids were washed with EtOAc. The organic solution was treated with a 4 M solution of HCl in dioxane (2.9 mL, 5.9 mmol) and was concentrated to give Intermediate III, 2,3,4,5-tetrahydro-1H-1,4-methanobenzo[c]azepine which was used directly. MS (EI) calc'd for C₁₁H₁₄N [M+H]⁺, 160. found, 160.

### Table 1 Compounds

### Example 1A. Preparation of 1-((2S,5S)-2,3-Dihydro-2,5-methanobenzo[f][1,4]oxazepin-4(5H)-yl)-2,2-dimethylpropan-1-one (1-1)

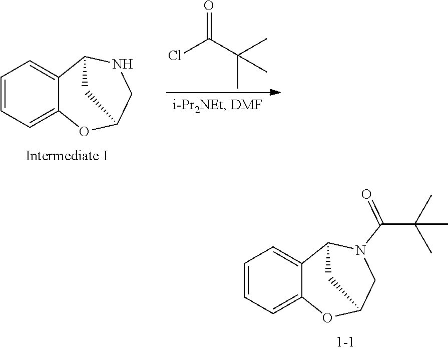

A mixture containing (2S,5S)-2,3,4,5-tetrahydro-2,5-methanobenzo[f][1,4]oxazepine, Intermediate I (10 mg, 0.062 mmol) in DMF (1 mL) was treated with N,N-diisopropylethylamine (0.020 mL, 0.12 mmol) and pivaloyl chloride (10 mg, 0.083 mmol). The mixture was stirred overnight, diluted with 1 mL of MeOH, and purified by reverse phase chromatography (gradient of 15-70% MeCN/water with 0.1% TFA) to provide 1-((2S,5S)-2,3-dihydro-2,5-methanobenzo[f][1,4]oxazepin-4(5H)-yl)-2,2-dimethylpropan-1-one. ¹H NMR (500 MHz, DMSO-d₆) δ 7.10-7.13 (m, 2H), 6.73-6.78 (m, 2H), 5.18 (s, 1H), 5.05 (s, 1H), 3.92 (m, 2H), 2.09 (m, 1H), 2.00 (m, 1H), 1.10 (s, 9H); MS (EI) calculated for C₁₅H₂₀NO₂ [M+H]⁺, 246. found, 246.

### Example 1B. Preparation of 1-((2S,5S)-2,3-Dihydro-2,5-methanobenzo[f][1,4]oxazepin-4(5H)-yl)-3,3-difluoro-2,2-dimethylpropan-1-one (1-2)

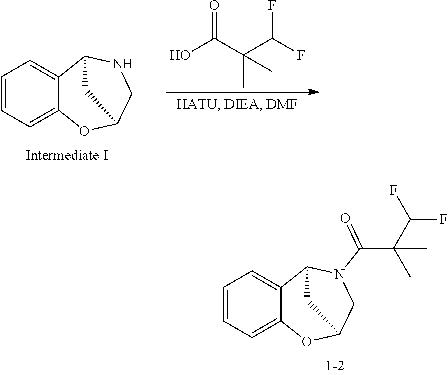

A mixture containing 3,3-difluoro-2,2-dimethylpropanoic acid (14 mg, 0.10 mmol), HATU (53 mg, 0.14 mmol) and DIEA (0.033 mL, 0.19 mmol) in DMF (1 mL) was treated with (2S,5S)-2,3,4,5-tetrahydro-2,5-methanobenzo[f][1,4]oxazepine, Intermediate I (15 mg, 0.093 mmol) and stirred for 16 h. The mixture was concentrated and purified by reverse phase chromatography (gradient of 47-67% MeCN/water with 0.1% TFA) to give 1-((2S,5S)-2,3-dihydro-2,5-methanobenzo[f][1,4]oxazepin-4(5H)-yl)-3,3-difluoro-2,2-dimethylpropan-1-one. ¹H NMR (400 MHz, CD₃OD) δ 7.06-7.25 (m, 2H), 6.70-6.83 (m, 2H), 5.94-6.25 (m, 1H), 5.26 (s, 1H), 5.04 (s, 1H), 4.00 (s, 2H), 2.15 (s, 2H), 1.30 (s, 3H), 1.16 (s, 3H); MS (EI) calculated for C₁₅H₁₈F₂NO₂ [M+H]⁺, 282. found, 282.

### Structure of 1-((2S,5S)-2,3-Dihydro-2,5-methanobenzo[f][1,4]oxazepin-4(5H)-yl)-3,3-difluoro-2,2-dimethylpropan-1-one (1-2)

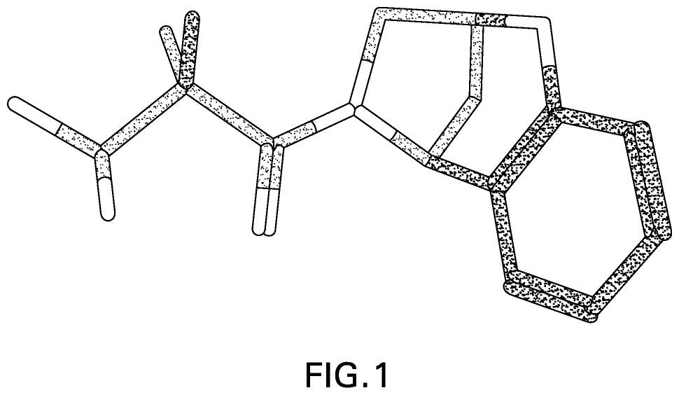

The absolute stereochemistry of 1-((2S,5S)-2,3-dihydro-2,5-methanobenzo[f][1,4]oxazepin-4(5H)-yl)-3,3-difluoro-2,2-dimethylpropan-1-one (1-2) was confirmed by crystallography. The X-ray crystal structure of human RIPK1 kinase domain (residues 1-294 containing 4 mutations: C34A, C127A, C233A, C240A) in complex with compound 1-2 indicated the binding mode, orientation and conformation of the ligand bound to the protein. The structure was solved at 2.32 Å resolution, and the electron density map indicated an (S,S)-configuration for 1-2, as shown in FIG. 1. There were two monomers in the asymmetric unit with the same overall configuration. Data collection and processing statistics are outlined in Table A. The structure was obtained by Proteros Biostructures GmbH (Martinsried, Germany).

FIG. 1 shows the structure of 1-((2S,5S)-2,3-Dihydro-2,5-methanobenzo[f][1,4]oxazepin-4(5H)-yl)-3,3-difluoro-2,2-dimethylpropan-1-one (1-2) from X-ray crystal structure complex with human RIPK1 kinase domain, indicating an S,S stereochemical assignment.

TABLE A
Data Collection and Processing Statistics.

| X-ray source | DLS i03 | |
|---|---|---|
| Wavelength [Å] | 1.0001 | |
| Detector | EIGER | |
| Temperature [K] | 100 | |
| Space group | P 21 21 21 | |
| Cell: a; b; c; [Å] | 47.31; 95.16; 128.12 | |
| α; β; γ; [*] | 90.0; 90.0; 90.0 | |
| Resolution [Å] | 2.32 | (2.36-2.32) |
| Unique reflections | 25627 | (1226) |
| Multiplicity | 7.9 | (3.8) |
| Completeness [%] | 99.6 | (95.8) |
| Rpim [%] | 2.8 | (40.9) |
| Rsym [%] | 9.2 | (72.0) |
| Rmeas [%] | 9.6 | (83.3) |
| CC1/2 [%] | 99.80 | (96.20) |
| Mean(I)/sd5 | 16.5 | (1.8) |

The phase information necessary to determine and analyze the structure was obtained by molecular replacement. A previously solved structure of human RIPK1 kinase domain was used as a search model. Subsequent model building and refinement was performed according to standard protocols with COOT and the software package CCP4, respectively. For the calculation of the free R-factor, a measure to cross-validate the correctness of the final model, about 4.5% of measured reflections were excluded from the refinement procedure (see Table B). TLS refinement (using REFMAC5, CCP4) has been carried out, which resulted in lower R-factors and higher quality of the electron density map. Automatically generated local NCS restraints have been applied (keyword “ncsr local” of newer REFMAC5 versions). The ligand parameterization and generation of the corresponding library files were carried out with CORINA. The water model was built with the “Find waters”-algorithm of COOT by putting water molecules in peaks of the Fo-Fc map contoured at 3.0 followed by refinement with REFMAC5 and checking all waters with the validation tool of COOT. The criteria for the list of suspicious waters were: B-factor greater 80 A2, 2Fo-Fc map less than 1.2 s, distance to closest contact less than 2.3 A or more than 3.5 A. The suspicious water molecules and those in the ligand binding site (distance to ligand less than 10 A) were checked manually. The Ramachandran Plot of the final model shows 92.1% of all residues in the most favored region, 7.9% in the additionally allowed region, and 0.0% in the generously allowed region. No residues are found in the disallowed region (Table B). Statistics of the final structure and the refinement process are listed in Table B.

TABLE B
Refinement Statistics for Ligand 1-2.

| | |
|---|---|
| Resolution [Å] | 76.40-2.32 |
| Number of reflections (working/test) | 22093/1034 |
| Rcryst [%] | 23.9 |
| Rfree[%] | 31.5 |
| Total number of atoms: | |
| Protein | 4372 |
| Water | 69 |
| Ligand | 40 |
| Iodide | 4 |
| Chloride | 1 |
| Deviation from ideal geometry: | |
| Bond lengths [Å] | 0.012 |
| Bond angles [°] | 1.58 |
| Bonded B′s [Å2] | 5.2 |
| Ramachandran plot: | |
| Most favored regions [%] | 92.1 |
| Additional allowed regions [%] | 7.9 |
| Generously allowed regions [%] | 0 |
| Disallowed regions [%] | 0 |

### Example 1C. Preparation of 1-[(2S,5S)-2,3-dihydro-2,5-methano-1,4-benzoxazepin-4(5H)-yl]-3-fluoro-2-(fluoromethyl)-2-methylpropan-1-one (1-33)

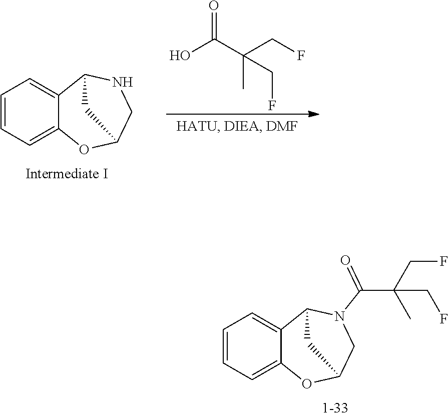

In a 8×30 mm vial, 3-fluoro-2-(fluoromethyl)-2-methylpropanoic acid 0.052 mL (0.026 mmol, 0.5 M DMF Sol'n.) was added with 0.046 mL of Hunig's base (0.046 mmol, 1.0 M DMF Sol'n.) and 0.160 mL of HATU reagent (0.032 mmol, 0.2 M DMF Sol'n.). The mixture was placed on a shaker for 20 min. at room temperature. (2S,5S)-2,3,4,5-tetrahydro-2,5-methanobenzo[f][1,4]oxazepine 0.100 mL (0.020 mmol, 0.2 M DMF Sol'n) was added to the mixture. The mixture was placed on a shaker overnight, at room temperature. The mixture was concentrated to dryness using a Genevac and reconstituted with 0.100 mL of DMSO. The crude mixture was purified via reverse phase chromatography using a Waters XSelect™ OBD C-18, 10×50 mm, 5 micron particle size, preparative HPLC column with 0.05% TFA in ACN and water using a 5-100% gradient. The product fractions were analyzed using a Waters Acquity I Class UPLC-MS equipped with PDA and SQD2 detectors (ESI positive ionization mode); column: Waters ACQUITY UPLC BEH C18 1.7 um column, 1.0×50 mm with (Waters #186002344) in tandem with a microflow capable (0.01-2.0 mL/min) ThermoCorona™ Veo™ RS Charged Aerosol Detector. After mass determination was calculated by CAD quantification of the product the product fractions were concentrated by Genevac and reconstituted with DMSO to provide the title compound 1-[(2S,5S)-2,3-dihydro-2,5-methano-1,4-benzoxazepin-4(5H)-yl]-3-fluoro-2-(fluoromethyl)-2-methylpropan-1-one as a 10 mM DMSO solution for biological screening. MS calculated for C₁₅H₁₈F₂NO₂ [M+H]⁺, 282. found, 282.

### Example 1D. Preparation of 6-(4-((2S,5S)-2,3,4,5-Tetrahydro-2,5-methanobenzo[f][1,4]oxazepine-4-carbonyl)piperidin-1-yl)pyrimidine-4-carbonitrile (1-36)

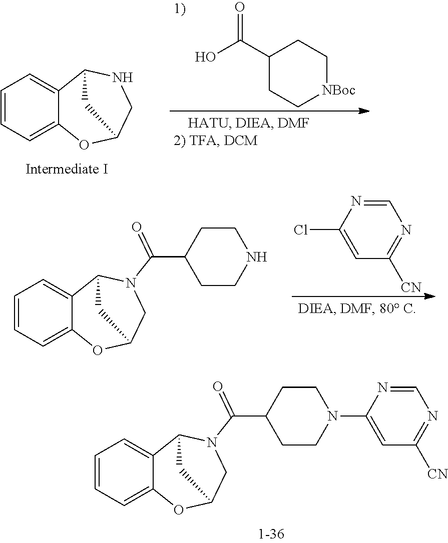 [SMILES: c1ccc2c(c1)O[C@@H]1CN[C@H]2C1 ; CC(C)(C)OC(=O)N1CCC(C(=O)O)CC1 ; O=C(C1CCNCC1)N1C[C@@H]2C[C@H]1c1ccccc1O2 ; N#Cc1cc(Cl)ncn1 ; N#Cc1cc(N2CCC(C(=O)N3C[C@@H]4C[C@H]3c3ccccc3O4)CC2)ncn1]

A solution of (2S,5S)-2,3,4,5-tetrahydro-2,5-methanobenzo[f][1,4]oxazepine, Intermediate I (100 mg, 0.62 mmol) in 2 mL of DMF was treated with DIEA (0.20 mL, 1.1 mmol), HATU (350 mg, 0.92 mmol) and 1-(tert-butoxycarbonyl)piperidine-4-carboxylic acid (180 mg, 0.79 mmol). The mixture was stirred overnight, diluted with DCM, washed with water, dried (Na₂SO₄) and concentrated. Chromatography on SiO₂ (gradient of 0-70% EtOAc/DCM, 24 g silica gel) gave the desired intermediate, tert-butyl 4-((2S,5S)-2,3,4,5-tetrahydro-2,5-methanobenzo[f][1,4]oxazepine-4-carbonyl)piperidine-1-carboxylate. The residue was dissolved in 2 mL of DCM, treated with 0.5 mL of TFA, and the resulting solution aged for 1 h. The solution was then concentrated to dryness, dissolved in 2 mL of 1:1 MeCN/water and lyophilized to obtain ((2S,5S)-2,3-dihydro-2,5-methanobenzo[f][1,4]oxazepin-4(5H)-yl)(piperidin-4-yl)methanone as a TFA salt. MS calculated for C₁₆H₂₁N₂O₂ [M+H]⁺, 273. found, 273. A solution of ((2S,5S)-2,3-dihydro-2,5-methanobenzo[f][1,4]oxazepin-4(5H)-yl)(piperidin-4-yl)methanone, TFA salt (20 mg, 0.052 mmol) in 1 mL of DMF was treated with 6-chloropyrimidine-4-carbonitrile (20 mg, 0.14 mmol) and DIEA (0.050 mL, 0.29 mmol) and then stirred overnight at 80° C. The mixture was cooled and purified by reverse phase chromatography (gradient of 15-70% MeCN/water with 0.1% TFA) to provide the desired product, 6-(4-((2S,5S)-2,3,4,5-tetrahydro-2,5-methanobenzo[f][1,4]oxazepine-4-carbonyl)piperidin-1-yl)pyrimidine-4-carbonitrile. ¹H NMR (500 MHz, DMSO-d₆) δ 8.54 (m, 1H), 7.56 (m, 1H), 7.38 (m, 1H), 7.06-7.23 (m, 1H), 6.86 (m, 1H), 6.77 (m, 1H), 5.01-5.37 (m, 2H), 3.83-3.92 (m, 2H), 3.61 (m, 1H), 3.47 (m, 1H), 3.19 (m, 1H), 3.02 (m, 1H), 2.68 (m, 1 H), 2.29 (m, 1H), 2.16 (m, 1H), 1.88 (m, 1H), 1.76 (m, 1H), 1.31-1.55 (m, 2H); MS (EI) calculated for C₂₁H₂₂N₅O₂ [M+H]⁺, 376. found, 376.

### Example 1E. Preparation of 6-(4-Fluoro-4-((2S,5S)-2,3,4,5-tetrahydro-2,5-methanobenzo[f][1,4]oxazepine-4-carbonyl)piperidin-1-yl)pyrimidine-4-carbonitrile (1-49)

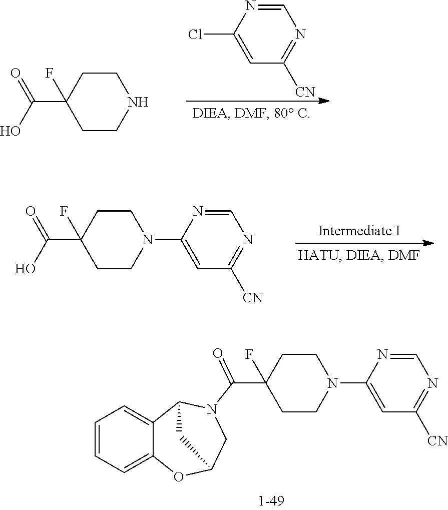 [SMILES: O=C(O)C1(F)CCNCC1 ; N#Cc1cc(Cl)ncn1 ; N#Cc1cc(N2CCC(F)(C(=O)O)CC2)ncn1 ; N#Cc1cc(N2CCC(F)(C(=O)N3C[C@@H]4C[C@H]3c3ccccc3O4)CC2)ncn1]

A mixture containing 6-chloropyrimidine-4-carbonitrile (400 mg, 2.87 mmol), 4-fluoropiperidine-4-carboxylic acid (500 mg, 2.72 mmol), DIEA (0.71 mL, 4.1 mmol) in DMF (5 mL) was stirred overnight. The mixture was diluted with DCM, washed with water, dried (Na₂SO₄) and concentrated. Reverse phase chromatography (gradient of 2-55% MeCN/water with 0.1% TFA) gave the desired product, 1-(6-cyanopyrimidin-4-yl)-4-fluoropiperidine-4-carboxylic acid, TFA salt. MS (EI) calculated for C₁₁H₁₂FN₄O₂ [M+H]⁺, 251. found, 251. A mixture containing 1-(6-cyanopyrimidin-4-yl)-4-fluoropiperidine-4-carboxylic acid, TFA salt (23 mg, 0.063 mmol), (2S,5S)-2,3,4,5-tetrahydro-2,5-methanobenzo[f][1,4]oxazepine, Intermediate I (10 mg, 0.062 mmol), DIEA (0.050 mL, 0.29 mmol) and HATU (30 mg, 0.079 mmol) in 1 mL of DMF was stirred overnight. The mixture was diluted with 1 mL of MeOH, filtered and purified by reverse phase chromatography (15-70% MeCN/water with 0.1% TFA) to provide 6-(4-fluoro-4-((2S,5S)-2,3,4,5-tetrahydro-2,5-methanobenzo[f][1,4]oxazepine-4-carbonyl)piperidin-1-yl)pyrimidine-4-carbonitrile.

¹H NMR (500 MHz, DMSO-d₆) δ 8.58 (m, 1H), 7.64 (m, 1H), 7.09-7.20 (m, 2H), 6.84 (m, 1H), 6.79 (m, 1H), 4.97-5.18 (m, 2H), 4.02 (m, 1H), 3.95 (m, 1H), 3.78 (m, 1H), 3.48 (m, 1H), 3.39 (m, 1H), 3.23 (m, 1H), 2.26 (m, 1H), 1.96-2.18 (m, 3H), 1.76-1.88 (m, 2H); MS (EI) calculated for C₂₁H₂₁FN₅O₂ [M+H]⁺, 394. found, 394.

Compounds 1-3 to 1-35, 1-38, 1-40 to 1-47, 1-50 to 1-60 were prepared in a fashion analogous to the procedure used to prepare 1-2, substituting 3,3-difluoro-2,2-dimethylpropanoic acid for the appropriate carboxylic acid.

Compounds 1-37 and 1-39 were prepared in a fashion analogous to the procedure used for the preparation of 1-36, substituting the reagent 6-chloropyrimidine-4-carbonitrile for reagents 7-chlorothiazolo[5,4-d]pyrimidine and 8-chloro-[1,2,4]triazolo[1,5-a]pyrazine, respectively.

Compound 1-48 was prepared in a fashion analogous to the procedure used for the synthesis of 1-49, substituting 4-fluoropiperidine-4-carboxylic acid for 4-methylpiperidine-4-carboxylic acid.

TABLE 1

| | | | Exact |
|---|---|---|---|
| Compound | | | Mass |
| Number | Structure | Name | [M + H]⁺ |
| 1-1 | 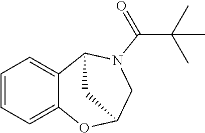 [SMILES: CC(C)(C)C(=O)N1C[C@@H]2C[C@H]1c1ccccc1O2] | 1-((2S,5S)-2,3-dihydro- 2,5- methanobenzo[f][1,4]oxaze- pin-4(5H)-yl)-2,2- dimethylpropan-1-one | Calculated 246, found 246 |
| 1-2 | 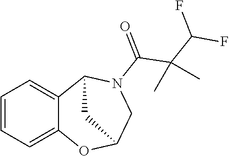 [SMILES: CC(C)(C(=O)N1C[C@@H]2C[C@H]1c1ccccc1O2)C(F)F] | 1-((2S,5S)-2,3-dihydro- 2,5- methanobenzo[f][1,4]oxaze- pin-4(5H)-yl)-3,3-difluoro- 2,2-dimethylpropan-1-one | Calculated 282, found 282 |
| 1-3 | 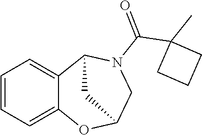 [SMILES: CC1(C(=O)N2C[C@@H]3C[C@H]2c2ccccc2O3)CCC1] | ((2S,5S)-2,3-dihydro-2,5- methanobenzo[f][1,4]oxaze- pin-4(5H)-yl)(1- methylcyclobutyl)methanone | Calculated 258, found 258 |
| 1-4 | 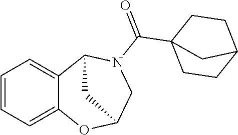 [SMILES: O=C(N1C[C@@H]2C[C@H]1c1ccccc1O2)C12CCC(CC1)C2] | bicyclo[2.2.1]heptan-1- yl((2S,5S)-2,3-dihydro-2,5- methanobenzo[f][1,4]oxaze- pin-4(5H)-yl)methanone | Calculated 284, found 284 |
| 1-5 | 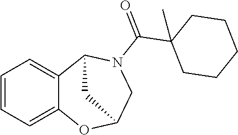 [SMILES: CC1(C(=O)N2C[C@@H]3C[C@H]2c2ccccc2O3)CCCCC1] | ((2S,5S)-2,3-dihydro-2,5- methanobenzo[f][1,4]oxaze- pin-4(5H)-yl)(1- methylcyclohexyl)methanone | Calculated 286, found 286 |
| 1-6 | 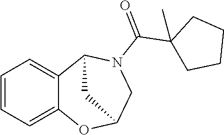 [SMILES: CC1(C(=O)N2C[C@@H]3C[C@H]2c2ccccc2O3)CCCC1] | ((2S,5S)-2,3-dihydro-2,5- methanobenzo[f][1,4]oxaze- pin-4(5H)-yl)(1- methylcyclopentyl) methanone | Calculated 272, found 272 |
| 1-7 | 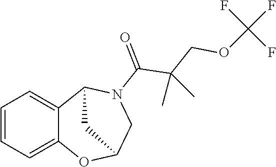 [SMILES: CC(C)(COC(F)(F)F)C(=O)N1C[C@@H]2C[C@H]1c1ccccc1O2] | 1-((2S,5S)-2,3-dihydro- 2,5- methanobenzo[f][1,4]oxaze- pin-4(5H)-yl)-2,2- dimethyl-3- (trifluoromethoxy)propan- 1-one | Calculated 330, found 330 |
| 1-8 | 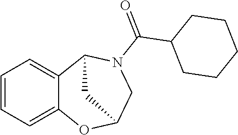 [SMILES: O=C(C1CCCCC1)N1C[C@@H]2C[C@H]1c1ccccc1O2] | cyclohexyl((2S,5S)-2,3- dihydro-2,5- methanobenzo[f][1,4]oxaze- pin-4(5H)-yl)methanone | Calculated 272, found 272 |
| 1-9 | 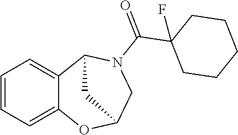 [SMILES: O=C(N1C[C@@H]2C[C@H]1c1ccccc1O2)C1(F)CCCCC1] | ((2S,5S)-2,3-dihydro-2,5- methanobenzo[f][1,4]oxaze- pin-4(5H)-yl)(1- fluorocyclohexyl)methanone | Calculated 290, found 290 |
| 1-10 | 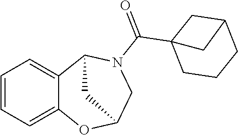 [SMILES: O=C(N1C[C@@H]2C[C@H]1c1ccccc1O2)C12CCCC(C1)C2] | bicyclo[3.1.1]heptan-1- yl((2S,5S)-2,3-dihydro-2,5- methanobenzo[f][1,4]oxaze- pin-4(5H)-yl)methanone | Calculated 284, found 284 |
| 1-11 | 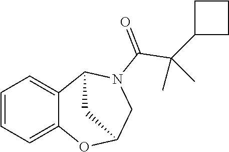 [SMILES: CC(C)(C(=O)N1C[C@@H]2C[C@H]1c1ccccc1O2)C1CCC1] | 2-cyclobutyl-1-[(2S,5S)- 2,3-dihydro-2,5-methano- 1,4-benzoxazepin-4(5H)- yl]-2-methylpropan-1-one | Calculated 286, found 286 |
| 1-12 | 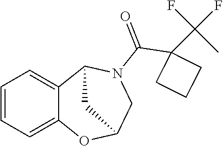 [SMILES: CC(F)(F)C1(C(=O)N2C[C@@H]3C[C@H]2c2ccccc2O3)CCC1] | [1-(1,1- difluoroethyl)cyclobutyl] [(2S,5S)-2,3-dihydro-2,5- methano-1,4- benzoxazepin-4(5H)- yl]methanone | Calculated 308, found 308 |
| 1-13 | 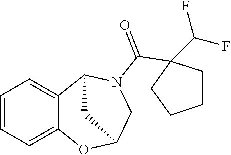 [SMILES: O=C(N1C[C@@H]2C[C@H]1c1ccccc1O2)C1(C(F)F)CCCC1] | [1- (difluoromethyl)cyclopentyl] [(2S,5S)-2,3-dihydro-2,5- methano-1,4- benzoxazepin-4(5H)- yl]methanone | Calculated 308, found 308 |
| 1-14 | 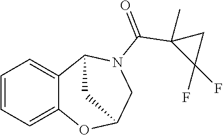 [SMILES: CC1(C(=O)N2C[C@@H]3C[C@H]2c2ccccc2O3)CC1(F)F] | [(R and S)-2,2-difluoro-1- methylcyclopropyl]- [(2S,5S)-2,3-dihydro-2,5- methano-1,4- benzoxazepin-4(5H)- yl]methanone | Calculated 280, found 280 |
| 1-15 | 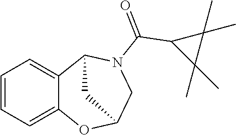 [SMILES: CC1(C)C(C(=O)N2C[C@@H]3C[C@H]2c2ccccc2O3)C1(C)C] | [(2S,5S)-2,3-dihydro-2,5- methano-1,4- benzoxazepin-4(5H)- yl](2,2,3,3- tetramethylcyclopropyl) methanone | Calculated 286, found 286 |
| 1-16 | 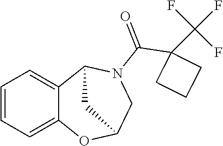 [SMILES: O=C(N1C[C@@H]2C[C@H]1c1ccccc1O2)C1(C(F)(F)F)CCC1] | [(2S,5S)-2,3-dihydro-2,5- methano-1,4- benzoxazepin-4(5H)-yl][1- (trifluoromethyl)cyclobutyl] methanone | Calculated 312, found 312 |
| 1-17 | 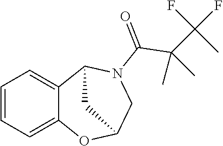 [SMILES: CC(F)(F)C(C)(C)C(=O)N1C[C@@H]2C[C@H]1c1ccccc1O2] | 1-[(2S,5S)-2,3-dihydro- 2,5-methano-1,4- benzoxazepin-4(5H)-yl]- 3,3-difluoro-2,2- dimethylbutan-1-one | Calculated 296, found 296 |
| 1-18 | 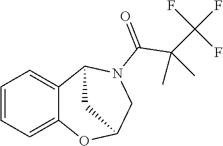 [SMILES: CC(C)(C(=O)N1C[C@@H]2C[C@H]1c1ccccc1O2)C(F)(F)F] | 1-[(2S,5S)-2,3-dihydro- 2,5-methano-1,4- benzoxazepin-4(5H)-yl]- 3,3,3-trifluoro-2,2- dimethylpropan-1-one | Calculated 300, found 300 |
| 1-19 | 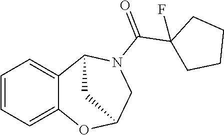 [SMILES: O=C(N1C[C@@H]2C[C@H]1c1ccccc1O2)C1(F)CCCC1] | [(2S,5S)-2,3-dihydro-2,5- methano-1,4- benzoxazepin-4(5H)-yl](1- fluorocyclopentyl)methanone | Calculated 276, found 276 |
| 1-20 | 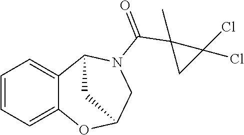 [SMILES: CC1(C(=O)N2C[C@@H]3C[C@H]2c2ccccc2O3)CC1(Cl)Cl] | [(R and S)-2,2-dichloro-1- methylcyclopropyl]- [(2S,5S)-2,3-dihydro-2,5- methano-1,4- benzoxazepin-4(5H)- yl]methanone | Calculated 312, found 312 |
| 1-21 | 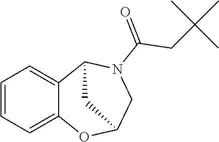 [SMILES: CC(C)(C)CC(=O)N1C[C@@H]2C[C@H]1c1ccccc1O2] | 1-[(2S,5S)-2,3-dihydro- 2,5-methano-1,4- benzoxazepin-4(5H)-yl]- 3,3-dimethylbutan-1-one | Calculated 260, found 260 |
| 1-22 | 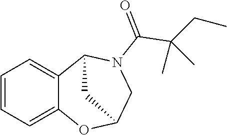 [SMILES: CCC(C)(C)C(=O)N1C[C@@H]2C[C@H]1c1ccccc1O2] | 1-[(2S,5S)-2,3-dihydro- 2,5-methano-1,4- benzoxazepin-4(5H)-yl]- 2,2-dimethylbutan-1-one | Calculated 260, found 260 |
| 1-23 | 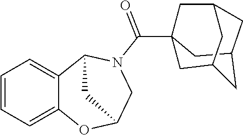 [SMILES: O=C(N1C[C@@H]2C[C@H]1c1ccccc1O2)C12CC3CC(CC(C3)C1)C2] | [(2S,5S)-2,3-dihydro-2,5- methano-1,4- benzoxazepin-4(5H)- yl][(3R,5R,7R)- tricyclo[3.3.1.1~3,7~] decan-1-yl]methanone | Calculated 324, found 324 |
| 1-24 | 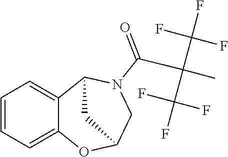 [SMILES: CC(C(=O)N1C[C@@H]2C[C@H]1c1ccccc1O2)(C(F)(F)F)C(F)(F)F] | 1-[(2S,5S)-2,3-dihydro- 2,5-methano-1,4- benzoxazepin-4(5H)-yl]- 3,3,3-trifluoro-2-methyl-2- (trifluoromethyl)propan-1- one | Calculated 354, found 354 |
| 1-25 | 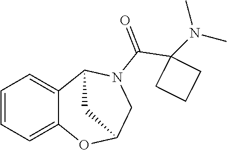 [SMILES: CN(C)C1(C(=O)N2C[C@@H]3C[C@H]2c2ccccc2O3)CCC1] | [(2S,5S)-2,3-dihydro-2,5- methano-1,4- benzoxazepin-4(5H)-yl][1- (dimethylamino)cyclobutyl] methanone | Calculated 287, found 287 |
| 1-26 | 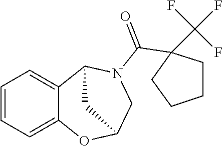 [SMILES: O=C(N1C[C@@H]2C[C@H]1c1ccccc1O2)C1(C(F)(F)F)CCCC1] | [(2S,5S)-2,3-dihydro-2,5- methano-1,4- benzoxazepin-4(5H)-yl][1- (trifluoromethyl) cyclopentyl]methanone | Calculated 326, found 326 |
| 1-27 | 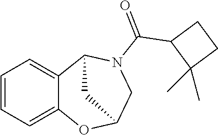 [SMILES: CC1(C)CCC1C(=O)N1C[C@@H]2C[C@H]1c1ccccc1O2] | [(2S,5S)-2,3-dihydro-2,5- methano-1,4- benzoxazepin-4(5H)-yl]- [(R and S)-2,2- dimethylcyclobutyl] methanone | Calculated 272, found 272 |
| 1-28 | 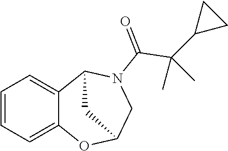 [SMILES: CC(C)(C(=O)N1C[C@@H]2C[C@H]1c1ccccc1O2)C1CC1] | 2-cyclopropyl-1-[(2S,5S)- 2,3-dihydro-2,5-methano- 1,4-benzoxazepin-4(5H)- yl]-2-methylpropan-1-one | Calculated 272, found 272 |
| 1-29 | 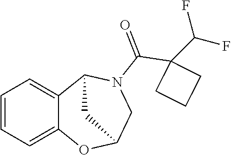 [SMILES: O=C(N1C[C@@H]2C[C@H]1c1ccccc1O2)C1(C(F)F)CCC1] | [1- (difluoromethyl)cyclobutyl] [(2S,5S)-2,3-dihydro-2,5- methano-1,4- benzoxazepin-4(5H)- yl]methanone | Calculated 294, found 294 |
| 1-30 | 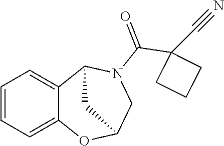 [SMILES: N#CC1(C(=O)N2C[C@@H]3C[C@H]2c2ccccc2O3)CCC1] | 1-[(2S,5S)-2,3-dihydro- 2,5-methano-1,4- benzoxazepine-4(5H)- carbonyl]cyclobutane-1- carbonitrile | Calculated 269, found 269 |
| 1-31 | 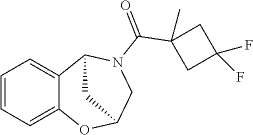 [SMILES: CC1(C(=O)N2C[C@@H]3C[C@H]2c2ccccc2O3)CC(F)(F)C1] | (3,3-difluoro-1- methylcyclobutyl)[(2S,5S)- 2,3-dihydro-2,5-methano- 1,4-benzoxazepin-4(5H)- yl]methanone | Calculated 294, found 294 |
| 1-32 | 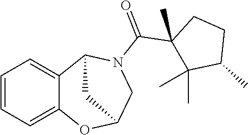 [SMILES: C[C@H]1CC[C@@](C)(C(=O)N2C[C@@H]3C[C@H]2c2ccccc2O3)C1(C)C] | [(2S,5S)-2,3-dihydro-2,5- methano-1,4- benzoxazepin-4(5H)- yl][(1R,3S and 1S,3R)- 1,2,2,3- tetramethylcyclopentyl] methanone | Calculated 314, found 314 |
| 1-33 | 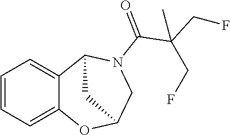 [SMILES: CC(CF)(CF)C(=O)N1C[C@@H]2C[C@H]1c1ccccc1O2] | 1-[(2S,5S)-2,3-dihydro- 2,5-methano-1,4- benzoxazepin-4(5H)-yl]-3- fluoro-2-(fluoromethyl)-2- methylpropan-1-one | Calculated 282, found 282 |
| 1-34 | 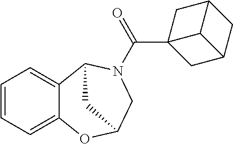 [SMILES: O=C(N1C[C@@H]2C[C@H]1c1ccccc1O2)C12CC3CC(C1)C32] | [(2S,5S)-2,3-dihydro-2,5- methano-1,4- benzoxazepin-4(5H)- yl](tricyclo[3.1.1.0~3,6~] heptan-6-yl)methanone | Calculated 282, found 282 |
| 1-35 | 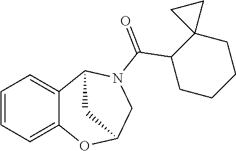 [SMILES: O=C(C1CCCCC12CC2)N1C[C@@H]2C[C@H]1c1ccccc1O2] | [(2S,5S)-2,3-dihydro-2,5- methano-1,4- benzoxazepin-4(5H)-yl]- [(R and S)-spiro[2.5]octan- 4-yl]methanone | Calculated 298, found 298 |
| 1-36 | 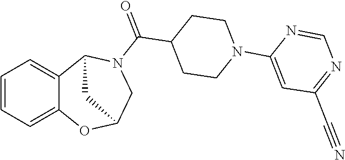 [SMILES: N#Cc1cc(N2CCC(C(=O)N3C[C@@H]4C[C@H]3c3ccccc3O4)CC2)ncn1] | 6-{4-[(2S,5S)-2,3-dihydro- 2,5-methano-1,4- benzoxazepine-4(5H)- carbonyl]piperidin-1- yl}pyrimidine-4- carbonitrile | Calculated 376, found 376 |
| 1-37 | 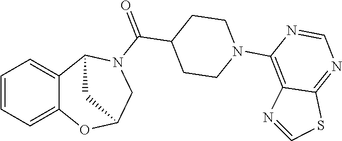 [SMILES: O=C(C1CCN(c2ncnc3scnc23)CC1)N1C[C@@H]2C[C@H]1c1ccccc1O2] | [(2S,5S)-2,3-dihydro-2,5- methano-1,4- benzoxazepin-4(5H)-yl][1- ([1,3]thiazolo[5,4- d]pyrimidin-7-yl)piperidin- 4-yl]methanone | Calculated 408, found 408 |
| 1-38 | 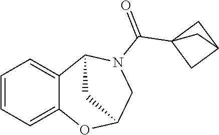 [SMILES: O=C(N1C[C@@H]2C[C@H]1c1ccccc1O2)C12CC(C1)C2] | (bicyclo[1.1.1]pentan-1- yl)[(2S,5S)-2,3-dihydro- 2,5-methano-1,4- benzoxazepin-4(5H)- yl]methanone | Calculated 256, found 256 |
| 1-39 | 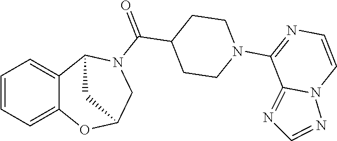 [SMILES: O=C(C1CCN(c2nccn3ncnc23)CC1)N1C[C@@H]2C[C@H]1c1ccccc1O2] | [(2S,5S)-2,3-dihydro-2,5- methano-1,4- benzoxazepin-4(5H)-yl][1- ([1,2,4]triazolo[1,5- a]pyrazin-8-yl)piperidin-4- yl]methanone | Calculated 391, found 391 |
| 1-40 | 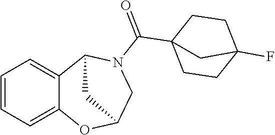 [SMILES: O=C(N1C[C@@H]2C[C@H]1c1ccccc1O2)C12CCC(F)(CC1)C2] | ((2S,5S)-2,3-dihydro-2,5- methanobenzo[f][1,4]oxaze- pin-4(5H)-yl)(4- fluorobicyclo[2.2.1]heptan- 1-yl)methanone | Calculated 302, found 302 |
| 1-41 | 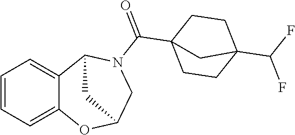 [SMILES: O=C(N1C[C@@H]2C[C@H]1c1ccccc1O2)C12CCC(C(F)F)(CC1)C2] | (4- (difluoromethyl)bicyclo [2.2.1]heptan-1-yl)((2S,5S)- 2,3-dihydro-2,5- methanobenzo[f][1,4]oxaze- pin-4(5H)-yl)methanone | Calculated 334, found 334 |
| 1-42 | 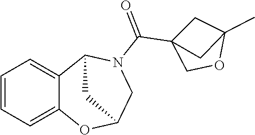 [SMILES: CC12CC(C(=O)N3C[C@@H]4C[C@H]3c3ccccc3O4)(CO1)C2] | [(2S,5S)-2,3-dihydro-2,5- methano-1,4- benzoxazepin-4(5H)-yl](1- methyl-2- oxabicyclo[2.1.1]hexan-4- yl)methanone | Calculated 286, found 286 |
| 1-43 | 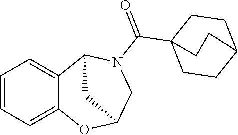 [SMILES: O=C(N1C[C@@H]2C[C@H]1c1ccccc1O2)C12CCC(CC1)CC2] | (bicyclo[2.2.2]octan-1- yl)[(2S,5S)-2,3-dihydro- 2,5-methano-1,4- benzoxazepin-4(5H)- yl]methanone | Calculated 298, found 298 |
| 1-44 | 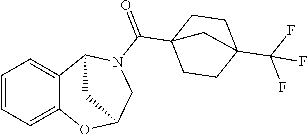 [SMILES: O=C(N1C[C@@H]2C[C@H]1c1ccccc1O2)C12CCC(C(F)(F)F)(CC1)C2] | [(2S,5S)-2,3-dihydro-2,5- methano-1,4- benzoxazepin-4(5H)-yl][4- (trifluoromethyl)bicyclo [2.2.1]heptan-1-yl]methanone | Calculated 352, found 352 |
| 1-45 | 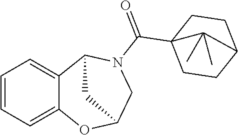 [SMILES: CC1(C)C2CCC1(C(=O)N1C[C@@H]3C[C@H]1c1ccccc1O3)CC2] | [(2S,5S)-2,3-dihydro-2,5- methano-1,4- benzoxazepin-4(5H)- yl](7,7- dimethylbicyclo[2.2.1] heptan-1-yl)methanone | Calculated 312, found 312 |
| 1-46 | 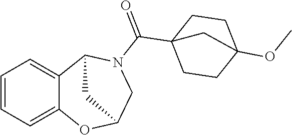 [SMILES: COC12CCC(C(=O)N3C[C@@H]4C[C@H]3c3ccccc3O4)(CC1)C2] | [(2S,5S)-2,3-dihydro-2,5- methano-1,4- benzoxazepin-4(5H)-yl](4- methoxybicyclo[2.2.1] heptan-1-yl)methanone | Calculated 314, found 314 |
| 1-47 | 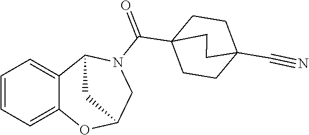 [SMILES: N#CC12CCC(C(=O)N3C[C@@H]4C[C@H]3c3ccccc3O4)(CC1)CC2] | 4-[(2S,5S)-2,3-dihydro- 2,5-methano-1,4- benzoxazepine-4(5H)- carbonyl]bicyclo[2.2.2] octane-1-carbonitrile | Calculated 323, found 323 |
| 1-48 | 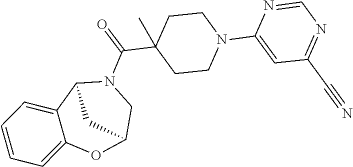 [SMILES: CC1(C(=O)N2C[C@@H]3C[C@H]2c2ccccc2O3)CCN(c2cc(C#N)ncn2)CC1] | 6-{4-[(2S,5S)-2,3-dihydro- 2,5-methano-1,4- benzoxazepine-4(5H)- carbonyl]-4- methylpiperidin-1- yl}pyrimidine-4- carbonitrile | Calculated 390, found 390 |
| 1-49 | 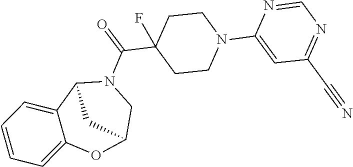 [SMILES: N#Cc1cc(N2CCC(F)(C(=O)N3C[C@@H]4C[C@H]3c3ccccc3O4)CC2)ncn1] | 6-{4-[(2S,5S)-2,3-dihydro- 2,5-methano-1,4- benzoxazepine-4(5H)- carbonyl]-4- fluoropiperidin-1- yl}pyrimidine-4- carbonitrile | Calculated 394, found 394 |
| 1-50 | 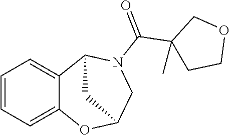 [SMILES: CC1(C(=O)N2C[C@@H]3C[C@H]2c2ccccc2O3)CCOC1] | [(2S,5S)-2,3-dihydro-2,5- methano-1,4- benzoxazepin-4(5H)-yl]- [(R and S)-3- methyloxolan-3- yl]methanone | Calculated 274, found 274 |
| 1-51 | 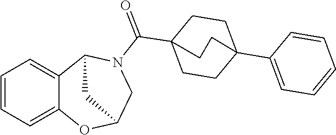 [SMILES: O=C(N1C[C@@H]2C[C@H]1c1ccccc1O2)C12CCC(c3ccccc3)(CC1)CC2] | [(2S,5S)-2,3-dihydro-2,5- methano-1,4- benzoxazepin-4(5H)-yl](4- phenylbicyclo[2.2.2]octan- 1-yl)methanone | Calculated 374, found 374 |
| 1-52 | 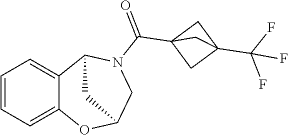 [SMILES: O=C(N1C[C@@H]2C[C@H]1c1ccccc1O2)C12CC(C(F)(F)F)(C1)C2] | [(2S,5S)-2,3-dihydro-2,5- methano-1,4- benzoxazepin-4(5H)-yl][3- (trifluoromethyl)bicyclo [1.1.1]pentan-1-yl]methanone | Calculated 324, found 324 |
| 1-53 | 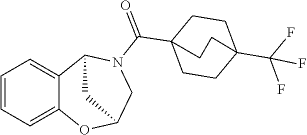 [SMILES: O=C(N1C[C@@H]2C[C@H]1c1ccccc1O2)C12CCC(C(F)(F)F)(CC1)CC2] | [(2S,5S)-2,3-dihydro-2,5- methano-1,4- benzoxazepin-4(5H)-yl][4- (trifluoromethyl)bicyclo [2.2.2]octan-1-yl]methanone | Calculated 366, found 366 |
| 1-54 | 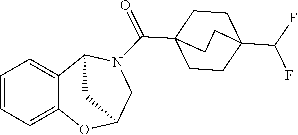 [SMILES: O=C(N1C[C@@H]2C[C@H]1c1ccccc1O2)C12CCC(C(F)F)(CC1)CC2] | [4- (difluoromethyl)bicyclo [2.2.2]octan-1-yl][(2S,5S)- 2,3-dihydro-2,5-methano- 1,4-benzoxazepin-4(5H)- yl]methanone | Calculated 348, found 348 |
| 1-55 | 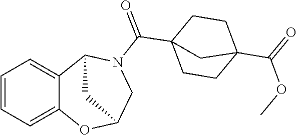 | methyl 4-[(2S,5S)-2,3- dihydro-2,5-methano-1,4- benzoxazepine-4(5H)- carbonyl]bicyclo[2.2.1] heptane-1-carboxylate | Calculated 342, found 342 |
| 1-56 | 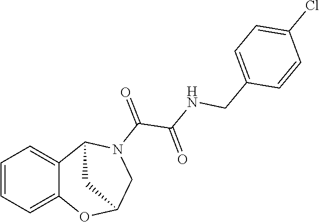 [SMILES: O=C(NCc1ccc(Cl)cc1)C(=O)N1C[C@@H]2C[C@H]1c1ccccc1O2] | N-[(4- chlorophenyl)methyl]-2- [(2R,5S)-2,3-dihydro-2,5- methano-1,4- benzoxazepin-4(5H)-yl]-2- oxoacetamide | Calculated 357, found 357 |
| 1-57 | 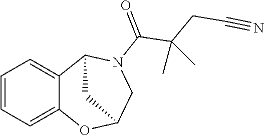 [SMILES: CC(C)(CC#N)C(=O)N1C[C@@H]2C[C@H]1c1ccccc1O2] | 4-[(2S,5S)-2,3-dihydro- 2,5-methano-1,4- benzoxazepin-4(5H)-yl]- 3,3-dimethyl-4- oxobutanenitrile | Calculated 271, found 271 |
| 1-58 | 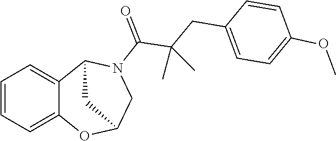 [SMILES: COc1ccc(CC(C)(C)C(=O)N2C[C@@H]3C[C@H]2c2ccccc2O3)cc1] | 1-[(2S,5S)-2,3-dihydro- 2,5-methano-1,4- benzoxazepin-4(5H)-yl]-3- (4-methoxyphenyl)-2,2- dimethylpropan-1-one | Calculated 352, found 352 |
| 1-59 | 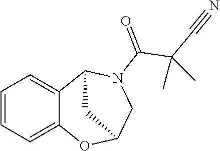 [SMILES: CC(C)(C#N)C(=O)N1C[C@@H]2C[C@H]1c1ccccc1O2] | 3-[(2S,5S)-2,3-dihydro- 2,5-methano-1,4- benzoxazepin-4(5H)-yl]- 2,2-dimethyl-3- oxopropanenitrile | Calculated 257, found 257 |
| 1-60 | 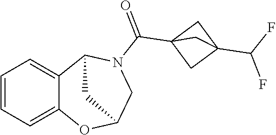 [SMILES: O=C(N1C[C@@H]2C[C@H]1c1ccccc1O2)C12CC(C(F)F)(C1)C2] | [3- (difluoromethyl)bicyclo [1.1.1]pentan-1-yl][(2S,5S)- 2,3-dihydro-2,5-methano- 1,4-benzoxazepin-4(5H)- yl]methanone | Calculated 306, found 306 |

### Table 2 Compounds

### Example 2A. Preparation of 1-((2S,5S)-7-Chloro-2,3-dihydro-2,5-methanobenzo[f][1,4]oxazepin-4(5H)-yl)-3,3-difluoro-2,2-dimethylpropan-1-one (2-1)

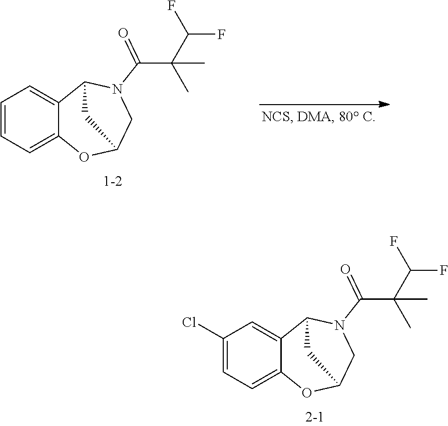 [SMILES: CC(C)(C(=O)N1C[C@@H]2C[C@H]1c1ccccc1O2)C(F)F ; CC(C)(C(=O)N1C[C@@H]2C[C@H]1c1cc(Cl)ccc1O2)C(F)F]

A solution of 1-((2S,5S)-2,3-dihydro-2,5-methanobenzo[f][1,4]oxazepin-4(5H)-yl)-3,3-difluoro-2,2-dimethylpropan-1-one (20 mg, 0.071 mmol) in DMA (0.5 mL) was treated with NCS (11 mg, 0.082 mmol) and stirred for 1 h at 80° C. The reaction was diluted with DMA (0.5 mL) and purified by reverse phase chromatography (gradient of 30-95% MeCN/water with 0.1% TFA) to provide 1-((2S,5S)-7-chloro-2,3-dihydro-2,5-methanobenzo[f][1,4]oxazepin-4(5H)-yl)-3,3-difluoro-2,2-dimethylpropan-1-one, 2-1. ¹H NMR (500 MHz, DMSO-d₆) δ 7.17 (m, 2H), 6.79 (d, J=8.7 Hz, 1H), 6.23 (t, J=57 Hz, 1H), 5.11 (m, 2H), 3.94 (s, 2H), 2.17 (ddd, J=12.4, 4.9, 2.8 Hz, 1H), 2.02 (d, J=12.4 Hz, 1H), 1.21 (s, 3H), 1.08 (s, 3H); MS (EI) calculated for C₁₅H₁₇ClF₂NO₂ [M+H]⁺, 316. found, 316.

### Example 2B. Preparation of (2S,5S)-4-(3,3-Difluoro-2,2-dimethylpropanoyl)-2,3,4,5-tetrahydro-2,5-methanobenzo[f][1,4]oxazepine-7-carbonitrile (2-2)

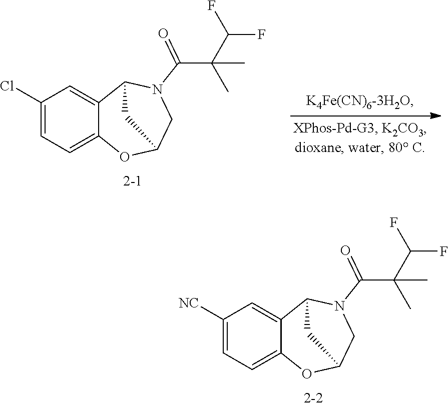 [SMILES: CC(C)(C(=O)N1C[C@@H]2C[C@H]1c1cc(Cl)ccc1O2)C(F)F ; CC(C)(C(=O)N1C[C@@H]2C[C@H]1c1cc(C#N)ccc1O2)C(F)F]

A mixture containing K₂CO₃ (1 mg, 0.007 mmol), K₄Fe(CN)₆·3H₂O (10 mg, 0.024 mmol) and 1-((2S,5S)-7-chloro-2,3-dihydro-2,5-methanobenzo[f][1,4]oxazepin-4(5H)-yl)-3,3-difluoro-2,2-dimethylpropan-1-one, 2-1 (15 mg, 0.048 mmol) in dioxane (0.5 mL) was treated with XPHOS-Pd-G3 (8 mg, 0.009 mmol) and water (0.5 mL). The mixture was stirred for 2 h at 80° C., diluted with 1 mL of MeOH, filtered and purified by reverse phase chromatography (gradient of 15-70% MeCN/water with 0.1% TFA) to give the desired product, (2S,5S)-4-(3,3-difluoro-2,2-dimethylpropanoyl)-2,3,4,5-tetrahydro-2,5-methanobenzo[f][1,4]oxazepine-7-carbonitrile, 2-2. ¹H NMR (500 MHz, DMSO-d₆) δ 7.45-7.68 (m, 2H), 6.95 (d, J=8.5 Hz, 1H), 6.22 (t, J=57 Hz, 1H), 5.19 (s, 2H), 3.97 (s, 2H), 2.25 (ddd, J=13, 5.0, 2.7 Hz, 1H), 2.07 (d, J=13 Hz, 1H), 1.21 (s, 3H), 1.07 (s, 3H); MS (EI) calculated for C₁₆H₁₇F₂N₂O₂ [M+H]⁺, 307. found, 307.

### Example 2C. Preparation of 3,3-Difluoro-2,2-dimethyl-1-((2S,5S)-7-methyl-2,3-dihydro-2,5-methanobenzo[f][1,4]oxazepin-4(5H)-yl)propan-1-one (2-3)

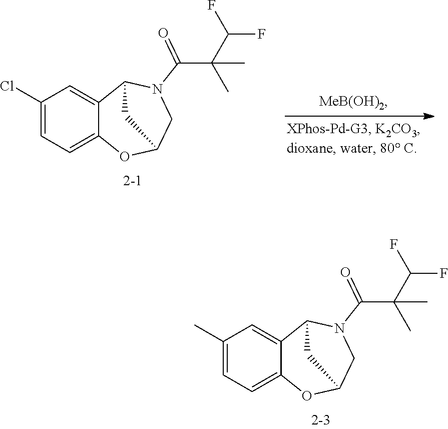 [SMILES: CC(C)(C(=O)N1C[C@@H]2C[C@H]1c1cc(Cl)ccc1O2)C(F)F ; Cc1ccc2c(c1)[C@@H]1C[C@@H](CN1C(=O)C(C)(C)C(F)F)O2]

A mixture containing 1-((2S,5S)-7-chloro-2,3-dihydro-2,5-methanobenzo[f][1,4]oxazepin-4(5H)-yl)-3,3-difluoro-2,2-dimethylpropan-1-one (15 mg, 0.048 mmol), XPhos-Pd-G3 (8 mg, 0.009 mmol), K₂CO₃ (10 mg, 0.07 mmol), methylboronic acid (14 mg, 0.23 mmol) in dioxane (0.8 mL) and water (0.2 mL) was stirred overnight at 80° C. The crude reaction mixture was diluted with 1 mL of DMSO, filtered and purified by reverse phase chromatography (gradient of 30-95% MeCN/water with 0.1% TFA) to provide 3,3-difluoro-2,2-dimethyl-1-((2S,5S)-7-methyl-2,3-dihydro-2,5-methanobenzo[f][1,4]oxazepin-4(5H)-yl)propan-1-one. ¹H NMR (500 MHz, DMSO-d₆) δ 6.94 (m, 2H), 6.65 (d, J=8.1 Hz, 1H), 6.23 (t, J=57 Hz, 1H), 5.12 (s, 1H), 5.03 (s, 1H), 3.90 (m, 2H), 2.18 (s, 3H), 2.10 (ddd, J=12, 4.7, 2.9 Hz, 1H), 1.98 (d, J=12 Hz, 1H), 1.19 (s, 3H), 1.11 (s, 3H); MS (EI) calculated for C₁₆H₂₀F₂NO₂ [M+H]⁺, 296. found, 296.

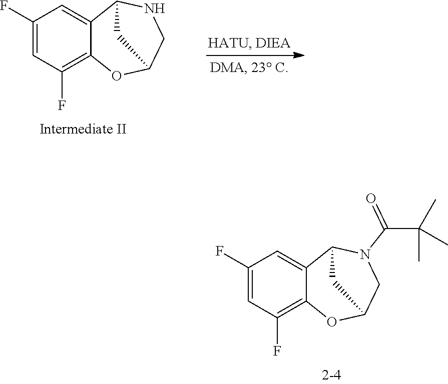 [SMILES: Fc1cc(F)c2c(c1)[C@@H]1C[C@@H](CN1)O2 ; CC(C)(C)C(=O)N1C[C@@H]2C[C@H]1c1cc(F)cc(F)c1O2]

### Example 2D. Preparation of 1-[(2S,5S)-7,9-difluoro-2,3-dihydro-2,5-methano-1,4-benzoxazepin-4(5H)-yl]-2,2-dimethylpropan-1-one (2-4)

(2S,5S)-7,9-difluoro-2,3,4,5-tetrahydro-2,5-methanobenzo[f][1,4]oxazepine, Intermediate II (9.6 mg, 0.05 mmol) in DMF (2000 μL), was treated with DIEA (19.56 μL, 0.112 mmol) and pivaloyl chloride (13.78 μL, 0.112 mmol). The reaction mixture was stirred at 23° C. for 1 hour. The reaction was then diluted with DMSO (2 mL), filtered, and purified by reverse phase chromatography (30-95% MeCN/water with 0.1% TFA) to provide 1-[(2S,5S)-7,9-difluoro-2,3-dihydro-2,5-methano-1,4-benzoxazepin-4(5H)-yl]-2,2-dimethylpropan-1-one. ¹H NMR (500 MHz, DMSO-d₆) δ 7.14 (s, 1H), 6.85 (s, 1H), 6.54 (s, 1H), 5.18 (s, 2H), 3.95 (s, 1H), 2.18 (d, J=12.4 Hz, 1H), 2.06 (d, J=11.4 Hz, 1H), 1.11 (s, 9H); MS (EI) calculated for C₁₅H₁₈F₂NO₂ [M+H]⁺, 282. found, 282.

### Example 2E. Preparation of (2S,5S)-4-(3,3-Difluoro-2,2-dimethylpropanoyl)-7-fluoro-2,3,4,5-tetrahydro-2,5-methanobenzo[f][1,4]oxazepine-9-carbonitrile (2-11)

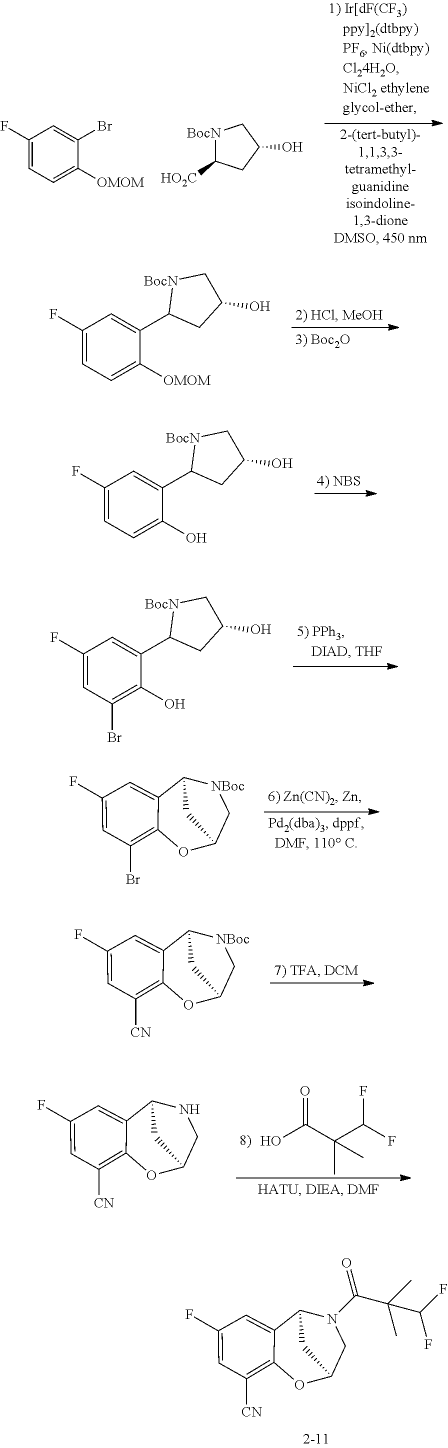

Step 1. A solution of (2S,4R)-1-(tert-butoxycarbonyl)-4-hydroxypyrrolidine-2-carboxylic acid (295 mg, 1.28 mmol), 2-bromo-4-fluoro-1-(methoxymethoxy)benzene (300 mg, 1.28 mmol), isoindoline-1,3-dione (188 mg, 1.28 mmol), Ni(dtbpy)Cl₂·4H₂O (102 mg, 0.255 mmol), NiCl₂ ethylene glycol dimethylether complex (42 mg, 0.19 mmol), 2-(tert-butyl)-1,1,3,3-tetramethylguanidine (328 mg, 1.91 mmol) in DMSO (20 mL) was treated with Ir[dF(CF₃)ppy]₂(dtbpy)·PF₆ (29 mg, 0.026 mmol). The reaction mixture was stirred for 4 h with 450 nm irradiation. The mixture was poured into water and extracted with EtOAc. The combined organic layers were washed with brine, dried over Na₂SO₄, filtered and concentrated. Reverse phase chromatography (gradient of 38-68% MeCN/water with 0.1% TFA) provided tert-butyl (4R)-2-(5-fluoro-2-(methoxymethoxy)phenyl)-4-hydroxypyrrolidine-1-carboxylate. MS (EI) calculated for [M-tBu+H]⁺, 286. found, 286.

Step 2. A solution of tert-butyl (4R)-2-(5-fluoro-2-(methoxymethoxy)phenyl)-4-hydroxypyrrolidine-1-carboxylate (220 mg, 0.451 mmol) in MeOH/HCl (3 mL) was stirred for 2 h and concentrated to give (3R)-5-(5-fluoro-2-hydroxyphenyl)pyrrolidin-3-ol. MS (EI) calculated for C₁₀H₁₃FNO₃ [M+H]⁺, 198. found, 198.

Step 3. A solution of (3R)-5-(5-fluoro-2-hydroxyphenyl)pyrrolidin-3-ol (137 mg, 0.452 mmol) in MeOH (4 mL) was treated with di-tert-butyl dicarbonate (99 mg, 0.45 mmol) and TEA (0.19 mL, 1.4 mmol). The mixture was stirred for 15 h and concentrated. Reverse phase chromatography (gradient of 25-55% MeCN/water with 0.05% NH₃ and 10 mM NH₄HCO₃) to give tert-butyl (4R)-2-(5-fluoro-2-hydroxyphenyl)-4-hydroxypyrrolidine-1-carboxylate. MS (EI) calculated for C₁₁H₁₃FNO₄ [M-tBu+H]⁺, 242. found, 242.

Step 4. A solution of tert-butyl (4R)-2-(5-fluoro-2-hydroxyphenyl)-4-hydroxypyrrolidine-1-carboxylate (56 mg, 0.19 mmol) in DMF (2 mL) was treated with NBS (34 mg, 0.19 mmol) and stirred for 1 h. The mixture was purified by reverse phase chromatography (gradient of 30-60% MeCN/water with 0.05% NH₃ and 10 mM NH₄HCO₃) to give product tert-butyl (4R)-2-(3-bromo-5-fluoro-2-hydroxyphenyl)-4-hydroxypyrrolidine-1-carboxylate. MS (EI) calculated for C₁₁H₁₂BrFNO₄ [M-tBu+H]⁺, 320, 322. found, 320, 322.

Step 5. A solution of tert-butyl (4R)-2-(3-bromo-5-fluoro-2-hydroxyphenyl)-4-hydroxypyrrolidine-1-carboxy late (50 mg, 0.13 mmol), triphenylphosphine (210 mg, 0.80 mmol) in THF (3 mL) was treated with diisopropyl azodicarboxylate (0.16 mL, 0.80 mmol). The mixture was stirred for 15 h, concentrated, and purified by chromatography on SiO₂ (3:1 petroleum ether/EtOAc) to give tert-butyl (2S,5S)-9-bromo-7-fluoro-2,3-dihydro-2,5-methanobenzo[f][1,4]oxazepine-4(5H)-carboxylate. MS (EI) calculated for C₁₁H₁₀BrFNO₃ [M-tBu+H]⁺, 302, 304. found, 302, 304.

Step 6. A solution of tert-butyl (2S,5S)-9-bromo-7-fluoro-2,3-dihydro-2,5-methanobenzo[f][1,4]oxazepine-4(5H)-carboxylate (50 mg, 0.14 mmol) in DMF (1.5 mL) was treated with dicyanozinc (49 mg, 0.42 mmol), zinc (1.8 mg, 0.028 mmol), 1,1′-bis(diphenylphosphino)ferrocene (15 mg, 0.028 mmol) and tris(dibenzylideneacetone)dipalladium(0) (13 mg. 0.014 mmol). The mixture was stirred at 110 ºC for 12 h, quenched with water, and extracted with EtOAc. The combined organic layers were washed with brine, dried over Na₂SO₄, filtered and concentrated. The residue was purified by chromatography on SiO₂ (3:1 petroleum ether/EtOAc) to give tert-butyl (2S,5S)-9-cyano-7-fluoro-2,3-dihydro-2,5-methanobenzo[f][1,4]oxazepine-4(5H)-carboxylate. MS (EI) calculated for C₁₂H₁₀FN₂O₃ [M-tBu+H]⁺, 249. found, 249.

Step 7. A solution of tert-butyl (2S,5S)-9-cyano-7-fluoro-2,3-dihydro-2,5-methanobenzo[f][1,4]oxazepine-4(5H)-carboxylate (40 mg, 0.13 mmol) in DCM (1.5 mL) and TFA (0.8 mL) was stirred for 1 h and concentrated to give (2S,5S)-7-fluoro-2,3,4,5-tetrahydro-2,5-methanobenzo[f][1,4]oxazepine-9-carbonitrile. MS (EI) calculated for C₁₁H₁₀FN₂O [M+H]⁺, 205. found, 205.

Step 8. A mixture of 3,3-difluoro-2,2-dimethylpropanoic acid (25 mg, 0.18 mmol), HATU (75 mg, 0.20 mmol) and DIEA (0.086 mL, 0.49 mmol) in DMF (1 mL) was treated with (2S,5S)-7-fluoro-2,3,4,5-tetrahydro-2,5-methanobenzo[f][1,4]oxazepine-9-carbonitrile (20 mg, 0.098 mmol) and stirred for 16 h. The mixture was concentrated and purified by reverse phase chromatography (gradient of 35-65% MeCN/water with 0.1% TFA) to provide (2S,5S)-4-(3,3-difluoro-2,2-dimethylpropanoyl)-7-fluoro-2,3,4,5-tetrahydro-2,5-methanobenzo[f][1,4]oxazepine-9-carbonitrile. ¹H NMR (400 MHz, CDCl₃) δ 7.28 (m, 1H), 7.13 (dd, J=8.0, 2.8 Hz, 1H), 6.05 (m, 1H), 5.28 (m, 1H), 5.24 (m, 1H), 4.07 (m, 1H), 3.93 (m, 1H), 2.21 (s, 2H), 1.32 (s, 3H), 1.20 (s, 3H); MS (EI) calculated for C₁₆H₁₆F₃N₂O₂ [M+H]⁺, 325. found, 325.

### Example 2F. Preparation of 1-((2S,5S)-9-Chloro-7-fluoro-2,3-dihydro-2,5-methanobenzo[f][1,4]oxazepin-4(5H)-yl)-3,3-difluoro-2,2-dimethylpropan-1-one (2-24)

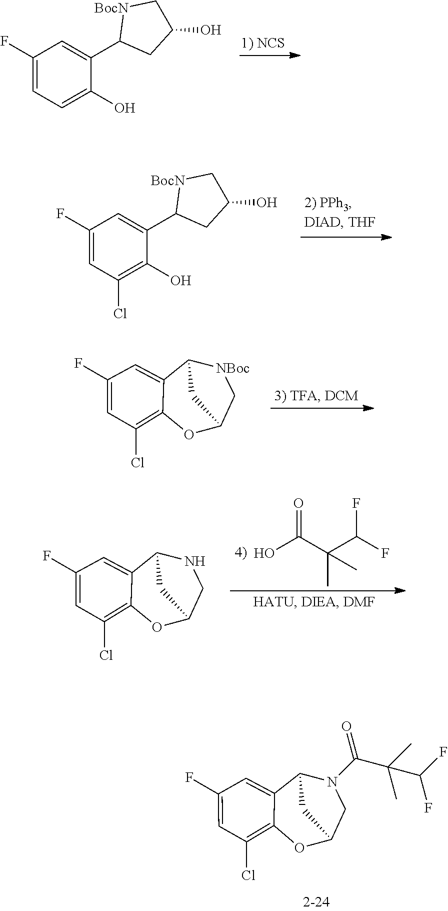

Step 1. A solution of tert-butyl (4R)-2-(5-fluoro-2-hydroxyphenyl)-4-hydroxypyrrolidine-1-carboxylate (50 mg, 0.17 mmol) in DMF (2 mL) was treated with NBS (27 mg, 0.20 mmol) and stirred for 1 h. The mixture was purified by reverse phase chromatography (gradient of 30-60% MeCN/water with 0.05% NH₃ and 10 mM NH₄HCO₃) to give product tert-butyl (4R)-2-(3-chloro-5-fluoro-2-hydroxyphenyl)-4-hydroxypyrrolidine-1-carboxylate. ¹H NMR (400 MHz, CDCl₃) δ 6.99 (m, 1H), 6.80 (m, 1H), 5.24 (m, 1H), 4.54 (m, 1H), 3.82 (m, 1H), 3.78 (m, 1H), 3.57 (m, 1H), 2.63 (m, 1H), 2.43 (m, 1H), 1.40 (s, 9H); MS (EI) calculated for C₁₁H₁₂ClFNO₄ [M-tBu+H]⁺, 258. found, 258.

Step 2. A solution of tert-butyl (4R)-2-(3-chloro-5-fluoro-2-hydroxyphenyl)-4-hydroxypyrrolidine-1-carboxylate (30 mg, 0.090 mmol), triphenylphosphine (140 mg, 0.54 mmol) in THF (3 mL) was treated dropwise with diisopropyl azodicarboxylate (0.11 mL, 0.54 mmol). The mixture was stirred for 15 h, concentrated, and purified by chromatography on SiO₂ (3:1 petroleum ether/EtOAc) to give tert-butyl (2S,5S)-9-chloro-7-fluoro-2,3-dihydro-2,5-methanobenzo[f][1,4]oxazepine-4(5H)-carboxylate. MS (EI) calculated for C₁₁H₁₀ClFNO₃ [M-tBu+H]⁺, 258. found, 258.

Step 3. A solution of tert-butyl (2S,5S)-9-chloro-7-fluoro-2,3-dihydro-2,5-methanobenzo[f][1,4]oxazepine-4(5H)-carboxylate (20 mg, 0.064 mmol) in DCM (3 mL) and TFA (1 mL) was stirred for 1 h and concentrated to give (2S,5S)-9-chloro-7-fluoro-2,3,4,5-tetrahydro-2,5-methanobenzo[f][1,4]oxazepine.

Step 4. A mixture of (2S,5S)-9-chloro-7-fluoro-2,3,4,5-tetrahydro-2,5-methanobenzo[f][1,4]oxazepine (13 mg, 0.061 mmol), HATU (46 mg, 0.12 mmol) and DIEA (0.053 mL, 0.31 mmol) in DMF (1 mL) was treated with 3,3-difluoro-2,2-dimethylpropanoic acid (25 mg, 0.18 mmol) and stirred for 1 h. The mixture was concentrated and the residue purified by reverse phase chromatography (gradient of 42-72% MeCN/water with 0.1% TFA) to give 1-((2S,5S)-9-chloro-7-fluoro-2,3-dihydro-2,5-methanobenzo[f][1,4]oxazepin-4(5H)-yl)-3,3-difluoro-2,2-dimethylpropan-1-one. ¹H NMR (500 MHz, CD₃OD) δ 7.06 (m, 1H), 6.96 (m, 1H), 6.10 (t, J=57 Hz, 1H), 5.25 (m, 1H), 5.13 (m, 1H), 4.04 (m, 2H), 2.24 (m, 1H), 2.16 (m, 1H), 1.26 (s, 3H), 1.18 (s, 3H); MS (EI) calculated for C₁₅H₁₆ClF₃NO₂ [M+H]⁺, 334. found, 334.

### Example 2G. Preparation of 1-((2S,5S)-9-Chloro-2,3-dihydro-2,5-methanobenzo[f][1,4]oxazepin-4(5H)-yl)-3,3-difluoro-2,2-dimethylpropan-1-one (2-20) and 1-

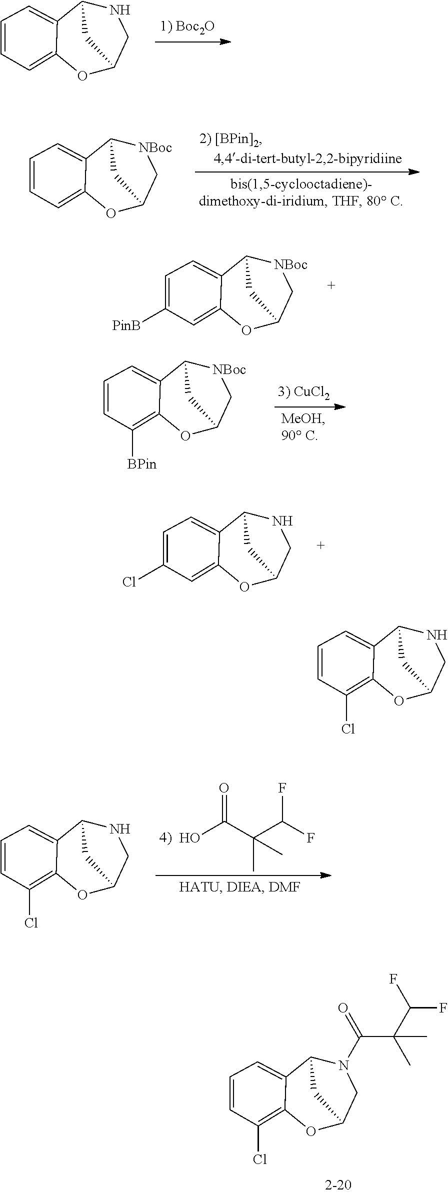

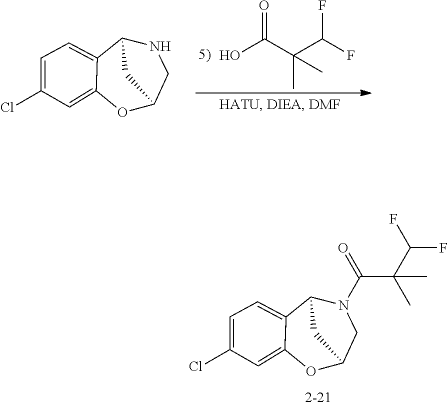

### ((2S,5S)-8-Chloro-2,3-dihydro-2,5-methanobenzo[f][1,4]oxazepin-4(5H)-yl)-3,3-difluoro-2,2-dimethylpropan-1-one (2-21)

Step 1. A mixture of (2S,5S)-2,3,4,5-tetrahydro-2,5-methanobenzo[f][1,4]oxazepine, Intermediate I (180 mg, 1.12 mmol) in DCM (15 mL) was treated with di-tert-butyl-dicarbonate (1.3 mL, 5.6 mmol) and DIEA (0.98 mL, 5.6 mmol), and stirred for 15 h. The mixture was concentrated and purified by reverse phase chromatography (gradient of 40-70% MeCN/water with 10 mM NH₄HCO₃ to give product tert-butyl (2S,5S)-2,3-dihydro-2,5-methanobenzo[f][1,4]oxazepine-4(5H)-carboxylate. ¹H NMR (500 MHz, CDCl₃) δ 7.04-7.23 (m, 2H), 6.76-6.85 (m, 2H), 4.73-5.01 (m, 2H), 3.56-3.78 (m, 2H), 2.08-2.28 (m, 2H), 1.41-1.49 (m, 9H); MS (EI) calculated for C₁₁H₁₂NO₃ [M-tBu+H]⁺, 206. found, 206.

Step 2. A mixture of tert-butyl (2S,5S)-2,3-dihydro-2,5-methanobenzo[f][1,4]oxazepine-4(5H)-carboxy late (250 mg, 0.957 mmol) in THF (10 mL) was treated with 4,4′-di-tert-butyl-2,2′-bipyridine (26 mg, 0.097 mmol), 4,4,4′,4′,5,5,5′,5′-octamethyl-2,2′-bi(1,3,2-dioxaborolane) (219 mg, 0.861 mmol), bis(1,5-cyclooctadiene)-dimethoxy-di-iridium (32 mg, 0.048 mmol). The mixture was stirred at 80° C. for 16 h, quenched with water, and extracted with DCM. The combined organic layers were dried over Na₂SO₄, filtered and concentrated give a 2:1 mixture of tert-butyl (2S,5S)-8-(4,4,5,5-tetramethyl-1,3,2-dioxaborolan-2-yl)-2,3-dihydro-2,5-methanobenzo[f][1,4]oxazepine-4(5H)-carboxylate and tert-butyl (2S,5S)-9-(4,4,5,5-tetramethyl-1,3,2-dioxaborolan-2-yl)-2,3-dihydro-2,5-methanobenzo[f][1,4]oxazepine-4(5H)-carboxylate. MS (EI) calculated for C₁₇H₂₃BNO₅ [M-tBu+H]⁺, 332. found, 332.

Step 3. A solution of tert-butyl (2S,5S)-7-(4,4,5,5-tetramethyl-1,3,2-dioxaborolan-2-yl)-2,3-dihydro-2,5-methanobenzo[f][1,4]oxazepine-4(5H)-carboxylate and tert-butyl (2S,5S)-8-(4,4,5,5-tetramethyl-1,3,2-dioxaborolan-2-yl)-2,3-dihydro-2,5-methanobenzo[f][1,4]oxazepine-4(5H)-carboxylate (2:1 ratio, 300 mg, 0.387 mmol) in MeOH (4 mL) was treated with copper chloride (182 mg, 1.36 mmol), and the mixture was heated at 90° C. for 3 h. The crude mixture was partitioned between EtOAc and water. The organic layer was washed with brine, dried (Na₂SO₄), and concentrated. The residue was purified by reverse phase chromatography (gradient of 10-40% MeCN/water with 0.1% TFA) to give (2S,5S)-8-chloro-2,3,4,5-tetrahydro-2,5-methanobenzo[f][1,4]oxazepine and (2S,5S)-9-chloro-2,3,4,5-tetrahydro-2,5-methanobenzo[f][1,4]oxazepine. MS (EI) calculated for C₁₀H₁₁ClNO [M+H]⁺, 195. found, 196.

Step 4. A mixture of (2S,5S)-9-chloro-2,3,4,5-tetrahydro-2,5-methanobenzo[f][1,4]oxazepine (20 mg, 0.10 mmol), 3,3-difluoro-2,2-dimethylpropanoic acid (40 mg, 0.29 mmol), DIEA (0.071 mL, 0.41 mmol) and HATU (117 mg, 0.307 mmol) in DMF (2 mL) was stirred for 13 h. Reverse phase chromatography (gradient of 47-67% MeCN/water with 0.1% TFA) to afford 1-((2S,5S)-9-chloro-2,3-dihydro-2,5-methanobenzo[f][1,4]oxazepin-4(5H)-yl)-3,3-difluoro-2,2-dimethylpropan-1-one, 2-20. ¹H NMR (400 MHz, CD₃OD) δ 7.19 (d, J=8.4 Hz, 1H), 7.13 (d, J=6.8 Hz, 1H), 6.75 (t, J=7.6 Hz, 1H), 6.75 (t, J=57 Hz, 1H), 5.27 (m, 1H), 5.17 (m, 1H), 4.01 (m, 2H), 2.10-2.22 (m, 2H), 1.27 (s, 3H), 1.15 (s, 3H); MS (EI) calculated for C₁₅H₁₇ClF₂NO₂ [M+H]⁺, 316. found, 316.

Step 5. A mixture of (2S,5S)-8-chloro-2,3,4,5-tetrahydro-2,5-methanobenzo[f][1,4]oxazepine (45 mg, 0.23 mmol), 3,3-difluoro-2,2-dimethylpropanoic acid (59 mg, 0.43 mmol), DIEA (0.16 mL, 0.92 mmol) and HATU (219 mg, 0.575 mmol) in DMF (2 mL) was stirred for 13 h. Reverse phase chromatography (gradient of 51-71% MeCN/water with 0.1% TFA) to afford 1-((2S,5S)-8-chloro-2,3-dihydro-2,5-methanobenzo[f][1,4]oxazepin-4(5H)-yl)-3,3-difluoro-2,2-dimethylpropan-1-one, 2-21. ¹H NMR (400 MHz, CD₃OD) δ 7.15 (d, J=7.2 Hz, 1H), 6.76 (m, 2H), 6.07 (t, J=57 Hz, 1H), 5.22 (m, 1H), 5.05 (m, 1H), 3.98 (m, 2H), 2.12-2.19 (m, 2H), 1.27 (s, 3H), 1.14 (s, 3H); MS (EI) calculated for C₁₅H₁₇ClF₂NO₂ [M+H]⁺, 316. found, 316.

### Example 2H. Preparation of (2S,5S)-4-(3,3-Difluoro-2,2-dimethylpropanoyl)-2,3,4,5-tetrahydro-2,5-methanobenzo[f][1,4]oxazepine-9-carbonitrile (2-19)

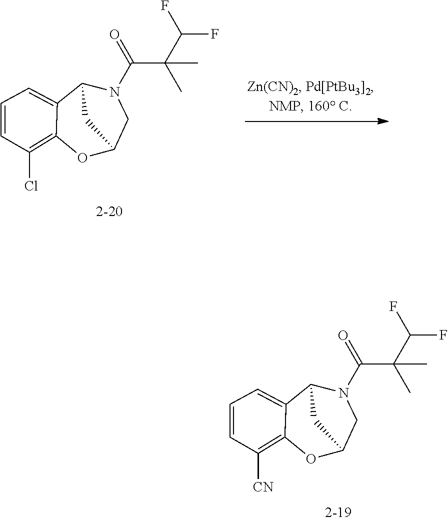 [SMILES: CC(C)(C(=O)N1C[C@@H]2C[C@H]1c1cccc(Cl)c1O2)C(F)F ; CC(C)(C(=O)N1C[C@@H]2C[C@H]1c1cccc(C#N)c1O2)C(F)F]

A solution of 1-((2S,5S)-9-chloro-2,3-dihydro-2,5-methanobenzo[f][1,4]oxazepin-4(5H)-yl)-3,3-difluoro-2,2-dimethylpropan-1-one (6 mg, 0.020 mmol) in n-methyl-2-pyrrolidone (0.5 mL) was treated with dicyanozinc (16 mg, 0.14 mmol) and bis(tri-tert-butylphosphine)palladium (0) (5 mg, 0.01 mmol), then stirred at 160° C. for 0.5 h. The mixture was purified by reverse phase chromatography (gradient of 38-68% MeCN/water with 0.1% TFA) to give (2S,5S)-4-(3,3-difluoro-2,2-dimethylpropanoyl)-2,3,4,5-tetrahydro-2,5-methanobenzo[f][1,4]oxazepine-9-carbonitrile. ¹H NMR (400 MHz, DMSO-d₆) δ 7.45-7.49 (m, 2H), 6.92 (t, J=7.6 Hz, 1H), 6.07 (t, J=57 Hz, 1H), 5.30 (m, 1H), 5.26 (m, 1H), 4.06 (m, 2H), 2.28 (m, 1H), 2.17 (m, 1H), 1.22 (s, 3H), 1.16 (s, 3H); MS (EI) calculated for C₁₆H₁₇F₂N₂O₂ [M+H]⁺, 307. found, 307.

### Example 2I. Preparation of 4-[(2S,5S)-7-fluoro-2,3-dihydro-2,5-methano-1,4-benzoxazepin-4(5H)-yl]-3,3-dimethyl-4-oxobutanenitrile (2-17) and 4-[(2S,5S)-9-fluoro-2,3-dihydro-2,5-methano-1,4-benzoxazepin-4(5H)-yl]-3,3-dimethyl-4-oxobutanenitrile (2-18)

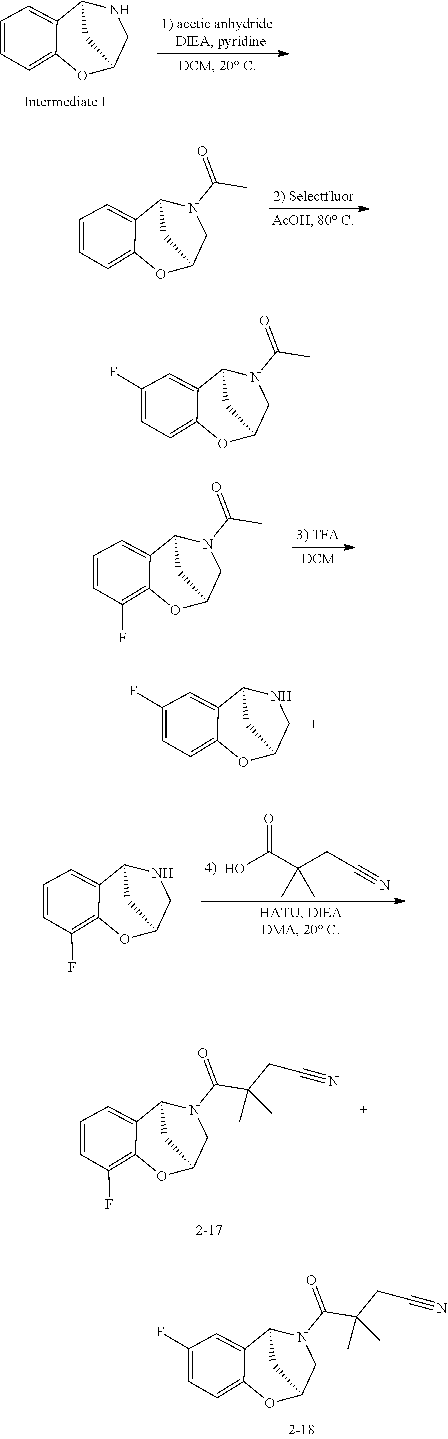

Step 1. A mixture containing (2S,5S)-2,3,4,5-tetrahydro-2,5-methanobenzo[f][1,4]oxazepine, Intermediate I (3.20 g, 16.19 mmol), DIEA (4.30 mL, 24.61 mmol), acetic anhydride (2.154 mL, 22.83 mmol), pyridine (1.440 mL, 17.81 mmol) in DCM (81 mL) was stirred for 1 hour at 20° C. The reaction was diluted with DCM, quenched with water, and extracted with DCM. The combined organic layer was washed with brine, dried with Na₂SO₄, filtered, and concentrated to dryness. Purified by flash column chromatography (0-100% EtOAc:Hex) to give 1-((2S,5S)-2,3-dihydro-2,5-methanobenzo[f][1,4]oxazepin-4(5H)-yl)ethan-1-one. MS (EI) calculated for C₁₂H₁₄NO₂ [M+H]⁺, 204. found, 204.

Step 2. A vial charged with a mixture of bis-fluorinated 1-((2S,5S)-2,3-dihydro-2,5-methanobenzo[f][1,4]oxazepin-4(5H)-yl)ethan-1-one (3.10 g, 15.25 mmol) and Selectfluor (5.94 g, 16.78 mmol) in AcOH (76 mL) was stirred at rt for 1.5 h. LCMS showed only starting material, so the reaction was heated to 80° C. for 48 hours. The reaction was cooled to rt, and then diluted with DCM. Quenched with 2M NaOH. The crude mixture was extracted with DCM. The combined organic layer was washed with brine, dried over Na₂SO₄, filtered and concentrated to give a mixture of 1-((2S,5S)-7-fluoro-2,3-dihydro-2,5-methanobenzo[f][1,4]oxazepin-4(5H)-yl)ethan-1-one and 1-((2S,5S)-9-fluoro-2,3-dihydro-2,5-methanobenzo[f][1,4]oxazepin-4(5H)-yl)ethan-1-one. Proceeded to the next step without purification. MS (EI) calculated for C₁₂H₁₃FNO₂ [M+H]⁺, 222. found, 222.

Step 3. To a mixture of 1-((2S,5S)-7-fluoro-2,3-dihydro-2,5-methanobenzo[f][1,4]oxazepin-4(5H)-yl)ethan-1-one and 1-((2S,5S)-9-fluoro-2,3-dihydro-2,5-methanobenzo[f][1,4]oxazepin-4(5H)-yl)ethan-1-one in dioxane (10 mL) and water (10 mL) was added LiOH, H₂O (4.31 g, 103 mmol), and the resulting mixture was heated to 115° C. The reaction was allowed to stir at this temperature for 48 h until full consumption of starting material was observed. The biphasic reaction was allowed to cool to room temperature and then water was added. The mixture was extracted with DCM, washed with brine and dried over Na₂SO₄. The crude mixture was concentrated to dryness under reduced pressure and purified by reverse phase chromatography (2-95% MeCN/water with 0.1% TFA) to give a mixture of (2S,5S)-7-fluoro-2,3,4,5-tetrahydro-2,5-methanobenzo[f][1,4]oxazepine and (2S,5S)-9-fluoro-2,3,4,5-tetrahydro-2,5-methanobenzo[f][1,4]oxazepine. MS (EI) calculated for C₁₀H₁₁FNO [M+H]⁺, 180. found, 180.

Step 4. A mixture of (2S,5S)-7-fluoro-2,3,4,5-tetrahydro-2,5-methanobenzo[f][1,4]oxazepine and (2S,5S)-9-fluoro-2,3,4,5-tetrahydro-2,5-methanobenzo[f][1,4]oxazepine (22.5 mg, 0.126 mmol) dissolved in DMA (1256 μL) was treated with HATU (86 mg, 0.226 mmol), DIEA (50.4 μL, 0.289 mmol) and 3-cyano-2,2-dimethylpropanoic acid (36.7 mg, 0.289 mmol). The reaction mixture was stirred at 23° C. overnight. The reaction was diluted with MeOH, filtered and purified by reverse phase chromatography (2-95% MeCN/water with 0.1% TFA). The regioisomers were separated by chiral SFC to obtain 4-((2S,5S)-7-fluoro-2,3-dihydro-2,5-methanobenzo[f][1,4]oxazepin-4(5H)-yl)-3,3-dimethyl-4-oxobutanenitrile as the first eluting regioisomer and 4-((2S,5S)-9-fluoro-2,3-dihydro-2,5-methanobenzo[f][1,4]oxazepin-4(5H)-yl)-3,3-dimethyl-4-oxobutanenitrile as the second eluting regioisomer.

Regioisomer 1 (2-17): 4-((2S,5S)-7-fluoro-2,3-dihydro-2,5-methanobenzo[f][1,4]oxazepin-4(5H)-yl)-3,3-dimethyl-4-oxobutanenitrile: Chiral SFC Ret Time 1.90 min [AD-H 21×250 mm, 5.0 μM, 25% MeOH with 0.1% NH₄OH]; ¹H NMR (500 MHz, DMSO-d₆) δ 7.14-6.91 (m, 2H), 6.79 (s, 1H), 5.20 (s, 2H), 3.98 (s, 2H), 2.71 (d, J=16.4 Hz, 1H), 2.61 (d, J=16.4 Hz, 1H), 2.20 (ddd, J=12.4, 4.9, 2.8 Hz, 1H), 2.07 (d, J=12.4 Hz, 1H), 1.55-1.01 (m, 6H); MS (EI) calculated for C₁₆H₁₈FN₂O₂ [M+H]⁺, 298. found, 298.

Regioisomer 2 (2-18): 4-((2S,5S)-9-fluoro-2,3-dihydro-2,5-methanobenzo[f][1,4]oxazepin-4(5H)-yl)-3,3-dimethyl-4-oxobutanenitrile: Chiral SFC Ret Time 2.25 min [AD-H 21×250 mm, 5.0 μM, 25% MeOH with 0.1% NH₄OH]; ¹H NMR (500 MHz, DMSO-d₆) δ 6.97 (s, 2H), 6.84-6.70 (m, 1H), 5.11 (d, J=42.5 Hz, 2H), 3.94 (s, 2H), 2.72 (d, J=16.4 Hz, 1H), 2.61 (d, J=16.6 Hz, 1H), 2.16 (ddd, J=12.3, 4.9, 2.8 Hz, 1H), 2.03 (d, J=12.4 Hz, 1H), 1.46-1.03 (m, 6H); MS (EI) calculated for C₁₆H₁₈FN₂O₂ [M+H]⁺, 298. found, 298.

### Example 2J. Preparation of 2,2-dimethyl-1-((2S,3R,5S)-3-methyl-2,3-dihydro-2,5-methanobenzo[f][1,4]oxazepin-4(5H)-yl)propan-1-one (2-31)

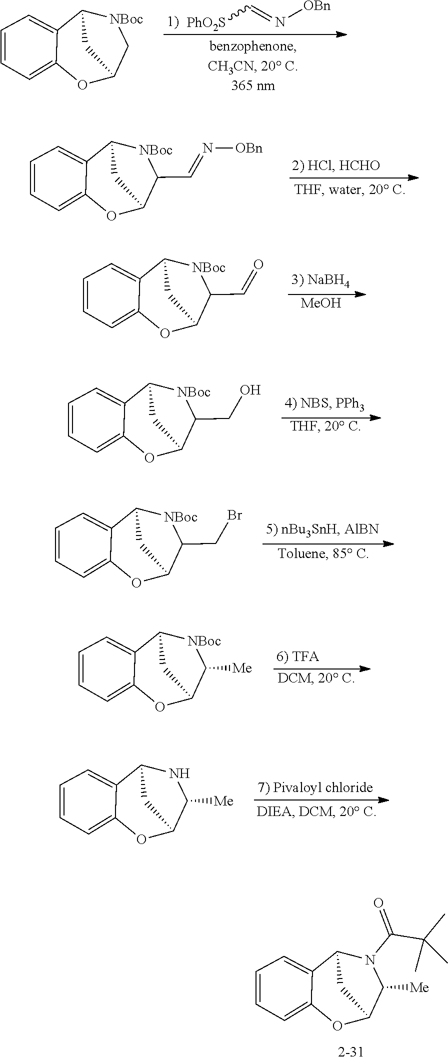

Step 1. A mixture of tert-butyl (2S,5S)-2,3-dihydro-2,5-methanobenzo[f][1,4]oxazepine-4(5H)-carboxylate (496 mg, 1.9 mmol), and (phenylsulfonyl)methanal O-benzyl oxime (784 mg, 2.9 mmol) in CH₃CN (1.9 mL) was treated with benzophenone (346 mg, 1.9 mmol) under N₂ at 20° C., and the reaction mixture was stirred for 24 h with 365 nm irradiation. The reaction was filtered, concentrated to dryness and purified by flash column chromatography (0-100% EtOAc:Hex) to give tert-butyl (2S,5S)-3-((E)-((benzyloxy)imino)methyl)-2,3-dihydro-2,5-methanobenzo[f][1,4]oxazepine-4(5H)-carboxylate. MS (EI) calculated for C₂₃H₂₇N₂O₄ [M+H]⁺, 395. found, 395.

Step 2. A vial charged with a mixture of tert-butyl (2S,5S)-3-((E)-((benzyloxy)imino)methyl)-2,3-dihydro-2,5-methanobenzo[f][1,4]oxazepine-4(5H)-carboxy late (500 mg, 1.3 mmol) and formaldehyde (37 wt % in water, 545 mg, 6.7 mmol) in THF (2.0 mL) was treated with 1N HCl in water (0.50 mL), and was stirred at room temperature for 16 h. The reaction was quenched with sat. aqueous Na₂CO₃. The crude mixture was extracted with EtOAc. The combined organic layer was washed with brine, dried over Na₂SO₄, filtered, concentrated to dryness and purified by flash column chromatography (0-100% EtOAc:Hex) to give tert-butyl (2S,5S)-3-formyl-2,3-dihydro-2,5-methanobenzo[f][1,4]oxazepine-4(5H)-carboxylate. MS (EI) calculated for C₁₆H₁₉NO₄Na [M+Na]⁺, 312. found, 312.

Step 3. A mixture of tert-butyl (2S,5S)-3-formyl-2,3-dihydro-2,5-methanobenzo[f][1,4]oxazepine-4(5H)-carboxylate (300 mg, 1.0 mmol) in methanol (10 mL) was treated with NaBH₄ (471 mg, 12 mmol) over 5 min at −15° C., and the resulting mixture was stirred at −15° C. for 2 h. The reaction mixture was quenched with sat. aqueous NH₄Cl. The crude mixture was extracted with EtOAc. The combined organic layer was washed with brine, dried over Na₂SO₄, filtered, concentrated to dryness and purified by flash column chromatography (0-100% EtOAc:Hex) to give tert-butyl (2S,5S)-3-(hydroxymethyl)-2,3-dihydro-2,5-methanobenzo[f][1,4]oxazepine-4(5H)-carboxylate. MS (EI) calculated for C₁₆H₂₁NO₄Na [M+Na]⁺, 314. found, 314.

Step 4. A mixture of tert-butyl (2S,5S)-3-(hydroxymethyl)-2,3-dihydro-2,5-methanobenzo[f][1,4]oxazepine-4(5H)-carboxy late (75 mg, 0.26 mmol) in THF (2.6 mL) and CH₂Cl₂ (2.6 mL) was treated with PPh₃ (338 mg, 1.3 mmol), and NBS (220 mg, 1.2 mmol) at 0° C. The reaction mixture was stirred at 0° C. overnight. The reaction was diluted with CH₂Cl₂, filtered, concentrated to dryness and purified by flash column chromatography (0-100% EtOAc:Hex) to give tert-butyl (2S,5S)-3-(bromomethyl)-2,3-dihydro-2,5-methanobenzo[f][1,4]oxazepine-4(5H)-carboxylate. MS (EI) calculated for C₁₂H₁₃BrNO₃ [M-C₄H₈+H]⁺, 298. found, 298.

Step 5. A mixture of tert-butyl (2S,5S)-3-(bromomethyl)-2,3-dihydro-2,5-methanobenzo[f][1,4]oxazepine-4(5H)-carboxylate (75 mg, 0.16 mmol) in toluene (0.64 mL) under N₂ was treated with AlBN (1.3 mg, 8.0 μmol), and tributyltin hydride (56 mg, 0.19 mmol) at 20° C. The reaction mixture was stirred at 85° C. for 1 h. The reaction was cooled to 20° C., concentrated to dryness and purified by flash column chromatography (0-100% EtOAc:Hex) to give tert-butyl (2S,3R,5S)-3-methyl-2,3-dihydro-2,5-methanobenzo[f][1,4]oxazepine-4(5H)-carboxylate. MS (EI) calculated for C₁₂H₁₄NO₃ [M-C+H₈+H]⁺, 220. found, 220.

Step 6. A mixture of tert-butyl (2S,3R,5S)-3-methyl-2,3-dihydro-2,5-methanobenzo[f][1,4]oxazepine-4(5H)-carboxylate (25 mg, 0.091 mmol) in CH₂Cl₂ (1.0 mL) was treated with TFA (1.0 mL) at 0° C. The reaction mixture was stirred at 20° C. for 2 h. The reaction was diluted with CH₂Cl₂, and concentrated to dryness to yield crude (2S,3R,5S)-3-methyl-2,3,4,5-tetrahydro-2,5-methanobenzo[f][1,4]oxazepine, which was used directly in the next step without further purification. MS (EI) calculated for C₁₁H₁₄NO [M+H]⁺, 176. found, 176.

Step 7. A mixture of crude (2S,3R,5S)-3-methyl-2,3,4,5-tetrahydro-2,5-methanobenzo[f][1,4]oxazepine (26 mg, 0.091 mmol) in CH₂Cl₂ (0.91 mL) was treated with DIEA (35 mg, 0.27 mmol), and pivaloyl chloride (16 mg, 0.14 mmol) at 0° C. The reaction mixture was stirred at 0° C. overnight. The reaction was diluted with CH₂Cl₂, concentrated to dryness and purified by flash column chromatography (0-100% EtOAc:Hex) to give 2,2-dimethyl-1-((2S,3R,5S)-3-methyl-2,3-dihydro-2,5-methanobenzo[f][1,4]oxazepin-4(5H)-yl)propan-1-one. ¹H NMR (499 MHz, DMSO-d6) δ 7.38-6.98 (m, 2H), 6.80-6.57 (m, 2H), 5.35 (s, 1H), 4.58 (s, 1H), 4.14 (s, 1H), 2.47-2.40 (m, 1H), 2.00-1.93 (m, 1H), 1.30-1.05 (m, 12H). MS (EI) calculated for C₁₆H₂₂NO₂ [M+H]⁺, 260. found, 260.

### Example 2K. Preparation of ((2S,5S)-7-Chloro-2,3-dihydro-2,5-methanobenzo[f][1,4]oxazepin-4(5H)-yl)(4-fluorobicyclo[2.2.1]heptan-1-yl)methanone (2-33)

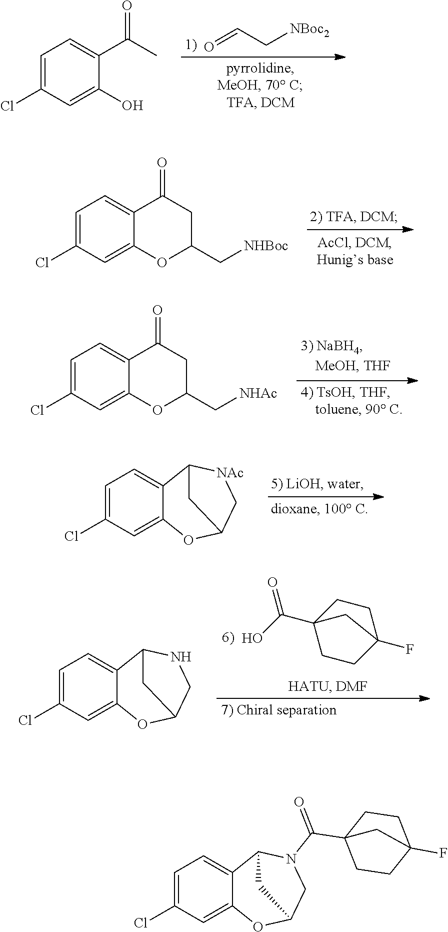

Step 1. A mixture containing 1-(4-chloro-2-hydroxyphenyl)ethan-1-one (350 mg, 2.05 mmol) in MeOH (10 mL) was treated with pyrrolidine (0.40 mL, 4.84 mmol) followed by tert-butyl (tert-butoxycarbonyl)(2-oxoethyl)carbamate (500 mg, 1.93 mmol). The mixture was stirred for 2 h at 70° C., then diluted with DCM. The organic layer was washed with water, then dried (Na₂SO₄) and concentrated. The residue was dissolved in DCM (10 mL) and added TFA (0.1 mL), aged for 60 min, concentrated to dryness, and purified by chromatography on silica gel (0-40% EtOAc/DCM; 24 g silica gel) to provide tert-butyl ((7-chloro-4-oxochroman-2-yl)methyl)carbamate as a yellow oil. MS (EI) calc'd for C₁₅H₁₈ClNO₄Na [M+Na]⁺, 334. found, 334.

Step 2. A mixture containing tert-butyl ((7-chloro-4-oxochroman-2-yl)methyl)carbamate (340 mg, 1.09 mmol) in DCM (1 mL) was treated with 0.5 mL of TFA, aged for 30 min, and concentrated to dryness. The dried material was redissolved in DCM, washed with sat. NaHCO₃, dried (Na₂SO₄) and concentrated. The dried material was redissolved in DCM (1 mL), treated with Hunig's base (0.30 mL, 1.7 mmol) and Ac₂O (0.15 mL, 1.6 mmol), stirred for 1 h, and concentrated to an oil. The oil was redissolved in DCM and washed with 1H HCl, 1 N NaOH, and then water. The organic layer was dried (Na₂SO₄) and concentrated to provide N-((7-chloro-4-oxochroman-2-yl)methyl)acetamide. MS (EI) calc'd for C₂₁H₁₃ClNO₃ [M+H]⁺, 254. found, 254.

Steps 3 and 4. A mixture containing N-((7-chloro-4-oxochroman-2-yl)methyl)acetamide (200 mg, 0.788 mmol) in THF (2 mL) and MeOH (0.2 mL) was treated with NaBH₄ (45 mg, 1.19 mmol) and stirred for 2 h. The reaction was quenched with water (1 mL), stirred for 10 min, and extracted with DCM 3×. The combined organic layer was dried (Na₂SO₄) and concentrated. To dry further, it was dissolved in THF (2 mL) and toluene (2 mL), then concentrated again to dryness. The resulting material was redissolved in THF (2 mL) and toluene (2 mL) with gentle warming, treated with TsOH (37 mg, 0.20 mmol), warmed to 90° C., and stirred for 60 min. Another 75 mg of TsOH (0.39 mmol) was added and stirred 2 h, then cooled to RT, and concentrated to dryness. The dried material was diluted with DCM, washed with 1 N NaOH, dried (Na₂SO₄) and concentrated. NMR analysis indicated mostly the desired product (1-(8-chloro-2,3-dihydro-2,5-methanobenzo[f][1,4]oxazepin-4(5H)-yl)ethan-1-one versus the elimination product. ¹H NMR (500 MHz, CDCl₃) δ 6.98-7.18 (m, 1H), 6.80-6.85 (m, 2H), 4.80-5.30 (m, 2H), 3.70-3.80 (m, 2H), 1.95-2.25 (m, 2H), 1.97 and 2.22 (2s, 3H); MS (EI) calc'd for C₁₂H₁₃ClNO₂ [M+H]⁺, 238. found, 238.

Step 5. A mixture containing 1-(8-chloro-2,3-dihydro-2,5-methanobenzo[f][1,4]oxazepin-4(5H)-yl)ethan-1-one (90 mg, 0.38 mmol) in dioxane (1 mL), MeOH (1 mL), water (1 mL) was treated with LiOH hydrate (160 mg, 3.81 mmol) and stirred for 8 h at 100° C. The resulting material was diluted with DCM and washed with water. The organic layer was dried (Na₂SO₄) and concentrated giving an oil (8-chloro-2,3,4,5-tetrahydro-2,5-methanobenzo[f][1,4]oxazepine. ¹H NMR (500 MHz, DMSO-d₆) δ 7.27 (d, J=10 Hz, 1H), 6.92 (m, 1H), 6.86 (d, J=5 Hz, 1H), 5.40 (m, 1H), 4.59 (m, 1H), 4.05-4.22 (m, 2H), 2.74-2.84 (m, 2H); MS (EI) calc'd for C₁₀H₁₁ClNO [M+H]⁺, 196. found, 196.

Steps 6 and 7. A mixture containing 4-fluorobicyclo[2.2.1]heptane-1-carboxylic acid (250 mg, 1.58 mmol), HATU (600 mg, 1.58 mmol) in DMF (3 mL) was treated with Hunig's Base (0.50 mL, 2.86 mmol) and 8-chloro-2,3,4,5-tetrahydro-2,5-methanobenzo[f][1,4]oxazepine (335 mg, 1.28 mmol). The moisture was stirred overnight at RT, filtered, and purified by reverse phase chromatography (30-95% MeCN/water, 0.1% TFA) to give the desired product as a racemate. MS (EI) calc'd for C₁₈H₂OClFNO₂ [M+H]⁺, 336. found, 336. The material was resolved using chiral SCF column chromatography [Column: (R,R)-Whelk-O, 21×250 mm, 5 um; detection 215 nm; 70 mL/min 15% MeOH/CO₂ with 0.1% NH₄OH] providing the desired enantiomer at 6.90 min and the undesired enantiomer at 7.85 min; both isolated as solids. Characterization data for ((2S,5S)-8-chloro-2,3-dihydro-2,5-methanobenzo[f][1,4]oxazepin-4(5H)-yl)(4-fluorobicyclo[2.2.1]heptan-1-yl)methanone (2-32); ¹H NMR (500 Hz, CDCl₃) δ 7.19 (d, J=10 Hz, 1H), 6.77-6.83 (m, 2H), 5.27 (m, 1H), 5.04 (m, 1H), 3.76-3.90 (m, 2H), 2.14-2.21 (m, 2H), 2.05-2.10 (m, 2H), 2.04 (br s, 2H), 1.75-1.86 (m, 4H); MS (EI) calc'd for C₁₈H₂₀ClFNO₂ [M+H]⁺, 336. found, 336.

### Example 2L. Preparation of 4-((2S,5S)-7,9-Difluoro-2,3,4,5-tetrahydro-2,5-methanobenzo[f][1,4]oxazepine-4-carbonyl)bicyclo[2.1.1]hexane-1-carbonitrile (2-36)

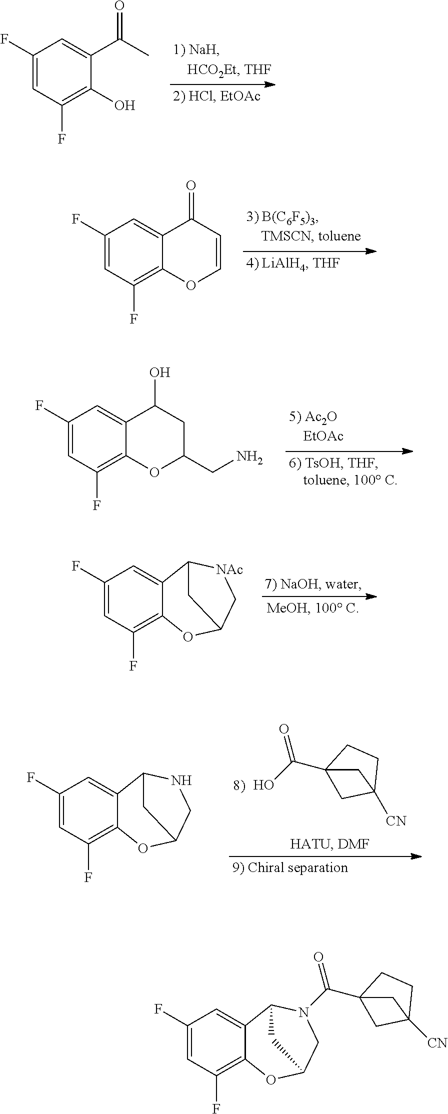

Steps 1 and 2. A round bottom flask containing 60% NaH (7.25 g, 181 mmol) in 260 mL of THF was cooled to 15° C. and treated with 1-(3,5-difluoro-2-hydroxyphenyl)ethan-1-one (13.0 g, 76.0 mmol). The mixture was stirred for 30 min at RT, then treated with HCO₂Et (11.2 g, 151 mmol) and warmed to 50° C., and stirred for 1.5 h. The reaction was cooled to RT, diluted with 130 mL of EtOAc followed by 4 M HCl in EtOAc (130 mL, 420 mmol) at 0° C. The mixture was stirred at RT for 6 h. The suspension was filtered and the filtrate was collected. The liquid was neutralized to pH=7 by the addition of sat. Na₂CO₃ (aq). The organic phase was washed with water, dried (Na₂SO₄), filtered and concentrated. The product was further purified by a slurry in petroleum ether (90 mL) and collected as a solid. ¹H NMR (400 MHz, CDCl₃) δ 7.91 (d, J=6.0 Hz, 1H), 7.63-7.70 (m, 1H), 7.22-7.30 (m, 1H), 6.39 (d, J=6.0 Hz, 1H); MS (EI) calc'd for C₉H₅F₂O₂ [M+H]⁺, 283. found, 283.

Step 3. Five reactions were performed in parallel. A mixture containing 6,8-difluoro-4H-chromen-4-one (2.00 g, 11.0 mmol), B(C₆F₅)₃ (280 mg, 0.55 mmol) and TMSCN (280 mg, 0.55 mmol) in 22 mL of toluene was prepared and stirred for 6 h at 30° C. The five reactions were combined and concentrated. The residue was dissolved in 12 mL of MeCN and 3 mL of 0.1% TFA, then purified by reverse phase chromatography (MeCN/water with 0.1% TFA) to provide 6,8-difluoro-4-oxochromane-2-carbonitrile. ¹H NMR (400 MHz, CDCl₃) δ 7.44 (ddd, J=7.8, 3.0, 1.8 Hz, 1H), 7.15-7.26 (m, 1H), 5.53 (dd, J=6.0, 5.2 Hz, 1H), 3.24 (dd, J=17.4, 5.2 Hz, 1H), 3.14 (dd, J=17.4, 6.2 Hz, 1H). MS (EI) calc'd for C₁₀H₅F₂NO₂Na [M+Na]⁺, 242. found, 242.

Steps 4 and 5. A mixture containing LiAlH₄ (2.32 g, 61.2 mmol) in THF (128 mL) was cooled to 0° C. and treated dropwise with a mixture of 6,8-difluoro-4-oxochromane-2-carbonitrile (6.40 g, 30.6 mmol) in 64 mL of THF, warmed to RT and stirred for 2 h. The reaction was quenched with 200 mL of 5% NaHCO₃ (aq) and sat. Rochelle salt solution (300 mL). The mixture was stirred for 30 min, then extracted with EtOAc. The organic layer was washed with brine, dried (Na₂SO₄), filtered and concentrated. The crude solid was used as is. The residue (6.90 g, 30.6 mmol) was dissolved in EtOAc (64 mL) and treated with acetic anhydride (2.32 g, 33.6 mmol) and stirred for 1h. The reaction was quenched with the addition of 5% K₂CO₃ aq., the organic layer was washed with brine, dried (Na₂SO₄) and concentrated. Chromatography on SiO₂ (100% EtOAc to 10% MeOH/DCM) gave N-((6,8-difluoro-4-hydroxychroman-2-yl)methyl)acetamide. ¹H NMR (400 MHz, DMSO-d₆) δ 8.11 (br t, J=5.6 Hz, 1H), 7.12 (ddd, J=11.2, 8.6, 3.2 Hz, 1H), 6.94-7.05 (m, 1H), 5.71 (d, J=6.4 Hz, 1H), 4.60-4.82 (m, 1H), 4.20-4.29 (m, 1H), 3.26-3.50 (m, 1H), 2.10-2.33 (m, 1H), 1.86 (s, 3H), 1.54-1.77 (m, 1H). MS (EI) calc'd for C₁₂H₁₄F₂NO₃ [M+H]⁺, 258. found, 258.

Step 6. This step was performed in five parallel reactions. A mixture containing N-((6,8-difluoro-4-hydroxychroman-2-yl)methyl)acetamide (1.52 g, 5.91 mmol), TsOH-H₂O (787 mg, 4.14 mmol) in THF (3 mL) and toluene (30 mL) was heated to 100° C. and stirred for 3 h. The five reactions were combined, diluted with EtOAc and washed with 5% NaHCO₃ aq. The organic layer was dried (Na₂SO₄), filtered, concentrated and purified by chromatography on SiO₂ (50% EtOAc/DCM to 10% MeOH/DCM) to provide 1-(7,9-difluoro-2,3-dihydro-2,5-methanobenzo[f][1,4]oxazepin-4(5H)-yl)ethan-1-one as an oil. ¹H NMR (400 MHz, CDCl₃) δ 6.54-6.92 (m, 2H), 4.75-5.29 (m, 2H), 3.66-3.93 (m, 2H), 2.18-2.35 (m, 3H), 1.98 (s, 2H). MS (EI) calc'd for C₁₂H₁₂F₂NO₂ [M+H]⁺, 240. found, 240.

Step 7. This step was performed in ten parallel reactions. A mixture containing 1-(7,9-difluoro-2,3-dihydro-2,5-methanobenzo[f][1,4]oxazepin-4(5H)-yl)ethan-1-one (76 mg, 0.32 mmol) in 2.3 mL of MeOH was treated with NaOH (127 mg, 3.18 mmol) in 2.3 mL of water. The mixture was stirred at 100° C. for 16 h. The reactions were combined and extracted with DCM. The organic layer was washed with brine, dried (Na₂SO₄), filtered and concentrated. The residue was purified by reverse phase chromatography (MeCN/water with 0.05% NH₄OH) to provide 7,9-difluoro-2,3,4,5-tetrahydro-2,5-methanobenzo[f][1,4]oxazepine as a racemate. ¹H NMR (400 MHz, DMSO-d₆, 80° C.) δ 6.94 (ddd, J=11.2, 8.8, 2.8 Hz, 1H), 6.70-6.80 (m, 1H), 5.01 (br s, 1H), 4.09 (br d, J=3.6 Hz, 1H), 3.08-3.20 (m, 2H), 2.08 (br d, J=12.0 Hz, 1H), 1.94 (dt, J=12.0, 3.6 Hz, 1H). MS (EI) calc'd for C₁₀H₁₀F₂NO [M+H]⁺, 198. found, 198.

Steps 8 and 9. A solution containing 7,9-difluoro-2,3,4,5-tetrahydro-2,5-methanobenzo[f][1,4]oxazepine (40 mg, 0.20 mmol), 4-cyanobicyclo[2.1.1]hexane-1-carboxylic acid (50 mg, 0.33 mmol), HATU (120 mg, 0.32 mmol), Hunig's Base (0.10 mL, 0.57 mmol) in DMF (2 mL) was stirred overnight at 20° C., filtered, and purified by reverse phase chromatography (15-70% MeCN/water with 0.1% TFA) to provide a racemic product. The material was resolved using chiral SCF column chromatography [Column: (R,R)-Whelk-O, 21×250 mm, 5 um; detection 215 nm; 50 mL/min 30% MeOH/CO₂ with 0.1% NH₄OH] providing the desired enantiomer at 3.60 min and the undesired enantiomer at 4.40 min; both isolated as oils. Characterization data for 4-(7,9-difluoro-2,3,4,5-tetrahydro-2,5-methanobenzo[f][1,4]oxazepine-4-carbonyl)bicyclo[2.1.1]hexane-1-carbonitrile (2-35); ¹H NMR (500 Hz, DMSO-d₆) δ 7.15 (t, J=10 Hz, 1H), 6.82 (d, J=10 Hz, 1H), 4.95-5.25 (m, 2H), 3.67-2.81 (m, 2H), 2.0-2.3 (m, 7H), 1.76-1.83 (m, 3H); MS (EI) calc'd for C₁₈H₁₇F₂N₂O₂ [M+H]⁺, 331. found, 331.

Compounds 2-5 and 2-6 were prepared in a fashion similar to the procedure used for the preparation of 2-17 and 2-18. Regioisomer 1 (2-5): 1-[(2S,5S)-7-fluoro-2,3-dihydro-2,5-methano-1,4-benzoxazepin-4(5H)-yl]-2,2-dimethylpropan-1-one: Chiral SFC Ret Time 2.2 min [Lux-4 21×250 mm, 5.0 μM, 15% MeOH with 0.1% NH₄OH]; ¹H NMR (500 MHz, DMSO-d₆) δ 6.96 (t, J=7.7 Hz, 2H), 6.75 (dd, J=9.1, 4.5 Hz, 1H), 5.14 (s, 1H), 5.04 (s, 1H), 4.00-3.75 (m, 2H), 2.12 (ddd, J=12.3, 4.9, 2.9 Hz, 1H), 2.00 (d, J=12.3 Hz, 1H), 1.10 (s, 9H); MS (EI) calculated for C₁₅H₁₉FNO₂ [M+H]⁺, 264. found, 264. Regioisomer 2 (2-6): 1-[(2S,5S)-9-fluoro-2,3-dihydro-2,5-methano-1,4-benzoxazepin-4(5H)-yl]-2,2-dimethylpropan-1-one: Chiral SFC Ret Time 2.5 min [Lux-4 21×250 mm, 5.0 μM, 15% MeOH with 0.1% NH₄OH]; ¹H NMR (500 MHz, DMSO-d₆) δ 7.11-7.02 (m, 1H), 6.98 (s, 1H), 6.77 (q, J=7.8 Hz, 1H), 5.21 (d, J=26.5 Hz, 2H), 3.96 (d, J=10.0 Hz, 2H), 2.15 (ddd, J=12.4, 4.9, 2.8 Hz, 1H), 2.05 (d, J=12.3 Hz, 1H), 1.11 (s, 9H); MS (EI) calculated for C₁₅H₁₉FNO₂ [M+H]⁺, 264. found, 264.

Compounds 2-7 and 2-8 were prepared in a fashion similar to the procedure used for the preparation of 2-17 and 2-18. Regioisomer 1 (2-7): 3,3-difluoro-1-[(2S,5S)-7-fluoro-2,3-dihydro-2,5-methano-1,4-benzoxazepin-4(5H)-yl]-2,2-dimethylpropan-1-one: Chiral SFC Ret Time 1.75 min [Lux-4 21×250 mm, 5.0 μM, 15% MeOH with 0.1% NH₄OH]; ¹H NMR (500 MHz, DMSO-d₆) δ 6.96 (d, J=8.3 Hz, 2H), 6.77 (dd, J=8.7, 4.5 Hz, 1H), 6.23 (t, J=56.6 Hz, 1H), 5.14 (s, 1H), 5.06 (s, 1H), 3.94 (s, 2H), 2.16 (ddd, J=12.3, 4.9, 2.8 Hz, 1H), 2.03 (d, J=12.4 Hz, 1H), 1.23 (d, J=15.1 Hz, 3H), 1.08 (s, 3H); MS (EI) calculated for C₁₅H₁₇F₃NO₂ [M+H]⁺, 300. found, 300. Regioisomer 2 (2-8): 3,3-difluoro-1-[(2S,5S)-9-fluoro-2,3-dihydro-2,5-methano-1,4-benzoxazepin-4(5H)-yl]-2,2-dimethylpropan-1-one: Chiral SFC Ret Time 2.0) min [Lux-4 21×250 mm, 5.0 μM, 15% MeOH with 0.1% NH₄OH]; ¹H NMR (600 MHz, DMSO-d₆) δ 7.11-7.02 (m, 1H), 7.00 (d, J=7.3 Hz, 1H), 6.84-6.71 (m, 1H), 6.19 (t, J=56.6 Hz, 1H), 5.28 (s, 1H), 5.20 (s, 1H), 4.05-3.93 (m, 1H), 3.88 (s, 1H), 2.20 (ddd, J=12.5, 4.7, 2.8 Hz, 1H), 2.07 (dd, J=25.6, 12.2 Hz, 1H), 1.25 (d, J=13.3 Hz, 3H), 1.14 (s, 3H); MS (EI) calculated for C₁₅H₁₇F₃NO₂ [M+H]⁺, 300. found, 300.

Compounds 2-9 and 2-10 were prepared in a fashion similar to the procedure used for the preparation of 2-17 and 2-18. Regioisomer 1 (2-9): [(2S,5S)-7-fluoro-2,3-dihydro-2,5-methano-1,4-benzoxazepin-4(5H)-yl](1-methylcyclobutyl)methanone: Chiral SFC Ret Time 3.75 min [IG 21×250 mm, 5.0 μM, 20% MeOH with 0.1% NH₄OH]; ¹H NMR (500 MHz, DMSO-d₆) (rotamers) δ 7.15 and 7.06 (2m, 2H), 6.93 (d, J=7.5 Hz, 1H), 6.85 and 6.77 (2m, 1H), 5.18 (s, 1H), 5.14 and 4.93 (2d, J=4.8 Hz and J=3.9 Hz, 1H), 3.82 and 3.73 (2dd, J=13.9, 4.4 Hz and J=12.1, 3.5 Hz, 1H), 3.60 (d, J=12.2 Hz, 1H), 2.46-2.24 (m, 1H), 2.23-1.82 (m, 4H), 1.73-1.49 (m, 2H), 1.41 and 1.19 (2s, 3H); MS (EI) calculated for C₁₆H₁₉FNO₂ [M+H]⁺, 276. found, 276. Regioisomer 2 (2-10): [(2S,5S)-9-fluoro-2,3-dihydro-2,5-methano-1,4-benzoxazepin-4(5H)-yl](1-methylcyclobutyl)methanone: Chiral SFC Ret Time 4.4 min [IG 21×250 mm, 5.0 μM, 20% MeOH with 0.1% NH₄OH]; ¹H NMR (500 MHz, DMSO-d₆) (rotamers) δ 7.11-6.85 (m, 2H), 6.81 and 6.75 (s and dd, J=8.8, 4.5 Hz, 1H), 5.05 (d, J=4.4 Hz, 2H), 4.86 (d, J=4.0) Hz, 1H), 3.79 and 3.69 (2dd, J=13.6, 4.2 Hz, and J=11.7, 3.1 Hz, 1H), 3.55 and 3.29 (2d, J=12.0 Hz and J=13.4 Hz, 1H), 2.46-2.24 (m, 1H), 2.24-1.80 (m, 4H), 1.65-1.48 (m, 2H), 1.41 and 1.18 (2s, 3H); MS (EI) calculated for C₁₆H₁₉FNO₂ [M+H]⁺, 276. found, 276.

Compounds 2-12 and 2-13 were prepared in a fashion similar to the procedure used for the preparation of 2-17 and 2-18. Regioisomer 1 (2-12): 3,3-difluoro-1-[(2S,5S)-9-fluoro-2,3-dihydro-2,5-methano-1,4-benzoxazepin-4(5H)-yl]-2,2-dimethylbutan-1-one: Chiral SFC Ret Time 2.8 min [IC 21×250 mm, 5.0 μM, 5% MeOH with 0.1% NH₄OH]; ¹H NMR (500 MHz, DMSO-d₆) δ 6.96 (s, 2H), 6.75 (dd, J=9.7, 4.5 Hz, 1H), 5.16 (s, 1H), 5.04 (s, 1H), 3.92 (d, J=8.3 Hz, 2H), 2.13 (d, J=12.5 Hz, 1H), 2.03 (d, J=12.4 Hz, 1H), 1.53-1.19 (m, 9H); MS (EI) calculated for C₁₆H₁₉F₃NO₂ [M+H]⁺, 314. found, 314. Regioisomer 2 (2-13): 3,3-difluoro-1-[(2S,5S)-7-fluoro-2,3-dihydro-2,5-methano-1,4-benzoxazepin-4(5H)-yl]-2,2-dimethylbutan-1-one: Chiral SFC Ret Time 4.3 min [IC 21×250 mm, 5.0 μM, 5% MeOH with 0.1% NH₄OH]; ¹H NMR (500 MHz, DMSO-d₆) δ 7.12-7.02 (m, 1H), 6.99 (s, 1H), 6.77 (d, J=4.7 Hz, 1H), 5.26 (s, 1H), 5.17 (s, 1H), 3.96 (d, J=7.8 Hz, 2H), 2.16 (d, J=12.6 Hz, 1H), 2.08 (d, J=12.5 Hz, 1H), 1.53-1.19 (m, 9H); MS (EI) calculated for C₁₆H₁₉F₃NO₂ [M+H]⁺, 314. found, 314.

Compounds 2-14 and 2-15 were prepared in a fashion similar to the procedure used for the preparation of 2-17 and 2-18. Regioisomer 1 (2-14): (bicyclo[2.2.1]heptan-1-yl)[(2S,5S)-7-fluoro-2,3-dihydro-2,5-methano-1,4-benzoxazepin-4(5H)-yl]methanone: Chiral SFC Ret Time 3.5 min [Lux-4 21×250 mm, 5.0 μM, 20% MeOH with 0.1% NH₄OH]; ¹H NMR (500 MHz, DMSO-d₆) δ 6.93 (dd, J=21.9, 8.6 Hz, 2H), 6.75 (dd, J=8.4, 4.5 Hz, 1H), 5.10 (d, J=4.4 Hz, 1H), 5.05 (s, 1H), 3.95-3.74 (m, 2H), 2.12 (s, 2H), 2.01 (d, J=12.4 Hz, 1H), 1.70-1.30 (m, 10H); MS (EI) calculated for C₁₈H₂₁FNO₂ [M+H]⁺, 302. found, 302. Regioisomer 2 (2-15): (bicyclo[2.2.1]heptan-1-yl)[(2S,5S)-9-fluoro-2,3-dihydro-2,5-methano-1,4-benzoxazepin-4(5H)-yl]methanone: Chiral SFC Ret Time 4.15 min [Lux-4 21×250 mm, 5.0 μM, 20% MeOH with 0.1% NH₄OH]; ¹H NMR (500 MHz, DMSO-d₆) δ 7.08-7.00 (m, 1H), 6.95 (d, J=7.2 Hz, 1H), 6.76 (d, J=5.2 Hz, 1H), 5.19 (s, 2H), 3.98-3.80 (m, 2H), 2.26-1.99 (m, 4H), 1.74-1.25 (m, 9H); MS (EI) calculated for C₁₈H₂₁FNO₂ [M+H]⁺, 302. found, 302.

Compound 2-16 and 2-23 were prepared in a fashion similar to the procedure used for the preparation of 2-21, substituting 3,3-difluoro-2,2-dimethylpropanoic acid for 3,3-difluoro-2,2-dimethylbutanoic acid or 3-cyano-2,2-dimethylpropanoic acid. Compound 2-16: Enantiomer 2 1-((2S,5S)-8-chloro-2,3-dihydro-2,5-methanobenzo[f][1,4]oxazepin-4(5H)-yl)-3,3-difluoro-2,2-dimethylbutan-1-one: Chiral SFC Ret Time 2.2 min [AD-H 21×250 mm, 5.0 μM, 20% MeOH with 0.1% NH₄OH]; ¹H NMR (500 MHz, Methanol-d₄) δ 7.19 (d, J=8.3 Hz, 1H), 6.84-6.75 (m, 2H), 5.28 (s, 1H), 5.05 (s, 1H), 4.16 (s, 1H), 4.01 (dd, J=12.1, 3.3 Hz, 1H), 2.24-2.08 (m, 2H), 1.37 (d, J=21.8 Hz, 9H); MS (EI) calculated for C₁₆H₁₉ClF₂NO₂ [M+H]⁺, 330. found, 330.

Compound 2-23. Enantiomer 1 4-((2S,5S)-8-chloro-2,3-dihydro-2,5-methanobenzo[f][1,4]oxazepin-4(5H)-yl)-3,3-dimethyl-4-oxobutanenitrile: Chiral SFC Ret Time 3.8 min [Lux-2 21×250 mm, 5.0 μM, 20% MeOH with 0.1% NH₄OH]; ¹H NMR (500 MHz, DMSO-d₆) δ 7.17 (s, 1H), 6.85 (s, 2H), 5.18 (s, 1H), 5.11 (s, 1H), 3.94 (s, 2H), 2.71 (d, J=16.4 Hz, 1H), 2.60 (d, J=16.4 Hz, 1H), 2.17 (ddd, J=12.3, 4.9, 2.8 Hz, 1H), 2.02 (d, J=12.4 Hz, 1H), 1.38-1.04 (m, 6H); MS (EI) calculated for C₁₆H₁₈ClN₂O₂ [M+H]⁺, 305. found, 305.

Compound 2-22 was prepared from 2-21 in a fashion similar to the procedure used for the preparation of 2-19 from 2-20.

Compound 2-25 was prepared in a fashion analogous to the procedure used for the synthesis of 2-11, substituting 3,3-difluoro-2,2-dimethylpropanoic acid for 4-fluorobicyclo[2.2.1]heptane-1-carboxylic acid.

Compounds 2-26 and 2-27 were prepared in a fashion similar to the procedure used for the preparation of 2-4.

Compound 2-28 was prepared in a fashion similar to the procedure used for the preparation of 2-17 and 2-18. Regioisomer 2 (2-28): {1-[(2S,5S)-9-fluoro-2,3-dihydro-2,5-methano-1,4-benzoxazepine-4(5H)-carbonyl]cyclobutyl}acetonitrile: Chiral SFC Ret Time 3.25 min [OJ-H 21×250 mm, 5.0 μM, 15% MeOH with 0.1% NH₄OH]; ¹H NMR (500 MHz, DMSO-d₆) (rotamers) δ 7.17 and 7.08 (s and t, J=9.6 Hz, 1H), 6.96 (d, J=7.1 Hz, 1H), 6.85-6.71 (m, 1H), 5.19 and 4.97 (2s, 2H), 4.97 (s, 1H), 3.91-3.76 (m, 1H), 3.72 and 3.39 (2d, J=12.0 Hz and J=14.3 Hz, 1H), 3.20-2.86 (m, 2H), 2.45-1.77 (m, 5H), 1.75-1.54 (m, 2H); MS (EI) calculated for C₁₇H₁₈FN₂O₂ [M+H]⁺, 301. found, 301.

Compounds 2-29 and 2-30 were prepared in a fashion similar to the procedure used for the preparation of 2-17 and 2-18. Regioisomer 1 (2-29): 3-((2S,5S)-7-fluoro-2,3,4,5-tetrahydro-2,5-methanobenzo[f][1,4]oxazepine-4-carbonyl)-3-methylcyclobutane-1-carbonitrile: Chiral SFC Ret Time 1.85 min [OJ-H 21×250 mm, 5.0 μM, 20% MeOH with 0.1% NH₄OH]; ¹H NMR (500 MHz, DMSO-d₆) (rotamers) δ 7.16 and 6.91 (2dd, J=8.8, 2.4 Hz and J=8.3, 3.0 Hz, 1H), 7.05 and 6.96 (2td, J=8.6, 2.9 Hz and J=8.8, 3.1 Hz, 1H), 6.81 and 6.76 (2dd, J=8.8, 4.6 Hz and J=8.8, 4.5 Hz, 1H), 5.06 and 4.84 (s and d, J=3.7 Hz, 2H), 3.80 and 3.70 (2dd, J=13.6, 4.0 Hz and J=11.8, 3.3 Hz, 1H), 3.57 (d, J=11.7 Hz, 1H), 3.21-3.04 (m, 1H), 2.96-2.79 (m, 1H), 2.69 (t, J=11.0 Hz, 1H), 2.48-2.28 (m, 1H), 2.20-2.10 (m, 1H), 2.03 (d, J=12.3 Hz, 2H), 1.58 and 1.34 (2s, 3H); MS (EI) calculated for C₁₇H₁₈FN₂O₂ [M+H]⁺, 301. found, 301. Regioisomer 2 (2-30): 3-((2S,5S)-9-fluoro-2,3,4,5-tetrahydro-2,5-methanobenzo[f][1,4]oxazepine-4-carbonyl)-3-methylcyclobutane-1-carbonitrile: Chiral SFC Ret Time 2.50 min [OJ-H 21×250 mm, 5.0 μM, 20% MeOH with 0.1% NH₄OH]; ¹H NMR (500 MHz, DMSO-d₆) (rotamers) δ 7.20-7.02 (m, 1H), 6.93 (d, J=7.5 Hz, 1H), 6.89-6.81 (m, 1H), 6.81-6.72 (m, 1H), 5.20 (s, 1H), 5.15 and 4.92 (d and s, J=4.5 Hz, 1H), 3.83 and 3.74 (2dd, J=13.8, 4.2 Hz and J=12.0, 3.4 Hz, 1H), 3.63 (d, J=11.9 Hz, 1H), 3.21-3.02 (m, 1H), 2.91 (t, J=9.4 Hz, 1H), 2.82 (t, J=10.9 Hz, 1H), 2.71 (s, 1H), 2.47-2.26 and 2.23-1.98 (2m, 2H), 1.58 and 1.35 (2s, 3H); MS (EI) calculated for C₁₇H₁₈FN₂O₂ [M+H]⁺, 301. found, 301.

Compound 2-32 was prepared in a fashion analogous to the preparation of 2-1, substituting the carboxylic acid for 4-fluorobicyclo[2.2.1]heptane-1-carboxylic acid.

Compound 2-33 was prepared in a fashion analogous to the preparation of 2-35, substituting the carbocylic acid for 1-(4-cyanopyrimidin-2-yl)-4-fluoropiperidine-4-carboxylic acid. The racemic material was resolved using chiral column chromatography [IA column, 21×250 mm, 5 um; detection 215 nm, 70 mL/min 40% MeOH/CO₂ with 0.1% NH₄OH] giving the desired more active enantiomer at 3.50 min and the undesired, less active enantiomer at 5.75 min.

Compound 2-34 was prepared in a fashion analogous to the preparation of 2-35, substituting the carbocylic acid for 4-fluorobicyclo[2.2.1]heptane-1-carboxylic acid. The racemic material was resolved using chiral column chromatography [(R,R)-Whelk-O column, 21×250 mm, 5 um; detection 215 nm, 70 mL/min 25% MeOH/CO₂ with 0.1% NH₄OH] giving the desired more active enantiomer at 2.65 min and the undesired, less active enantiomer at 3.45 min.

TABLE 2

| | | | Exact |
|---|---|---|---|
| Compound | | | Mass |
| Number | Structure | Name | [M + H]⁺ |
| 2-1 | 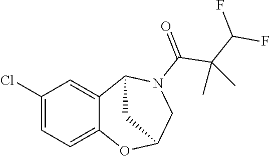 [SMILES: CC(C)(C(=O)N1C[C@@H]2C[C@H]1c1cc(Cl)ccc1O2)C(F)F] | 1-[(2S,5S)-7-chloro-2,3- dihydro-2,5-methano-1,4- benzoxazepin-4(5H)-yl]- 3,3-difluoro-2,2- dimethylpropan-1-one | Calculated 316, found 316 |
| 2-2 | 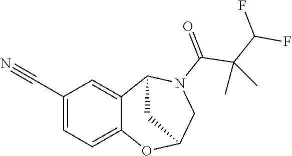 [SMILES: CC(C)(C(=O)N1C[C@@H]2C[C@H]1c1cc(C#N)ccc1O2)C(F)F] | (2S,5S)-4-(3,3-difluoro- 2,2-dimethylpropanoyl)- 2,3,4,5-tetrahydro-2,5- methano-1,4- benzoxazepine-7- carbonitrile | Calculated 307, found 307 |
| 2-3 | 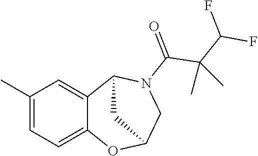 [SMILES: Cc1ccc2c(c1)[C@@H]1C[C@@H](CN1C(=O)C(C)(C)C(F)F)O2] | 3,3-difluoro-2,2-dimethyl- 1-[(2S,5S)-7-methyl-2,3- dihydro-2,5-methano-1,4- benzoxazepin-4(5H)- yl]propan-1-one | Calculated 296, found 296 |
| 2-4 | 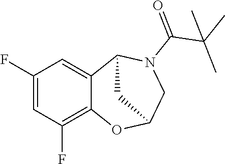 [SMILES: CC(C)(C)C(=O)N1C[C@@H]2C[C@H]1c1cc(F)cc(F)c1O2] | 1-[(2S,5S)-7,9-difluoro- 2,3-dihydro-2,5-methano- 1,4-benzoxazepin-4(5H)- yl]-2,2-dimethylpropan-1- one | Calculated 282, found 282 |
| 2-5 | 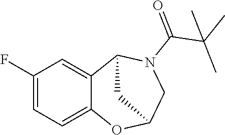 [SMILES: CC(C)(C)C(=O)N1C[C@@H]2C[C@H]1c1cc(F)ccc1O2] | 1-[(2S,5S)-7-fluoro-2,3- dihydro-2,5-methano-1,4- benzoxazepin-4(5H)-yl]- 2,2-dimethylpropan-1-one | Calculated 264, found 264 |
| 2-6 | 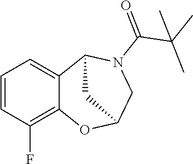 [SMILES: CC(C)(C)C(=O)N1C[C@@H]2C[C@H]1c1cccc(F)c1O2] | 1-[(2S,5S)-9-fluoro-2,3- dihydro-2,5-methano-1,4- benzoxazepin-4(5H)-yl]- 2,2-dimethylpropan-1-one | Calculated 264, found 264 |
| 2-7 | 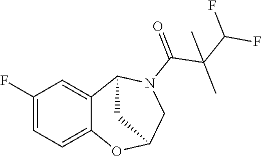 [SMILES: CC(C)(C(=O)N1C[C@@H]2C[C@H]1c1cc(F)ccc1O2)C(F)F] | 3,3-difluoro-1-[(2S,5S)-7- fluoro-2,3-dihydro-2,5- methano-1,4- benzoxazepin-4(5H)-yl]- 2,2-dimethylpropan-1-one | Calculated 300, found 300 |
| 2-8 | 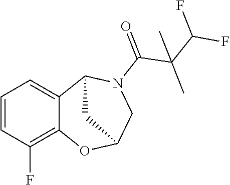 [SMILES: CC(C)(C(=O)N1C[C@@H]2C[C@H]1c1cccc(F)c1O2)C(F)F] | 3,3-difluoro-1-[(2S,5S)-9- fluoro-2,3-dihydro-2,5- methano-1,4- benzoxazepin-4(5H)-yl]- 2,2-dimethylpropan-1-one | Calculated 300, found 300 |
| 2-9 | 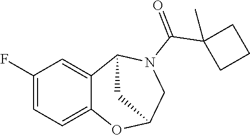 [SMILES: CC1(C(=O)N2C[C@@H]3C[C@H]2c2cc(F)ccc2O3)CCC1] | [(2S,5S)-7-fluoro-2,3- dihydro-2,5-methano-1,4- benzoxazepin-4(5H)-yl](1- methylcyclobutyl)methanone | Calculated 276, found 276 |
| 2-10 | 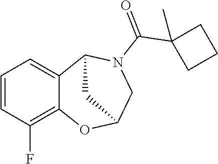 [SMILES: CC1(C(=O)N2C[C@@H]3C[C@H]2c2cccc(F)c2O3)CCC1] | [(2S,5S)-9-fluoro-2,3- dihydro-2,5-methano-1,4- benzoxazepin-4(5H)-yl](1- methylcyclobutyl)methanone | Calculated 276, found 276 |
| 2-11 | 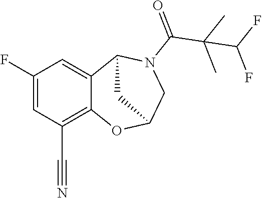 [SMILES: CC(C)(C(=O)N1C[C@@H]2C[C@H]1c1cc(F)cc(C#N)c1O2)C(F)F] | (2S,5S)-4-(3,3-difluoro- 2,2-dimethylpropanoyl)-7- fluoro-2,3,4,5-tetrahydro- 2,5-methano-1,4- benzoxazepine-9- carbonitrile | Calculated 325, found 325 |
| 2-12 | 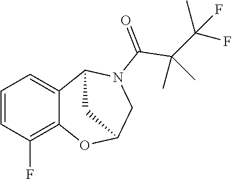 [SMILES: CC(F)(F)C(C)(C)C(=O)N1C[C@@H]2C[C@H]1c1cccc(F)c1O2] | 3,3-difluoro-1-[(2S,5S)-9- fluoro-2,3-dihydro-2,5- methano-1,4- benzoxazepin-4(5H)-yl]- 2,2-dimethylbutan-1-one | Calculated 314, found 314 |
| 2-13 | 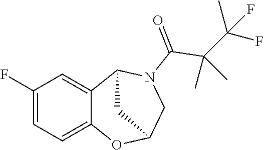 [SMILES: CC(F)(F)C(C)(C)C(=O)N1C[C@@H]2C[C@H]1c1cc(F)ccc1O2] | 3,3-difluoro-1-[(2S,5S)-7- fluoro-2,3-dihydro-2,5- methano-1,4- benzoxazepin-4(5H)-yl]- 2,2-dimethylbutan-1-one | Calculated 314, found 314 |
| 2-14 | 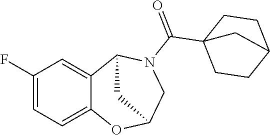 [SMILES: O=C(N1C[C@@H]2C[C@H]1c1cc(F)ccc1O2)C12CCC(CC1)C2] | (bicyclo[2.2.1]heptan-1- yl)[(2S,5S)-7-fluoro-2,3- dihydro-2,5-methano-1,4- benzoxazepin-4(5H)- yl]methanone | Calculated 302, found 302 |
| 2-15 | 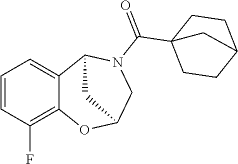 [SMILES: O=C(N1C[C@@H]2C[C@H]1c1cccc(F)c1O2)C12CCC(CC1)C2] | (bicyclo[2.2.1]heptan-1- yl)[(2S,5S)-9-fluoro-2,3- dihydro-2,5-methano-1,4- benzoxazepin-4(5H)- yl]methanone | Calculated 302, found 302 |
| 2-16 | 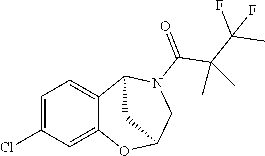 [SMILES: CC(F)(F)C(C)(C)C(=O)N1C[C@@H]2C[C@H]1c1ccc(Cl)cc1O2] | 1-((2S,5S)-8-chloro-2,3- dihydro-2,5- methanobenzo[f][1,4]oxaze- pin-4(5H)-yl)-3,3-difluoro- 2,2-dimethylbutan-1-one | Calculated 330, found 330 |
| 2-17 | 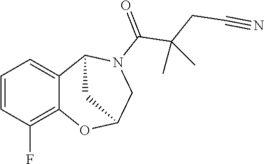 [SMILES: CC(C)(CC#N)C(=O)N1C[C@@H]2C[C@H]1c1cccc(F)c1O2] | 4-[(2S,5S)-7-fluoro-2,3- dihydro-2,5-methano-1,4- benzoxazepin-4(5H)-yl]- 3,3-dimethyl-4- oxobutanenitrile | Calculated 289, found 289 |
| 2-18 | 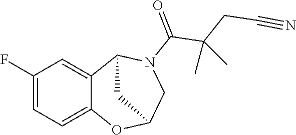 [SMILES: CC(C)(CC#N)C(=O)N1C[C@@H]2C[C@H]1c1cc(F)ccc1O2] | 4-[(2S,5S)-9-fluoro-2,3- dihydro-2,5-methano-1,4- benzoxazepin-4(5H)-yl]- 3,3-dimethyl-4- oxobutanenitrile | Calculated 289, found 289 |
| 2-19 | 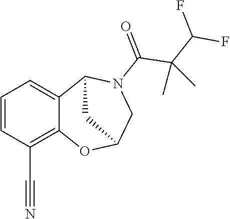 [SMILES: CC(C)(C(=O)N1C[C@@H]2C[C@H]1c1cccc(C#N)c1O2)C(F)F] | (2S,5S)-4-(3,3-difluoro- 2,2-dimethylpropanoyl)- 2,3,4,5-tetrahydro-2,5- methano-1,4- benzoxazepine-9- carbonitrile | Calculated 307, found 307 |
| 2-20 | 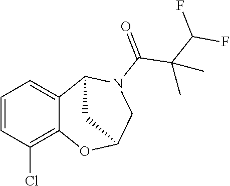 [SMILES: CC(C)(C(=O)N1C[C@@H]2C[C@H]1c1cccc(Cl)c1O2)C(F)F] | 1-[(2S,5S)-9-chloro-2,3- dihydro-2,5-methano-1,4- benzoxazepin-4(5H)-yl]- 3,3-difluoro-2,2- dimethylpropan-1-one | Calculated 316, found 316 |
| 2-21 | 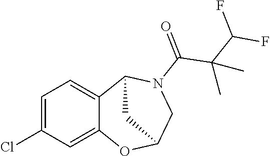 [SMILES: CC(C)(C(=O)N1C[C@@H]2C[C@H]1c1ccc(Cl)cc1O2)C(F)F] | 1-[(2S,5S)-8-chloro-2,3- dihydro-2,5-methano-1,4- benzoxazepin-4(5H)-yl]- 3,3-difluoro-2,2- dimethylpropan-1-one | Calculated 316, found 316 |
| 2-22 | 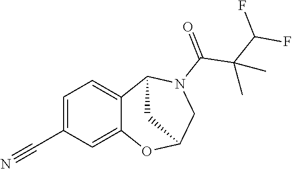 [SMILES: CC(C)(C(=O)N1C[C@@H]2C[C@H]1c1ccc(C#N)cc1O2)C(F)F] | (2S,5S)-4-(3,3-difluoro- 2,2-dimethylpropanoyl)- 2,3,4,5-tetrahydro-2,5- methano-1,4- benzoxazepine-8- carbonitrile | Calculated 307, found 307 |
| 2-23 | 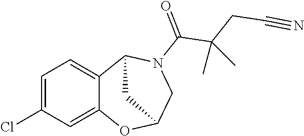 [SMILES: CC(C)(CC#N)C(=O)N1C[C@@H]2C[C@H]1c1ccc(Cl)cc1O2] | 4-((2S,5S)-8-chloro-2,3- dihydro-2,5- methanobenzo[f][1,4]oxaze- pin-4(5H)-yl)-3,3- dimethyl-4- oxobutanenitrile | Calculated 305, found 305 |
| 2-24 | 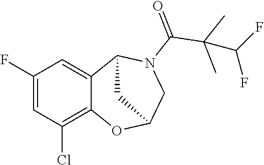 [SMILES: CC(C)(C(=O)N1C[C@@H]2C[C@H]1c1cc(F)cc(Cl)c1O2)C(F)F] | 1-[(2S,5S)-9-chloro-7- fluoro-2,3-dihydro-2,5- methano-1,4- benzoxazepin-4(5H)-yl]- 3,3-difluoro-2,2- dimethylpropan-1-one | Calculated 334, found 334 |
| 2-25 | 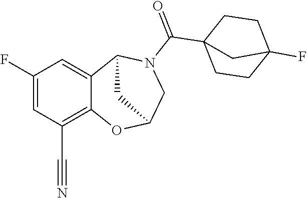 [SMILES: N#Cc1cc(F)cc2c1O[C@H]1C[C@@H]2N(C(=O)C23CCC(F)(CC2)C3)C1] | (2S,5S)-7-fluoro-4-(4- fluorobicyclo[2.2.1]heptane- 1-carbonyl)-2,3,4,5- tetrahydro-2,5-methano- 1,4-benzoxazepine-9- carbonitrile | Calculated 345, found 345 |
| 2-26 | 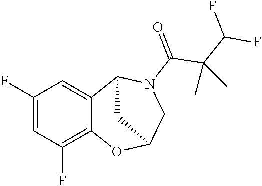 [SMILES: CC(C)(C(=O)N1C[C@@H]2C[C@H]1c1cc(F)cc(F)c1O2)C(F)F] | 1-[(2S,5S)-7,9-difluoro- 2,3-dihydro-2,5-methano- 1,4-benzoxazepin-4(5H)- yl]-3,3-difluoro-2,2- dimethylpropan-1-one | Calculated 318, found 318 |
| 2-27 | 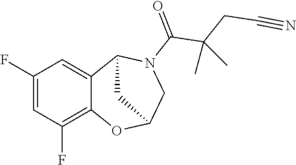 [SMILES: CC(C)(CC#N)C(=O)N1C[C@@H]2C[C@H]1c1cc(F)cc(F)c1O2] | 4-[(2S,5S)-7,9-difluoro- 2,3-dihydro-2,5-methano- 1,4-benzoxazepin-4(5H)- yl]-3,3-dimethyl-4- oxobutanenitrile | Calculated 307, found 307 |
| 2-28 | 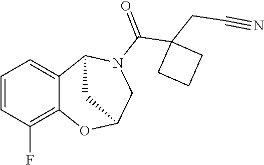 [SMILES: N#CCC1(C(=O)N2C[C@@H]3C[C@H]2c2cccc(F)c2O3)CCC1] | {1-[(2S,5S)-9-fluoro-2,3- dihydro-2,5-methano-1,4- benzoxazepine-4(5H)- carbonyl]cyclobutyl} acetonitrile | Calculated 301, found 301 |
| 2-29 | 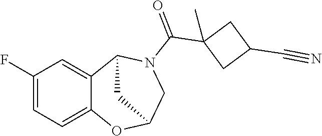 [SMILES: CC1(C(=O)N2C[C@@H]3C[C@H]2c2cc(F)ccc2O3)CC(C#N)C1] | 3-((2S,5S)-7-fluoro- 2,3,4,5-tetrahydro-2,5- methanobenzo[f][1,4]oxaze- pine-4-carbonyl)-3- methylcyclobutane-1- carbonitrile | Calculated 301, found 301 |
| 2-30 | 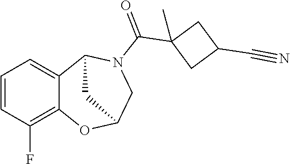 [SMILES: CC1(C(=O)N2C[C@@H]3C[C@H]2c2cccc(F)c2O3)CC(C#N)C1] | 3-((2S,5S)-9-fluoro- 2,3,4,5-tetrahydro-2,5- methanobenzo[f][1,4]oxaze- pine-4-carbonyl)-3- methylcyclobutane-1- carbonitrile | Calculated 301, found 301 |
| 2-31 | 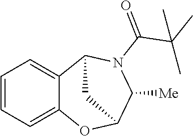 [SMILES: C[C@@H]1[C@@H]2C[C@@H](c3ccccc3O2)N1C(=O)C(C)(C)C] | 2,2-dimethyl-1- ((2S,3R,5S)-3-methyl-2,3- dihydro-2,5- methanobenzo[f][1,4]oxaze- pin-4(5H)-yl)propan-1-one | Calculated 260, found 260 |
| 2-32 | 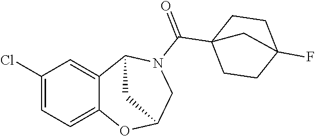 [SMILES: O=C(N1C[C@@H]2C[C@H]1c1cc(Cl)ccc1O2)C12CCC(F)(CC1)C2] | ((2S,5S)-7-chloro-2,3- dihydro-2,5- methanobenzo[f][1,4]oxaze- pin-4(5H)-yl)(4- fluorobicyclo[2.2.1]heptan- 1-yl)methanone | Calc'd 336, found 336 |
| 2-33 | 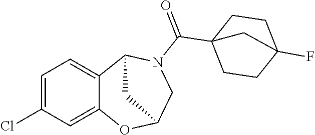 [SMILES: O=C(N1C[C@@H]2C[C@H]1c1ccc(Cl)cc1O2)C12CCC(F)(CC1)C2] | ((2S,5S)-8-chloro-2,3- dihydro-2,5- methanobenzo[f][1,4]oxaze- pin-4(5H)-yl)(4- fluorobicyclo[2.2.1]heptan- 1-yl)methanone | Calc'd 336, found 336 |
| 2-34 | 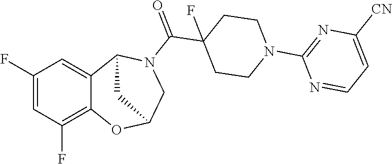 [SMILES: N#Cc1ccnc(N2CCC(F)(C(=O)N3C[C@@H]4C[C@H]3c3cc(F)cc(F)c3O4)CC2)n1] | 2-(4-((2S,5S)-7,9-difluoro- 2,3,4,5-tetrahydro-2,5- methanobenzo[f][1,4]oxaze- pine-4-carbonyl)-4- fluoropiperidin-1- yl)pyrimidine-4- carbonitrile | Calc'd 430, found 430 |
| 2-35 | 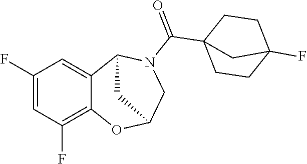 [SMILES: O=C(N1C[C@@H]2C[C@H]1c1cc(F)cc(F)c1O2)C12CCC(F)(CC1)C2] | ((2S,5S)-7,9-difluoro-2,3- dihydro-2,5- methanobenzo[f][1,4]oxaze- pin-4(5H)-yl)(4- fluorobicyclo[2.2.1]heptan- 1-yl)methanone | Calc'd 338, found 338 |
| 2-36 | 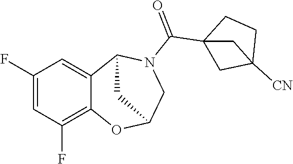 [SMILES: N#CC12CCC(C(=O)N3C[C@@H]4C[C@H]3c3cc(F)cc(F)c3O4)(C1)C2] | 4-((2S,5S)-7,9-difluoro- 2,3,4,5-tetrahydro-2,5- methanobenzo[f][1,4]oxaze- pine-4- carbonyl)bicyclo[2.1.1] hexane-1-carbonitrile | Calc'd 331, found 331 |

### Table 3 Compounds

### Example 3A. Preparation of 2,2-Dimethyl-1-((3S,6R or 3R,6S)-3,4,5,6-tetrahydro-2H-3,6-epiminobenzo[b]oxocin-11-yl)propan-1-one (3-1)

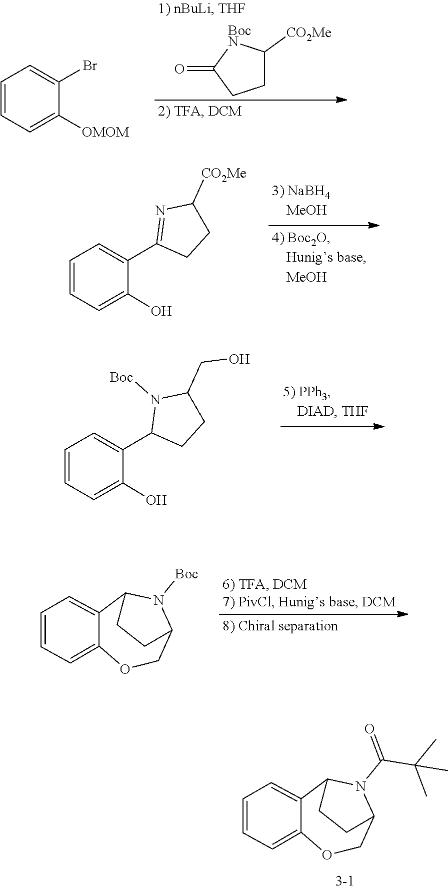

Step 1. A solution of 1-bromo-2-(methoxymethoxy)benzene (1.00 g, 4.61 mmol) in THF (20 mL) was treated at −78° C. with a 2.5 M solution of butyllithium (2.76 mL, 6.91 mmol) and stirred for 30 min. Next, 1-(tert-butyl)-2-methyl-5-oxopyrrolidine-1,2-dicarboxylate (1.35 g, 5.53 mmol) in THF (20 mL) was added, and the mixture stirred at −78° C. for 2 h. The reaction was quenched with NH₄Cl (50 mL), extracted with EtOAc (2×50 mL) which was washed with brine (2×50 mL), and the combined organic layers were dried over Na₂SO₄, filtered and concentrated. The crude residue was purified by chromatography on silica gel (0-30% EtOAc/pet ether, 25 g silica gel) to give methyl 2-((tert-butoxycarbonyl) amino)-5-(2-(methoxymethoxy) phenyl)-5-oxopentanoate. ¹H NMR (500 MHz, CDCl₃) δ 7.67 (br d, J=7.5 Hz, 1H), 7.39-7.46 (m, 1H), 7.18 (d, J=8.5 Hz, 1H), 7.05 (t, J=7.5 Hz, 1H), 5.23-5.32 (m, 2H), 5.06-5.20 (m, 1H), 4.31-4.39 (m, 1H), 3.70-3.77 (m, 3H), 3.52 (s, 3H), 3.03-3.18 (m, 2H), 2.21-2.31 (m, 1H), 2.02-2.13 (m, 1H), 1.42 (s, 9H); MS (EI) calculated for C₁₅H₂₀NO₇ [M-tBu+H]⁺, 326. found, 326.

Step 2. A solution of methyl 2-((tert-butoxycarbonyl)amino)-5-(2-(methoxymethoxy)phenyl)-5-oxopentanoate (450 mg, 1.18 mmol) in DCM (4 mL) and TFA (2 mL) was stirred for 0.5 h and then concentrated to give methyl 5-(2-hydroxyphenyl)-3,4-dihydro-2H-pyrrole-2-carboxylate. MS (EI) calculated for C₁₂H₁₃NO₃ [M+H]⁺, 220. found, 220.

Step 3. A solution of methyl 5-(2-hydroxyphenyl)-3,4-dihydro-2H-pyrrole-2-carboxylate (200 mg, 0.912 mmol) in MeOH (3 mL) was treated with NaBH₄ (345 mg, 9.12 mmol). The mixture was stirred for 1 h and quenched by the addition of H₂O (1 mL) and concentrated, giving 2-(5-(hydroxymethyl)pyrrolidin-2-yl)phenol.

Step 4. A solution of 2-(5-(hydroxymethyl)pyrrolidin-2-yl)phenol (150 mg, 0.776 mmol) in MeOH (3 mL) was treated with (Boc)₂O (0.180 mL, 0.776 mmol) and DIEA (0.678 mL, 3.88 mmol). The mixture was stirred at 20° C. for 1 h, diluted with water (10 mL) and extracted with DCM (3×10 mL). The combined organic layers were washed with brine (20 mL), dried over anhydrous Na₂SO₄, filtered and concentrated. The residue was purified by chromatography on silica gel to give cis-tert-butyl-2-(hydroxymethyl)-5-(2-hydroxyphenyl)pyrrolidine-1-carboxylate and trans-tert-butyl-2-(hydroxy methyl)-5-(2-hydroxy phenyl)pyrrolidine-1-carboxylate. MS (EI) calculated for C₁₂H₁₆NO+[M-tBu+H]⁺, 238. found, 238.

Step 5. A mixture containing cis-tert-butyl-2-(hydroxymethyl)-5-(2-hydroxyphenyl)pyrrolidine-1-carboxylate (80 mg, 0.27 mmol), triphenylphosphane (358 mg, 1.36 mmol) in THF (4 mL) was treated with DIAD (0.265 mL, 1.36 mmol), and the mixture was stirred for 16 h. The mixture was concentrated and purified by chromatography on silica gel to give tert-butyl 3,4,5,6-tetrahydro-2H-3,6-epiminobenzo[b]oxocine-11-carboxylate. ¹H NMR (400 MHz, CDCl₃) δ 7.11-7.21 (m, 2H), 6.95-7.06 (m, 2H), 4.79 (br d, J=9.2 Hz, 1H), 4.36 (br s, 1H), 4.13-4.20 (m, 1H), 3.92 (br d, J=12.4 Hz, 1H), 2.39-2.54 (m, 1H), 1.95-2.19 (m, 3H), 1.34 (s, 9H); MS (EI) calculated for C₁₂H₁₄NO₃ [M-tBu+H]⁺, 220. found, 220.

Step 6. A mixture containing tert-butyl 3,4,5,6-tetrahydro-2H-3,6-epiminobenzo[b]oxocine-11-carboxylate (30 mg, 0.10 mmol) in 1 mL of DCM and 0.5 mL of TFA was stirred for 30 min. The mixture was concentrated to provide 3,4,5,6-tetrahydro-2H-3,6-epiminobenzo[b]oxocine.

Steps 7 and 8. A solution of 3,4,5,6-tetrahydro-2H-3,6-epiminobenzo[b]oxocine (20 mg, 0.11 mmol) in DCM (0.5 mL) was treated with pivaloyl chloride (0.021 mL, 0.17 mmol), DIEA (0.20 mL, 1.14 mmol). The mixture was stirred for 1 h, concentrated and purified by reverse phase chromatography (43-63% MeCN/water with 0.1% TFA) followed by chiral SFC chromatography [Regis (s,s) WHELK-O1 250 mm×30 mm column, 50% EtOH/CO2 with 0.1% NH₃H₂O] to give the undesired enantiomer of (3S,6R or 3R,6S)-2,2-dimethyl-1-(3,4,5,6-tetrahydro-2H-3,6-epiminobenzo[b]oxocin-11-yl)propan-1-one (Ret time 2.94 min, 5.5 mg, 0.021 mmol) followed then by the desired bioactive enantiomer of (3S,6R or 3R,6S)-2,2-dimethyl-1-(3,4,5,6-tetrahydro-2H-3,6-epiminobenzo[b]oxocin-11-yl)propan-1-one (Ret time 4.53 min, 5.8 mg, 0.022 mmol;), both as white solids. ¹H NMR (400 MHz, CDCl₃) δ 7.35 (br d, J=7.6 Hz, 1H), 7.11-7.19 (m, 1 H), 6.97-7.05 (m, 2H), 5.38 (br d, J=9.2 Hz, 1H), 4.76 (br d, J=5.2 Hz, 1H), 4.29 (dd, J=12.0, 3.2 Hz, 1H), 3.86 (d, J=12.0 Hz, 1H), 2.29-2.41 (m, 1H), 2.18-2.27 (m, 1H), 2.07-2.16 (m, 1H), 1.95-2.05 (m, 1H), 1.23 (s, 9H); MS (EI) calculated for C₁₆H₂₂NO₂ [M+H]⁺, 260. found, 260.

### Example 3B. Preparation of 2,2-Dimethyl-1-((1S,4S)-1,3,4,5-tetrahydro-2H-1,4-methanobenzo[c]azepin-2-yl)propan-1-one (3-2)

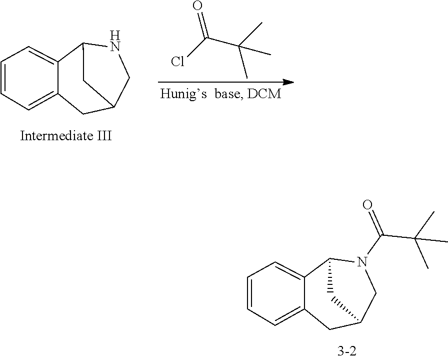

Intermediate III (50 mg, 0.256 mmol) was dissolved in DCM (1278 μL), then treated with Hunig's Base (134 μL, 0.767 mmol) and pivaloyl chloride (47 μL, 0.38 mmol) and allowed to stir at RT for 2 h. The reaction was concentrated then purified by reverse phase chromatography (MeCN/water with 0.1% TFA) to afford 2,2-dimethyl-1-(1,3,4,5-tetrahydro-2H-1,4-methanobenzo[c]azepin-2-yl)propan-1-one as a racemate. The racemate was then separated by SFC, chiral SFC Ret Time 1.85 min [(R,R)-Whelk-O, 21×250 mm, 5 μm, 25% MeOH with 0.1% NH₄OH] giving the desired more active enantiomer at 1.85 min and the undesired, less active enantiomer at 2.25 min. 2,2-dimethyl-1-((1S,4S)-1,3,4,5-tetrahydro-2H-1,4-methanobenzo[c]azepin-2-yl)propan-1-one. ¹H NMR (500 MHz, DMSO-d₆) δ 7.24-7.11 (m, 2H), 7.08 (d, J=7.2 Hz, 1H), 7.03 (t, J=7.1 Hz, 1H), 5.04 (s, 1H), 3.93-3.80 (m, 1H), 3.48 (s, 1H), 3.24-3.08 (m, 1H), 2.84 (d, J=17.5 Hz, 1H), 2.80 (d, J=4.1 Hz, 1H), 1.93 (dt, J=10.1, 4.8 Hz, 1H), 1.68 (d, J=11.1 Hz, 1H), 1.06 (s, 9H). MS (EI) calc'd for C₁₆H₂₂NO [M+H]⁺, 244. found, 244.

### Example 3C. Preparation of 3,3-Dimethyl-4-oxo-4-((1S,4S)-1,3,4,5-tetrahydro-2H-1,4-methanobenzo[c]azepin-2-yl)butanenitrile (3-3)

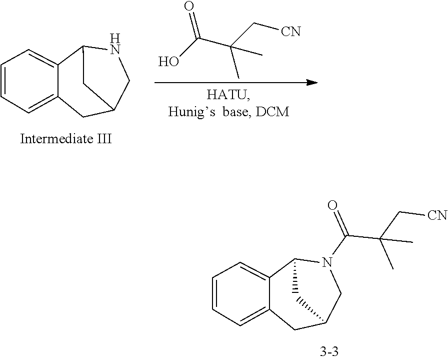

A solution of 3-cyano-2,2-dimethylpropanoic acid (114 mg, 0.9 mmol) in DMF (1.2 mL) was treated with Hunig's Base (520 μL, 3.0 mmol) then HATU (460 mg, 1.2 mmol) and stirred at RT for 10 min. Intermediate III (117 mg, 0.60 mmol) was added, and the reaction was allowed to stir at RT overnight. The crude mixture was filtered and purified by reverse phase chromatography (MeCN/water with 0.1% TFA) to afford 3,3-dimethyl-4-oxo-4-(1,3,4,5-tetrahydro-2H-1,4-methanobenzo[c]azepin-2-yl)butanenitrile as a racemate. The racemate was then separated by SFC, chiral SFC Ret Time 2.70 min [Lux-2 column, 21×250 mm, 5 μm, 25% MeOH with 0.1% NH₄OH] giving the desired more active enantiomer at 2.70 min and the undesired, less active enantiomer at 3.00 min 3,3-dimethyl-4-oxo-4-((1S,4S)-1,3,4,5-tetrahydro-2H-1,4-methanobenzo[c]azepin-2-yl)butanenitrile. ¹H NMR (500 MHz, DMSO-d₆) δ 7.15 (dd, J=16.6, 9.1 Hz, 2H), 7.09 (d, J=7.4 Hz, 1H), 7.04 (d, J=7.2 Hz, 1H), 5.04 (s, 1H), 3.85 (s, 1H), 3.52 (s, 1H), 3.14 (d, J=17.3 Hz, 1H), 2.86 (d, J=17.5 Hz, 1H), 2.82 (s, 1H), 2.66 (d, J=16.3 Hz, 1H), 2.57 (d, J=16.3 Hz, 1H), 2.19-1.92 (m, 1H), 1.71 (d, J=10.8 Hz, 1H), 1.29 (s, 3H), 1.07 (s, 3H). MS (EI) Calculated for C₁₇H₂₁N₂O [M+H]⁺, 269. found, 269.

### Example 3D. Preparation of 2,2-Dimethyl-1-((2S,5S)-2-methyl-2,3-dihydro-2,5-methanobenzo[f][1,4]oxazepin-4(5H)-yl)propan-1-one (3-5)

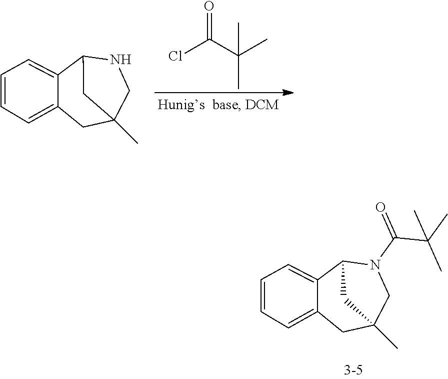

2-Methyl-2,3,4,5-tetrahydro-2,5-methanobenzo[f][1,4]oxazepine (70 mg, 0.399 mmol) was dissolved in DCM (2.0 mL), then treated with Hunig's Base (209 μL, 1.198 mmol) and pivaloyl chloride (93.7 μL, 0.599 mmol), and allowed to stir at RT for 2 h. The material was loaded directly onto silica and purified by chromatography on silica gel (4 g silica gel, 0-100% EtOAc/Hexanes) giving 2,2-dimethyl-1-(2-methyl-2,3-dihydro-2,5-methanobenzo[f][1,4]oxazepin-4(5H)-yl)propan-1-one as a racemate. The racemic material was resolved using chiral column chromatography [IC column, 21×250 mm, 5 μm; detection 215 nm, 70 mL/min 15% MeOH/CO₂ with 0.1% NH₄OH] giving the desired more active enantiomer at 2.90 min and the undesired, less active enantiomer at 2.20 min 2,2-dimethyl-1-((2S,5S)-2-methyl-2,3-dihydro-2,5-methanobenzo[f][1,4]oxazepin-4(5H)-yl)propan-1-one. ¹H NMR (500 MHz, Chloroform-d) δ 7.25 (d, J=5.0 Hz, 1H), 7.12 (t, J=7.5 Hz, 1H), 6.79 (t, J=7.4 Hz, 1H), 6.74 (d, J=8.1 Hz, 1H), 5.36-5.05 (m, 1H), 3.94 (s, 1H), 3.72 (d, J=11.4 Hz, 1H), 2.05 (d, J=3.5 Hz, 2H), 1.64 (s, 3H), 1.18 (s, 9H). MS (EI) calc'd for C₁₆H₂₂NO₂ [M+H]⁺, 260. found, 260.

### Example 3E. Preparation of 3,3-Difluoro-2,2-dimethyl-1-((2S,5S)-2-methyl-2,3-dihydro-2,5-methanobenzo[f][1,4]oxazepin-4(5H)-yl)propan-1-one (3-6)

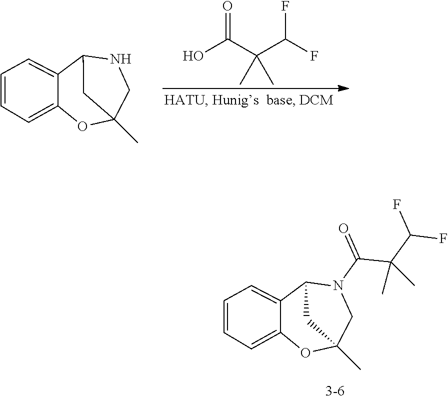

A solution of 3,3-difluoro-2,2-dimethylpropanoic acid (31.1 mg, 0.225 mmol) in DMF (3.0 mL) was treated with Hunig's Base (131 μL, 0.75 mmol) then HATU (114 mg, 0.3 mmol) and stirred at RT for 10 min. 2-Methyl-2,3,4,5-tetrahydro-2,5-methanobenzo[f][1,4]oxazepine (31.8 mg, 0.15 mmol) was added, and the reaction was allowed to stir at RT overnight. The crude mixture was filtered and purified by reverse phase chromatography (MeCN/water with 0.1% TFA) to afford 3,3-difluoro-2,2-dimethyl-1-(2-methyl-2,3-dihydro-2,5-methanobenzo[f][1,4]oxazepin-4(5H)-yl)propan-1-one as a racemate. The racemate was then separated by SFC, chiral SFC Ret Time 2.25 min [AD-H column, 21×250 mm, 5 μm, 10% MeOH with 0.1% NH₄OH] giving the desired more active enantiomer at 2.25 min and the undesired, less active enantiomer at 1.82 min 3,3-difluoro-2,2-dimethyl-1-((2S,5S)-2-methyl-2,3-dihydro-2,5-methanobenzo[f][1,4]oxazepin-4(5H)-yl)propan-1-one. ¹H NMR (500 MHz, DMSO-d₆) δ 7.11 (m, 2H), 6.73 (m, 2H), 6.20 (t, J=56.8 Hz, 1H), 5.14 (s, 1H), 3.91 (s, 1H), 3.83 (s, 1H), 2.20 (d, J=7.9 Hz, 1H), 1.93 (d, J=11.9 Hz, 1H), 1.59 (s, 3H), 1.19 (s, 3H), 1.03 (s, 3H). MS (EI) Calculated for C₁₆H₂₀F₂NO₂ [M+H]⁺, 296. found, 296.

Compound 3-4 was prepared in a fashion analogous to the preparation of 3-3, substituting the carbocylic acid for 4-(trifluoromethyl)bicyclo[2.2.1]heptane-1-carboxylic acid. The racemic material was resolved using chiral column chromatography [AD-H column, 21×250 mm, 5 μm; detection 215 nm, 70 mL/min 25% MeOH/CO₂ with 0.1% NH₄OH] giving the desired more active enantiomer at 2.23 min and the undesired, less active enantiomer at 1.96 min.

Compound 3-7 was prepared in a fashion analogous to the preparation of 3-6, substituting the carbocylic acid for bicyclo[2.2.1]heptane-1-carboxylic acid. The racemic material was resolved using chiral column chromatography [Lux-4 column, 21×250 mm, 5 μm; detection 215 nm, 70 mL/min 15% MeOH/CO₂ with 0.1% NH₄OH] giving the desired more active enantiomer at 4.55 min and the undesired, less active enantiomer at 5.05 min.

Compound 3-8 was prepared in a fashion analogous to the preparation of 3-6, substituting the carbocylic acid for 4-fluorobicyclo[2.2.1]heptane-1-carboxylic acid. The racemic material was resolved using chiral column chromatography [Lux-2 column, 21×250 mm, 5 μm; detection 215 nm, 70 mL/min 15% MeOH/CO₂ with 0.1% NH₄OH] giving the desired more active enantiomer at 4.10 min and the undesired, less active enantiomer at 4.75 min.

Compound 3-9 was prepared in a fashion analogous to the preparation of 3-6, substituting the carbocylic acid for 4-(trifluoromethyl)bicyclo[2.2.1]heptane-1-carboxylic acid. The racemic material was resolved using chiral column chromatography [AD-H column, 21×250 mm, 5 μm; detection 215 nm, 70 mL/min 10% MeOH/CO₂ with 0.1% NH₄OH] giving the desired more active enantiomer at 3.74 min and the undesired, less active enantiomer at 3.20 min.

TABLE 3

| | | | |
|---|---|---|---|
| 3-1 | 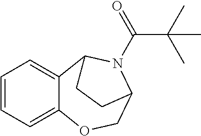 [SMILES: CC(C)(C)C(=O)N1C2CCC1c1ccccc1OC2] | 2,2-dimethyl-1-((3S,6R or 3R,6S)-3,4,5,6-tetrahydro- 2H-3,6- epiminobenzo[b]oxocin- 11-yl)propan-1-one | Calc'd 260, found 260 |
| 3-2 | 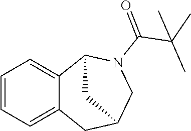 [SMILES: CC(C)(C)C(=O)N1C[C@H]2Cc3ccccc3[C@@H]1C2] | 2,2-dimethyl-1-((1S,4S)- 1,3,4,5-tetrahydro-2H-1,4- methanobenzo[c]azepin-2- yl)propan-1-one | Calc'd 244, found 244 |
| 3-3 | 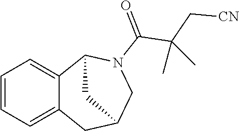 [SMILES: CC(C)(CC#N)C(=O)N1C[C@H]2Cc3ccccc3[C@@H]1C2] | 3,3-dimethyl-4-oxo-4- ((1S,4S)-1,3,4,5- tetrahydro-2H-1,4- methanobenzo[c]azepin-2- yl)butanenitrile | Calc'd 269, found 269 |
| 3-4 | 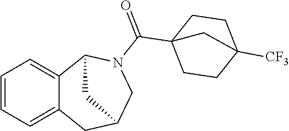 [SMILES: O=C(N1C[C@H]2Cc3ccccc3[C@@H]1C2)C12CCC(C(F)(F)F)(CC1)C2] | ((1S,4S)-1,3,4,5- tetrahydro-2H-1,4- methanobenzo[c]azepin-2- yl)(4-(trifluoromethyl) bicyclo[2.2.1] heptan-1-yl)methanone | Calc'd 350, found 350 |
| 3-5 | 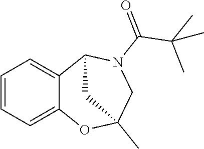 [SMILES: CC(C)(C)C(=O)N1C[C@]2(C)C[C@H]1c1ccccc1O2] | 2,2-dimethyl-1-((2S,5S)-2- methyl-2,3-dihydro-2,5- methanobenzo[f][1,4]oxaze- pin-4(5H)-yl)propan-1-one | Calc'd 260, found 260 |
| 3-6 | 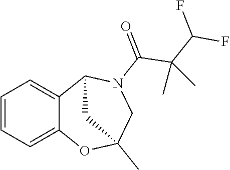 [SMILES: CC(C)(C(=O)N1C[C@]2(C)C[C@H]1c1ccccc1O2)C(F)F] | 3,3-difluoro-2,2-dimethyl- 1-((2S,5S)-2-methyl-2,3- dihydro-2,5- methanobenzo[f][1,4]oxaze- pin-4(5H)-yl)propan-1-one | Calc'd 296, found 296 |
| 3-7 | 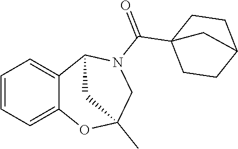 [SMILES: C[C@@]12C[C@@H](c3ccccc3O1)N(C(=O)C13CCC(CC1)C3)C2] | bicyclo[2.2.1]heptan-1- yl((2S,5S)-2-methyl-2,3- dihydro-2,5- methanobenzo[f][1,4]oxaze- pin-4(5H)-yl)methanone | Calc'd 298, found 298 |
| 3-8 | 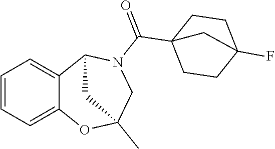 [SMILES: C[C@@]12C[C@@H](c3ccccc3O1)N(C(=O)C13CCC(F)(CC1)C3)C2] | (4- fluorobicyclo[2.2.1]heptan- 1-yl)((2S,5S)-2-methyl- 2,3-dihydro-2,5- methanobenzo[f][1,4]oxaze- pin-4(5H)-yl)methanone | Calc'd 316, found 316 |
| 3-9 | 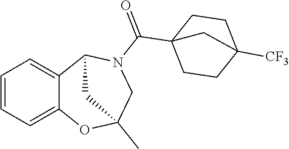 [SMILES: C[C@@]12C[C@@H](c3ccccc3O1)N(C(=O)C13CCC(C(F)(F)F)(CC1)C3)C2] | ((2S,5S)-2-methyl-2,3- dihydro-2,5- methanobenzo[f][1,4]oxaze- pin-4(5H)-yl)(4- (trifluoromethyl)bicyclo [2.2.1]heptan-1- yl)methanone | Calc'd 366, found 366 |

### RIPK1-ADP-Glo Enzymatic Assay

The enzymatic activity of RIPK1 is measured using an assay derived from ADP-Glo kit (™Promega), which provides a luminescent-based ADP detection system. Specifically, the ADP generated by RIPK1 kinase is proportionally detected as luminescent signals in a homogenous fashion. In this context, the assessment of the inhibitory effect of small molecules (EC₅₀) is measured by the effectiveness of the compounds to inhibit the ATP to ADP conversion by RIPK1.

In this assay, the potency (EC₅₀) of each compound was determined from a ten-point (1:3 serial dilution; top compound concentration of 100000 nM) titration curve using the following outlined procedure. To each well of a white ProxiPlus 384 well-plate, 30 nL of compound (1% DMSO in final assay volume of 3 μL) was dispensed, followed by the addition of 2 μL of 1× assay buffer (25 mM Hepes 7.3, 20 mM MgCl₂, 50 mM NaCl, 1 mM DTT, 0.005% Tween20, and 0.02% BSA) containing 37.5 nM of GST-RIPK1 (recombinant GST-RIPK1 kinase domain (residues 1-327) enzyme produced from baculovirus-transfected Sf21 cells: MW=62 kDa). Plates were placed in an ambient temperature humidified chamber for a 30 minutes pre-incubation with compound. Subsequently, each reaction was initiated by the addition of 1 μL 1× assay buffer containing 900 μM ATP and 3 μM dephosphorylated-MBP substrate. The final reaction in each well of 3 μL consists of 25 nM of GST-RIPK1, 300 μM ATP, and 3 μM dephosphorylated-MBP. Kinase reactions were allowed to proceed for 150 minutes prior to adding ADP-Glo reagents per Promega's outlined kit protocol. Dose-response curves were generated by plotting percent effect (% product conversion; Y-axis) vs. Log₁₀ compound concentrations (X-axis). EC₅₀ values were calculated using a non-linear regression, four-parameters sigmoidal dose-response model.

### RIPK1-ADP-Glo Enzymatic Assay Data

| Compound | RIPK1-ADP-Glo |
|---|---|
| Number | IC₅₀ (nM) |
| 1-1 | 48 |
| 1-2 | 61 |
| 1-3 | 97 |
| 1-4 | 33 |
| 1-5 | 42 |
| 1-6 | 43 |
| 1-7 | 217 |
| 1-8 | 641 |
| 1-9 | 381 |
| 1-10 | 138 |
| 1-11 | 244 |
| 1-12 | 86 |
| 1-13 | 104 |
| 1-14 | 313 |
| 1-15 | 981 |
| 1-16 | 96 |
| 1-17 | 47 |
| 1-18 | 73 |
| 1-19 | 499 |
| 1-20 | 62 |
| 1-21 | 888 |
| 1-22 | 21 |
| 1-23 | 234 |
| 1-24 | 69 |
| 1-25 | 140 |
| 1-26 | 60 |
| 1-27 | 921 |
| 1-28 | 22 |
| 1-29 | 58 |
| 1-30 | 76 |
| 1-31 | 157 |
| 1-32 | 391 |
| 1-33 | 881 |
| 1-34 | 174 |
| 1-35 | 220 |
| 1-36 | 327 |
| 1-37 | 124 |
| 1-38 | 146 |
| 1-39 | 213 |
| 1-40 | 23 |
| 1-41 | 13 |
| 1-42 | 922 |
| 1-43 | 18 |
| 1-44 | 17 |
| 1-45 | 168 |
| 1-46 | 117 |
| 1-47 | 281 |
| 1-48 | 375 |
| 1-49 | 117 |
| 1-50 | 393 |
| 1-51 | 723 |
| 1-52 | 199 |
| 1-53 | 510 |
| 1-54 | 263 |
| 1-55 | 43 |
| 1-56 | 863 |
| 1-57 | 56 |
| 1-58 | 887 |
| 1-59 | 159 |
| 1-60 | 512 |
| 2-1 | 61 |
| 2-2 | 158 |
| 2-3 | 143 |
| 2-4 | 60 |
| 2-5 | 34 |
| 2-6 | 43 |
| 2-7 | 25 |
| 2-8 | 20 |
| 2-9 | 63 |
| 2-10 | 30 |
| 2-11 | 26 |
| 2-12 | 20 |
| 2-13 | 23 |
| 2-14 | 15 |
| 2-15 | 24 |
| 2-16 | 35 |
| 2-17 | 51 |
| 2-18 | 25 |
| 2-19 | 18 |
| 2-20 | 52 |
| 2-21 | 37 |
| 2-22 | 388 |
| 2-23 | 49 |
| 2-24 | 30 |
| 2-25 | 10 |
| 2-26 | 12 |
| 2-27 | 16 |
| 2-28 | 809 |
| 2-29 | 55 |
| 2-30 | 75 |
| 2-31 | 132 |
| 2-32 | 39 |
| 2-33 | 26 |
| 2-34 | 27 |
| 2-35 | 12 |
| 2-36 | 19 |
| 3-1 | 281 |
| 3-2 | 2242 |
| 3-3 | 1490 |
| 3-4 | 267 |
| 3-5 | 17830 |
| 3-6 | 14670 |
| 3-7 | 6584 |
| 3-8 | 3975 |
| 3-9 | 2408 |

1. A compound of Formula I: 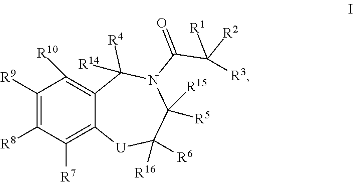
or a pharmaceutically acceptable salt thereof, wherein:
U is O, S, NR¹¹ or CR¹²R¹³,
R¹ is hydrogen, OH, C₁-C₆alkylOH, CN, C₁-C₆alkylCN, C₁-C₆alkyl, C₃-C₆cycloalkyl, haloC₁-C₆alkyl, halogen, NH₂, N(C₁-C₆alkyl)₂, NH(C₁-C₆alkyl) or alkoxy, or wherein R¹ is taken with R² and forms an oxo group, or wherein when R¹ is taken with R² or R² and R³ and forms a C₃-C₁₀cycloalkyl, aryl, heteroaryl or heterocycloalkyl, wherein the C₃-C₁₀cycloalkyl, aryl, heteroaryl or heterocycloalkyl is unsubstituted or substituted with one to four substituents selected from the group consisting of halogen, C₁-C₆alkyl, haloC₁-C₆alkyl, alkoxy, CN, COOC₁-C₆alkyl, COaryl, aryl, and heteroaryl, wherein the heteroaryl is unsubstituted or substituted with one to four substituents selected from the group consisting of halogen, CN, C₁-C₆alkyl, alkoxy, aryl, C₃-C₆cycloalkyl and haloC₁-C₆alkyl;
R² is hydrogen, OH, C₁-C₆alkylOH, CN, C₁-C₆alkylCN, C₁-C₆alkyl, C₃-C₆cycloalkyl, haloC₁-C₆alkyl, halogen, NH₂, N(C₁-C₆alkyl)₂, NH(C₁-C₆alkyl) or alkoxy, or wherein R² is taken with R¹ and forms an oxo group, or wherein when R² is taken with R¹ or R¹ and R³ and forms a C₃-C₁₀cycloalkyl, aryl, heteroaryl or heterocycloalkyl, wherein the C₃-C₁₀cycloalkyl, aryl, heteroaryl or heterocycloalkyl is unsubstituted or substituted with one to four substituents selected from the group consisting of halogen, C₁-C₆alkyl, haloC₁-C₆alkyl, alkoxy, CN, COOC₁-C₆alkyl, COaryl, aryl, and heteroaryl, wherein the heteroaryl is unsubstituted or substituted with one to four substituents selected from the group consisting of halogen, CN, C₁-C₆alkyl, alkoxy, aryl, C₃-C₆cycloalkyl and haloC₁-C₆alkyl;
R³ is OH, C₁-C₆alkylOH, CN, C₁-C₆alkylCN, C₁-C₆alkyl, C₃-C₆cycloalkyl, haloC₁-C₆alkyl, halogen, C₁-C₆alkylOhaloC₁-C₆alkyl, alkoxy, NHC₁-C₆alkylaryl, CONHC₁-C₆alkylaryl, aryl, or C₁-C₆alkylaryl, wherein the NHC₁-C₆alkylaryl, CONHC₁-C₆alkylaryl, aryl, or C₁-C₆alkylaryl is unsubstituted or substituted with one to four substituents selected from the group consisting of halogen, C₁-C₆alkyl, alkoxy and haloC₁-C₆alkyl, or wherein when R³ is taken with R² or R¹ and R², forms a C₃-C₁₀cycloalkyl, aryl, heteroaryl or heterocycloalkyl, wherein the C₃-C₁₀cycloalkyl, aryl, heteroaryl or heterocycloalkyl is unsubstituted or substituted with one to four substituents selected from the group consisting of halogen, C₁-C₆alkyl, haloC₁-C₆alkyl, alkoxy, CN, COOC₁-C₆alkyl, COaryl, aryl, and heteroaryl, wherein the heteroaryl is unsubstituted or substituted with one to four substituents selected from the group consisting of halogen, CN, C₁-C₆alkyl, alkoxy, aryl, C₃-C₆cycloalkyl and haloC₁-C₆alkyl;
R⁴ is hydrogen, C₁-C₆alkyl, or wherein when taken with R⁵ or R⁶ form a —CH₂— or —CH₂CH₂— bridge;
R⁵ is hydrogen, C₁-C₆alkyl, or wherein when taken with R⁴ form a —CH₂— or —CH₂CH₂— bridge;
R⁶ is hydrogen, C₁-C₆alkyl, or wherein when taken with R⁴ form a —CH₂— or —CH₂CH₂— bridge;
R⁷ is hydrogen, OH, C₁-C₆alkylOH, CN, C₁-C₆alkylCN, C₁-C₆alkyl, C₁-C₆alkylaryl, C₂-C₆alkynyl, haloC₁-C₆alkyl, halogen, or alkoxy, wherein the C₂-C₆alkynyl is unsubstituted or substituted with one to three substituents selected from the group consisting of OH, C₁-C₆alkylOH, CN, C₁-C₆alkylCN, NH₂, NHC₁-C₆alkyl, N(C₁-C₆alkyl)₂, haloC₁-C₆alkyl, halogen, alkoxy, haloalkoxy, aryl, C₃-C₁₀cycloalkyl, heteroaryl and heterocycloalkyl, wherein the aryl, C₃-C₁₀cycloalkyl, heteroaryl and heterocycloalkyl are unsubstituted or substituted with one to three substituents selected from the group consisting of OH, C₁-C₆alkylOH, CN, C₁-C₆alkylCN, C₁-C₆alkyl, NH₂, NHC₁-C₆alkyl, N(C₁-C₆alkyl)₂, haloC₁-C₆alkyl, halogen, alkoxy, haloalkoxy;
R⁸ is hydrogen, OH, C₁-C₆alkylOH, CN, C₁-C₆alkylCN, C₁-C₆alkyl, C₁-C₆alkylaryl, C₂-C₆alkynyl, haloC₁-C₆alkyl, halogen, or alkoxy;
R⁹ is hydrogen, OH, C₁-C₆alkylOH, CN, C₁-C₆alkylCN, C₁-C₆alkyl, C₁-C₆alkylaryl, C₂-C₆alkynyl, haloC₁-C₆alkyl, halogen, or alkoxy;
R¹⁰ is hydrogen, OH, C₁-C₆alkylOH, CN, C₁-C₆alkylCN, C₁-C₆alkyl, C₁-C₆alkylaryl, C₂-C₆alkynyl, haloC₁-C₆alkyl, halogen, or alkoxy;
R¹¹ is hydrogen, C₁-C₆alkyl, or C₃-C₆cycloalkyl;
R¹² is hydrogen, OH, C₁-C₆alkylOH, CN, C₁-C₆alkylCN, C₁-C₆alkyl, haloC₁-C₆alkyl, halogen, or alkoxy;
R¹³ is hydrogen, OH, C₁-C₆alkylOH, CN, C₁-C₆alkylCN, C₁-C₆alkyl, haloC₁-C₆alkyl, halogen, or alkoxy;
R¹⁴ is hydrogen, OH, C₁-C₆alkylOH, CN, C₁-C₆alkylCN, C₁-C₆alkyl, haloC₁-C₆alkyl, halogen, or alkoxy;
R¹⁵ is hydrogen, OH, C₁-C₆alkylOH, CN, C₁-C₆alkylCN, C₁-C₆alkyl, haloC₁-C₆alkyl, halogen, or alkoxy; and
R¹⁶ is hydrogen, OH, C₁-C₆alkylOH, CN, C₁-C₆alkylCN, C₁-C₆alkyl, haloC₁-C₆alkyl, halogen, or alkoxy.

2. The compound of claim 1, wherein when U is O or CR¹²R¹³, R⁴ forms a —CH₂— or —CH₂CH₂— bridge with R⁵ or R⁶, or a pharmaceutically acceptable salt thereof.

3. The compound of claim 1, wherein U is O, or a pharmaceutically acceptable salt thereof.

4. The compound of claim 1, wherein R¹ and R² are independently selected from the group consisting of methyl, t-butyl, fluoromethyl, difluoromethyl, fluorine, difluoroethyl, trifluoromethyl, ethyl, N(CH₃)₂, CN and CH₂CN, and R³ is independently selected from the group consisting of methyl, difluoromethyl, fluorine, difluoroethyl, trifluoromethyl and ethyl,
or a pharmaceutically acceptable salt thereof.

5. The compound of claim 1, wherein R² is taken with R³ or R¹ and R³ and forms a C₃-C₁₀cycloalkyl or heterocycloalkyl, wherein the C₃-C₁₀cycloalkyl or heterocycloalkyl is unsubstituted or substituted with one to four substituents selected from the group consisting of halogen, C₁-C₆alkyl, haloC₁-C₆alkyl, alkoxy, CN, COOC₁-C₆alkyl, and heteroaryl, wherein the heteroaryl is unsubstituted or substituted with one to four substituents selected from the group consisting of halogen, CN, C₁-C₆alkyl and haloC₁-C₆alkyl, or a pharmaceutically acceptable salt thereof.

6. The compound of claim 1, wherein when R³ is taken with R² or R¹ and R² and forms a C₃-C₁₀cycloalkyl or heterocycloalkyl, wherein the C₃-C₁₀cycloalkyl or heterocycloalkyl is unsubstituted or substituted with one to four substituents selected from the group consisting of halogen, C₁-C₆alkyl, haloC₁-C₆alkyl, alkoxy, CN, COOC₁-C₆alkyl, and heteroaryl, wherein the heteroaryl is unsubstituted or substituted with one to four substituents selected from the group consisting of halogen, CN, C₁-C₆alkyl and haloC₁-C₆alkyl, or a pharmaceutically acceptable salt thereof.

7. The compound of claim 5, wherein the C₃-C₁₀cycloalkyl is 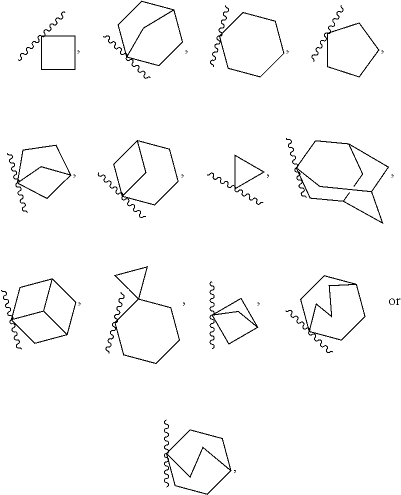
wherein the C₃-C₁₀cycloalkyl is unsubstituted or substituted with one to four substituents selected from the group consisting of halogen, C₁-C₆alkyl, haloC₁-C₆alkyl, alkoxy, CN, COOC₁-C₆alkyl, or a pharmaceutically acceptable salt thereof.

8. The compound of claim 5, wherein the heterocycloalkyl is 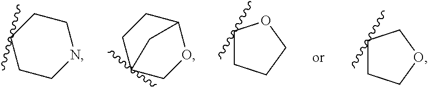
wherein the heterocycloalkyl is unsubstituted or substituted with one to four substituents selected from the group consisting of halogen, C₁-C₆alkyl, haloC₁-C₆alkyl, alkoxy, CN, COOC₁-C₆alkyl, and heteroaryl, wherein the heteroaryl is unsubstituted or substituted with one to four substituents selected from the group consisting of halogen, CN, C₁-C₆alkyl and haloC₁-C₆alkyl, or a pharmaceutically acceptable salt thereof.

9. The compound of claim 8, wherein when the heterocycloalkyl is 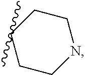
and wherein the heterocycloalkyl is substituted with heteroaryl selected from 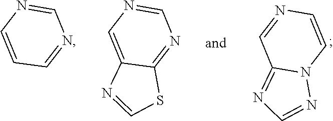
wherein the heteroaryl is unsubstituted or substituted with CN, or a pharmaceutically acceptable salt thereof.

10. The compound of claim 1, wherein R³ is methyl, difluoromethyl, CH₂OCF₃, cyclobutyl, difluoroethyl, ethyl, cyclopropyl, CN, CH₂CN, CH₂phenyl, CONHCH₂phenyl, wherein the CH₂phenyl or CONHCH₂phenyl is substituted with one substituent selected from the group consisting of chlorine and methoxy, or a pharmaceutically acceptable salt thereof.

11. A compound of Formula II: 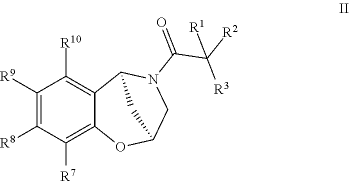
or a pharmaceutically acceptable salt thereof, wherein:
R¹ is hydrogen, OH, C₁-C₆alkylOH, CN, C₁-C₆alkylCN, C₁-C₆alkyl, C₃-C₆cycloalkyl, haloC₁-C₆alkyl, halogen, NH₂, N(C₁-C₆alkyl)₂, NH(C₁-C₆alkyl) or alkoxy, or wherein R¹ is taken with R² and forms an oxo group, or wherein when R¹ is taken with R² or R² and R³ and forms a C₃-C₁₀cycloalkyl, aryl, heteroaryl or heterocycloalkyl, wherein the C₃-C₁₀cycloalkyl, aryl, heteroaryl or heterocycloalkyl is unsubstituted or substituted with one to four substituents selected from the group consisting of halogen, C₁-C₆alkyl, haloC₁-C₆alkyl, alkoxy, CN, COOC₁-C₆alkyl, COaryl, aryl, and heteroaryl, wherein the heteroaryl is unsubstituted or substituted with one to four substituents selected from the group consisting of halogen, CN, C₁-C₆alkyl, alkoxy, aryl, C₃-C₆cycloalkyl and haloC₁-C₆alkyl;
R² is hydrogen, OH, C₁-C₆alkylOH, CN, C₁-C₆alkylCN, C₁-C₆alkyl, C₃-C₆cycloalkyl, haloC₁-C₆alkyl, halogen, NH₂, N(C₁-C₆alkyl)₂, NH(C₁-C₆alkyl) or alkoxy, or wherein R² is taken with R¹ and forms an oxo group, or wherein when R² is taken with R¹ or R¹ and R³ and forms a C₃-C₁₀cycloalkyl, aryl, heteroaryl or heterocycloalkyl, wherein the C₃-C₁₀cycloalkyl, aryl, heteroaryl or heterocycloalkyl is unsubstituted or substituted with one to four substituents selected from the group consisting of halogen, C₁-C₆alkyl, haloC₁-C₆alkyl, alkoxy, CN, COOC₁-C₆alkyl, COaryl, aryl, and heteroaryl, wherein the heteroaryl is unsubstituted or substituted with one to four substituents selected from the group consisting of halogen, CN, C₁-C₆alkyl, alkoxy, aryl, C₃-C₆cycloalkyl and haloC₁-C₆alkyl;
R³ is OH, C₁-C₆alkylOH, CN, C₁-C₆alkylCN, C₁-C₆alkyl, C₃-C₆cycloalkyl, haloC₁-C₆alkyl, halogen, C₁-C₆alkylOhaloC₁-C₆alkyl, alkoxy, NHC₁-C₆alkylaryl, CONHC₁-C₆alkylaryl, aryl, or C₁-C₆alkylaryl, wherein the NHC₁-C₆alkylaryl, CONHC₁-C₆alkylaryl, aryl, or C₁-C₆alkylaryl is unsubstituted or substituted with one to four substituents selected from the group consisting of halogen, C₁-C₆alkyl, alkoxy and haloC₁-C₆alkyl, or wherein when R³ is taken with R² or R¹ and R², forms a C₃-C₁₀cycloalkyl, aryl, heteroaryl or heterocycloalkyl, wherein the C₃-C₁₀cycloalkyl, aryl, heteroaryl or heterocycloalkyl is unsubstituted or substituted with one to four substituents selected from the group consisting of halogen, C₁-C₆alkyl, haloC₁-C₆alkyl, alkoxy, CN, COOC₁-C₆alkyl, COaryl, aryl, and heteroaryl, wherein the heteroaryl is unsubstituted or substituted with one to four substituents selected from the group consisting of halogen, CN, C₁-C₆alkyl, alkoxy, aryl, C₃-C₆cycloalkyl and haloC₁-C₆alkyl;
R⁷ is hydrogen, OH, C₁-C₆alkylOH, CN, C₁-C₆alkylCN, C₁-C₆alkyl, C₁-C₆alkylaryl, C₁-C₆alkylaryl, C₂-C₆alkynyl, haloC₁-C₆alkyl, halogen, or alkoxy, wherein the C₂-C₆alkynyl is unsubstituted or substituted with one to three substituents selected from the group consisting of OH, C₁-C₆alkylOH, CN, C₁-C₆alkylCN, NH₂, NHC₁-C₆alkyl, N(C₁-C₆alkyl)₂, haloC₁-C₆alkyl, halogen, alkoxy, haloalkoxy, aryl, C₃-C₁₀cycloalkyl, heteroaryl and heterocycloalkyl, wherein the aryl, C₃-C₁₀cycloalkyl, heteroaryl and heterocycloalkyl are unsubstituted or substituted with one to three substituents selected from the group consisting of OH, C₁-C₆alkylOH, CN, C₁-C₆alkylCN, C₁-C₆alkyl, NH₂, NHC₁-C₆alkyl, N(C₁-C₆alkyl)₂, haloC₁-C₆alkyl, halogen, alkoxy, haloalkoxy;
R⁸ is hydrogen, OH, C₁-C₆alkylOH, CN, C₁-C₆alkylCN, C₁-C₆alkyl, C₁-C₆alkylaryl, alkynyl, alkynylaryl, haloC₁-C₆alkyl, halogen, or alkoxy;
R⁹ is hydrogen, OH, C₁-C₆alkylOH, CN, C₁-C₆alkylCN, C₁-C₆alkyl, C₁-C₆alkylaryl, alkynyl, alkynylaryl, haloC₁-C₆alkyl, halogen, or alkoxy;
R¹⁰ is hydrogen, OH, C₁-C₆alkylOH, CN, C₁-C₆alkylCN, C₁-C₆alkyl, C₁-C₆alkylaryl, alkynyl, alkynylaryl, haloC₁-C₆alkyl, halogen, or alkoxy.

12. A compound of Formula III: 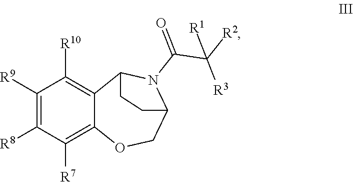
or a pharmaceutically acceptable salt thereof, wherein:
R¹ is hydrogen, OH, C₁-C₆alkylOH, CN, C₁-C₆alkylCN, C₁-C₆alkyl, C₃-C₆cycloalkyl, haloC₁-C₆alkyl, halogen, NH₂, N(C₁-C₆alkyl)₂, NH(C₁-C₆alkyl) or alkoxy, or wherein R¹ is taken with R² and forms an oxo group, or wherein when R¹ is taken with R² or R² and R³ and forms a C₃-C₁₀cycloalkyl, aryl, heteroaryl or heterocycloalkyl, wherein the C₃-C₁₀cycloalkyl, aryl, heteroaryl or heterocycloalkyl is unsubstituted or substituted with one to four substituents selected from the group consisting of halogen, C₁-C₆alkyl, haloC₁-C₆alkyl, alkoxy, CN, COOC₁-C₆alkyl, COaryl, aryl, and heteroaryl, wherein the heteroaryl is unsubstituted or substituted with one to four substituents selected from the group consisting of halogen, CN, C₁-C₆alkyl, alkoxy, aryl, C₃-C₆cycloalkyl and haloC₁-C₆alkyl;
R² is hydrogen, OH, C₁-C₆alkylOH, CN, C₁-C₆alkylCN, C₁-C₆alkyl, C₃-C₆cycloalkyl, haloC₁-C₆alkyl, halogen, NH₂, N(C₁-C₆alkyl)₂, NH(C₁-C₆alkyl) or alkoxy, or wherein R² is taken with R¹ and forms an oxo group, or wherein when R² is taken with R¹ or R¹ and R³ and forms a C₃-C₁₀cycloalkyl, aryl, heteroaryl or heterocycloalkyl, wherein the C₃-C₁₀cycloalkyl, aryl, heteroaryl or heterocycloalkyl is unsubstituted or substituted with one to four substituents selected from the group consisting of halogen, C₁-C₆alkyl, haloC₁-C₆alkyl, alkoxy, CN, COOC₁-C₆alkyl, COaryl, aryl, and heteroaryl, wherein the heteroaryl is unsubstituted or substituted with one to four substituents selected from the group consisting of halogen, CN, C₁-C₆alkyl, alkoxy, aryl, C₃-C₆cycloalkyl and haloC₁-C₆alkyl;
R³ is OH, C₁-C₆alkylOH, CN, C₁-C₆alkylCN, C₁-C₆alkyl, C₃-C₆cycloalkyl, haloC₁-C₆alkyl, halogen, C₁-C₆alkylOhaloC₁-C₆alkyl, alkoxy, NHC₁-C₆alkylaryl, CONHC₁-C₆alkylaryl, aryl, or C₁-C₆alkylaryl, wherein the NHC₁-C₆alkylaryl, CONHC₁-C₆alkylaryl, aryl, or C₁-C₆alkylaryl is unsubstituted or substituted with one to four substituents selected from the group consisting of halogen, C₁-C₆alkyl, alkoxy and haloC₁-C₆alkyl, or wherein when R³ is taken with R² or R¹ and R², forms a C₃-C₁₀cycloalkyl, aryl, heteroaryl or heterocycloalkyl, wherein the C₃-C₁₀cycloalkyl, aryl, heteroaryl or heterocycloalkyl is unsubstituted or substituted with one to four substituents selected from the group consisting of halogen, C₁-C₆alkyl, haloC₁-C₆alkyl, alkoxy, CN, COOC₁-C₆alkyl, COaryl, aryl, and heteroaryl, wherein the heteroaryl is unsubstituted or substituted with one to four substituents selected from the group consisting of halogen, CN, C₁-C₆alkyl, alkoxy, aryl, C₃-C₆cycloalkyl and haloC₁-C₆alkyl;
R⁷ is hydrogen, OH, C₁-C₆alkylOH, CN, C₁-C₆alkylCN, C₁-C₆alkyl, C₁-C₆alkylaryl, C₁-C₆alkylaryl, C₂-C₆alkynyl, haloC₁-C₆alkyl, halogen, or alkoxy, wherein the C₂-C₆alkynyl is unsubstituted or substituted with one to three substituents selected from the group consisting of OH, C₁-C₆alkylOH, CN, C₁-C₆alkylCN, NH₂, NHC₁-C₆alkyl, N(C₁-C₆alkyl)₂, haloC₁-C₆alkyl, halogen, alkoxy, haloalkoxy, aryl, C₃-C₁₀cycloalkyl, heteroaryl and heterocycloalkyl, wherein the aryl, C₃-C₁₀cycloalkyl, heteroaryl and heterocycloalkyl are unsubstituted or substituted with one to three substituents selected from the group consisting of OH, C₁-C₆alkylOH, CN, C₁-C₆alkylCN, C₁-C₆alkyl, NH₂, NHC₁-C₆alkyl, N(C₁-C₆alkyl)₂, haloC₁-C₆alkyl, halogen, alkoxy, haloalkoxy;
R⁸ is hydrogen, OH, C₁-C₆alkylOH, CN, C₁-C₆alkylCN, C₁-C₆alkyl, C₁-C₆alkylaryl, alkynyl, alkynylaryl, haloC₁-C₆alkyl, halogen, or alkoxy;
R⁹ is hydrogen, OH, C₁-C₆alkylOH, CN, C₁-C₆alkylCN, C₁-C₆alkyl, C₁-C₆alkylaryl, alkynyl, alkynylaryl, haloC₁-C₆alkyl, halogen, or alkoxy;
R¹⁰ is hydrogen, OH, C₁-C₆alkylOH, CN, C₁-C₆alkylCN, C₁-C₆alkyl, C₁-C₆alkylaryl, alkynyl, alkynylaryl, haloC₁-C₆alkyl, halogen, or alkoxy.

13. The compound of claim 1, or a pharmaceutically acceptable salt thereof, wherein R⁷ is hydrogen chlorine, CN, methyl or fluorine.

14. The compound of claim 1, or a pharmaceutically acceptable salt thereof, wherein R⁸ is hydrogen, CN or chlorine.

15. The compound of claim 1, or a pharmaceutically acceptable salt thereof, wherein R⁹ is hydrogen chlorine, CN, methyl or fluorine.

16. The compound of claim 1, or a pharmaceutically acceptable salt thereof, wherein R¹⁰ is hydrogen.

17. The compound of claim 1 selected from: 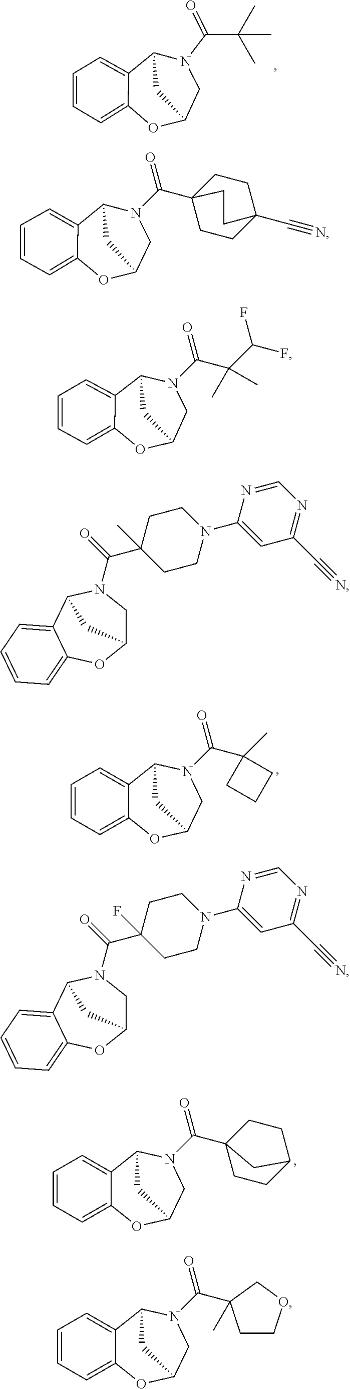 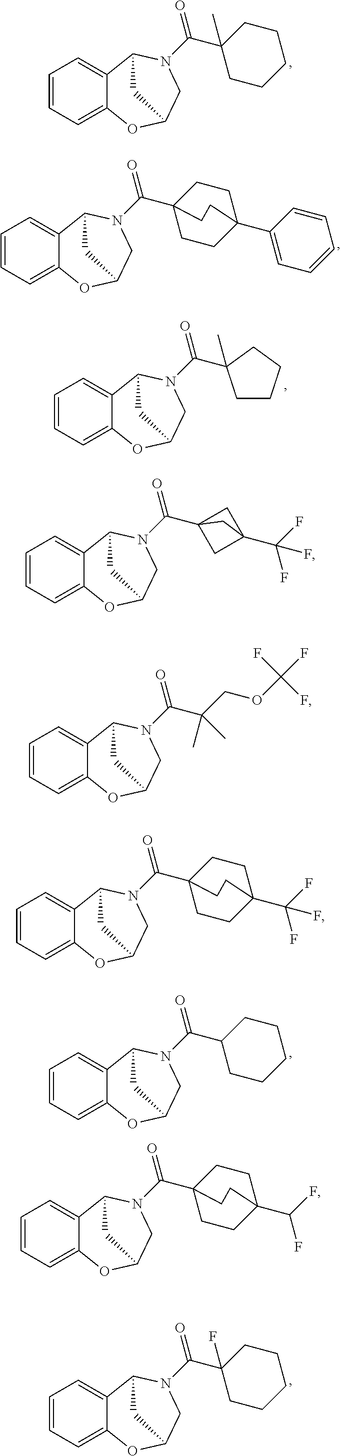 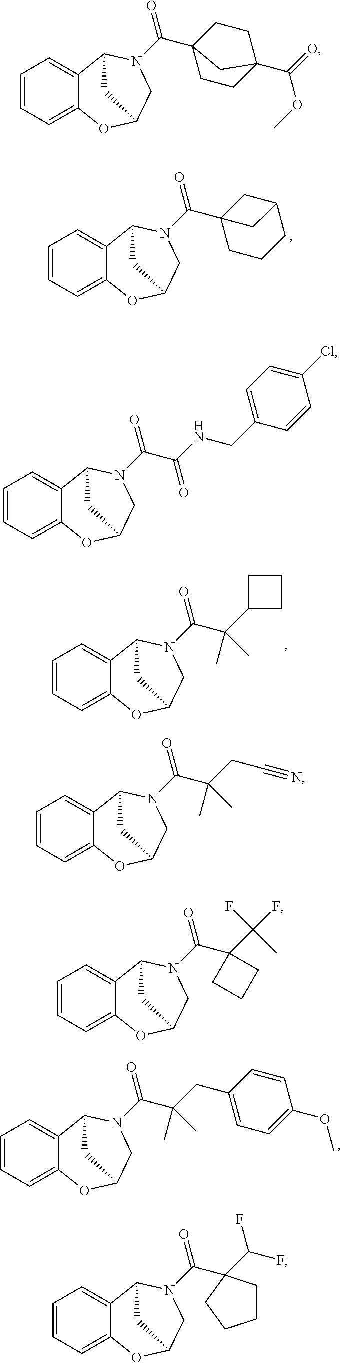 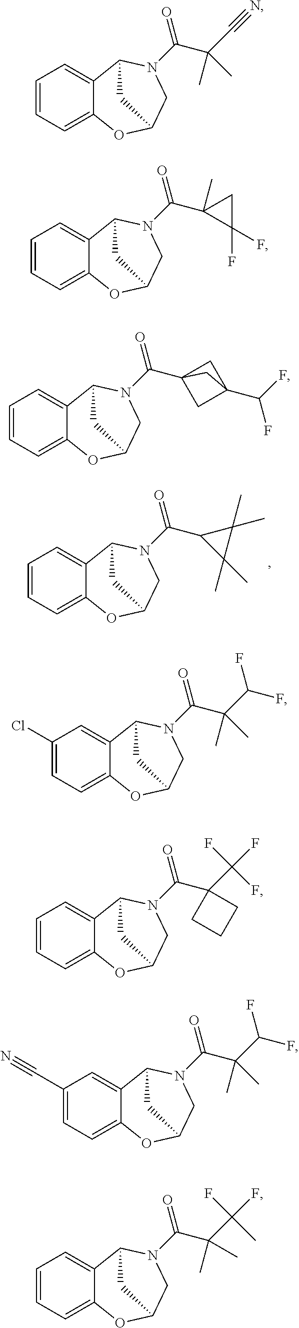 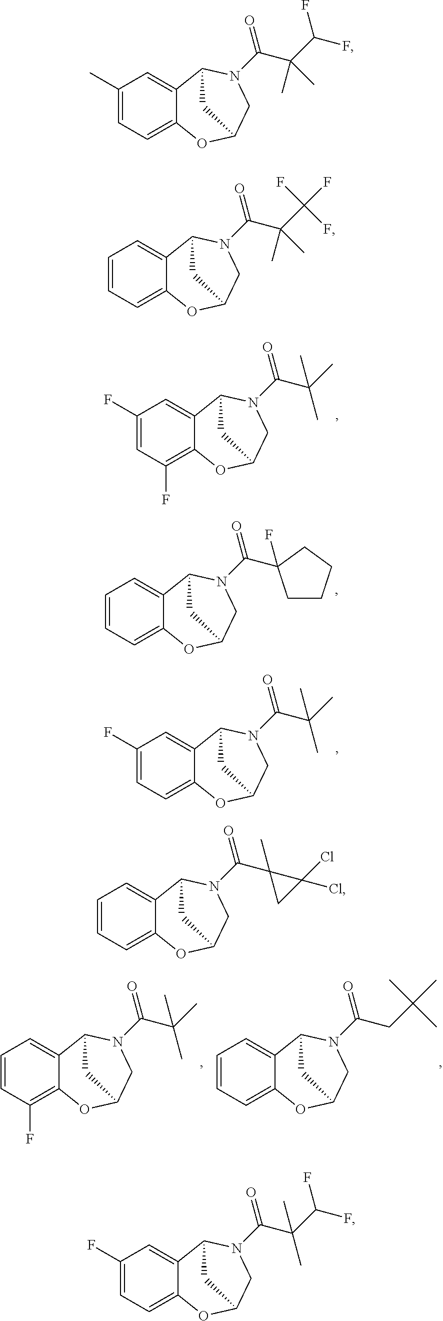 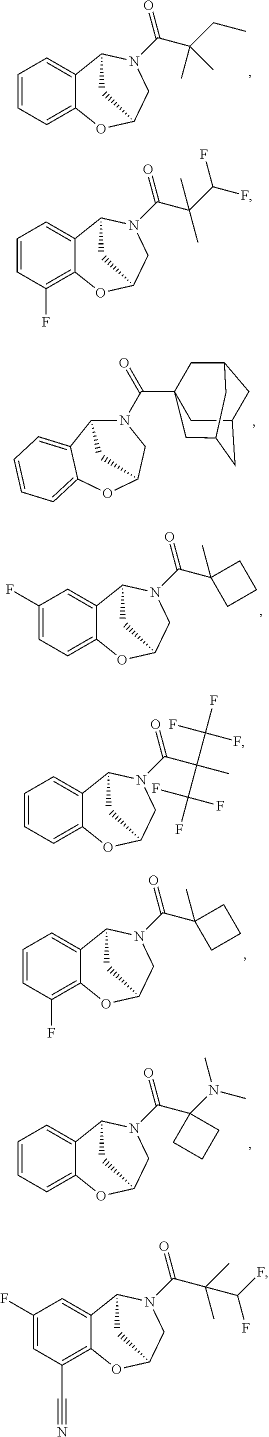 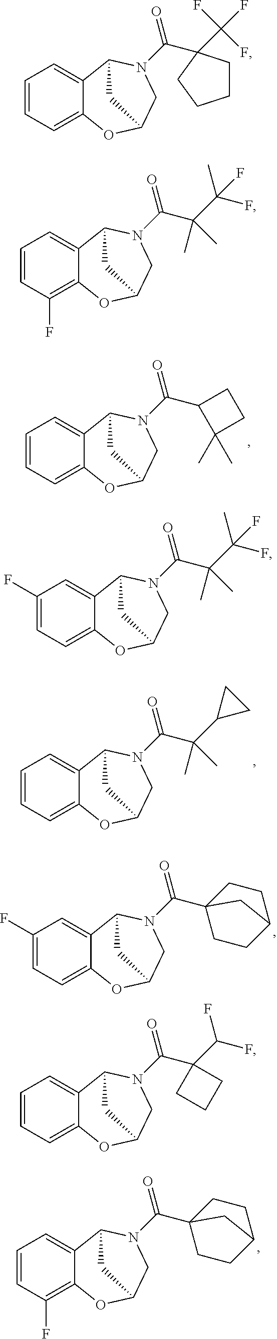 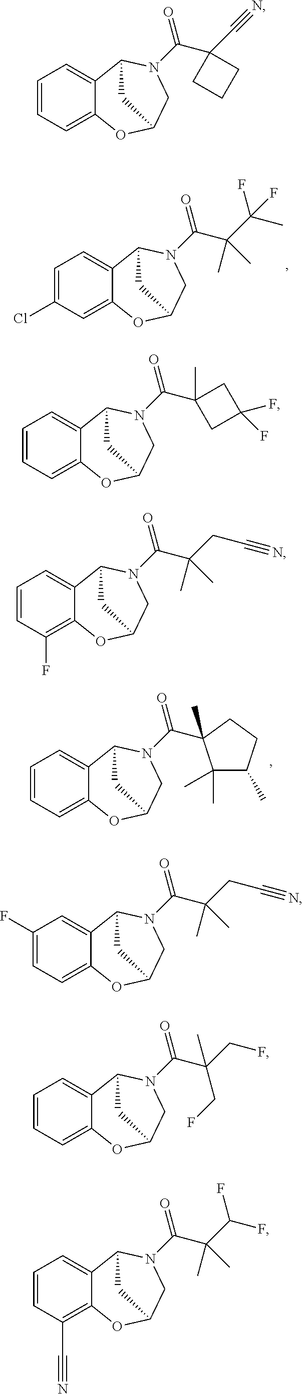 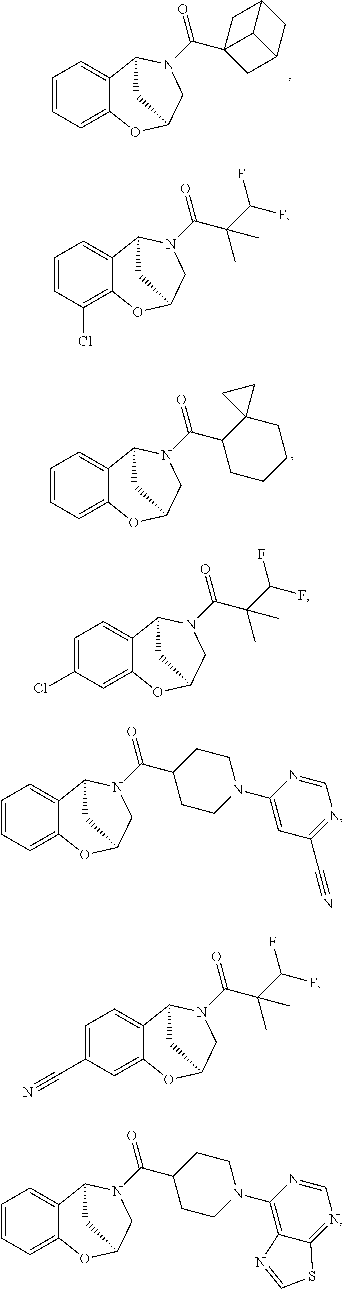 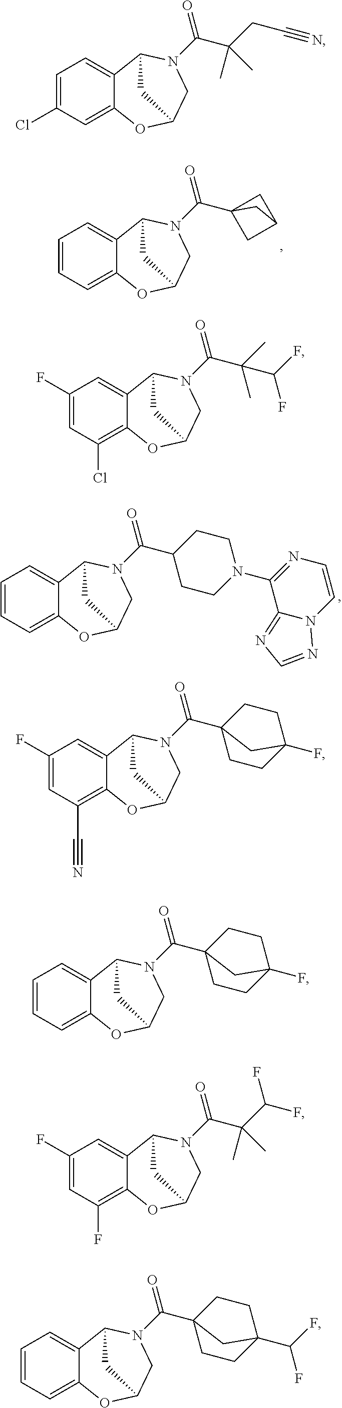 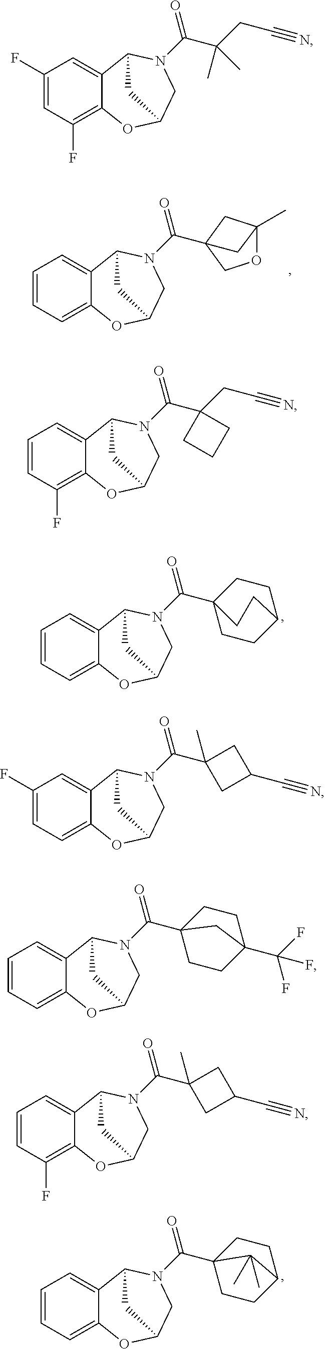 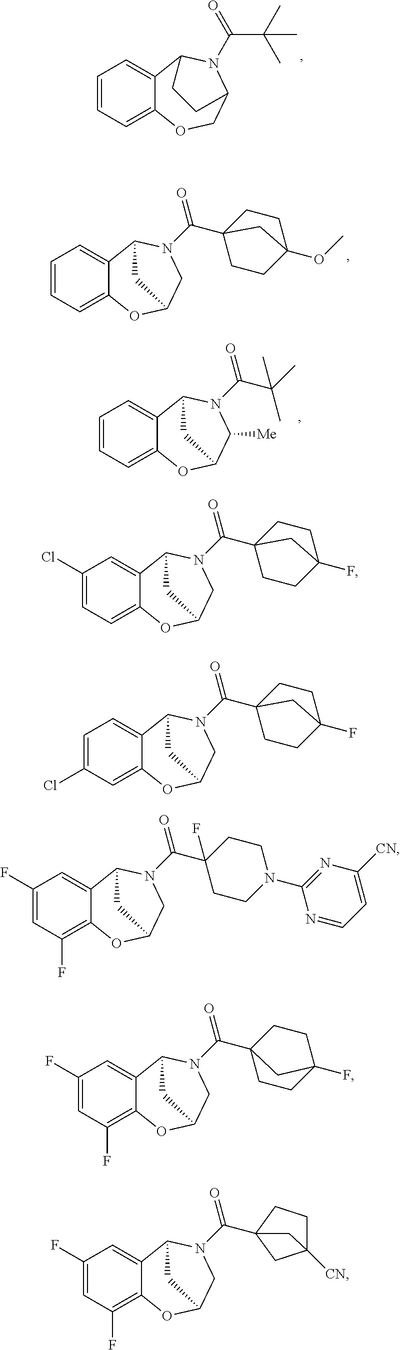 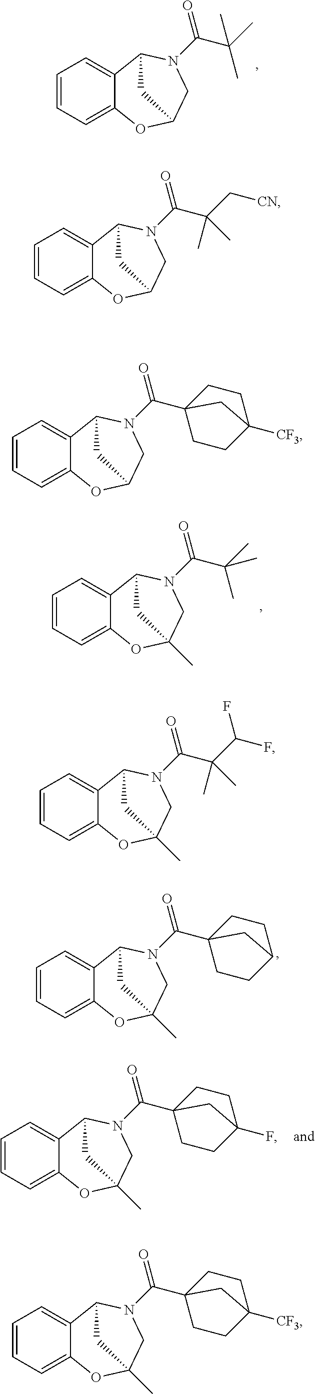
or a pharmaceutically acceptable salt thereof.

18. A method for treating RIPK1 dependent inflammation and cell death that occurs in inherited and sporadic diseases including Alzheimer's disease, amyotrophic lateral sclerosis, multiple sclerosis, Parkinson's disease, chronic traumatic encephalopathy, rheumatoid arthritis, ulcerative colitis, inflammatory bowel disease, psoriasis as well as acute tissue injury caused by stroke, traumatic brain injury, encephalitis comprising administering to a patient in need thereof a compound, or pharmaceutically acceptable salt thereof, of claim 1.

19. A method of treating amyotrophic lateral sclerosis comprising administering to a patient in need thereof a compound, or pharmaceutically acceptable salt thereof, of claim 1.

20. (canceled)

21. A pharmaceutical composition comprising a compound of claim 1, or a pharmaceutically acceptable salt thereof and a pharmaceutically acceptable carrier.

22. (canceled)

23. The compound of claim 6, wherein the C₃-C₁₀cycloalkyl is 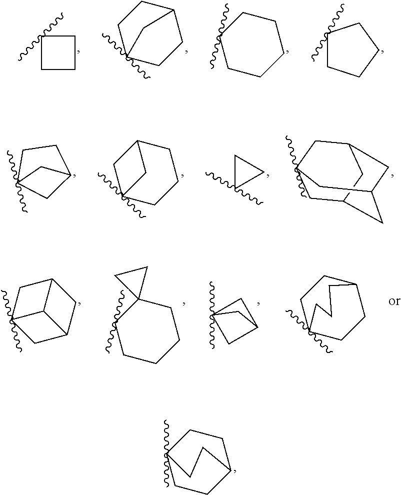
wherein the C₃-C₁₀cycloalkyl is unsubstituted or substituted with one to four substituents selected from the group consisting of halogen, C₁-C₆alkyl, haloC₁-C₆alkyl, alkoxy, CN, COOC₁-C₆alkyl, or a pharmaceutically acceptable salt thereof.

24. The compound of claim 6, wherein the heterocycloalkyl is 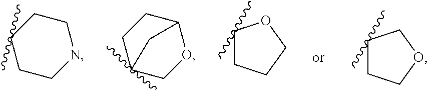
wherein the heterocycloalkyl is unsubstituted or substituted with one to four substituents selected from the group consisting of halogen, C₁-C₆alkyl, haloC₁-C₆alkyl, alkoxy, CN, COOC₁-C₆alkyl, and heteroaryl, wherein the heteroaryl is unsubstituted or substituted with one to four substituents selected from the group consisting of halogen, CN, C₁-C₆alkyl and haloC₁-C₆alkyl, or a pharmaceutically acceptable salt thereof.

25. The compound of claim 24, wherein when the heterocycloalkyl is 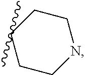
and wherein the heterocycloalkyl is substituted with heteroaryl selected from 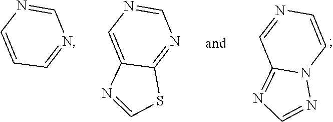
wherein the heteroaryl is unsubstituted or substituted with CN, or a pharmaceutically acceptable salt thereof.
